SEQUENCE LISTING (37 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  Description of Artificial Sequence Synthetic adalimumab light chain variable region CDR3
QRYNRAPYX

SEQ ID NO: 4 - protein, 12 aa, Artificial Sequence
  Description of Artificial Sequence Synthetic adalimumab heavy chain variable region CDR3
VSYLSTASSLDX

SEQ ID NO: 5 - protein, 7 aa, Artificial Sequence
  Description of Artificial Sequence Synthetic adalimumab light chain variable region CDR2
AASTLQS

SEQ ID NO: 6 - protein, 17 aa, Artificial Sequence
  Description of Artificial Sequence Synthetic adalimumab heavy chain variable region CDR2
AITWNSGHIDYADSVEG

SEQ ID NO: 7 - protein, 11 aa, Artificial Sequence
  Description of Artificial Sequence Synthetic adalimumab light chain variable region CDR1
RASQGIRNYLA

SEQ ID NO: 8 - protein, 5 aa, Artificial Sequence
  Description of Artificial Sequence Synthetic adalimumab heavy chain variable region CDR1
DYAMH

SEQ ID NO: 9 - protein, 107 aa, Artificial Sequence
  Description of Artificial Sequence Synthetic 2SD4 light chain variable region
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial Sequence
  Description of Artificial Sequence Synthetic 2SD4 heavy chain variable region
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial Sequence
  Description of Artificial Sequence Synthetic 2SD4 light chain variable region CDR3
QKYNSAPYA

SEQ ID NO: 12 - protein, 9 aa, Artificial Sequence
  Description of Artificial Sequence Synthetic EP B12 light chain variable region CDR3
QKYNRAPYA

SEQ ID NO: 13 - protein, 9 aa, Artificial Sequence
  Description of Artificial Sequence Synthetic VL10E4 light chain variable region CDR3
QKYQRAPYT

SEQ ID NO: 14 - protein, 9 aa, Artificial Sequence
  Description of Artificial Sequence Synthetic VL100A9 light chain variable region CDR3
QKYSSAPYT

SEQ ID NO: 15 - protein, 9 aa, Artificial Sequence
  Description of Artificial Sequence Synthetic VLL100D2 light chain variable region CDR3
QKYNSAPYT

SEQ ID NO: 16 - protein, 9 aa, Artificial Sequence
  Description of Artificial Sequence Synthetic VLL0F4 light chain variable region CDR3
QKYNRAPYT

SEQ ID NO: 17 - protein, 9 aa, Artificial Sequence
  Description of Artificial Sequence Synthetic LOE5 light chain variable region CDR3
QKYNSAPYY

SEQ ID NO: 18 - protein, 9 aa, Artificial Sequence
  Description of Artificial Sequence Synthetic VLLOG7 light chain variable region CDR3
QKYNSAPYN

SEQ ID NO: 19 - protein, 9 aa, Artificial Sequence
  Description of Artificial Sequence Synthetic VLLOG9 light chain variable region CDR3
QKYTSAPYT

SEQ ID NO: 20 - protein, 9 aa, Artificial Sequence
  Description of Artificial Sequence Synthetic VLLOH1 light chain variable region CDR3
QKYNRAPYN

SEQ ID NO: 21 - protein, 9 aa, Artificial Sequence
  Description of Artificial Sequence Synthetic VLLOH10 light chain variable region CDR3
QKYNSAAYS

SEQ ID NO: 22 - protein, 9 aa, Artificial Sequence
  Description of Artificial Sequence Synthetic VL1B7 light chain variable region CDR3
QQYNSAPDT

SEQ ID NO: 23 - protein, 9 aa, Artificial Sequence
  Description of Artificial Sequence Synthetic VL1C1 light chain variable region CDR3
QKYNSDPYT

SEQ ID NO: 24 - protein, 9 aa, Artificial Sequence
  Description of Artificial Sequence Synthetic VL0.1F4 light chain variable region CDR3
QKYISAPYT

SEQ ID NO: 25 - protein, 9 aa, Artificial Sequence
  Description of Artificial Sequence Synthetic VL0.1H8 light chain variable region CDR3
QKYNRPPYT

SEQ ID NO: 26 - protein, 9 aa, Artificial Sequence
  Description of Artificial Sequence Synthetic LOE7.A light chain variable region CDR3
QRYNRAPYA

SEQ ID NO: 27 - protein, 12 aa, Artificial Sequence
  Description of Artificial Sequence Synthetic 2SD4 heavy chain variable region CDR3
ASYLSTSSSLDN

SEQ ID NO: 28 - protein, 12 aa, Artificial Sequence
  Description of Artificial Sequence Synthetic VH1B11 heavy chain variable region CDR3
ASYLSTSSSLDK

SEQ ID NO: 29 - protein, 12 aa, Artificial Sequence
  Description of Artificial Sequence Synthetic VH1D8 heavy chain variable region CDR3
ASYLSTSSSLDY

SEQ ID NO: 30 - protein, 12 aa, Artificial Sequence
  Description of Artificial Sequence Synthetic VH1A11 heavy chain variable region CDR3
ASYLSTSSSLDD

SEQ ID NO: 31 - protein, 12 aa, Artificial Sequence
  Description of Artificial Sequence Synthetic VH1B12 heavy chain variable region CDR3
ASYLSTSFSLDY

SEQ ID NO: 32 - protein, 12 aa, Artificial Sequence
  Description of Artificial Sequence Synthetic VH1E4 heavy chain variable region CDR3
ASYLSTSSSLHY

SEQ ID NO: 33 - protein, 12 aa, Artificial Sequence
  Description of Artificial Sequence Synthetic VH1F6 heavy chain variable region CDR3
ASFLSTSSSLEY

SEQ ID NO: 34 - protein, 12 aa, Artificial Sequence
  Description of Artificial Sequence Synthetic 3C-H2 heavy chain variable region CDR3
ASYLSTASSLEY

SEQ ID NO: 35 - protein, 12 aa, Artificial Sequence
  Description of Artificial Sequence Synthetic VH1-D2.N heavy chain variable region CDR3
VSYLSTASSLDN

SEQ ID NO: 36 - DNA, 321 bp, Artificial Sequence
  Description of Artificial Sequence Synthetic adalimumab light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 37 - DNA, 363 bp, Artificial Sequence
  Description of Artificial Sequence Synthetic adalimumab heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt

(19) United States
(12) Patent Application Publication
(10) Pub. No.: US 2008/0131374 A1
(43) Pub. Date: Jun. 5, 2008

(54) Uses and compositions for treatment of rheumatoid arthritis

(76) Inventors: John R. Medich, East Hanover, NJ (US); Hartmut Kupper, Mutterstadt (DE); Jeffrey D. Kent, Deerfield, IL (US); John L. Perez, Doylestown, PA (US); Eric H. Sasso, Mountain Lakes, NJ (US); Oscar Segurado, Lake Bluff, IL (US); George Spencer-Green, Mountain Lakes, NJ (US); Theodore Pincus, Hastings-on-Hudson, NY (US)

Correspondence Address: LAHIVE & COCKFIELD, LLP, ONE POST OFFICE SQUARE, BOSTON, MA, 02109-2127, US

(21) Appl. No.: 11/800,531

(22) Filed: May 4, 2007

Related U.S. Application Data
(63) Continuation-in-part of application No. 11/788,740, filed on Apr. 19, 2007.
(60) Provisional application No. 60/793,737, filed on Apr. 19, 2006.
(60) Provisional application No. 60/798,149, filed on May 4, 2006.
(60) Provisional application No. 60/801,584, filed on May 17, 2006.
(60) Provisional application No. 60/812,705, filed on Jun. 8, 2006.
(60) Provisional application No. 60/857,352, filed on Nov. 6, 2006.
(60) Provisional application No. 60/858,328, filed on Nov. 10, 2006.
(60) Provisional application No. 60/872,753, filed on Dec. 4, 2006.
(60) Provisional application No. 60/798,149, filed on May 4, 2006.
(60) Provisional application No. 60/801,584, filed on May 17, 2006.
(60) Provisional application No. 60/812,705, filed on Jun. 8, 2006.
(60) Provisional application No. 60/857,352, filed on Nov. 6, 2006.
(60) Provisional application No. 60/858,328, filed on Nov. 10, 2006.
(60) Provisional application No. 60/872,753, filed on Dec. 4, 2006.

(51) Int. Cl. A61K 49/00; A61K 39/395; A61K 31/44; A61P 29/00

(52) U.S. Cl. 424/9.2; 424/172.1; 514/354; 424/133.1

(57) ABSTRACT

The invention provides methods, uses and compositions for the treatment of rheumatoid arthritis. The invention describes methods and uses for treating rheumatoid arthritis wherein a TNFα inhibitor, such as a human TNFα antibody, or antigen-binding portion thereof. Also described are methods for determining the efficacy of a TNFα inhibitor for treatment of rheumatoid arthritis in a subject.

### RELATED APPLICATIONS

This application is a continuation-in-part of U.S. application Ser. No. 11/______, filed on Apr. 19, 2007 (Attorney Docket No. BBI-291, entitled “Uses and Compositions for Treatment of Rheumatoid Arthritis”). U.S. application Ser. No. 11/______, filed on Apr. 19, 2007 (Attorney Docket No. BBI-291, entitled “Uses and Compositions for Treatment of Rheumatoid Arthritis”) claims the benefit of priority to U.S. provisional patent application No. 60/793,737 filed on Apr. 19, 2006; U.S. provisional patent application No. 60/798,149 filed on May 4, 2006; U.S. provisional patent application No. 60/801,584 filed on May 17, 2006; U.S. provisional patent application No. 60/812,705, filed on Jun. 8, 2006; U.S. provisional patent application No. 60/857,352 filed on Nov. 6, 2006; U.S. provisional patent application No. 60/858,328 file on Nov. 10, 2006; and U.S. provisional patent application number 60/872,753 filed on Dec. 4, 2006. This application also claims the benefit of priority to U.S. provisional patent application No. 60/798,149 filed on May 4, 2006; U.S. provisional patent application No. 60/801,584 filed on May 17, 2006; U.S. provisional patent application No. 60/812,705, filed on Jun. 8, 2006; U.S. provisional patent application No. 60/857,352 filed on Nov. 6, 2006; U.S. provisional patent application No. 60/858,328 file on Nov. 10, 2006; and U.S. provisional patent application No. 60/872,753 filed on Dec. 4, 2006. The contents of all the above-mentioned priority applications are hereby incorporated by reference in their entirety.

### BACKGROUND OF THE INVENTION

Rheumatoid arthritis (RA) is considered a chronic, inflammatory autoimmune disorder. RA is a disabling and painful inflammatory condition which can lead to the substantial loss of mobility due to pain and joint destruction. RA leads to the soft-tissue swelling of joints. Rheumatoid arthritis is three times more common in women as in men. It afflicts people of all races equally. The disease can begin at any age, but most often starts after age forty and before sixty. In some families, multiple members can be affected, suggesting a genetic basis for the disorder.

There is no known cure for rheumatoid arthritis. Acetylsalicylate, naproxen, ibuprofen, and etodolac are examples of nonsteroidal anti-inflammatory drugs (NSAIDs) often used to treat RA by reducing tissue inflammation, pain and swelling, but are not cortisone. Corticosteroids are also often used to reduce pain and inflammation associated with RA. Corticosteroids are more potent than NSAIDs in reducing inflammation, and in restoring joint mobility and function. Corticosteroids are useful for short periods during severe flares of disease activity, or when the disease is not responding to NSAIDs. However, corticosteroids can have serious side effects, especially when given in high doses for long periods of time. Other drugs, such as gold, methotrexate and hydroxychloroquine (Plaquenil) promote disease remission and prevent progressive joint destruction, but they are not anti-inflammatory agents

Tumor necrosis factor (TNF) has been identified as a pivotal cytokine in the pathogenesis of rheumatoid arthritis (RA). In recent years biologic response modifiers that inhibit TNF activity have become established therapies for RA. Adalimumab, etanercept, and infliximab have demonstrated marked improvements in treating RA, including when used in combination with methotrexate (Breedveld et al, 2006; Genovese et al, 2005; Keystone et al, 2004; Navarro-Sarabia et al, 2005; Smolen et al, 2006; St. Clair et al, 2004; van der Heijde et al, 2006).

### SUMMARY OF THE INVENTION

Although TNFα inhibitors are effective at treating RA, there remains a need for a more effective treatment option for subjects suffering from rheumatoid arthritis (RA), especially in certain subpopulations of RA patients, e.g., subjects who lose responsiveness to a TNFα inhibitor, subjects with early RA. There also remains a need for improved methods and compositions that provide a safe and effective treatment of RA using TNFα inhibitors.

The instant invention provides improved methods and compositions for treating RA. The invention further provides a means for treating certain subpopulations of patients who have RA, including subjects or patients who have failed therapy or lost responsiveness to treatment with TNFα inhibitors. The invention further provides a means by which the efficacy of a TNFα inhibitor for the treatment of RA can be determined. The invention also includes methods for treating certain types of RA, e.g., early RA. The invention further provides methods for identifying subjects who Kits and labels which provide information pertaining to the methods, uses, and compositions of the invention are also described herein. Each of the examples described herein describes methods and compositions which can be used to determine whether a TNFα inhibitor is effective for treating the given disorder, i.e. RA.

In one embodiment, the invention provides a method of determining the efficacy of a TNFα inhibitor, e.g., a human TNFα antibody, or antigen-binding portion thereof, for treating RA in a subject comprising determining an ACR20 response of a patient population having RA and who was administered the human TNFα antibody, or antigen-binding portion thereof, wherein an ACR20 response in at least about 50% of the patient population indicates that the human TNFα antibody, or antigen-binding portion thereof, is an effective human TNFα antibody, or antigen-binding portion thereof, for the treatment of RA in a subject.

The invention includes a method of treating RA in a subject comprising administering an effective human TNFα antibody, or antigen-binding portion thereof, wherein the effective human TNFα antibody, or antigen-binding portion thereof, was identified as providing an ACR20 response in at least about 33% of a patient population who received the effective TNFα inhibitor for the treatment of RA.

In one embodiment, the ACR20 response in the patient population is selected from the group of at least about 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, and 79%, 80%, 81%, 82%, 83%, 84%, 85%, 86%, 87%, 88%, and 89%.

In one embodiment, the invention provides a method of determining the efficacy of a TNFα inhibitor, e.g., human TNFα antibody, or antigen-binding portion thereof, for treating RA in a subject comprising determining an ACR50 response of a patient population having RA and who was administered the human TNFα antibody, or antigen-binding portion thereof, wherein an ACR50 response in at least about 30% of the patient population indicates that the human TNFα antibody, or antigen-binding portion thereof, is an effective human TNFα antibody, or antigen-binding portion thereof, for the treatment of RA in a subject.

The invention further provides a method of treating RA in a subject comprising administering an effective human TNFα antibody, or antigen-binding portion thereof, wherein the effective human TNFα antibody, or antigen-binding portion thereof, was identified as providing an ACR50 response in at least about 30% of a patient population who received the effective TNFα inhibitor for the treatment of RA.

In one embodiment, the ACR50 response in the patient population is selected from the group of at least about 18%, 19%, 20%, 21%, 22%, 23%, 24%, 25%, 26%, 27%, 28%, 29%, 30%, 31%, 32%, 33%, 34%. 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, and 89%.

In one embodiment, the invention provides a method of determining the efficacy of a TNFα inhibitor, e.g. human TNFα antibody, or antigen-binding portion thereof, for treating RA in a subject comprising determining an ACR70 response of a patient population having RA and who was administered the human TNFα antibody, or antigen-binding portion thereof, wherein an ACR70 response in at least about 19% of the patient population indicates that the human TNFα antibody, or antigen-binding portion thereof, is an effective human TNFα antibody, or antigen-binding portion thereof, for the treatment of RA in a subject.

In one embodiment, the invention provides a method of treating RA in a subject comprising administering an effective human TNFα antibody, or antigen-binding portion thereof, wherein the effective human TNFα antibody, or antigen-binding portion thereof, was identified as providing an ACR70 response in at least about 19% of a patient population who received the effective TNFα inhibitor for the treatment of RA.

In one embodiment, the ACR70 response in the patient population is selected from the group of at least about 11%, 12%, 13%, 14%, 15%, 16%, 17%., 18%, 19%, 20%, 21%, 22%, 23%, 24%, 25%, 26%, 27%, 28%, 29%, 30% 31%, 32%, 33%, 34%. 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, and 79%, 80%, 81%, 82%, 83%, 84%, 85%, 86%, 87%, 88%, and 89%.

The invention also includes a method of determining the efficacy of a TNFα antibody, or antigen-binding portion thereof, for treating RA in a subject comprising determining an ACR70 response of a patient population having RA and who was administered the TNFα antibody, or antigen-binding portion thereof, wherein an ACR70 response in at least about 38% of the patient population indicates that the TNFα antibody, or antigen-binding portion thereof, is an effective TNFα antibody, or antigen-binding portion thereof, for the treatment of RA in a subject.

In yet another embodiment, the invention provides a method for determining the efficacy of a TNFα inhibitor, e.g. human TNFα antibody, or antigen-binding portion thereof, for treating RA in a subject comprising determining an ACR90 response of a patient population having RA and who was administered the human TNFα antibody, or antigen-binding portion thereof, wherein an ACR90 response in at least about 8% of the patient population indicates that the human TNFα antibody, or antigen-binding portion thereof, is an effective human TNFα antibody, or antigen-binding portion thereof, for the treatment of RA in a subject.

The invention also includes a method of treating RA in a subject comprising administering an effective human TNFα antibody, or antigen-binding portion thereof, wherein the effective human TNFα antibody, or antigen-binding portion thereof, was identified as providing an ACR90 response in at least about 8% of a patient population who received the effective TNFα inhibitor for the treatment of RA.

In one embodiment, the ACR90 response in the patient population is selected from the group of at least about 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, and 79%, 80%, 81%, 82%, 83%, 84%, 85%, 86%, 87%, 88%, and 89%.

The invention further comprises administering the effective TNFα inhibitor, e.g., human TNFα antibody, or antigen-binding portion thereof, to a subject for the treatment of RA.

The invention also includes methods of treating RA comprising administering an effective TNFα inhibitor identified using any of the methods described herein.

In one embodiment, the invention includes a method for determining the efficacy of a human TNFα antibody, or antigen-binding portion thereof, for treating RA in a subject comprising determining a moderate EULAR response of a patient population having RA and who was administered the human TNFα antibody, or antigen-binding portion thereof, wherein a moderate EULAR response in at least about 76% of the patient population indicates that the human TNFα antibody, or antigen-binding portion thereof, is an effective human TNFα antibody, or antigen-binding portion thereof, for the treatment of RA. In one embodiment, a moderate EULAR response occurs in a percentage of the patient population selected from the group of at least about 11%, 12%, 13%, 14%, 15%, 16%, 17%, 18%, 19%, 20%, 21%, 22%, 23%, 24%, 25%, 26%, 27%, 28%, 29%, 30% 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, and 79%, 80%, 81%, 82%, 83%, 84%, 85%, 86%, 87%, 88%, and 89%.

The invention also provides a method for determining the efficacy of a human TNFα antibody, or antigen-binding portion thereof, for treating RA in a subject comprising determining a good EULAR response of a patient population having RA and who was administered the human TNFα antibody, or antigen-binding portion thereof, wherein a good EULAR response in at least about 18% of the patient population indicates that the human TNFα antibody, or antigen-binding portion thereof, is an effective human TNFα antibody, or antigen-binding portion thereof, for the treatment of RA. In one embodiment, a good EULAR response occurs in a percentage of the patient population selected from the group of at least about 11%, 12%, 13%, 14%, 15%, 16%, 17%, 18%, 19%, 20%, 21%, 22%, 23%, 24%, 25%, 26%, 27%, 28%, 29%, 30% 31%, 32%, 33%, 34%, 35%, 36%, 37%, 38%, 39%.

In one embodiment, the effective human TNFα antibody, or antigen-binding portion thereof, is administered to a subject for the treatment of RA.

In one embodiment, the patient population or subject previously failed a different TNFα inhibitor. In one embodiment, the TNFα inhibitor which the patient or patient population failed was infliximab or etanercept. In another embodiment, the patient population or subject previously failed DMARD therapy.

The invention also provides a method for determining the efficacy of a TNFα inhibitor for the treatment of finger or hand joint inflammation associated with a TNFα-related disorder selected from the group consisting of rheumatoid arthritis (RA), psoriatic arthritis (PsA), and juvenile rheumatoid arthritis (JRA), comprising determining a median synovitis MRI score (determined using the OMERACT semiquantitative scoring system) of a patient population who was administered the TNFα inhibitor, wherein a decrease of at least about 2 in the indicates that the TNFα inhibitor is an effective TNFα inhibitor for the treatment of finger or hand joint inflammation associated with the TNFα-related disorder.

The invention includes a method for determining the efficacy of a TNFα inhibitor for the treatment of finger or hand joint inflammation associated with a TNFα-related disorder selected from the group consisting of rheumatoid arthritis (RA), psoriatic arthritis (PsA), and juvenile rheumatoid arthritis (JRA), comprising determining a tenosynovitis MRI score of a patient who was administered the TNFα inhibitor, wherein a decrease of at least about 1.5 in the median tenosynovitis MRA score (determined using the OMERACT semiquantitative scoring system) of the patient population indicates that the TNFα inhibitor an effective TNFα inhibitor for the treatment of finger or hand joint inflammation associated with the TNFα-related disorder.

The invention provides a method of treating a subject having RA who has failed a prior biologic comprising administering a human TNFα antibody, or antigen-binding portion thereof, to the subject such that RA is treated. In one embodiment, the prior biologic is selected from the group consisting of etanercept, infliximab, and anakinra.

The invention also includes a method of treating a subject having recent-onset RA comprising administering a human TNFα antibody, or antigen-binding portion thereof, to the subject such that recent-onset RA is treated. In one embodiment, the invention further comprises inhibiting radiographic progression in the subject.

The invention describes a method of achieving a major clinical response in a subject having RA comprising administering a human TNFα antibody, or antigen-binding portion thereof, to the subject such that the major clinical response is achieved.

The invention also provides a method for inhibiting radiographic progression of rheumatoid arthritis (RA) in a subject having early or recent-onset RA comprising administering a human TNFα antibody, or antigen-binding portion thereof, to a subject having early or recent-onset RA, such that radiographic progression is inhibited.

The invention also provides a method for testing the efficacy of a combination of a TNFα antibody, or antigen-binding portion thereof, and a disease-modifying anti-rheumatic drug (DMARD) for inhibiting radiographic progression of rheumatoid arthritis (RA) in a subject having early or recent-onset RA comprising determining a radiographic progression score of a population who was administered the combination of the TNFα antibody, or antigen-binding portion thereof, and the DMARD, wherein no radiographic progression in at least about 61% of the patient population indicates that the combination of the TNFα antibody, or antigen-binding portion thereof, and the DMARD is an effective combination for the treatment of early or recent-onset RA in combination. In one embodiment, no radiographic progression is defined as ΔTSS≦0.5. In another embodiment, the subject or patient population has RA for less than 3 years. the DMARD is methotrexate.

The invention provides a method of treating a human subject having rheumatoid arthritis (RA) comprising administering a TNFα inhibitor to the subject, wherein the subject has previously failed an anti-TNFα therapy comprising administration of an alternate TNFα antagonist. In one embodiment, the alternate TNFα antagonist is a biologic agent. In one embodiment, the biologic agent comprises infliximab or etanercept or anakinra. In one embodiment, the alternate TNFα antagonist was discontinued for a reason selected from the group consisting of no response, lost efficacy, and intolerance.

The invention provides a method for treating a human subject suffering from rheumatoid arthritis who has been identified as having a baseline health assessment questionnaire (HAQ) score of at least about 1.4 comprising administering to the subject a TNFα inhibitor, such that the HAQ score of the subject is decreased by at least about 0.49 points.

The invention further provides an effective method of treatment for RA for patients who have failed previous biologic and/or DMARD therapy.

The invention also describes a method of treating a human subject suffering from rheumatoid arthritis comprising identifying a subject with a HAQ score of at least about 1.4; and administering to the subject a TNFα inhibitor such that the HAQ score of the subject is decreased by at least about 0.49 points.

The invention further provides a method of decreasing a HAQ score by at least about 0.49 points in about 25-28% of a preselected patient population comprising administering a TNFα inhibitor to the patient population until a decrease of least about 0.49 points in the HAQ score of about 25-28% of the patient population is achieved, where the patient population has been preselected for having rheumatoid arthritis and a baseline HAQ score of at least about 1.4.

The invention also includes a method for monitoring the effectiveness of an anti-TNFα regimen for treating rheumatoid arthritis (RA) comprising administering to a subject a TNFα inhibitor in accordance with the anti-TNFα regimen, wherein the subject has a baseline HAQ score of at least about 1.4; obtaining an HAQ score from the subject; and determining a change in the HAQ score from the baseline HAQ score to the HAQ score of (b), wherein a decrease in the HAQ score by at least 0.49 points indicates that the anti-TNFα regimen is effective at treating RA.

In one embodiment, the HAQ score is decreased by at least about 0.55 points. In another embodiment, the decrease in the HAQ score is achieved within 12 weeks from the initial administration of the TNFα inhibitor.

The invention also describes a method for monitoring the effectiveness of an anti-TNFα regimen for treating rheumatoid arthritis (RA) comprising administering to a subject a TNFα inhibitor in accordance with the anti-TNFα regimen, wherein the subject has a baseline tender count (TJC) of at least about 17 and/or a baseline swollen joint count (SJC) of at least 14; determining the TJC and/or SJC score in the subject; and determining changes in the TJC and/or SJC score between the baseline TJC and/or SJC score and the TJC and/or SJC score of (b), wherein a decrease of at least 10 points in the TJC score and/or a decrease of at least 7 in the SJC score indicates that the anti-TNFα regimen is effective at treating RA.

The invention provides a method for monitoring the effectiveness of an anti-TNFα regimen for treating rheumatoid arthritis (RA) comprising administering to a subject a TNFα inhibitor in accordance with the an anti-TNFα regimen, wherein the subject has a baseline DAS28 score of at least about 6.5; and determining the DAS28 score in the subject; and determining changes in the baseline DAS28 and the DAS28 score from (b), wherein a decrease of at least 2.1 points in the DAS28 score indicates that the anti-TNFα regimen is effective at treating RA.

The invention provides a method for determining or monitoring the effectiveness of a TNFα inhibitor for the treatment of a TNFα-related disorder using magnetic resonance imaging (MRI).

The invention provides a method for determining the efficacy of a TNFα inhibitor for the treatment of finger or hand joint inflammation associated with a TNFα-related disorder selected from the group consisting of rheumatoid arthritis (RA), psoriatic arthritis (PsA), and juvenile rheumatoid arthritis (JRA), comprising determining the effectiveness of the TNFα inhibitor using a baseline median synovitis magnetic resonance imaging (MRI) score of a preselected patient population having joint inflammation and the TNFα-related disorder and a median synovitis MRI score of the patient population following administration of the TNFα inhibitor, wherein a decrease of at least about 2 in the median synovitis MRI score (determined using the OMERACT semiquantitative scoring system) of the patient population indicates that the TNFα inhibitor is efficacious for the treatment of finger or hand joint inflammation associated with the TNFα-related disorder.

The invention also provides method for determining the efficacy of a TNFα inhibitor for the treatment of finger or hand joint inflammation associated with TNFα-related disorder selected from the group consisting of rheumatoid arthritis (RA), psoriatic arthritis (PsA), and juvenile rheumatoid arthritis (JRA), comprising determining the effectiveness of the TNFα inhibitor using a baseline median tenosynovitis magnetic resonance imaging (MRI) score of a preselected patient population having joint inflammation and the TNFα-related disorder and a median tenosynovitis MRI score of the patient population following administration of the TNFα inhibitor, wherein a decrease of at least about 1.5 in the median tenosynovitis score (determined using the OMERACT semiquantitative scoring system) of the patient population indicates that the TNFα inhibitor is efficacious for the treatment of finger or hand joint inflammation associated with the TNFα-related disorder.

The invention also includes a method of achieving an improved DAS28 score and an improved median synovitis magnetic resonance imaging (MRI) score in a preselected patient population having finger or hand joint inflammation and a TNFα-related disorder selected from the group consisting of rheumatoid arthritis (RA), psoriatic arthritis (PsA), and juvenile rheumatoid arthritis (JRA), comprising administering a TNFα inhibitor to the patient population such that the DAS28 score and the median synovitis MRI score are both improved. In one embodiment, the improvement in the median synovitis score is a decrease of at least about 2 (determined using the OMERACT semiquantitative scoring system). In one embodiment, the improvement in the DAS28 score is a decrease of at least about 2. In a further embodiment, the DAS28 score is a decrease of at least about 2.3.

The invention also provides a method for monitoring the effectiveness of a TNFα antibody, or an antigen binding portion thereof, for reducing inflammation in a metacarpophalangeal or interphalangeal joint of a patient population having a TNFα-related disorder comprising determining the effectiveness of the TNFα antibody, or antigen binding portion thereof, using a baseline median synovitis score magnetic resonance imaging (MRI) score of the patient and a median synovitis score MRI score of the patient population following administration of the TNFα antibody, or antigen binding portion thereof, wherein a decrease of at least about 2 in the median synovitis score (determined using the OMERACT-based semiquantitative scoring system) indicates that the TNFα antibody, or antigen binding portion thereof, is effective for reducing inflammation in the metacarpophalangeal joint.

The invention further provides a method for monitoring the effectiveness of a TNFα antibody, or an antigen binding portion thereof, for reducing inflammation in a metacarpophalangeal or interphalangeal joint of a patient population having a TNFα-related disorder comprising determining the effectiveness of the TNFα antibody, or antigen binding portion thereof, using a baseline median tenosynovitis score MRI score of the patient population and a median tenosynovitis score MRI score of the patient population following administration of the TNFα antibody, or antigen binding portion thereof, wherein a decrease of at least about 1.5 in the median tenosynovitis score (determined using the OMERACT-based semiquantitative scoring system) indicates that the TNFα antibody, or antigen binding portion thereof, is effective for reducing inflammation in the metacarpophalangeal joint.

In one embodiment, the TNFα-related disorder is selected from the group consisting of rheumatoid arthritis (RA), psoriatic arthritis (PsA), juvenile rheumatoid arthritis (JRA).

The invention provides a method for treating a human subject having rheumatoid arthritis (RA) who has failed Disease-Modifying Anti-Rheumatic Drug (DMARD) therapy comprising administering to the subject a TNFα inhibitor, such that RA is treated. In one embodiment of the invention, the TNFα inhibitor is a TNFα antibody, or antigen-binding portion thereof.

In one embodiment, the human subject has long-standing, severe RA. In another embodiment, the human subject has had RA for at least about 11 years. In still another embodiment, the human subject has a tender joint count (TJC) of about 34. IN yet another embodiment, the human subject has a Health Assessment Questionnaire (HAQ) Score of about 1.9. In one embodiment, the human subject has a C-reactive protein (CRP) score of about 56 mg/L.

In one embodiment, the failed DMARD therapy is failed methotrexate therapy.

In one embodiment, the TNFα antibody, or antigen-binding portion thereof, is administered as a monotherapy without administration of an additional therapeutic agent.

The invention also provides a method for determining the efficacy of a TNFα inhibitor for improving health utility in a subject having RA who has failed methotrexate therapy comprising determining the efficacy of the TNFα inhibitor using a Health Utilities Index Mark 3 (HUI3) score of a patient population having RA and having failed methotrexate therapy and a HUI3 score of the patient population following administration of the TNFα antibody, or antigen-binding portion thereof, wherein an increase in the HUI3 score indicates that the TNFα inhibitor, is efficacious for improving health utility in a subject having RA who has failed methotrexate therapy.

In one embodiment of the invention, the HUI3 score of the patient population following administration of the TNFα antibody, or antigen-binding portion thereof, increases by at least about 0.1. In another embodiment of the invention, the HUI3 score of the patient population following administration of the TNFα antibody, or antigen-binding portion thereof, is at least about 43.

The invention also provides a method for determining the efficacy of a TNFα inhibitor for decreasing fatigue in a subject having RA who has failed methotrexate therapy comprising determining the efficacy of the TNFα inhibitor using a Functional Assessment of Chronic Illness Therapy—Fatigue (FACIT-F) score of a patient population having RA and having failed methotrexate therapy and a FACIT-F score of the patient population following administration of the TNFα inhibitor wherein an increase in the FACIT-F score indicates that the TNFα antibody, or antigen-binding portion thereof, is efficacious for decreasing fatigue in a subject having RA who has failed methotrexate therapy.

In one embodiment of the invention, the TNFα inhibitor is a TNFα antibody, or antigen-binding portion thereof.

In one embodiment of the invention, the FACIT-F score of the patient population following administration of the TNFα antibody, or antigen-binding portion thereof, increases by at least about 5.4. In another embodiment of the invention, the FACIT-F score of the patient population following administration of the TNFα antibody, or antigen-binding portion thereof, is at least about 32.

The invention describes a method for determining the efficacy of a TNFα inhibitor for improving the overall well-being in a subject having RA who has failed methotrexate therapy comprising determining the efficacy of the TNFα inhibitor using an overall SF-36 score of a patient population having RA and having failed methotrexate therapy and an overall SF-36 score of the patient population following administration of the TNFα inhibitor wherein an increase in all of the subscales of the SF-36 score indicates that the TNFα inhibitor is efficacious for improving overall well-being in a subject having RA who has failed methotrexate therapy.

In one embodiment of the invention, the TNFα inhibitor is a TNFα antibody, or antigen-binding portion thereof.

In one embodiment of the invention, the human subject has long-standing, severe RA. In one embodiment, the human subject has had RA for at least about 11 years. In another embodiment, the human subject has at least one score selected from the group consisting of a tender joint count (TJC) of about 34; a Health Assessment Questionnaire (HAQ) Score of about 1.9; and a C-reactive protein (CRP) score of about 56 mg/L.

The invention also includes an article of manufacture comprising a packaging material; a TNFα antibody, or antigen-binding portion thereof; and a label or package insert contained within the packaging material indicating that the TNFα antibody, or antigen-binding portion thereof, can be used for the treatment of rheumatoid arthritis in patients who have failed methotrexate therapy.

The invention includes a method for testing the efficacy of a TNFα inhibitor and a disease-modifying anti-rheumatic drug (DMARD) for inhibiting radiographic progression of rheumatoid arthritis (RA) in patients having early or recent-onset RA comprising using a mean baseline radiographic progression score of a preselected patient population having early or recent-onset RA and a mean radiographic progression score of the patient population following administration of the TNFα inhibitor and the DMARD, wherein no radiographic progression in at least about 61% of the patient population indicates that the TNFα inhibitor is effective for the treatment of early or recent-onset RA in combination with a DMARD.

In one embodiment, no radiographic progression is defined as ΔTSS≦0.5. IN another embodiment, the subject or patient population has had RA for less than about 3 years. In one embodiment, the DMARD is methotrexate.

The invention provides a method for inhibiting radiographic progression of rheumatoid arthritis (RA) in a subject having very early RA comprising administering an TNFα antibody, or an antigen-binding portion thereof, to a subject having very early RA, such that radiographic progression is inhibited. In one embodiment, the subject has had RA for less than 6 months.

The invention also includes a method for identifying a patient having RA who is a candidate for treatment with a TNFα inhibitor, comprising determining whether the patient has a DAS28 score of at least about 5.1 and a RAPID score of at least about 5.0, wherein said DAS28 score and said RAPID score indicate the patient having RA is a candidate for treatment with a TNFα inhibitor.

The invention further provides a method for predicting the efficacy of a TNFα inhibitor for treating a subject having RA comprising comparing a predetermined baseline C-reactive protein (CRP) level of the subject to a CRP level of the patient following treatment with the TNFα inhibitor, wherein a decrease in the CRP level of at least about 20% indicates the TNFα inhibitor will be efficacious at treating RA.

The invention provides method for monitoring the effectiveness of a TNFα inhibitor for the treatment of fatigue in a subject having RA comprising using a predetermined baseline FACIT-F score and a FACIT-F score following administration of the TNFα inhibitor, wherein an improvement of at least about 7.1 indicates that the TNFα inhibitor is effective at reducing fatigue in a subject having RA. In one embodiment, the improvement in the FACIT-F scores is at least about 8.1.

The invention includes a method for testing the efficacy of a TNFα inhibitor and a disease-modifying anti-rheumatic drug (DMARD) for inhibiting radiographic progression of rheumatoid arthritis (RA) in patients having long-standing RA comprising using a mean baseline radiographic progression score of a preselected patient population having long-standing RA and a mean radiographic progression score of the patient population following administration of the TNFα inhibitor and the DMARD, wherein no radiographic progression in at least about 62% of the patient population indicates that the TNFα inhibitor is effective for the treatment of long-standing RA in combination with a DMARD.

In one embodiment, the radiographic progression is determined using either a mean Total Sharp Score or a mean joint erosion score.

The invention includes a method for treating a human subject having rheumatoid arthritis (RA) who has failed previous anti-TNFα therapy comprising administering an alternate TNFα inhibitor to the subject. In one embodiment, the previous anti-TNFα therapy was a biologic agent. In one embodiment, the biologic agent comprises infliximab or etanercept. In one embodiment, the previous anti-TNFα therapy was discontinued for a reason selected from the group consisting of no response, lost efficacy, and intolerance.

The invention includes a method for predicting whether a subject having recent-onset RA will be responsive to treatment with a TNFα inhibitor for inhibition of radiographic progression associated with RA, comprising determining a mean baseline CRP level of the subject, wherein an abnormal baseline CRP level indicates that the subject will not be responsive to treatment with the TNFα inhibitor.

The invention describes a method for predicting whether a subject having recent-onset RA will be responsive to treatment with a TNFα inhibitor for inhibition of radiographic progression associated with RA, comprising determining a mean baseline CRP level of the subject wherein a normal baseline CRP level indicates that the subject will be responsive to treatment with the TNFα inhibitor.

The invention also provides a method of inhibiting reactivation of latent tuberculosis in a patient receiving a TNFα inhibitor comprising delivering a isoniazid (INH) prophylaxis to the subject, such that reactivation of latent tuberculosis is inhibited.

In one embodiment, the patient or patient population is administered methotrexate in combination with the TNFα inhibitor.

The invention also includes an article of manufacture comprising
a) a packaging material;
b) a TNFα antibody; and
c) a label or package insert contained within the packaging material indicating that the TNFα antibody may be administered in combination with methotrexate, wherein the methotrexate is administered via a route selected from the group consisting of oral, intramuscular (im), subcutaneous (sc), and intravenous (iv).

The invention further provides an article of manufacture comprising
a) a packaging material;
b) a TNFα antibody; and
c) a label or package insert contained within the packaging material indicating that the TNFα antibody may be administered in combination with methotrexate, wherein the methotrexate is administered in a dose ranging from less than about 7.5 mg to more than about 20 mg.

The invention also includes an article of manufacture comprising
a) a packaging material;
b) a TNFα antibody; and
c) a label or package insert contained within the packaging material indicating that in studies of the TNFα antibody for the treatment of rheumatoid arthritis (RA) serious adverse events (SAEs) included a disorder selected from the group consisting of tuberculosis, lymphomas, congestive heart failure, demyelinating disease, systemic lupus erthematosus, opportunistic infections, and blood dyscasias.

The invention further provides an article of manufacture comprising
a) a packaging material;
b) a TNFα antibody; and
c) a label or package insert contained within the packaging material indicating that the TNFα antibody is safe for the treatment of both early and long-standing rheumatoid arthritis (RA).

In one embodiment, the TNFα antibody, or antigen-binding portion thereof, is selected from the group consisting of a chimeric antibody, a humanized antibody, and a multivalent antibody.

In one embodiment, the TNFα antibody, or antigen-binding portion thereof, is a human antibody.

The invention includes a method of testing the effectiveness of a TNFα inhibitor for decreasing fatigue in a patient having rheumatoid arthritis (RA), comprising comparing a pre-determined FACIT-fatigue score following treatment of the patient with the TNFα inhibitor, with a pre-determined FACIT-fatigue baseline score, wherein a change of at least about 8 indicates the TNFα inhibitor is effective for decreasing fatigue in a patient having RA.

The invention includes a method of testing the effectiveness of a TNFα inhibitor for improving health utility in a patient having rheumatoid arthritis (RA), comprising comparing a pre-determined HUI3 score following treatment of the patient with the TNFα inhibitor, with a pre-determined HUI3 baseline score, wherein a change of at least about 0.18 indicates the TNFα inhibitor is effective improving health utility in a patient having RA.

The invention also includes an article of manufacture comprising
a) a packaging material;
b) a TNFα antibody; and
c) a label or package insert contained within the packaging material describing any of the examples described herein.

In one embodiment, the TNFα inhibitor is administered weekly to the patient population. In one embodiment, the TNFα inhibitor is administered biweekly to the patient population.

In another embodiment, the TNFα inhibitor is administered in a multiple variable dose regimen. In one embodiment, the TNFα inhibitor is administered in a biweekly dosing regimen.

In one embodiment, the TNFα inhibitor is administered as a monotherapy.

In another embodiment, the TNFα inhibitor is administered with an additional therapeutic agent. In one embodiment, the TNFα inhibitor is administered with methotrexate. In one embodiment, the patient or patient population is administered methotrexate in combination with the TNFα inhibitor.

The invention also describes an article of manufacture comprising a packaging material; a TNFα inhibitor; and a label or package insert contained within the packaging material indicating that patients with rheumatoid arthritis (RA) who previously failed therapy with etanercept or infliximab may benefit from treatment of RA with the human TNFα antibody.

The invention includes a method of promoting a human TNFα antibody to a recipient, the method comprising conveying to the recipient that patients with rheumatoid arthritis (RA) who previously failed therapy with etanercept or infliximab may benefit from treatment of RA with the human TNFα antibody.

The invention also includes an article of manufacture comprising a packaging material; a human TNFα inhibitor; and a label or package insert contained within the packaging material indicating that patients with rheumatoid arthritis (RA) taking the human TNFα antibody and concomitant corticosteroids have a higher risk of developing a serious infection.

The invention also includes a rapid method of determining the efficacy of a TNFα inhibitor for the treatment of RA in a patient, said rapid method comprising determining a Routine Apgar-Like Patient Index Data (RAPID) Score of the patient using a patient questionnaire which includes the following scales:
a) a scale for physical function;
b) a pain visual analog scale (VAS); and
c) a global assessment VAS.

The invention includes a method for testing the efficacy of a TNFα inhibitor for improving the quality of life of a subject having an autoimmune disease comprising using a predetermined baseline AIMS2 score and a AIMS2 score following administration of the TNFα inhibitor to the subject, wherein at least one improvement selected from the group consisting of the following:
a) a decrease of at least about 25% in the physical domain component;
b) a decrease of at least about 43% in the symptoms domain component;
c) a decrease of at least about 11% in the affect domain component; and
d) a decrease of at least about 16% in the work domain component;
   indicates that the TNFα inhibitor is effective at improving the quality of life of a subject having the autoimmune disease.

In one embodiment, the autoimmune disease is rheumatoid arthritis (RA).

In one embodiment, the AIMS2 test used is the AIMS2-SF.

In one embodiment, the TNFα inhibitor further improves at least one of the following scores in the subject having RA: DAS28 score, ACR response, EULAR response, and/or and HAQ score.

In one embodiment, the TNFα inhibitor is administered weekly to the patient population.

In one embodiment, the TNFα inhibitor is administered biweekly to the patient population.

In one embodiment, the patient or patient population is administered methotrexate in combination with the TNFα inhibitor.

In one embodiment, the subject having rheumatoid arthritis (RA) has failed previous anti-TNFα therapy. In one embodiment, the previous anti-TNFα therapy was a biologic agent. In one embodiment, the biologic agent comprises infliximab or etanercept. In one embodiment, the previous anti-TNFα therapy was discontinued for a reason selected from the group consisting of no response, lost efficacy, and intolerance.

In one embodiment, the invention includes treating any of the patient subpopulations described in the Examples of this application, e.g., patients who failed prior TNF biologic therapy patients with early RA, patients with longstanding RA.

In one embodiment, the TNFα inhibitor is administered weekly to the patient population. In one embodiment, the TNFα inhibitor is administered biweekly to the patient population.

In another embodiment, the TNFα inhibitor is administered in a multiple variable dose regimen. In one embodiment, the TNFα inhibitor is administered in a biweekly dosing regimen.

In one embodiment, the TNFα inhibitor is administered as a monotherapy.

In another embodiment, the TNFα inhibitor is administered with an additional therapeutic agent. In one embodiment, the TNFα inhibitor is administered with methotrexate. In one embodiment, the patient or patient population is administered methotrexate in combination with the TNFα inhibitor.

In one embodiment, the TNFα inhibitor is selected from the group consisting of a TNFα antibody, or an antigen-binding portion thereof, a TNF fusion protein, or a recombinant TNF binding protein.

In one embodiment, the TNFα antibody, or antigen-binding portion thereof, is selected from the group consisting of a chimeric antibody, a humanized antibody, and a multivalent antibody. In one embodiment, the anti-TNFα antibody, or antigen-binding portion thereof, is selected from the group consisting of infliximab, golimumab, and adalimumab.

In one embodiment, the TNFα antibody, or antigen-binding portion thereof, is a human antibody.

In one embodiment, the TNFα antibody, or antigen-binding portion thereof, is an isolated human antibody that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

In one embodiment, the TNFα antibody is an isolated human antibody, or antigen-binding portion thereof, with the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

In one embodiment, the TNFα antibody is an isolated human antibody, or an antigen binding portion thereof, with a light chain variable region (LCVR) comprising the amino acid sequence of SEQ ID NO: 1 and a heavy chain variable region (HCVR) comprising the amino acid sequence of SEQ ID NO: 2

In one embodiment, the human TNFα antibody, or antigen-binding portion thereof, is adalimumab.

In one embodiment, the TNFα antibody, or antigen-binding portion thereof, is a 40 mg dose.

In one embodiment, the TNFα antibody, or antigen-binding portion thereof, is administered subcutaneously.

In one embodiment, the TNFα antibody, or antigen-binding portion thereof, is infliximab or golimumab.

### BRIEF DESCRIPTION OF THE FIGURES

FIG. 1 shows ACR responses at 12 weeks (observed values).

FIG. 2 shows EULAR responses at 12 weeks (observed values).

FIG. 3 shows TJC and SJC improvement at 12 weeks (observed values; median).

FIG. 4 shows DAS28 improvement at 12 weeks (observed values; median).

FIG. 5 graphically depicts the ACR20 response at 12 weeks for various concomitant DMARD therapy.

FIG. 6 graphically depicts the percentage of responders with and without prior biologics.

FIG. 7 graphically depicts the median change in DAS28 in patients who were treated with and without prior biologics.

FIG. 8 graphically depicts the TJC and SJC in patients who were treated with and without prior biologics.

FIG. 9 graphically depicts the median change in HAQ score in patients who were treated with and without prior biologics.

FIG. 10 graphically depicts the change in DAS28 in patients who were treated with and without prior biologics specifying the number of prior biologics.

FIG. 11 graphically depicts the median change in DAS28 in patients who were treated with and without prior biologics specifying the type of prior biologic.

FIG. 12 shows the study design for Study 4.

FIG. 13 shows the trend in ACR20, ACR50, and ACR70 responses in patient populations taking adalimumab for treatment of RA over 4 years.

FIG. 14 shows the TJC and SJC scores through the fourth year of adalimumab treatment.

FIG. 15 graphically depicts the mean HUI3 score of ADA treated patients and those given placebo treatment.

FIG. 16 graphically depicts the mean FACIT-F score of ADA treated patients and those given placebo treatment.

FIG. 17 graphically depicts the SF-36 LOCF of ADA treated patients and those given placebo treatment.

FIG. 18 shows the design of study J. Study J examined the efficacy and safety of adalimumab plus methotrexate versus adalimumab alone or methotrexate alone in the early treatment of RA as described in Example 7.

FIG. 19 graphically depicts the ACR 20/50/70 responses at Week 52 by Week 12 CRP categories.

FIG. 20 shows the study design of Study 1.

FIG. 21 shows the study design of Study A, including the extension period following the open-label period.

FIG. 22 shows radiographic progression by disease duration.

FIGS. 23 and 24 show the tender joint count and swollen joint count improvement over time in patients given ada who had previously failed infliximab treatment.

FIG. 25 depicts the study design for both Study C and Study A in a comparative view.

FIG. 26 graphically depicts the median TJC/SJC and reduction at 12 weeks.

FIG. 27a depicts the impact of screening on TB rates in adalimumab RA clinical trials.

FIG. 27b shows TB rates through December 2004 after screening initiated in adalimumab RA clinical trials.

FIG. 28 shows the design of Study I.

FIG. 29 shows the FACIT-F scores over 3 years.

FIG. 30 shows the SF-36 domain scores of ada treated patients over 3 years.

FIG. 31 shows the mean changes in SF-36 domain scores at week 12.

FIG. 32 shows the mean changes in SF-36 domain scores at week 104.

FIG. 33 shows the mean SF-36 summary scores at baseline, week 12, and week 104.

FIG. 34 shows the mean changes in SF-36 summary scores at week 12 and week 104.

FIG. 35 graphically represents ACR response rates by prior ETA and/or INF experience and by exclusive reasons for discontinuation.

FIG. 36 graphically represents EULAR response rates by prior ETA and INF experience and by exclusive reasons for discontinuation.

FIG. 37 depicts the mean change in DAS28 at Week 12.

FIG. 38 depicts the mean change in HAQ at 12 weeks.

FIG. 39 graphically depicts ACR Responses through 52 weeks of adalimumab therapy.

FIG. 40 depicts the change in total Sharp Score at 6 months in patients treated with adalimumab and MTX, and with MTX alone.

FIG. 41 graphically depicts the percentage of patients with a first SI, according to the duration of adalimumab exposure at the time of event.

FIG. 42 graphically depicts the percentage of patients who developed SI, according to age at the time of study entry.

FIG. 43 graphically depicts patient subgroups by best ACR response prior to dosage escalation in the Study J study.

FIG. 44 depicts ACR response rates in ARMADA over 60 months.

FIG. 45 depicts mean DAS28 scores over 60 months.

FIG. 46 graphically depicts the percentage of patients achieving multiple indicators of excellent clinical response over 5 years.

FIG. 47 depicts significant changes in dosing of concomitant corticosteroids and/or MTX from baseline to the completion of the study period.

FIG. 48 shows the mean improvements in HAQ DI scores through 2 years.

FIG. 49 depicts mean improvements in fatigue (FACIT-F) through 2 years in the Study J study.

FIG. 50 depicts differences between treatment groups in mean improvements in HAQ DI scores over 2 years of the Study J study.

FIG. 51 depicts differences between treatment groups in mean improvements in FACIT-F scores over 2 years of the Study J study.

FIG. 52 describes the ACR response through 12 weeks of adalimumab therapy.

FIG. 53 shows EULAR responses through 12 weeks of adalimumab therapy.

FIG. 54 graphically depicts the change in DAS28 at week 2, 6, and 12.

FIG. 55 graphically depicts change in HAQ at week 2, 6, and 12.

FIG. 56 graphically depicts AIMS2-SF results at week 12.

### DETAILED DESCRIPTION OF THE INVENTION

### I. Definitions

The term “human TNFα” (abbreviated herein as hTNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term human TNFβ is intended to include recombinant human TNFα (rhTNFα), which can be prepared by standard recombinant expression methods or purchased commercially (R & D Systems, Catalog No. 210-TA, Minneapolis, Minn.). TNFα is also referred to as TNF.

The term “TNFα inhibitor” includes agents which interfere with TNFα activity. The term also includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. In another embodiment, the TNFα inhibitor is a TNF fusion protein, e.g., etanercept (Enbrel®, Amgen; described in WO 91/03553 and WO 09/406,476, incorporated by reference herein). In another embodiment, the TNFα inhibitor is a recombinant TNF binding protein (r-TBP-I) (Serono).

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The antibodies of the invention are described in further detail in U.S. Pat. Nos. 6,090,382; 6,258,562; and 6,509,015, each of which is incorporated herein by reference in its entirety.

The term “antigen-binding portion” or “antigen-binding fragment” of an antibody (or simply “antibody portion”), as used herein, refers to one or more fragments of an antibody that retain the ability to specifically bind to an antigen (e.g., hTNFα). It has been shown that the antigen-binding function of an antibody can be performed by fragments of a full-length antibody. Binding fragments include Fab, Fab′, F(ab′)₂, Fabc, Fv, single chains, and single-chain antibodies. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH or VL domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). The antibody portions of the invention are described in further detail in U.S. Pat. Nos. 6,090,382, 6,258,562, 6,509,015, each of which is incorporated herein by reference in its entirety.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In one embodiment, the invention features a chimeric antibody or antigen-binding fragment, in which the variable regions of both light and heavy chains mimics the variable regions of antibodies derived from one species of mammals, while the constant portions are homologous to the sequences in antibodies derived from another species. In a preferred embodiment of the invention, chimeric antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Such chimeric, humanized, human, and dual specific antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539.

An “isolated antibody”, as used herein, is intended to refer to an antibody that is substantially free of other antibodies having different antigenic specificities (e.g., an isolated antibody that specifically binds hTNFα is substantially free of antibodies that specifically bind antigens other than hTNFα). An isolated antibody that specifically binds hTNFα may, however, have cross-reactivity to other antigens, such as TNFα molecules from other species. Moreover, an isolated antibody may be substantially free of other cellular material and/or chemicals.

A “neutralizing antibody”, as used herein (or an “antibody that neutralized hTNFα activity”), is intended to refer to an antibody whose binding to hTNFα results in inhibition of the biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see U.S. Pat. No. 6,090,382). Preferably, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jönsson et al. (1993) Ann. Biol. Clin. 51:19; Jönsson et al. (1991) Biotechniques 11:620-627; Johnsson et al. (1995) J. Mol. Recognit. 8:125; and Johnnson et al. (1991) Anal. Biochem. 198:268.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The term “IC₅₀” as used herein, is intended to refer to the concentration of the inhibitor required to inhibit the biological endpoint of interest, e.g., neutralize cytotoxicity activity.

The term “dose,” as used herein, refers to an amount of TNFα inhibitor which is administered to a subject.

The term “dosing”, as used herein, refers to the administration of a substance (e.g., an anti-TNFα antibody) to achieve a therapeutic objective (e.g., treatment of rheumatoid arthritis).

A “dosing regimen” describes a treatment schedule for a TNFα inhibitor, e.g., a treatment schedule over a prolonged period of time and/or throughout the course of treatment, e.g. administering a first dose of a TNFα inhibitor at week 0 followed by a second dose of a TNFα inhibitor on a biweekly dosing regimen.

The terms “biweekly dosing regimen”, “biweekly dosing”, and “biweekly administration”, as used herein, refer to the time course of administering a substance (e.g., an anti-TNFα antibody) to a subject to achieve a therapeutic objective, e.g, throughout the course of treatment. The biweekly dosing regimen is not intended to include a weekly dosing regimen. Preferably, the substance is administered every 9-19 days, more preferably, every 11-17 days, even more preferably, every 13-15 days, and most preferably, every 14 days. In one embodiment, the biweekly dosing regimen is initiated in a subject at week 0 of treatment. In another embodiment, a maintenance dose is administered on a biweekly dosing regimen. In one embodiment, both the loading and maintenance doses are administered according to a biweekly dosing regimen. In one embodiment, biweekly dosing includes a dosing regimen wherein doses of a TNFα inhibitor are administered to a subject every other week beginning at week 0. In one embodiment, biweekly dosing includes a dosing regimen where doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing methods are also described in US 20030235585, incorporated by reference herein.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an anti-TNFα antibody and another drug. The other drug(s) may be administered concomitant with, prior to, or following the administration of an anti-TNFα antibody.

The term “treatment,” as used within the context of the present invention, is meant to include therapeutic treatment, as well as prophylactic or suppressive measures, for the treatment of rheumatoid arthritis. For example, the term treatment may include administration of a TNFα inhibitor prior to or following the onset of rheumatoid arthritis thereby preventing or removing signs of the disease or disorder. As another example, administration of a TNFα inhibitor after clinical manifestation of rheumatoid arthritis to combat the symptoms and/or complications and disorders associated with rheumatoid arthritis comprises “treatment” of the disease. Further, administration of the agent after onset and after clinical symptoms and/or complications have developed where administration affects clinical parameters of the disease or disorder and perhaps amelioration of the disease, comprises “treatment” of rheumatoid arthritis. In one embodiment, treatment of rheumatoid arthritis in a subject comprises reducing signs and symptoms. In another embodiment, treatment of rheumatoid arthritis in a subject comprises inducing major clinical response of rheumatoid arthritis. In another embodiment, treatment of rheumatoid arthritis in a subject comprises inhibiting the progression of structural damage. In one embodiment, treatment of rheumatoid arthritis comprises improving physical function in adult patients with moderately to severely active disease.

Those “in need of treatment” include mammals, such as humans, already having rheumatoid arthritis, including those in which the disease or disorder is to be prevented.

Various aspects of the invention are described in further detail herein.

The invention provides improved uses and compositions for treating rheumatoid arthritis with a TNFα inhibitor, e.g., a human TNFα antibody, or an antigen-binding portion thereof. Compositions and articles of manufacture, including kits, relating to the methods and uses for treating rheumatoid arthritis are also contemplated as part of the invention.

### II. TNF Inhibitors

A TNFα inhibitor which is used in the methods and compositions of the invention includes any agent which interferes with TNFα activity. In a preferred embodiment, the TNFα inhibitor can neutralize TNFα activity, particularly detrimental TNFα activity which is associated with rheumatoid arthritis, and related complications and symptoms.

In one embodiment, the TNFα inhibitor used in the invention is an TNFα antibody (also referred to herein as a TNFα antibody), or an antigen-binding fragment thereof, including chimeric, humanized, and human antibodies. Examples of TNFα antibodies which may be used in the invention include, but not limited to, infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein.

Other examples of TNFα inhibitors which may be used in the methods and compositions of the invention include etanercept (Enbrel, described in WO 91/03553 and WO 09/406,476), soluble TNF receptor Type I, a pegylated soluble TNF receptor Type I (PEGs TNF-R1), p55TNFR1gG (Lenercept), and recombinant TNF binding protein (r-TBP-I) (Serono).

In one embodiment, the term “TNFα inhibitor” excludes infliximab. In one embodiment, the term “TNFα inhibitor” excludes adalimumab. In another embodiment, the term “TNFα inhibitor” excludes adalimumab and infliximab.

In one embodiment, the term “TNFα inhibitor” excludes etanercept, and, optionally, adalimumab, infliximab, and adalimumab and infliximab.

In one embodiment, the term “TNFα antibody” excludes infliximab. In one embodiment, the term “TNFβ antibody” excludes adalimumab. In another embodiment, the term “TNFα antibody” excludes adalimumab and infliximab.

In one embodiment, the invention features uses and composition for treating or determining the efficacy of a TNFα inhibitor for the treatment of rheumatoid arthritis, wherein the TNFα antibody is an isolated human antibody, or antigen-binding portion thereof, that binds to human TNFα with high affinity and a low off rate, and also has a high neutralizing capacity. Preferably, the human antibodies used in the invention are recombinant, neutralizing human anti-hTNFα antibodies. The most preferred recombinant, neutralizing antibody of the invention is referred to herein as D2E7, also referred to as HUMIRA® or adalimumab (the amino acid sequence of the D2E7 VL region is shown in SEQ ID NO: 1; the amino acid sequence of the D2E7 VH region is shown in SEQ ID NO: 2). The properties of D2E7 (adalimumab/HUMIRA®) have been described in Salfeld et al., U.S. Pat. Nos. 6,090,382, 6,258,562, and 6,509,015, which are each incorporated by reference herein. The methods of the invention may also be performed using chimeric and humanized murine anti-hTNFα antibodies which have undergone clinical testing for treatment of rheumatoid arthritis (see e.g., Elliott, M. J., et al. (1994) Lancet 344:1125-1127; Elliot, M. J., et al. (1994) Lancet 344:1105-1110; Rankin, E. C., et al. (1995) Br. J. Rheumatol. 34:334-342).

In one embodiment, the method of the invention includes determining the efficacy of D2E7 antibodies and antibody portions, D2E7-related antibodies and antibody portions, or other human antibodies and antibody portions with equivalent properties to D2E7, such as high affinity binding to hTNFα with low dissociation kinetics and high neutralizing capacity, for the treatment of rheumatoid arthritis. In one embodiment, the invention provides treatment with an isolated human antibody, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. More preferably, the isolated human antibody, or antigen-binding portion thereof, dissociates from human TNFα with a K_{off} of 5×10⁻⁴ s⁻¹ or less, or even more preferably, with a K_{off} of 1×10⁻⁴ s⁻¹ or less. More preferably, the isolated human antibody, or antigen-binding portion thereof, neutralizes human TNFα cytotoxicity in a standard in vitro L929 assay with an IC₅₀ of 1×10⁻⁸ M or less, even more preferably with an IC₅₀ of 1×10⁻⁹ M or less and still more preferably with an IC₅₀ of 1×10⁻¹⁰ M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to treating Crohn's disease by administering human antibodies that have slow dissociation kinetics for association with hTNFα and that have light and heavy chain CDR3 domains that structurally are identical to or related to those of D2E7. Position 9 of the D2E7 VL CDR3 can be occupied by Ala or Thr without substantially affecting the K_{off}. Accordingly, a consensus motif for the D2E7 VL CDR3 comprises the amino acid sequence: Q-R-Y-N-R-A-P-Y-(T/A) (SEQ ID NO: 3). Additionally, position 12 of the D2E7 VH CDR3 can be occupied by Tyr or Asn, without substantially affecting the K_{off}. Accordingly, a consensus motif for the D2E7 VH CDR3 comprises the amino acid sequence: V-S-Y-L-S-T-A-S-S-L-D-(Y/N) (SEQ ID NO: 4). Moreover, as demonstrated in Example 2 of U.S. Pat. No. 6,090,382, the CDR3 domain of the D2E7 heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the K_{off}. Still further, the skilled artisan will appreciate that, given the amenability of the D2E7 VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the D2E7 VL and/or VH CDR3 domains. More preferably, no more than one to three conservative amino acid substitutions are made within the D2E7 VL and/or VH CDR3 domains. Additionally, conservative amino acid substitutions should not be made at amino acid positions critical for binding to hTNFα. Positions 2 and 5 of the D2E7 VL CDR3 and positions 1 and 7 of the D2E7 VH CDR3 appear to be critical for interaction with hTNFα and thus, conservative amino acid substitutions preferably are not made at these positions (although an alanine substitution at position 5 of the D2E7 VL CDR3 is acceptable, as described above) (see U.S. Pat. No. 6,090,382).

Accordingly, in another embodiment, the antibody or antigen-binding portion thereof preferably contains the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

More preferably, the antibody, or antigen-binding portion thereof, dissociates from human TNFα with a K_{off} of 5×10⁻⁴ s⁻¹ or less. Even more preferably, the antibody, or antigen-binding portion thereof, dissociates from human TNFα with a K_{off} of 1×10⁻⁴ s⁻¹ or less.

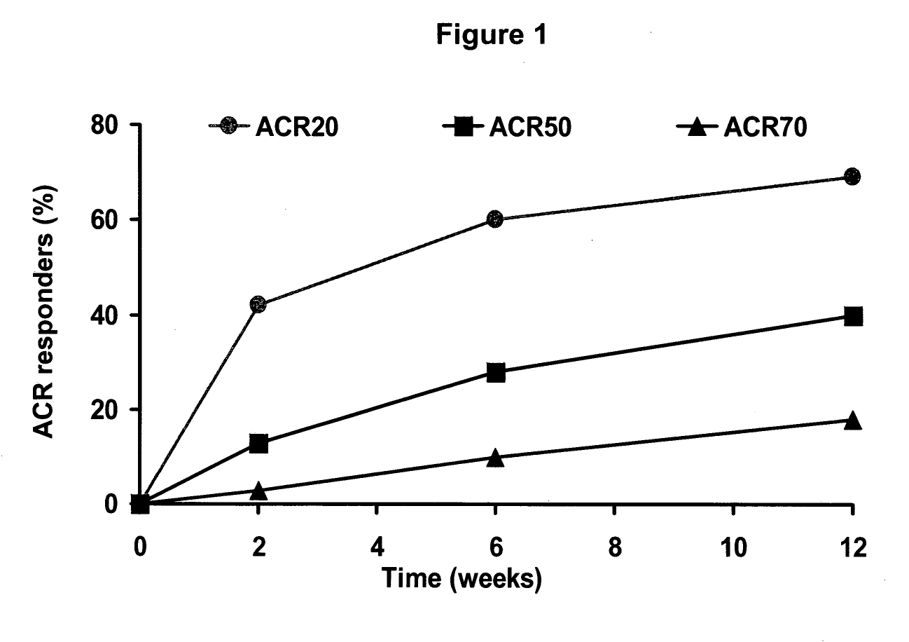

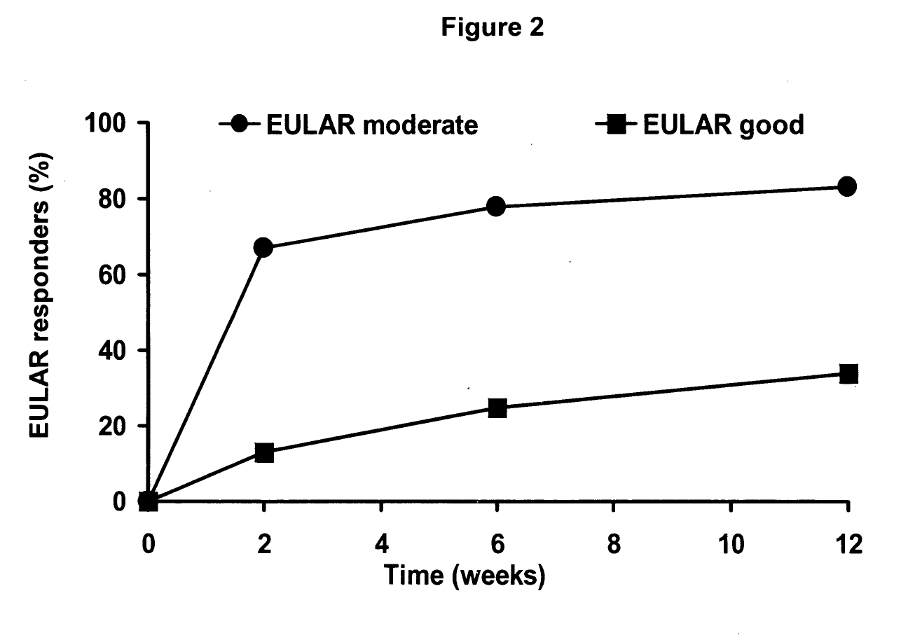

In yet another embodiment, the antibody or antigen-binding portion thereof preferably contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. Preferably, the LCVR further has a CDR2 domain comprising the amino acid sequence of SEQ ID NO: 5 (i.e., the D2E7 VL CDR2) and the HCVR further has a CDR2 domain comprising the amino acid sequence of SEQ ID NO: 6 (i.e., the D2E7 VH CDR2). Even more preferably, the LCVR further has CDR1 domain comprising the amino acid sequence of SEQ ID NO: 7 (i.e., the D2E7 VL CDR1) and the HCVR has a CDR1 domain comprising the amino acid sequence of SEQ ID NO: 8 (i.e., the D2E7 VH CDR1). The framework regions for VL preferably are from the V_{κ}I human germline family, more preferably from the A20 human germline Vk gene and most preferably from the D2E7 VL framework sequences shown in FIGS. 1A and 1B of U.S. Pat. No. 6,090,382. The framework regions for VH preferably are from the V_{H}3 human germline family, more preferably from the DP-31 human germline VH gene and most preferably from the D2E7 VH framework sequences shown in FIGS. 2A and 2B of U.S. Pat. No. 6,090,382.

Accordingly, in another embodiment, the antibody or antigen-binding portion thereof preferably contains a light chain variable region (LCVR) comprising the amino acid sequence of SEQ ID NO: 1 (i.e., the D2E7 VL) and a heavy chain variable region (HCVR) comprising the amino acid sequence of SEQ ID NO: 2 (i.e., the D2E7 VH). In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In still other embodiments, the invention includes uses of an isolated human antibody, or an antigen-binding portions thereof, containing D2E7-related VL and VH CDR3 domains. For example, antibodies, or antigen-binding portions thereof, with a light chain variable region (LCVR) having a CDR3 domain comprising an amino acid sequence selected from the group consisting of SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26 or with a heavy chain variable region (HCVR) having a CDR3 domain comprising an amino acid sequence selected from the group consisting of SEQ ID NO: 4, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

The TNFα antibody used in the methods and compositions of the invention may be modified for improved treatment of rheumatoid arthritis. In some embodiments, the TNFα antibody or antigen binding fragments thereof, is chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat rheumatoid arthritis by administration of the TNFα antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In yet another embodiment of the invention, TNFα antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity.

An antibody or antibody portion used in the methods of the invention can be derivatized or linked to another functional molecule (e.g., another peptide or protein). Accordingly, the antibodies and antibody portions of the invention are intended to include derivatized and otherwise modified forms of the human anti-hTNFα antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

An antibody, or antibody portion, used in the methods and compositions of the invention, can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express adalimumab (D2E7) or an adalimumab (D2E7)-related antibody, DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V₇₈ Segments Reveals a Strong Bias in their Usage” Eur. J. Immunol. 24:827-836; the contents of each of which are expressly incorporated herein by reference). To obtain a DNA fragment encoding the heavy chain variable region of D2E7, or a D2E7-related antibody, a member of the V_{H}3 family of human germline VH genes is amplified by standard PCR. Most preferably, the DP-31 VH germline sequence is amplified. To obtain a DNA fragment encoding the light chain variable region of D2E7, or a D2E7-related antibody, a member of the V_{κ}I family of human germline VL genes is amplified by standard PCR. Most preferably, the A20 VL germline sequence is amplified. PCR primers suitable for use in amplifying the DP-31 germline VH and A20 germline VL sequences can be designed based on the nucleotide sequences disclosed in the references cited supra, using standard methods.

Once the germline VH and VL fragments are obtained, these sequences can be mutated to encode the D2E7 or D2E7-related amino acid sequences disclosed herein. The amino acid sequences encoded by the germline VH and VL DNA sequences are first compared to the D2E7 or D2E7-related VH and VL amino acid sequences to identify amino acid residues in the D2E7 or D2E7-related sequence that differ from germline. Then, the appropriate nucleotides of the germline DNA sequences are mutated such that the mutated germline sequence encodes the D2E7 or D2E7-related amino acid sequence, using the genetic code to determine which nucleotide changes should be made. Mutagenesis of the germline sequences is carried out by standard methods, such as PCR-mediated mutagenesis (in which the mutated nucleotides are incorporated into the PCR primers such that the PCR product contains the mutations) or site-directed mutagenesis.

Moreover, it should be noted that if the “germline” sequences obtained by PCR amplification encode amino acid differences in the framework regions from the true germline configuration (i.e., differences in the amplified sequence as compared to the true germline sequence, for example as a result of somatic mutation), it may be desirable to change these amino acid differences back to the true germline sequences (i.e., “backmutation” of framework residues to the germline configuration).

Once DNA fragments encoding D2E7 or D2E7-related VH and VL segments are obtained (by amplification and mutagenesis of germline VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions used in the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the D2E7 or D2E7-related light or heavy chain sequences, the expression vector may already carry antibody constant region sequences. For example, one approach to converting the D2E7 or D2E7-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors used in the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr− CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hTNFα. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hTNFα by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In view of the foregoing, nucleic acid, vector and host cell compositions that can be used for recombinant expression of the antibodies and antibody portions used in the invention include nucleic acids, and vectors comprising said nucleic acids, comprising the human TNFα antibody adalimumab (D2E7). The nucleotide sequence encoding the D2E7 light chain variable region is shown in SEQ ID NO: 36. The CDR1 domain of the LCVR encompasses nucleotides 70-102, the CDR2 domain encompasses nucleotides 148-168 and the CDR3 domain encompasses nucleotides 265-291. The nucleotide sequence encoding the D2E7 heavy chain variable region is shown in SEQ ID NO: 37. The CDR1 domain of the HCVR encompasses nucleotides 91-105, the CDR2 domain encompasses nucleotides 148-198 and the CDR3 domain encompasses nucleotides 295-330. It will be appreciated by the skilled artisan that nucleotide sequences encoding D2E7-related antibodies, or portions thereof (e.g., a CDR domain, such as a CDR3 domain), can be derived from the nucleotide sequences encoding the D2E7 LCVR and HCVR using the genetic code and standard molecular biology techniques.

Recombinant human antibodies of the invention in addition to D2E7 or an antigen binding portion thereof, or D2E7-related antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating and screening antibody display libraries can be found in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-65; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982.

In a preferred embodiment, to isolate human antibodies with high affinity and a low off rate constant for hTNFα, a murine anti-hTNFα antibody having high affinity and a low off rate constant for hTNFα (e.g., MAK 195, the hybridoma for which has deposit number ECACC 87 050801) is first used to select human heavy and light chain sequences having similar binding activity toward hTNFα, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J 12:725-734. The scFv antibody libraries preferably are screened using recombinant human TNFα as the antigen.

Once initial human VL and VH segments are selected, “mix and match” experiments, in which different pairs of the initially selected VL and VH segments are screened for hTNFα binding, are performed to select preferred VL/VH pair combinations. Additionally, to further improve the affinity and/or lower the off rate constant for hTNFα binding, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to hTNFα and sequences that exhibit high affinity and a low off rate for hTNFα binding can be selected.

Following screening and isolation of an anti-hTNFα antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells, as described in further detail in above.

Methods of isolating human neutralizing antibodies with high affinity and a low off rate constant for hTNFα are described in U.S. Pat. Nos. 6,090,382, 6,258,562, and 6,509,015, each of which is incorporated by reference herein.

Antibodies, antibody-portions, and other TNFα inhibitors for use in the methods of the invention, can be incorporated into pharmaceutical compositions suitable for administration to a subject. Typically, the pharmaceutical composition comprises an antibody, antibody portion, or other TNFα inhibitor, and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody, antibody portion, or other TNFα inhibitor.

The compositions for use in the methods and compositions of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies or other TNFα inhibitors. The preferred mode of administration is parenteral (e.g., intravenous, subcutaneous, intraperitoneal, intramuscular). In a preferred embodiment, the antibody or other TNFα inhibitor is administered by intravenous infusion or injection. In another preferred embodiment, the antibody or other TNFα inhibitor is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody, antibody portion, or other TNFα inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In one embodiment, the invention includes pharmaceutical compositions comprising an effective TNFα inhibitor and a pharmaceutically acceptable carrier, wherein the effective TNFα inhibitor may be used to treat rheumatoid arthritis.

In one embodiment, the antibody or antibody portion for use in the methods of the invention is incorporated into a pharmaceutical formulation as described in PCT/IB03/04502 and U.S. Appln. No. 20040033228, incorporated by reference herein. This formulation includes a concentration 50 mg/ml of the antibody D2E7 (adalimumab), wherein one pre-filled syringe contains 40 mg of antibody for subcutaneous injection.

The antibodies, antibody-portions, and other TNFα inhibitors of the present invention can be administered by a variety of methods known in the art, although for many therapeutic applications, the preferred route/mode of administration is parenteral, e.g., subcutaneous injection. In another embodiment, administration is via intravenous injection or infusion.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, Robinson, ed., Dekker, Inc., New York, 1978.

In one embodiment, the TNFα antibodies and inhibitors used in the invention are delivered to a subject subcutaneously. In one embodiment, the subject administers the TNFα inhibitor, including, but not limited to, TNFα antibody, or antigen-binding portion thereof, to himself/herself.

The TNFα antibodies and inhibitors used in the invention may also be administered in the form of protein crystal formulations which include a combination of protein crystals encapsulated within a polymeric carrier to form coated particles. The coated particles of the protein crystal formulation may have a spherical morphology and be microspheres of up to 500 micro meters in diameter or they may have some other morphology and be microparticulates. The enhanced concentration of protein crystals allows the antibody of the invention to be delivered subcutaneously. In one embodiment, the TNFα antibodies of the invention are delivered via a protein delivery system, wherein one or more of a protein crystal formulation or composition, is administered to a subject with a TNFα-related disorder. Compositions and methods of preparing stabilized formulations of whole antibody crystals or antibody fragment crystals are also described in WO 02/072636, which is incorporated by reference herein. In one embodiment, a formulation comprising the crystallized antibody fragments described in PCT/IB03/04502 and U.S. Appln. No. 20040033228, incorporated by reference herein, are used to treat rheumatoid arthritis using the treatment methods of the invention.

In certain embodiments, an antibody, antibody portion, or other TNFα inhibitor of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion for use in the methods of the invention is coformulated with and/or coadministered with one or more additional therapeutic agents, including a rheumatoid arthritis inhibitor or antagonist. For example, an anti-hTNFα antibody or antibody portion of the invention may be coformulated and/or coadministered with one or more additional antibodies that bind other targets associated with TNFα related disorders (e.g., antibodies that bind other cytokines or that bind cell surface molecules), one or more cytokines, soluble TNFα receptor (see e.g., PCT Publication No. WO 94/06476) and/or one or more chemical agents that inhibit hTNFα production or activity (such as cyclohexane-ylidene derivatives as described in PCT Publication No. WO 93/19751) or any combination thereof. Furthermore, one or more antibodies of the invention may be used in combination with two or more of the foregoing therapeutic agents. Such combination therapies may advantageously utilize lower dosages of the administered therapeutic agents, thus avoiding possible side effects, complications or low level of response by the patient associated with the various monotherapies.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody, antibody portion, or other TNFα inhibitor may vary according to factors such as the disease state, age, sex, and weight of the individual, and the ability of the antibody, antibody portion, other TNFα inhibitor to elicit a desired response in the individual. A therapeutically effective amount is also one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Additional description regarding methods and uses of the invention comprising administration of a TNFα inhibitor are described in Part III of this specification.

The invention also pertains to packaged pharmaceutical compositions or kits for administering the anti-TNF antibodies of the invention for the treatment of rheumatoid arthritis. In one embodiment of the invention, the kit comprises a TNFα inhibitor, such as an antibody and instructions for administration of the TNFα inhibitor for treatment of rheumatoid arthritis. The instructions may describe how, e.g., subcutaneously, and when, e.g., at week 0, week 2, week 4, etc., the different doses of TNFα inhibitor shall be administered to a subject for treatment.

Another aspect of the invention pertains to kits containing a pharmaceutical composition comprising a TNFα inhibitor, such as an antibody, and a pharmaceutically acceptable carrier and one or more pharmaceutical compositions each comprising an additional therapeutic agent useful for treating rheumatoid arthritis, and a pharmaceutically acceptable carrier. Alternatively, the kit comprises a single pharmaceutical composition comprising an anti-TNFα antibody, one or more drugs useful for treating rheumatoid arthritis, and a pharmaceutically acceptable carrier. The instructions may describe how, e.g., subcutaneously, and when, e.g., at week 0, week 2, week 4, etc., the different doses of TNFα inhibitor and/or the additional therapeutic agent shall be administered to a subject for treatment.

The kit may contain instructions for dosing of the pharmaceutical compositions for the treatment of rheumatoid arthritis. Additional description regarding articles of manufacture of the invention are described in subsection III.

The package or kit alternatively can contain the TNFα inhibitor and it can be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### III. Uses and Compositions for Treating Rheumatoid Arthritis

Tumor necrosis factor has been implicated in playing a role in the pathophysiology of a variety of autoimmune diseases, including rheumatoid arthritis. TNFα is an important cytokine in the pathogenesis of rheumatoid arthritis, with elevated concentrations of TNFα playing a role in pathologic inflammation. TNFα has been implicated in activating tissue inflammation and causing joint destruction in rheumatoid arthritis (see e.g., Moeller, A., et al. (1990) Cytokine 2:162-169; U.S. Pat. No. 5,231,024 to Moeller et al.; European Patent Publication No. 260 610 B1 by Moeller, A.; Tracey and Cerami, supra; Arend, W. P. and Dayer, J-M. (1995) Arth. Rheum. 38:151-160; Fava, R. A., et al. (1993) Clin. Exp. Immunol. 94:261-266).

Tumor necrosis factor (TNF) is a pivotal cytokine in the pathogenesis of rheumatoid arthritis (RA). In recent years biologic response modifiers that inhibit TNF activity have become established therapies for RA. Adalimumab, etanercept, and infliximab have demonstrated marked improvements in both disease control and delay and prevention of radiographic damage among RA patients, particularly when used in combination with methotrexate (Breedveld et al, Arthritis Rheum 2006; 54:26-37; Genovese et al J Rheumatol 2005; 32:1232-42; Keystone et al, Arthritis Rheum 2004; 50:1400-11; Navarro-Sarabia et al, Cochrane Database Syst Rev 2005 Jul. 20; (3):CD005113; Smolen et al, Arthritis Rheum 2006; 54:702-10; St. Clair et al Arthritis Rheum 2004; 50:3432-43; van der Heijde et al, Arthritis Rheum 2006; 54:1063-74).

In one aspect, the invention discloses that adalimumab is safe in global clinical trials and has reduced mortality in RA. The invention further discloses the efficacy and safety of adalimumab in patients with RA who previously failed etanercept and/or infliximab in clinical practice and that efficacy and safety is maintained during long-term treatment of RA within a large cohort of patients (various age groups, including late-onset RA) in normal clinical practice across multiple countries. The invention also discloses that adalimumab is effective and safe with different traditional concomitant DMARDs in treating RA. Finally, the invention discloses that disease activity and physical function improve significantly in most patients with RA receiving adalimumab.

Infection with influenza virus and/or Streptococcus pneumoniae are prominent causes of morbidity and mortality in RA. Routine influenza and pneumococcal vaccinations are recommended to prevent these infections. However, treatment with corticosteroids, immunosuppressants, or TNF antagonists may potentially affect B-cell function and decrease protective antibody response. The invention describes combination uses of TNFα inhibitors treatments for rheumatoid arthritis and other disorders, including infectious disorders. In one embodiment, the invention provides a method of preventing Pneumococcal disease and treating rheumatoid arthritis (RA) in a subject comprising administering a pneumococcal vaccine and a human TNFα antibody, or antigen-binding portion thereof, to the subject, such that Pneumococcal disease is prevented and rheumatoid arthritis is treated. The invention also provides a use of a human TNFα antibody, or antigen-binding portion thereof, in the manufacture of a medicament for the treatment of RA in a subject, wherein the medicament is designed to be administered in combination with a pneumococcal vaccine for the prevention of Pneumococcal disease. In one embodiment, the human TNFα antibody, or antigen-binding portion thereof, is administered to the subject in a biweekly dosing regimen In another embodiment, the human TNFα antibody, or antigen-binding portion thereof, is administered to the subject in a dose of 40 mg. In one embodiment, the human TNFα antibody, or antigen-binding portion thereof, is administered to the subject subcutaneously.

In one embodiment, the invention provides a method of treating rheumatoid arthritis in a subject comprising administering a human TNFα antibody, or antigen-binding portion thereof, e.g., adalimumab, to the subject at week 0 on a biweekly dosing regimen. In one embodiment, the human TNFα antibody, or antigen-binding portion thereof, is administered subcutaneously. In one embodiment, rheumatoid arthritis is treated by administering a human TNFα antibody, or antigen-binding portion thereof, on biweekly dosing regimen for at least about 2 weeks, at least about 6 weeks, at least about 12 weeks, at least about 16 weeks, at least about 18 weeks, at least about 20 weeks, at least about 22 weeks, at least about 24 weeks, at least about 30 weeks, at least about 36 weeks, at least about 52 weeks at least about 72 weeks, at least about 96 weeks. Ranges of values between any of the above recited values are also intended to be included in the scope of the invention, e.g, 23 weeks, 60 week, 64 weeks, etc.

In one embodiment, rheumatoid arthritis is treated by administering a human TNFα antibody, or antigen-binding portion thereof, for at least about 2 weeks, at least about 6 weeks, at least about 12 weeks, at least about 16 weeks, at least about 18 weeks, at least about 20 weeks, at least about 22 weeks, at least about 24 weeks, at least about 30 weeks, at least about 36 weeks, at least about 52 weeks at least about 72 weeks, at least about 96 weeks. Ranges of values between any of the above recited values are also intended to be included in the scope of the invention, e.g, 23 weeks, 60 week, 64 weeks, etc.

In one embodiment, the TNFα inhibitor, e,g, antibody, or an antigen-binding portion thereof, may also be administered to a subject for the treatment of RA for a period defined in months, e.g., 3 months, 6 months, 12 months, 18 months, 24 months, 30 months, 36 months, 42 months, 48 months, 54 months, 60 months, etc. Ranges of values between any of the above recited values are also intended to be included in the scope of the invention, e.g, 38 months, 50 months, 52 months.

In one embodiment, the TNFα inhibitor, e,g, antibody, or an antigen-binding portion thereof, may also be administered to a subject for the treatment of RA for a period defined in years, e.g., 1 year, 2 years, 3 years, 4 years, 5 years, 6 years, 7 years, 8 years, etc. Ranges of values between any of the above recited values are also intended to be included in the scope of the invention, e.g, 1.5 years, 2.2 years, 3.5 years.

In one embodiment, treatment of rheumatoid arthritis is achieved by administering a human TNFα antibody, or an antigen-binding portion thereof, to a subject having rheumatoid arthritis, wherein the human TNFα antibody, or an antigen-binding portion thereof, is administered on a biweekly dosing regimen. In one embodiment, the human TNFα antibody, or an antigen-binding portion thereof, is administered in a dose of about 40 mg. In one embodiment, the human TNFα antibody, or an antigen-binding portion thereof, is adalimumab.

Methods of treatment described herein may include administration of a TNFα inhibitor to a subject to achieve a therapeutic goal, e.g., achieving a certain ACR response, e.g., ACR20, ACR50, ACR70, improving an MRI score, improving EULAR response, DAS28 score, RAPID score, CRP level, FACIT-F score, HAQ score, HUI3 score, TJC, SJC, change in TSS, SF-36 score, and AIMS2 score. Also included in the scope of the invention are uses of a TNFα inhibitor in the manufacture of a medicament to achieve a therapeutic goal, e.g., achieving a certain ACR response, e.g., ACR20, ACR50, ACR70, improving an MRI score, improving EULAR response, DAS28 score, RAPID score, CRP level, FACIT-F score, HAQ score, HUI3 score, TJC, SJC, change in TSS, SF-36 score, and AIMS2 score. Thus, where methods are described herein, it is also intended to be part of this invention that the use of the TNFα inhibitor in the manufacture of a medicament for the purpose of the method is also considered within the scope of the invention. Likewise, where a use of a TNFα inhibitor in the manufacture of a medicament for the purpose of achieving a therapeutic goal is described, methods of treatment resulting in the therapeutic goal are also intended to be part of the invention.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Dosage regimens described herein may be adjusted to provide the optimum desired response, e.g., treatment of rheumatoid arthritis, in consideration of the teachings herein. It is to be noted that dosage values may vary with the type and severity of rheumatoid arthritis. It is to be further understood that for any particular subject, specific dosage regimens may be adjusted over time according to the teachings of the specification and the individual need and the professional judgment of the person administering or supervising the administration of the compositions, and that dosage amounts and ranges set forth herein are exemplary only and are not intended to limit the scope or practice of the claimed invention.

### Subpopulations

The invention provides uses and methods for treating certain subpopulations of rheumatoid arthritis patients with a TNFα inhibitor. Also included in the invention are methods for determining whether a TNFα inhibitor, e.g., a TNFα antibody, or antigen-binding portion thereof, is effective for treating a certain subpopulation of RA patients. Thus, the invention also includes a method of treating a subject who is a member of a subpopulation of RA patients with a TNFα inhibitor which has been identified as being an effective TNFα inhibitor for the treatment of the given subpopulation.

In one embodiment, the invention provides methods and uses for treating subjects of a certain age range having rheumatoid arthritis. In one embodiment, the methods and uses of the invention are directed to treating subjects having early or recent-onset RA. As such, the invention provides a method of treating early or recent-onset, RA comprising administering a human TNFα antibody, or antigen-binding portion thereof, to a patient having early RA. In one embodiment, early RA is defined as RA in a subject who has had the disease for less than 3 years. In another embodiment, the invention provides a method of treating RA in a subject who has RA for a duration of less than 6 months comprising administering a human TNFα antibody, or antigen-binding portion thereof, to the subject. In another embodiment, the invention provides a method of treating RA in a subject who has RA for a duration of 6 months to 3 years, comprising administering a human TNFα antibody, or antigen-binding portion thereof, to the subject.

In another embodiment, the invention provides a method for treating a subject having long-standing RA.

In another embodiment, the invention provides a method for treating a subject having RA for less than or equal to 2 years. In another embodiment, the invention provides a method for treating a subject having RA for more than 2 years.

Although TNF antagonists are highly effective, a subset of patients with RA may be intolerant to one of these agents or may experience an inadequate response or a loss of response over time (Nurmohamed and Dijkmans, 2005). A relevant clinical question, therefore, is whether switching to a different TNF antagonist would be effective when the first has failed or resulted in intolerance. Clinical reports to date in mostly small numbers of patients suggest that a switch from one TNF antagonist to another is safe and effective, resulting in few withdrawals due to intolerance or lack of effectiveness (Brocq et al, Joint Bone Spine 2004; 71:601-3; Gomez-Reino et al, Arthritis Res Ther 2006; 8:R29; Hansen et al, J Rheumatol 2004; 31:1098-102; Haraoui et al, J Rheumatol 2004; 31:2356-9; Nikas et al, Ann Rheum Dis 2006; 65:257-60; van Vollenhoven et al, Ann Rheum Dis 2003; 62:1195-8). Most of these studies addressed switching between infliximab and etanercept. Data are very limited, however, regarding switching to adalimumab from one of these other TNF antagonists (Nikas et al, Ann Rheum Dis 2006; 65:257-60).

In one embodiment, the invention provides a method for treating a subpopulation of RA patients who are intolerant to or have lost response to a first TNFα inhibitor, e.g., infliximab, for the treatment of RA. In one embodiment, the invention provides a method for treating a subpopulation of patients having RA who failed prior treatment with a biologic, or prior biologic, including, for example, infliximab, etanercept, and ankinra.

In one embodiment, the invention also provides methods and compositions for use in a subject who has not previously been administered infliximab. Thus, in one embodiment, the methods and compositions of the invention are directed to a subpopulation of RA patients who have not previously received infliximab.

In one embodiment, the invention provides an article of manufacture comprising adalimumab and a package insert, wherein the package insert indicates that adalimumab may be used to treat RA in patients who have had an inadequate response to conventional therapy and/or who have lost response to or are intolerant to infliximab.

### Articles of Manufacture

The invention also provides a packaged pharmaceutical composition wherein the TNFα inhibitor, e.g., TNFα antibody, is packaged within a kit or an article of manufacture. The kit or article of manufacture of the invention contains materials useful for the treatment, including induction and/or remission, prevention and/or diagnosis of RA. The kit or article of manufacture comprises a container and a label or package insert or printed material on or associated with the container which provides information regarding use of the TNFα inhibitor, e.g., a TNFα antibody, for the treatment of RA.

A kit or an article of manufacture refers to a packaged product comprising components with which to administer a TNFα inhibitor for treatment of a RA. The kit preferably comprises a box or container that holds the components of the kit. The box or container is affixed with a label or a Food and Drug Administration approved label, including a protocol for administering the TNFα inhibitor. The box or container holds components of the invention which are preferably contained within plastic, polyethylene, polypropylene, ethylene, or propylene vessels. The vessels can be capped-tubes or bottles. The kit can also include instructions for administering the TNFα antibody of the invention. In one embodiment the kit of the invention includes the formulation comprising the human antibody adalimumab (or D2E7), as described in PCT/IB03/04502 and U.S. application Ser. No. 10/222,140, incorporated by reference herein.

The term “package insert” is used to refer to instructions customarily included in commercial packages of therapeutic products, that contain information about the indications, usage, dosage, administration, contraindications and/or warnings concerning the use of such therapeutic products.

In one embodiment, the article of manufacture of the invention comprises (a) a first container with a composition contained therein, wherein the composition comprises a TNFα antibody; and (b) a package insert indicating that the TNFα antibody may be used for reducing signs and symptoms of RA.

Suitable containers for the TNFα inhibitor, e.g., a TNFα antibody, include, for example, bottles, vials, syringes, pens, etc. The containers may be formed from a variety of materials such as glass or plastic. The container holds a composition which is by itself or when combined with another composition effective for treating, preventing and/or diagnosing the condition and may have a sterile access port.

In one embodiment, the article of manufacture comprises a TNFα inhibitor, e.g., a TNFα antibody, and a label which indicates to a subject who will be administering the TNFα inhibitor about using the TNFα inhibitor for the treatment of RA. The label may be anywhere within or on the article of manufacture. In one embodiment, the article of manufacture comprises a container, such as a box, which comprises the TNFα inhibitor and a package insert or label providing information pertaining to use of the TNFα inhibitor for the treatment of RA. In another embodiment, the information is printed on a label which is on the outside of the article of manufacture, in a position which is visible to prospective purchasers.

In one embodiment, the package insert of the invention informs a reader, including a subject, e.g., a purchaser, who will be administering the TNFα inhibitor for treatment, that the TNFα inhibitor, e.g., a TNFα antibody such as adalimumab, is an indicated treatment of RA, including of moderately to severely active disease in adult patients.

In one embodiment, the package insert describes certain patient populations who may respond favorably to the TNFα inhibitor within the article of manufacture. For example, the package insert may indicate that the TNFα antibody, e.g., adalimumab, may be used to treat RA in patients who have had an inadequate response to conventional therapy and/or who have lost response to or are intolerant to infliximab. The package insert may also indicate that the TNFα antibody, e.g., adalimumab, is suitable for treatment of patients who have failed treatment with a prior biologic. In another embodiment, the label of the invention indicates that adalimumab is indicated for treatment of early RA in adult patients who have had an inadequate response to conventional therapy. In another embodiment, the label of the invention indicates that the TNFα inhibitor, e.g., a TNFα antibody such as adalimumab, is also indicated for treatment in adult patients with early RA who have lost response to or are intolerant to infliximab.

In one embodiment, the package insert of the invention describes certain therapeutic benefits of the TNFα antibody, e.g., adalimumab, including specific symptoms of RA which may be reduced by using the TNFα antibody, e.g., adalimumab. It should be noted that the package insert may also contain information pertaining to other disorders which are treatable using the TNFα antibody, e.g., adalimumab. Information described herein which is provided in a package insert and pertains to other disorders, i.e., diseases other than RA, is also included within the scope of the invention. For example, the package insert may indicate that treatment with the TNFα antibody, e.g., adalimumab, improves radiographic progression in RA, psoriatic arthritis, and juvenile rheumatoid arthritis.

The package insert of the invention may also provide information to subjects who will be receiving adalimumab regarding combination uses for both safety and efficacy purposes. The package insert of the invention may contain warnings and precautions regarding the use of the TNFα inhibitor, e.g., a TNFα antibody such as adalimumab. In one embodiment, the invention provides an article of manufacture comprising a packaging material; a TNFα antibody, or antigen-binding portion thereof; and a label or package insert contained within the packaging material indicating that in studies of the TNFα antibody, or antigen-binding portion thereof, certain adverse events were observed, including any of those described in the Examples. In one embodiment, the label of the invention also describes rates of adverse events observed in patient populations.

The label of the invention may contain information regarding the use of the TNFα inhibitor, e.g., a TNFα antibody such as adalimumab, in clinical studies for RA. In one embodiment, the label of the invention describes the studies described herein as the Examples, either as a whole or in portion.

In one embodiment of the invention, the kit comprises a TNFα inhibitor, such as an antibody, an second pharmaceutical composition comprising an additional therapeutic agent, and instructions for administration of both agents for the treatment of RA. The instructions may describe how, e.g., subcutaneously, and when, e.g., at week 0, week 2, and biweekly thereafter, doses of TNFα antibody and/or the additional therapeutic agent shall be administered to a subject for treatment.

Another aspect of the invention pertains to kits containing a pharmaceutical composition comprising an anti-TNFα antibody and a pharmaceutically acceptable carrier and one or more additional pharmaceutical compositions each comprising a drug useful for treating a TNFα related disorder and a pharmaceutically acceptable carrier. Alternatively, the kit comprises a single pharmaceutical composition comprising an anti-TNFα antibody, one or more drugs useful for treating a TNFα related disorder and a pharmaceutically acceptable carrier. The kits further contain instructions for dosing of the pharmaceutical compositions for the treatment of a TNFα related disorder.

The package or kit alternatively may contain the TNFα inhibitor and it may be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### Additional Therapeutic Agents

Methods, uses, and compositions of the invention also include combinations of TNFα inhibitors, including antibodies, and other therapeutic agents. It should be understood that the antibodies of the invention or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent that imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations, which are part of this invention, can be the antibodies of the present invention and at least one additional agent selected from the lists below. The combination can also include more than one additional agent, e.g., two or three additional agents if the combination is such that the formed composition can perform its intended function.

Binding proteins described herein may be used in combination with additional therapeutic agents such as a Disease Modifying Anti-Rheumatic Drug (DMARD) or a Nonsteroidal Antiinflammatory Drug (NSAID) or a steroid or any combination thereof. Preferred examples of a DMARD are hydroxychloroquine, leflunomide, methotrexate, parenteral gold, oral gold and sulfasalazine. Preferred examples of non-steroidal anti-inflammatory drug(s) also referred to as NSAIDS include drugs like ibuprofen. Other preferred combinations are corticosteroids including prednisolone; the well known side effects of steroid use can be reduced or even eliminated by tapering the steroid dose required when treating patients in combination with the anti-TNFα antibodies of this invention. Non-limiting examples of therapeutic agents for rheumatoid arthritis with which an antibody, or antibody portion, of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-15, IL-16, IL-18, IL-21, IL-23, interferons, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists such as soluble p55 or p75 TNF receptors, derivatives, thereof, (p75TNFR1gG (Enbrel™) or p55TNFR1gG (Lenercept), chimeric, humanized or human TNF antibodies, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (Humira® Abbott Laboratories, a human anti-TNF mAb, described in U.S. Pat. No. 6,090,382 as D2E7). Additional TNF antibodies which can be used in the invention are described in U.S. Pat. Nos. 6,593,458; 6,498,237; 6,451,983; and 6,448,380, each of which is incorporated by reference herein. Other combinations including TNFα converting enzyme (TACE) inhibitors; IL-1 inhibitors (Interleukin-1-converting enzyme inhibitors, IL-1RA etc.) may be effective for the same reason. Other combinations include the IL-6 antibody tocilizumab (Actemra). Other preferred combinations include Interleukin 11. Yet another preferred combination are other key players of the autoimmune response which may act parallel to, dependent on or in concert with TNFα function; especially preferred are IL-18 antagonists including IL-18 antibodies or soluble IL-18 receptors, or IL-18 binding proteins. It has been shown that TNFα and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

The antibodies of the invention, or antigen binding portions thereof, may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-1β converting enzyme inhibitors, TNFα converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFR1gG (Enbrel and p55TNFR1gG (Lenercept)), sIL-1RI, sIL-1RII, sIL-6R), antiinflammatory cytokines (e.g. IL-4, IL-10, IL-11, IL-13 and TGFβ), tocilizumab (Actemra), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, CDC-801, and Mesopram. Preferred combinations include methotrexate or leflunomide and in moderate or severe rheumatoid arthritis cases, cyclosporine.

Nonlimiting additional agents which can also be used in combination with an TNFα antibody, or antigen-binding portion thereof, to treat rheumatoid arthritis include, but are not limited to, the following: non-steroidal anti-inflammatory drug(s) (NSAIDs); cytokine suppressive anti-inflammatory drug(s) (CSAIDs); CDP-571/BAY-10-3356 (humanized anti-TNFα antibody; Celltech/Bayer); cA2/infliximab (chimeric anti-TNFα antibody; Centocor); 75 kdTNFR-IgG/etanercept (75 kD TNF receptor-IgG fusion protein; Immunex; see e.g., Arthritis & Rheumatism (1994) Vol. 37, S295; J. Invest. Med. (1996) Vol. 44, 235A); 55 kdTNF-IgG (55 kD TNF receptor-IgG fusion protein; Hoffmann-LaRoche); IDEC-CE9.1/SB 210396 (non-depleting primatized anti-CD4 antibody; IDEC/SmithKiine; see e.g., Arthritis & Rheumatism (1995) Vol. 38, S185); DAB 486-IL-2 and/or DAB 389-IL-2 (IL-2 fusion proteins; Seragen; see e.g., Arthritis & Rheumatism (1993) Vol. 36, 1223); Anti-Tac (humanized anti-IL-2Rα; Protein Design Labs/Roche); IL-4 (anti-inflammatory cytokine; DNAX/Schering); IL-10 (SCH 52000; recombinant IL-10, anti-inflammatory cytokine; DNAX/Schering); IL-4; IL-10 and/or IL-4 agonists (e.g., agonist antibodies); IL-1RA (IL-1 receptor antagonist; Synergen/Amgen); anakinra (Kineret®/Amgen); TNF-bp/s-TNF (soluble TNF binding protein; see e.g., Arthritis & Rheumatism (1996) Vol. 39, No. 9 (supplement), S284; Amer. J. Physiol.—Heart and Circulatory Physiology (1995) Vol. 268, pp. 37-42); R973401 (phosphodiesterase Type IV inhibitor; see e.g., Arthritis & Rheumatism (1996) Vol. 39, No. 9 (supplement), S282); MK-966 (COX-2 Inhibitor; see e.g., Arthritis & Rheumatism (1996) Vol. 39, No. 9 (supplement), S81); Iloprost (see e.g., Arthritis & Rheumatism (1996) Vol. 39, No. 9 (supplement), S82); methotrexate; thalidomide (see e.g., Arthritis & Rheumatism (1996) Vol. 39, No. 9 (supplement), S282) and thalidomide-related drugs (e.g., Celgen); leflunomide (anti-inflammatory and cytokine inhibitor; see e.g., Arthritis & Rheumatism (1996) Vol. 39, No. 9 (supplement), S131; Inflammation Research (1996) Vol. 45, pp. 103-107); tranexamic acid (inhibitor of plasminogen activation; see e.g., Arthritis & Rheumatism (1996) Vol. 39, No. 9 (supplement), S284); T-614 (cytokine inhibitor; see e.g., Arthritis & Rheumatism (1996) Vol. 39 No. 9 (supplement), S282); prostaglandin E1 (see e.g., Arthritis & Rheumatism (1996) Vol. 39, No. 9 (supplement), S282); Tenidap (non-steroidal anti-inflammatory drug; see e.g., Arthritis & Rheumatism (1996) Vol. 39, No. 9 (supplement), S280); Naproxen (non-steroidal anti-inflammatory drug; see e.g., Neuro Report (1996) Vol. 7, pp. 1209-1213); Meloxicam (non-steroidal anti-inflammatory drug); Ibuprofen (non-steroidal anti-inflammatory drug); Piroxicam (non-steroidal anti-inflammatory drug); Diclofenac (non-steroidal anti-inflammatory drug); Indomethacin (non-steroidal anti-inflammatory drug); Sulfasalazine (see e.g., Arthritis & Rheumatism (1996) Vol. 39, No. 9 (supplement), S281); Azathioprine (see e.g., Arthritis & Rheumatism (1996) Vol. 39 No. 9 (supplement), S281); ICE inhibitor (inhibitor of the enzyme interleukin-1β converting enzyme); zap-70 and/or lck inhibitor (inhibitor of the tyrosine kinase zap-70 or lck); VEGF inhibitor and/or VEGF-R inhibitor (inhibitors of vascular endothelial cell growth factor or vascular endothelial cell growth factor receptor; inhibitors of angiogenesis); corticosteroid anti-inflammatory drugs (e.g., SB203580); TNF-convertase inhibitors; anti-IL-12 antibodies; anti-IL-18 antibodies; interleukin-11 (see e.g., Arthritis & Rheumatism (1996) Vol. 39, No. 9 (supplement), S296); interleukin-13 (see e.g., Arthritis & Rheumatism (1996) Vol. 39, No. 9 (supplement), S308); interleukin-17 inhibitors (see e.g., Arthritis & Rheumatism (1996) Vol. 39, No. 9 (supplement), S120); gold; penicillamine; chloroquine; chlorambucil; hydroxychloroquine; cyclosporine; cyclophosphamide; total lymphoid irradiation; anti-thymocyte globulin; anti-CD4 antibodies; CD5-toxins; orally-administered peptides and collagen; lobenzarit disodium; Cytokine Regulating Agents (CRAs) HP228 and HP466 (Houghten Pharmaceuticals, Inc.); ICAM-1 antisense phosphorothioate oligo-deoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline; anti-IL2R antibodies; marine and botanical lipids (fish and plant seed fatty acids; see e.g., DeLuca et al. (1995) Rheum. Dis. Clin. North Am. 21:759-777); auranofin; phenylbutazone; meclofenamic acid; flufenamic acid; intravenous immune globulin; zileuton; azaribine; mycophenolic acid (RS-61443); tacrolimus (FK-506); sirolimus (rapamycin); amiprilose (therafectin); cladribine (2-chlorodeoxyadenosine); methotrexate; antivirals; and immune modulating agents.

In one embodiment, the TNFα antibody, or antigen-binding portion thereof, is administered in combination with one of the following agents for the treatment of rheumatoid arthritis: small molecule inhibitor of KDR (ABT-123), small molecule inhibitor of Tie-2; methotrexate; prednisone; celecoxib; folic acid; hydroxychloroquine sulfate; rofecoxib; etanercept; infliximab; leflunomide; naproxen; valdecoxib; sulfasalazine; methylprednisolone; ibuprofen; meloxicam; methylprednisolone acetate; gold sodium thiomalate; aspirin; azathioprine; triamcinolone acetonide; propxyphene napsylate/apap; folate; nabumetone; diclofenac; piroxicam; etodolac; diclofenac sodium; oxaprozin; oxycodone hcl; hydrocodone bitartrate/apap; diclofenac sodium/misoprostol; fentanyl; anakinra, human recombinant; tramadol hcl; salsalate; sulindac; cyanocobalamin/fa/pyridoxine; acetaminophen; alendronate sodium; prednisolone; morphine sulfate; lidocaine hydrochloride; indomethacin; glucosamine sulfate/chondroitin; cyclosporine; amitriptyline hcl; sulfadiazine; oxycodone hcl/acetaminophen; olopatadine hcl; misoprostol; naproxen sodium; omeprazole; mycophenolate mofetil; cyclophosphamide; rituximab; IL-1 TRAP; MRA; CTLA4-IG; IL-18 BP; ABT-874; ABT-325 (anti-IL 18); anti-IL 15; BIRB-796; SCIO-469; VX-702; AMG-548; VX-740; Roflumilast; IC-485; CDC-801; and mesopram. In another embodiment, a TNF antibody, or antigen-binding portion thereof, is administered for the treatment of an TNF-related disorder in combination with one of the above mentioned agents for the treatment of rheumatoid arthritis.

The antibodies of the invention, or antigen binding portions thereof, may also be combined with agents, such as alemtuzumab, dronabinol, Unimed, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists, IL-4 agonists.

In one embodiment, the methods and compositions of the invention provide a combination use of a TNFα antibody, e.g., adalimumab, and a DMARD, e.g., methotrexate.

### IV. Efficacy of TNFα Inhibitor for Rheumatoid Arthritis

The invention also provides methods for determining whether a TNFα inhibitor is effective at treating rheumatoid arthritis (RA) in a subject. Such methods may be used to determine the efficacy of a TNFα inhibitor, including those which are unknown or unconfirmed to have such efficacy. Using the methods described herein, effective TNFα inhibitors may be determined or confirmed, and, subsequently, used in the method of treating RA.

In one embodiment, the invention provides a method for determining the efficacy of a TNFα inhibitor, including a human TNFα antibody, for treating RA in a subject, using the ACR response. The American College of Rheumatology preliminary criteria for improvement in Rheumatoid Arthritis (e.g., ACR20, 50, 70 responses) was developed to provide a efficacy measures for rheumatoid arthritis (RA) treatments. ACR20, ACR50 and ACR70 requires a greater than 20%, 50% and 70% improvement respectively. Response criteria are detailed in Felson D T, Anderson J J, Boers M, Bombardier C, Furst D, Goldsmith C, et al. American College of Rheumatology preliminary definition of improvement in rheumatoid arthritis. Arthritis Rheum 1995; 38:727-35, incorporated by reference herein. Generally, patients are examined clinically at screening, baseline, and frequently during treatment. The primary efficacy for signs and symptoms is measured via American College of Rheumatology preliminary criteria for improvement (ACR20) at 12 weeks. An additional primary endpoint includes evaluation of radiologic changes over 6 to 12 months to assess changes in structural damage. The efficacy of a TNFα inhibitor for treating RA may be determined by the ACR response of a patient population who may be evaluated by determining the percentage of the patient population in whom an ACR response occurs following administration of the TNFα inhibitor.

The ACR response (or any of the indices described herein) may be used as an index for measuring efficacy of a TNFα inhibitor in a patient population having RA, where attaining a certain percentage of patients within a population who were administered the TNFα inhibitor and who achieve an ACR response, i.e. ACR20, ACR50, ACR70, indicates that the TNFα inhibitor is effective for treating RA. In one embodiment, the invention provides a method for determining whether a human TNFα antibody is effective for treating RA.

In one embodiment, the invention provides a method of determining the efficacy of a TNFα inhibitor, e.g., an antibody, for treating RA in a subject comprising determining an ACR20 response of a patient population having RA and who was administered the TNFα inhibitor, wherein an ACR20 response in at least about 33% of the patient population indicates that the TNFα inhibitor is an effective TNFα inhibitor for the treatment of RA in a subject. In one embodiment, an ACR20 response in at least about 49% of the patient population indicates that the TNFα inhibitor is an effective TNFα inhibitor for the treatment of RA in a subject. In one embodiment, an ACR20 response in at least about 50% of the patient population indicates that the TNFα inhibitor is an effective TNFα inhibitor for the treatment of RA in a subject. In one embodiment, an ACR20 response in at least about 51% of the patient population indicates that the TNFα inhibitor is an effective TNFα inhibitor for the treatment of RA in a subject. In one embodiment, an ACR20 response in at least about 57% of the patient population indicates that the TNFα inhibitor is an effective TNFα inhibitor for the treatment of RA in a subject. In one embodiment, an ACR20 response in at least about 60% of the patient population indicates that the TNFα inhibitor is an effective TNFα inhibitor for the treatment of RA in a subject. In one embodiment, the invention provides a method of determining the efficacy of a TNFα antibody, for treating RA in a subject comprising determining an ACR20 response of a patient population having RA and who was administered the TNFα antibody, wherein an ACR20 response in at least about 67% of the patient population indicates that the TNFα inhibitor is an effective TNFα antibody for the treatment of RA in a subject. In one embodiment, an ACR20 response in at least about 67% of the patient population indicates that the TNFα antibody is an effective TNFα inhibitor for the treatment of RA in a subject. In one embodiment, an ACR20 response in at least about 33%, at least about 49%, at least about 50%, at least about 51%, at least about 55%, at least about 56%, at least about 57%, at least about 58%, at least about 60%, at least about 61%, at least about 63%, at least about 65%, at least about 64%, at least about 67%, at least about 69%, at least about 70%, at least about 72%, at least about 75%, at least about 79%, at least about 81%, at least about 82%, or at least about 85%, of the patient population indicates that the TNFα inhibitor is an effective TNFα inhibitor for the treatment of RA in a subject. Numbers intermediate to the above recited percentages, e.g., 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, as well as all other numbers recited herein, are also intended to be part of this invention. Ranges of values using a combination of any of the above recited values as upper and/or lower limits are intended to be included in the scope of the invention.

In one embodiment, the invention provides a method of determining the efficacy of a TNFα inhibitor for treating RA in a subject comprising determining an ACR50 response of a patient population having RA and who was administered the TNFα inhibitor, wherein an ACR50 response in at least about 30% of the patient population indicates that the TNFα inhibitor is an effective TNFα inhibitor for the treatment of RA in a subject. In one embodiment, an ACR50 response in at least about 35% of the patient population indicates that the TNFα inhibitor is an effective TNFα inhibitor for the treatment of RA in a subject. In one embodiment, an ACR50 response in at least about 18%, at least about 25%, at least about at 26%, at least about 30%, at least about 34%, at least about 36%, least about 39%, at least about 40%, at least about 41%, at least about 42%, at least about 43%, at least about 45%, at least about 48%, at least about 59%, at least about 60%, at least about 61%, at least about 62% of the patient population indicates that the TNFα inhibitor is an effective TNFα inhibitor for the treatment of RA in a subject. Numbers intermediate to the above recited percentages, e.g., 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, as well as all other numbers recited herein, are also intended to be part of this invention. Ranges of values using a combination of any of the above recited values as upper and/or lower limits are intended to be included in the scope of the invention.

In one embodiment, the invention provides a method for determining the efficacy of a human TNFα antibody, or antigen-binding portion thereof, for treating RA in a subject comprising determining an ACR70 response of a patient population having RA and who was administered the human TNFα antibody, or antigen-binding portion thereof, wherein an ACR70 response in at least about 19% of the patient population indicates that the human TNFα antibody, or antigen-binding portion thereof, is an effective human TNFα antibody, or antigen-binding portion thereof, for the treatment of RA in a subject. In one embodiment, an ACR70 response in at least about 20% of the patient population indicates that the human TNFα antibody, or antigen-binding portion thereof, is an effective human TNFα antibody, or antigen-binding portion thereof, for the treatment of RA in a subject. In one embodiment, an ACR70 response in at least about 23% of the patient population indicates that the human TNFα antibody, or antigen-binding portion thereof, is an effective human TNFα antibody, or antigen-binding portion thereof, for the treatment of RA in a subject. In one embodiment, an ACR70 response in at least about 28% of the patient population indicates that the human TNFα antibody, or antigen-binding portion thereof, is an effective human TNFα antibody, or antigen-binding portion thereof, for the treatment of RA in a subject. In one embodiment, an ACR70 response in at least about 38% of the patient population indicates that the human TNFα antibody, or antigen-binding portion thereof, is an effective human TNFα antibody, or antigen-binding portion thereof, for the treatment of RA in a subject. In one embodiment, an ACR70 response in at least about 11%, in at least about 18%, at least about 19%, at least about 20%, at least about 23%, at least about 24%, at least about 26%, at least about 28%, at least about 30%, at least about 34%, at least about 35%, at least about 38%, at least about 46%, at least about 47%, of the patient population indicates that the human TNFα antibody, or antigen-binding portion thereof, is an effective human TNFα antibody, or antigen-binding portion thereof, for the treatment of RA in a subject. Numbers intermediate to the above recited percentages, e.g., 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, as well as all other numbers recited herein, are also intended to be part of this invention. Ranges of values using a combination of any of the above recited values as upper and/or lower limits are intended to be included in the scope of the invention.

The invention also includes a method of determining the efficacy of a TNFα inhibitor, e.g., human TNFα antibody, or antigen-binding portion thereof, for treating RA in a subject comprising determining an ACR90 response of a patient population having RA and who was administered the human TNFα antibody, or antigen-binding portion thereof, wherein an ACR90 response in at least about 8% of the patient population indicates that the human TNFα antibody, or antigen-binding portion thereof, is an effective human TNFα antibody, or antigen-binding portion thereof, for the treatment of RA in a subject. In one embodiment, an ACR90 response in at least about 10% of the patient population indicates that the human TNFα antibody, or antigen-binding portion thereof, is an effective human TNFα antibody, or antigen-binding portion thereof, for the treatment of RA in a subject. In one embodiment, an ACR90 response in at least about 15% of the patient population indicates that the human TNFα antibody, or antigen-binding portion thereof, is an effective human TNFα antibody, or antigen-binding portion thereof, for the treatment of RA in a subject. In one embodiment, an ACR90 response in at least about 20% of the patient population indicates that the human TNFα antibody, or antigen-binding portion thereof, is an effective human TNFα antibody, or antigen-binding portion thereof, for the treatment of RA in a subject. In one embodiment, an ACR90 response in at least about 25% of the patient population indicates that the human TNFα antibody, or antigen-binding portion thereof, is an effective human TNFα antibody, or antigen-binding portion thereof, for the treatment of RA in a subject. In one embodiment, an ACR90 response in at least about 27% of the patient population indicates that the human TNFα antibody, or antigen-binding portion thereof, is an effective human TNFα antibody, or antigen-binding portion thereof, for the treatment of RA in a subject. Numbers intermediate to the above recited percentages, e.g., 8%, 9%, 10%, 11%, 12%, 13%, 14%, 15%, 16%, 17%, 18%, 19%, 20%, 21%, 22%, 23%, 24%, 25%, 26%, 27%, as well as all other numbers recited herein, are also intended to be part of this invention. Ranges of values using a combination of any of the above recited values as upper and/or lower limits are intended to be included in the scope of the invention.

The methods of the invention may also be used to determine efficacy of a TNFα inhibitor, e.g., a TNFα antibody, or antigen-binding portion thereof, in a subpopulation of RA patients, e.g., patients who have failed prior TNFα inhibitor therapy, using an ACR response, e.g., ACR20, ACR50, ACR70, ACR90.

In one embodiment, the invention provides a method for determining the efficacy of a human TNFα antibody, or antigen-binding portion thereof, for treating RA in a subject who has failed prior infliximab treatment comprising determining an ACR20 response of a patient population having RA who has failed previous infliximab or etanercept treatment and who was administered the human TNFα antibody, or antigen-binding portion thereof, wherein an ACR20 response in at least about 33% of the patient population indicates that the human TNFα antibody, or antigen-binding portion thereof, is an effective human TNFα antibody, or antigen-binding portion thereof, for the treatment of RA in a subject who has failed prior infliximab or etanercept treatment. In one embodiment, an ACR20 response in at least about 40% of the patient population indicates that the human TNFα antibody, or antigen-binding portion thereof, is an effective human TNFα antibody, or antigen-binding portion thereof, for the treatment of RA in a subject who has failed prior infliximab or etanercept treatment. In one embodiment, an ACR20 response in at least about 45% of the patient population indicates that the human TNFα antibody, or antigen-binding portion thereof, is an effective human TNFα antibody, or antigen-binding portion thereof, for the treatment of RA in a subject who has failed prior infliximab or etanercept treatment. In one embodiment, an ACR20 response in at least about 50% of the patient population indicates that the human TNFα antibody, or antigen-binding portion thereof, is an effective human TNFα antibody, or antigen-binding portion thereof, for the treatment of RA in a subject who has failed prior infliximab or etanercept treatment. In one embodiment, an ACR20 response in at least about 55% of the patient population indicates that the human TNFα antibody, or antigen-binding portion thereof, is an effective human TNFα antibody, or antigen-binding portion thereof, for the treatment of RA in a subject who has failed prior infliximab or etanercept treatment. In one embodiment, an ACR20 response in at least about 61% of the patient population indicates that the human TNFα antibody, or antigen-binding portion thereof, is an effective human TNFα antibody, or antigen-binding portion thereof, for the treatment of RA in a subject who has failed prior infliximab or etanercept treatment. It should be noted that the invention also includes the use of other of ACR responses, e.g., ACR50, ACR70, to determine the efficacy of a TNFα inhibitor for treating RA, like those described in the Examples provided below. Numbers intermediate to the above recited percentages, e.g., 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, as well as all other numbers recited herein, are also intended to be part of this invention. Ranges of values using a combination of any of the above recited values as upper and/or lower limits are intended to be included in the scope of the invention.

In one embodiment, the invention provides a method for determining the efficacy of a TNFα inhibitor, including a human TNFα antibody, for treating RA in a subject, using the EULAR response of a subject or patient population. European League Against Rheumatism (EULAR) Response Criteria uses a Disease Activity Score (DAS) for defining response. To be classified as responders, patients should have a significant change in DAS and also low current disease activity. Response is defined as both: (a) change in disease activity from baseline and (b) the level of disease activity reached during follow-up. Criteria used to define DAS include: Ritchie articular index, swollen joint count (44-joint count), erythrocyte sedimentation rate, and Health Assessment Questionnaire. A modified version of the DAS criteria, DAS28, uses a 28-joint count for swollen and tender joints. Response is defined as a combination of a significant change from baseline and the level of disease activity attained. Good response is defined as a significant decrease in DAS (>1.2) and a low level of disease activity (< or =2.4). Non-response is defined as a decrease < or =0.6, or a decrease >0.6 and < or =1.2 with an attained DAS >3.7. Any other scores are regarded as moderate responses. Thus, three categories are defined: good, moderate, and non-responders. For details of EULAR criteria see Van Gestel et al. (1996) 39(1):34-40, incorporated by reference herein.

In one embodiment, the invention provides a method for determining the efficacy of a human TNFα antibody, or antigen-binding portion thereof, for treating RA in a subject comprising determining a moderate EULAR response of a patient population having RA and who was administered the human TNFα antibody, or antigen-binding portion thereof, wherein a moderate EULAR response in at least about 46% of the patient population indicates that the human TNFα antibody, or antigen-binding portion thereof, is an effective human TNFα antibody, or antigen-binding portion thereof, for the treatment of RA. In one embodiment, a moderate EULAR response in at least about 75% of the patient population indicates that the human TNFα antibody, or antigen-binding portion thereof, is an effective human TNFα antibody, or antigen-binding portion thereof, for the treatment of RA. In one embodiment, a moderate EULAR response in at least about 80% of the patient population indicates that the human TNFα antibody, or antigen-binding portion thereof, is an effective human TNFα antibody, or antigen-binding portion thereof, for the treatment of RA. In one embodiment, a moderate EULAR response in at least about 84% of the patient population indicates that the human TNFα antibody, or antigen-binding portion thereof, is an effective human TNFα antibody, or antigen-binding portion thereof, for the treatment of RA. In one embodiment, a moderate EULAR response in at least about 46%, at least about 50%, at least about 55%, at least about 60%, at least about 65%, at least about 70%, at least about 74%, at least about 75%, at least about 76%, at least about 77%, at least about 79%, at least about 80%, at least about 81%, at least about 82%, at least about 83%, at least about 84%, at least about 85%, or at least about 90%, or at least about 92% of the patient population indicates that the human TNFα antibody, or antigen-binding portion thereof, is an effective human TNFα antibody, or antigen-binding portion thereof, for the treatment of RA. Numbers intermediate to the above recited percentages, e.g., 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, as well as all other numbers recited herein, are also intended to be part of this invention. Ranges of values using a combination of any of the above recited values as upper and/or lower limits are intended to be included in the scope of the invention.

In one embodiment, the invention provides a method for determining the efficacy of a human TNFα antibody, or antigen-binding portion thereof, for treating RA in a subject comprising determining a good EULAR response of a patient population having RA and who was administered the human TNFα antibody, or antigen-binding portion thereof, wherein a good EULAR response in at least about 18% of the patient population indicates that the human TNFα antibody, or antigen-binding portion thereof, is an effective human TNFα antibody, or antigen-binding portion thereof, for the treatment of RA in a subject. In one embodiment, a good EULAR response in at least about 20% of the patient population indicates that the human TNFα antibody, or antigen-binding portion thereof, is an effective human TNFα antibody, or antigen-binding portion thereof, for the treatment of RA in a subject. In one embodiment, a good EULAR response in at least about 8%, in at least about 11%, in at least about 18%, at least about 20%, at least about 22%, at least about 25%, at least about 30%, in at least about 31%, at least about 34%, at least about 35%, at least about 36%, at least about 39%, %, at least about 40%, of the patient population indicates that the human TNFα antibody, or antigen-binding portion thereof, is an effective human TNFα antibody, or antigen-binding portion thereof, for the treatment of RA in a subject. Numbers intermediate to the above recited percentages, e.g., 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, as well as all other numbers recited herein, are also intended to be part of this invention. Ranges of values using a combination of any of the above recited values as upper and/or lower limits are intended to be included in the scope of the invention.

Other indices described in the art, including those referenced in the Examples, may also be used to determine the efficacy of a TNFα inhibitor in accordance with the methods of the invention. For example, TJC and/or SJC counts may be used, HAQ scores may be used, and DAS scores may be used to determine whether a TNFα inhibitor is efficacious for treating RA in a subject. Other indices known in the art include SF-36, FACIT-F, HUI3, and HRQoL.

Swollen and tender joints are the most characteristic features of RA, as disease severity is directly related to the number of swollen and tender joints. Counting swollen and tender joints is a key component of the clinical assessment of RA. Tender joint count (TJC) is an assessment of 28 or more joints where several different aspects of tenderness are assessed by pressure and joint manipulation on physical examination. Swollen joint count (SJC): an assessment of 28 or more joints where joints are classified as either swollen or not swollen. For TJC and SJC scoring see Fuchs and Pincus, Arthritis Rheum 37:470-475, 1994; Arthritis Rheum 37:463-464, 1994).

In one embodiment, the invention provides a method for monitoring the effectiveness of an anti-TNFα regimen for treating rheumatoid arthritis (RA) comprising administering to a subject a TNFα inhibitor in accordance with the anti-TNFα regimen, wherein the subject has a baseline TJC of at least about 17 and/or a baseline SJC of at least 14. Following administration and during the course of treatment, the TJC and/or SJC score(s) of the subject of the subject are determined and are compared with the baseline scores. In one embodiment, a decrease of at least 10 points in the TJC score and/or a decrease of at least 7 in the SJC score indicates that the anti-TNFα regimen is effective at treating RA. In one embodiment, a TJC score of 0 in at least about 10%, in at least about 15%, in at least about 20%, at least about 23%, at least about 24%, at least about 25%, at least about 30%, at least about 35%, at least about 36%, of the patient population indicates that the human TNFα antibody, or antigen-binding portion thereof, is an effective human TNFα antibody, or antigen-binding portion thereof, for the treatment of RA in a subject. In one embodiment, an SJC score of 0 in at least about 7%, in at least about 10%, in at least about 15%, in at least about 20%, at least about 21%, at least about 25%, at least about 30%, at least about 32%, at least about 35%, at least about 39%, of the patient population indicates that the human TNFα antibody, or antigen-binding portion thereof, is an effective human TNFα antibody, or antigen-binding portion thereof, for the treatment of RA in a subject. Numbers intermediate to the above recited percentages, e.g., 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, as well as all other numbers recited herein, are also intended to be part of this invention. Ranges of values using a combination of any of the above recited values as upper and/or lower limits are intended to be included in the scope of the invention.

Efficacy of a treatment for rheumatoid arthritis can be determined by using the health assessment questionnaire (HAQ). Health Assessment Questionaire (HAQ) is a standardized disability questionnaire that was initially developed for use in rheumatoid arthritis (RA). A high HAQ score has been shown to be a strong predictor of morbidity and mortality in RA, and low HAQ scores are predictive of better outcomes (see Fries et al. Arthritis Rheum 1980; 23:137-45). The HAQ is a validated questionnaire designed to assess patients' ability to perform activities of daily living, particularly in adult arthritics. The instrument consists of the HAQ Disability Index (20 items), Pain Scale (1 item), and Global Health Status (1 item) that measure disability/physical functioning and quality of life (see Fries (1982) et al. J Rheumatol. 9:789).

In one embodiment, the invention includes a method for treating a human subject suffering from rheumatoid arthritis who has been identified as having a baseline health assessment questionnaire (HAQ) score of at least about 1.4 comprising administering to the subject a TNFα inhibitor, such that the HAQ score of the subject is decreased by at least about 0.49 points. In another embodiment, the invention provides method of treating a human subject suffering from rheumatoid arthritis, comprising identifying a subject with a HAQ score of at least about 1.4; and administering to the subject a TNFα inhibitor such that the HAQ score of the subject is decreased by at least about 0.49 points.

Efficacy of a treatment can also be determined by a decrease of at least about 0.49 points in the HAQ score in about 25-28% of a preselected patient population who have been administered a TNFα inhibitor. The baseline of such a patient population is an HAQ score of at least about 1.4.

The effectiveness of an anti-TNFα regimen for treating rheumatoid arthritis (RA) may be monitored by administering to a subject a TNFα inhibitor in accordance with an anti-TNFα regimen, wherein the subject has a baseline HAQ score of at least about 1.4. At a certain time point following administration, i.e., during the course of treatment, the subject's HAQ score is re-assessed. HAQ scores may be determined at any given interval during treatment, e.g., every week, every 4^{th} week in a 12 week treatment, etc. A decrease in the HAQ score by at least 0.49 points from the baseline HAQ score to the HAQ score determined during the course of treatment indicates that the anti-TNFα regimen is effective at treating RA. A decrease in the HAQ score at least about 0.55 points is also indicative of an effective treatment.

DAS28 (disease activity score) is also an accepted measure of the activity of rheumatoid arthritis in an affected subject. The DAS is a score is based on the Ritchie articular index, a 44 swollen joint count, ESR, and a general health assessment on a VAS. Range varies from 1 to 9. Serial measurements of the DAS and DAS28 are strong predictors of physical disability and radiological progression, and both indices are sensitive discriminators between patients with high and low disease activity and between active and placebo treated patient groups. The following parameters are included in the calculation: Number of joints tender to the touch (TEN); Number of swollen joints (SW); Erythrocyte sedimentation rate (ESR); Patient assessment of disease activity (VAS; mm) (see Van der Heijde et al. Ann Rheum Dis 1990; 49:916-20). In modified DAS (DAS28) 28 joints are assessed (see Prevoo M L L, et al. Arthritis Rheum 1995; 38:44-8).

In one embodiment, a DAS28 score of less than 2.6 in at least about 20%, in at least about 23%, at least about 25%, at least about 30%, at least about 33%, at least about 35%, at least about 40%, at least about 43%, at least about 49%, of the patient population indicates that the human TNFα antibody, or antigen-binding portion thereof, is an effective human TNFα antibody, or antigen-binding portion thereof, for the treatment of RA in a subject. Numbers intermediate to the above recited percentages, e.g., 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, and 49%, as well as all other numbers recited herein, are also intended to be part of this invention. Ranges of values using a combination of any of the above recited values as upper and/or lower limits are intended to be included in the scope of the invention.

In one embodiment, the invention provides a method for predicting whether a patient is a candidate for treatment with a TNFα inhibitor using a DAS28 score and a RAPID score of the patient. In one embodiment, a DAS28 score of at least about 5.1 and a RAPID score of at least about 5 indicates the subject is a good candidate for TNFα inhibitor therapy for RA.

In one embodiment, the invention provides a method for determining the efficacy of a TNFα inhibitor, including a human TNFα antibody, for treating RA in a subject, using the CRP level in correlation with a Patient Activity Score (PAS). The invention. The invention provides a method for predicting the efficacy of a TNFα inhibitor for the treatment of rheumatoid arthritis (RA) in a subject comprising using the combination of a C-reactive protein (CRP) level of the subject and a Patient Activity Score (PAS) of the subject, wherein an improvement in the CRP level and the PAS score early in the treatment of the patient with the TNFα inhibitor indicates that the TNFα inhibitor is an effective TNFα inhibitor for the treatment of RA in the subject. In one embodiment, the improvement in the CRP level and the PAS score early in the treatment of the subject occurs at about two weeks following initiation of the treatment in the subject. In one embodiment, the PAS score is determined using the Health Assessment Questionnaire (HAQ) of the subject. In one embodiment, the improvement in the CRP level is at least as described in the Examples below. In one embodiment, the improvement in the HAQ score is at least about 0.4.

FACIT-F (Functional Assessment of Chronic Illness Therapy—Fatigue) is a validated questionnaire designed to measure patients' assessment of fatigue-related factors in chronic illness (see Cella and Webster (1997) Oncology (Huntingt). 11:232 and Lai et al. (2003) Qual Life Res. 12(5):485). In one embodiment, the invention includes using a FACIT-F score of a patient population who has been administered a TNFα inhibitor to determine whether the TNFα inhibitor is effective at treating RA.

Efficacy of a treatment for rheumatoid arthritis can be determined by comparing AIMS2-SF scores given by subjects having rheumatoid arthritis. The AIMS2-SF is a shorter version of the AIMS2 (i.e., available in 2-page format) and has psychometric properties similar to those of the AIMS2 (see Guillemin et al. (1997) Arthritis Rheum. 40(7): 1267).

The invention also includes a method for determining the efficacy a TNFα inhibitor for the treatment of a TNFα-related disorder, e.g., RA, comprising determining whether the TNFα inhibitor is an effective TNFα inhibitor using a baseline magnetic resonance imaging (MRI) score of a patient or patient population in comparison with a score determined at a point following treatment in the patient or patient population with the TNFα inhibitor, e.g., a TNFα antibody. The invention also describes a method for monitoring the effectiveness of a TNFα inhibitor, e.g., a TNFα antibody, or antigen binding portion thereof, for reducing inflammation in a metacarpophalangeal or interphalangeal joint of a patient population having a TNFα-related disorder. In a preferred embodiment, inflammation in the hand joints of a subject may be determined using MRI.

Conventional radiography offers information about destructive joint changes and has been the mainstay in diagnostic imaging in inflammatory arthropathies. Magnetic Resonance Imaging (MRI) has brought advances to musculoskeletal imaging because of its ability to image soft tissue structures not visible on conventional radiographs. Thus, conventional radiographs that may be used to determine the efficacy of a TNFα inhibitor for the treatment of a TNFα-related disorder may fail to identify progress in the soft tissues of the relating to treatment with the inhibitor. Importantly, MRI is capable of detecting inflammation, as well as early disease in patients who are not yet symptomatic. MRI allows the joints to be visualized as a complete entity, and all the components of the joint including bone, cartilage, joint lining, ligaments, muscles, and soft tissue may be scrutinized for signs of arthritic change.

Magnetic Resonance Imaging (MRI) derives structural information from the density of protons in tissue and the relationship of these protons to their immediate surroundings. MRI involves changing the strength and timing of magnetic field gradients, as well as altering radiofrequency pulses and sampling the emitted energies.

In one embodiment, MRI is performed on hands of patients having inflammation resulting from a TNFα-related disorder, e.g., RA. MRI may be used to examine synovitis, bone oedema, bone erosion, or any combination thereof. In one embodiment, MRI is used to determine the efficacy of a TNFα inhibitor at decreasing inflammation in the metacarpophalangeal or interphalangeal joint.

Improvements in the patient, i.e., subject, treated with the TNFα inhibitor may be determined using a conventional MRI scoring system. In one embodiment, improvements in subjects having arthritis, e.g., rheumatoid arthritis, are determined using the OMERACT scoring system, as described in McQueen et al. (1998) Ann Rheum Dis 57:350 and Østergaard et al. (2003) J Rheumatol 30:1385-1386, each of which is incorporated by reference herein.

The term “synovitis score” or “synovitis MRI score,” as used herein, refers to a score calculated using MRI to examine synovitis associated with a certain joint. The term “tenosynovitis MRI score” or “tenosynovitis score,” as used herein, refers to a score calculated using MRI to examine tenosynovitis associated with a certain tendon.

The methods of the invention may be used to determine the efficacy of an anti-TNFα treatment for a certain disorder which is associated with joint inflammation. In one embodiment, joint inflammation is found in the hand, finger, or a combination thereof, of a subject having a TNFα-related disorder.

In one embodiment, MRI is used to determine the efficacy of a TNFα inhibitor for the treatment of a TNFα-related disorder by measuring a decrease in erosion, synovitis, tenosynovitis, or a combination thereof. MRI may also be used in the method of the invention for monitoring the effectiveness of a TNFα antibody, or an antigen binding portion thereof, for reducing inflammation in a metacarpophalangeal or interphalangeal joint of a patient population having a TNFα-related disorder. Inflammation in metacarpophalangeal and/or interphalangeal joints may be synovitis, tenosynovitis, or both.

Synovitis refers to inflammation of a synovial (joint-lining) membrane, usually painful, particularly on motion, and characterized by swelling, due to effusion (fluid collection) in a synovial sac. Synovitis is a major problem in many TNFα-related disorders, including, but not limited to, rheumatoid arthritis, juvenile arthritis, lupus, and psoriatic arthritis. The effectiveness of a TNFα inhibitor for the treatment of hand or finger inflammation (or inflammation in both the hand and finger) associated with a TNFα-related disorder may be determined using a baseline median synovitis MRI score of a patient population and a median synovitis MRI score of the patient population following administration of the TNFα inhibitor.

In one embodiment, a decrease of at least about 2 in the median synovitis MRI score of the patient population indicates that the TNFα inhibitor is efficacious for the treatment of the TNFα-related disorder and reducing inflammation in the given joint. In another embodiment, the median synovitis MRI score of the patient population which indicates efficacy of the TNFα inhibitor is between about 10.9 and about 9.

Tenosynovitis refers to inflammation of the lining of the sheath that surrounds a tendon (the cord that joins muscle to bone). Tenosynovitis can affect any lining of a tendon sheath in the body, but is possibly most commonly seen in the wrist and hand. Tenosynovitis may be found in subjects having RA. The effectiveness of a TNFα inhibitor for the treatment of hand or finger inflammation (or inflammation in both the hand and finger) associated with a TNFα-related disorder may be determined using a baseline median tenosynovitis MRI score of a patient population and a median tenosynovitis MRI score of the patient population following administration of the TNFα inhibitor.

In one embodiment, a decrease of at least about 1.5 in the median tenosynovitis score of the patient population indicates that the TNFα inhibitor is efficacious for the treatment of the TNFα-related disorder and reducing inflammation in the given joint. In another embodiment, the median tenosynovitis MRI score of the patient population which indicates efficacy of the TNFα inhibitor is between about 2.9 to about 1.5.

The invention also provides a method of achieving an improved disease activity score 28 (DAS28) score and an improved median synovitis magnetic resonance imaging (MRI) score in a preselected patient population having joint inflammation and a TNFα-related disorder selected from the group consisting of rheumatoid arthritis (RA), psoriatic arthritis (PsA), and juvenile rheumatoid arthritis (JRA), comprising administering a TNFα inhibitor to the patient population such that the DAS28 score and the median synovitis MRI score are both improved. In one embodiment, the improvement in the median synovitis score is a decrease of at least about 2. In one embodiment, the improvement in the DAS28 score is a decrease of at least about 2. in another embodiment, the DAS28 score determined following treatment using the TNFα inhibitor is between 6.0 to about 3.8

It should be noted that ranges intermediate to the above recited scores, e.g., about 10.9 to about 9, are also intended to be part of this invention. For example, ranges of values using a combination of any of the above recited values as upper and/or lower limits are intended to be included.

The invention also provides a method for determining the efficacy of a TNFα inhibitor for decreasing inflammation, i.e., treating synovitis or tenosynovitis, in a subject suffering from a TNFα-related disorder in which TNFα activity is detrimental. Examples of TNFα-related disorders which are associated with joint inflammation, including inflammation of the hand and/or finger joints, include rheumatoid arthritis (RA), psoriatic arthritis (PsA), and juvenile rheumatoid arthritis (JRA). Other examples include erosive polyarthritis and ankylosing spondylitis.

The invention also provides combined methods for determining the efficacy of a TNFα inhibitor for the treatment of a disorder in which TNFα activity is detrimental, wherein MRI scores are used in combination with a second score which is indicative of improved treatment. In one embodiment, an improved DAS28 score and an improved synovitis score in the hand/finger joints of a subject indicates that the TNFα antibody was effective for the treatment of hand/finger inflammation associated with RA.

Other scores which may be used in combination with the MRI analysis provided by the invention include any assay which measures the degree of joint destruction, including joint space narrowing and/or joint erosion. In one embodiment, joint destruction is measured using radiography. Such assays may be used to examine the efficacy of the TNFα inhibitor by determining whether an improvement occurs in a subject or patient population treated with the TNFα inhibitor. Generally, improvements are determined by comparing a baseline score determined prior to treatment, and a score determined at a time following treatment with the TNFα inhibitor.

The methods of the invention may also be used to determine efficacy of a TNFα inhibitor, e.g., a TNFα antibody, or antigen-binding portion thereof, in a subpopulation of RA patients, e.g., patients who have failed prior TNFα inhibitor therapy, using any of the indices described herein.

Also encompassed in the scope of the invention is administering the effective TNF inhibitor, e.g., human TNFα antibody, or antigen-binding portion thereof, to a subject for the treatment of RA, wherein the TNF inhibitor is identified as an effective TNF inhibitor using any of the methods and uses described herein, as well as those methods described in the Examples.

The invention further provides a method of treating RA in a subject comprising administering an effective human TNFα antibody, or antigen-binding portion thereof, for the treatment of RA in a subject, wherein the effective human TNFα antibody, or antigen-binding portion thereof, was identified as achieving a moderate EULAR response in at least about 83% of a patient population who was administered the human TNFα antibody, or antigen-binding portion thereof. In one embodiment, the invention provides a use of an effective human TNFα antibody, or antigen-binding portion thereof, in the manufacture of a medicament for treating RA in a subject, wherein the effective human TNFα antibody, or antigen-binding portion thereof, was identified as achieving a moderate EULAR response in at least about 83% of a patient population who was administered the human TNFα antibody, or antigen-binding portion thereof.

The invention also provides a method of treating RA based on the determination of a TNFα inhibitor as an effective TNFα inhibitor for achieving a certain ACR response in a patient population having taken the TNFα inhibitor. Thus, in one embodiment, the invention provides a method of treating in a subject comprising administering an effective TNFα inhibitor, wherein the effective TNFα inhibitor was identified as providing an ACR20 response in at least about 80% of a patient population who received the effective TNFα inhibitor for the treatment of RA. The invention also provides, in another embodiment, use of an effective TNFα inhibitor in the manufacture of a medicament for the treatment of RA in a subject, wherein the TNFα inhibitor was identified as providing an ACR20 response in at least about 80% of a patient population who received the effective TNFα inhibitor for the treatment of RA.

It should be noted that the Examples provided herein represent different methods of determining the efficacy of a TNFα inhibitor, such as a human TNFα antibody, or antigen-binding portion thereof. As such, data and results described in the Examples section which shows efficacy of a TNFα inhibitor, e.g., ability to treat RA, are included in the methods of determining efficacy of the invention.

Time points for determining efficacy will be understood by those of skill in the art to depend on the type of efficacy being determined, e.g., treatment of RA. In one embodiment, measurements in scores, e.g., an improvement in the ACR or EULAR response of a subject, may be measured against a subject's baseline score. Generally, a baseline refers to a measurement or score of a patient before treatment, i.e. week 0. Other time points may also be included as a starting point in determining efficacy, however. For example, in determining the efficacy of a TNFα inhibitor for treating RA in a patient population, a determination of the percentage of the patient population who is treated, e.g., improvement in ACR response, may be determined based on a time point from when treatment was initiated.

Patient populations described in the methods of the invention are generally selected based on common characteristics, such as, but not limited to, subjects diagnosed with RA who on a dosing regimen comprising a TNFα inhibitor. Such a patient population would be appropriate for determining the efficacy of the TNFα inhibitor for treating RA in the given patient population. In one embodiment, the patient population is an adult population. In another embodiment, members of a patient population have all been diagnosed with moderate to severe Ra, including moderate to severe active RA.

In one embodiment, the methods of the invention for determining whether a TNFα inhibitor is an effective TNFα inhibitor, include determining changes, improvements, measurements, etc., in RA using appropriate indices known in the art, e.g., ACR, EULAR, DAS, HAQ, FACIT-F, CRP level, MRI score, TSS change, TJC and/or SJC, from a patient population who has already been administered the TNFα inhibitor. Such a patient population may be pre-selected according to common characteristics, e.g., RA, loss of response to infliximab, and may have already been given the TNFα inhibitor. Administration of the TNFα inhibitor may or may not be performed by the same person of ordinary skill who is determining the efficacy of the TNFα inhibitor in accordance with the teachings of the specification.

In one embodiment, the methods of the invention comprise administering the TNFα inhibitor to the subjects of a patient population and determining the efficacy of the TNFα inhibitor by determining changes, improvements, measurements, etc., using RA indices known in the art, in the patient population in comparison to the Examples set forth below. For example, in one embodiment the invention includes a method for determining efficacy of a TNFα inhibitor for the treatment of RA comprising administering the TNFα inhibitor to a preselected patient population having RA; and determining the effectiveness of the TNFα inhibitor by using a mean baseline TJC or SJC score of the patient population and a mean TJC or SJC score following administration of the TNFα inhibitor.

Methods of the invention relating to determining efficacy, i.e., determining whether a TNFα inhibitor is an effective TNFα inhibitor, may also be applied to specific patient populations within the overall patient population who together have specific, common characteristics, i.e., a subpopulation. For example, the patient population may comprise patients who have failed prior infliximab treatment.

In addition, while the above methods are described in terms of patient populations, methods of efficacy described herein may also be applied to individual subjects. For example, a method for determining efficacy may comprise determining whether a subject having RA and who is on a dosage regimen, e.g., a biweekly dosing regimen, comprising a human TNFα antibody, is able to achieve an ACR50 response, wherein an ACR50 response would indicate that the human TNFα antibody is an effective human TNFα antibody.

The Examples and discoveries described herein are representative of a TNFα inhibitor, i.e., adalimumab, which is effective for treating RA, including reducing signs and symptoms of RA, inducing major clinical response in RA, inhibiting the radiographic progression of RA, and improving physical function in patients having RA. As such, the studies and results described in the Examples section herein may be used as a guideline for determining the efficacy of a TNFα inhibitor, i.e., whether a TNFα inhibitor is an effective TNFα inhibitor for the treatment of RA. In one embodiment, methods of determining efficacy described herein may be used to determine whether a TNFα inhibitor is bioequivalent to another TNFα inhibitor.

In one embodiment, the article of manufacture of the invention comprises instructions regarding how to determine the efficacy of the TNF inhibitor for the treatment of RA.

The present invention is further illustrated by the following examples which should not be construed as limiting in any way.

### EXAMPLE 1

### Efficacy and Safety of Adalimumab (HUMIRA)

An open-label, multi-center study (Study A) was performed to assess the safety and efficacy of the fully human, anti-TNF monoclonal antibody adalimumab when added to insufficient standard anti-rheumatic therapies in patients with active rheumatoid arthritis. The following study included 6,610 patients who were enrolled in 11 European countries plus Australia, including 450 sites. The following study was an open-label trial with “real-life” character, wherein the inclusion requirements were close to national guidelines/reimbursement criteria; there was add-on of adalimumab to standard of care; and included a broad patient population, including previous biologic experience. 3,813 patients were analyzed for this study.

The study design included a 12 week open label treatment period followed by a continuation period examining safety and efficacy. Adalimumab was administered to patients subcutaneously (sc) at a dose of 40 mg every other week (eow) (also referred to as biweekly). The main inclusion criteria for the study were the following: males and females ≧18 years; rheumatoid arthritis (RA; defined by ACR criteria) for ≧3 months; unsatisfactory response (or intolerance) to at least one prior DMARD; and patients with active RA (DAS28≧3.2). Baseline demographics and disease severity is shown below in Table 1:

TABLE 1
Baseline demographics and disease severity

| | |
|---|---|
| | Concomitant |
| All | DMARD Information* |

Patients

| | 3813 | 1636 |
|---|---|---|
| Disease duration (years) | 11 | 10 |
| No. of prior DMARDs | N/A | 3.1 |
| DAS28 (mean) | 6.0 | 6.0 |
| TJC (mean, 0-28 joints) | 14 | 13 |
| SJC (mean, 0-28 joints) | 11 | 11 |
| HAQ (mean, 0-3) | 1.6 | 1.6 |
| CRP (mean, mg/L) | 26 | 25 |

*Information on previous and concomitant medication available

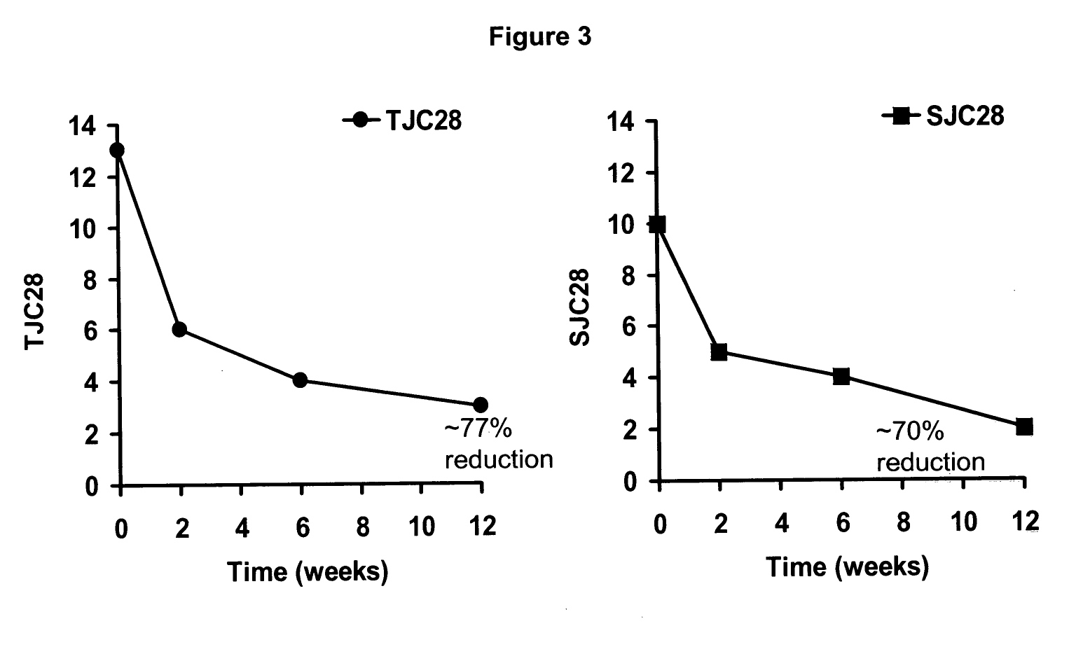

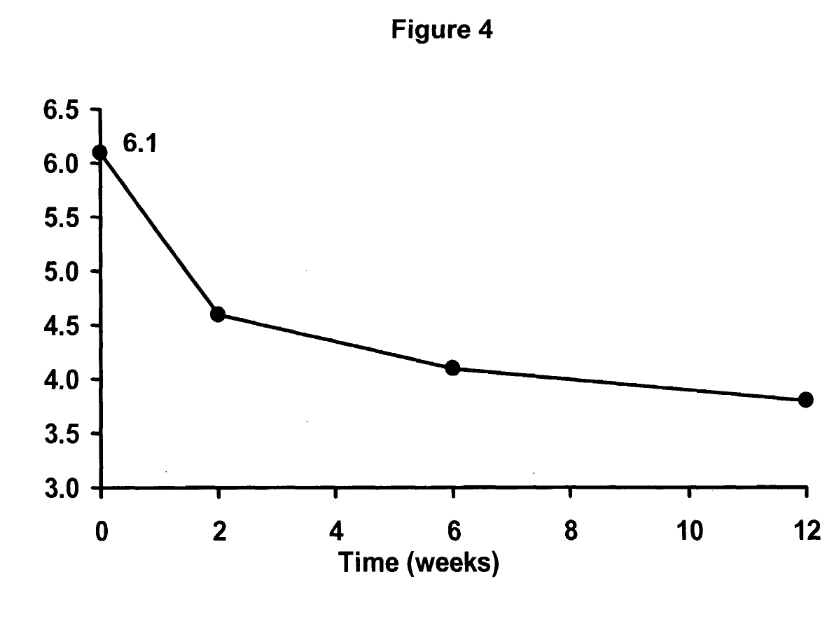

Efficacy of adalimumab treatment was determined by examining signs and symptoms of the patients. Various assays were used to determine efficacy, including ACR response, EULAR response, TJC and SJC count, and DAS28. FIG. 1 shows ACR responses of the patients at 12 weeks, where 69% of the population achieved an ACR20, 40% achieved ACR50, and 18% achieved an ACR70. FIG. 2 shows the EULAR response of the patient population at 12 weeks, wherein 83% showed a moderate EULAR response and 34% showed a good response. FIG. 3 shows the tender joint count (TJC) and swollen joint count (SJC) improvement in the patients at 12 weeks. As shown in FIG. 3, overall there was about a 77% reduction in the TJC28 and about a 70% reduction in the SJC28. In addition, 23% of the patients achieved TJC=0 at week 12, and 25% achieved SJC=0 at week 12. FIG. 4 shows the DAS28 improvement at 12 weeks. As shown in FIG. 4, 20% achieved DAS28<2.6 at week 12.

Efficacy of adalimumab treatment was also determined by examining physical function of the patients. Improvements in physical function were determined using the HAQ score. Adalimumab treatment improved the HAQ score in patients receiving treatment. The mean change HAQ score from baseline per week was as follows: week 2=−0.32; week 6=−0.44; and week 12=−0.51 (MCID was −0.22 (see Goldsmith et al. (1993) J Rheuma 20:561). Thus, 25% of patients achieved HAQ <0.5 at week 12, and 70% had a change in HAQ ≧−0.22.

Overall efficacy was also determined according to the number of concomitant DMARDs. Patients with concomitant therapy at the study entry included 1,636 patients with concomitant DMARD information, where the break down of the patient groups included 28% (n=455) had 0 DMARDs, 4% (n=62) had 3+DMARDS, 13% (n=217) had 2 DMARDs, and 55% (n=902) had 1 DMARD. The following numbers describe the types of concomitant therapy at study entry of patients with at least one DMARD: 66% methotrexate (n=777), 28% leflunomide (n=328), 12% antimalarials (n=138), 12% sulfasalazine (n=140), 4% gold parenteral (n=51), and 2% azathioprine (n=22).

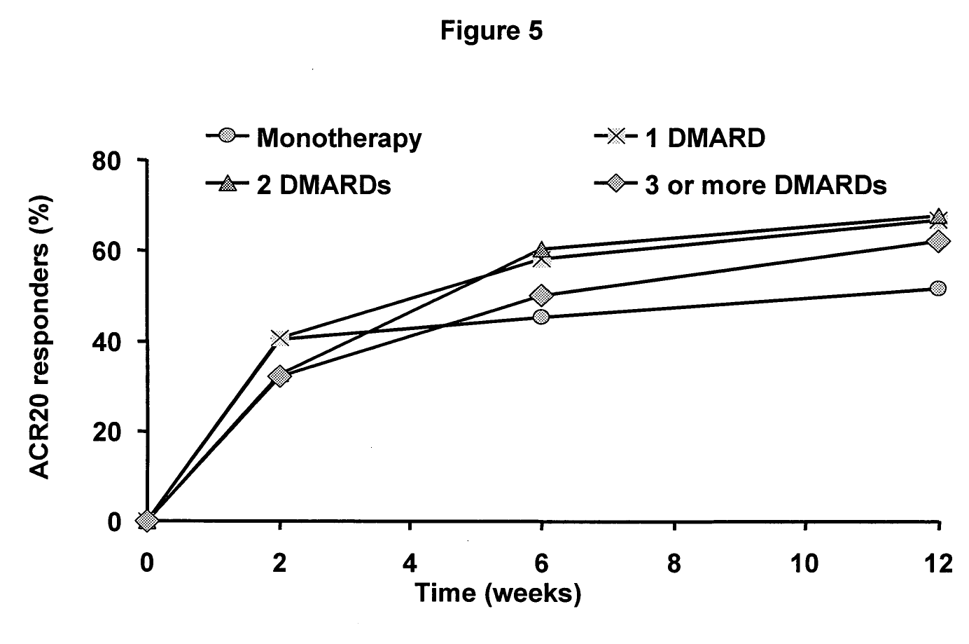

FIG. 5 describes the ACR20 response according to the concomitant therapy. DAS28 improvement at 12 weeks in patients with various DMARD concomitant therapy was also observed. The mean change in the DAS28 score from baseline for 0 DMARDs was −1.7 (n=410), 1 DMARD −2.1 (n=832), 2 DMARDSs −2.3 (n=206), and 3+DMARDs −2 (n=55). The remission rates per group (remission was defined as a DAS28<2.6) were 14% for 0 DMARDs, 21% 1 DMARD, 23% for 2 DMARDs, and 23% for 3+DMARDs. Table 2 below provides additional efficacy data with respect to patients having concomitant therapy:

TABLE 2
Baseline Demographics by Concomitant DMARD*

| n= | mtx (566) | lef (255) | ssz (38) | hcq (29) |
|---|---|---|---|---|
| # prior DMARDs | 2.7 | 3.2 | 3.1 | 3.3 |
| % steroids | 63 | 59 | 50 | 72 |
| SJC** | 10 | 11 | 11 | 12 |
| HAQ** | 1.5 | 1.6 | 1.7 | 1.8 |
| DAS28** | 5.9 | 6.0 | 6.3 | 6.2 |

*exclusively
**mean values

Table 3 shows efficacy by concomitant DMARD, including methotrexate (mtx), leflunomide (lef), sulfasalazine (slz), and hydroxychloroquine (hcq).

TABLE 3
Efficacy by concomitant DMARD

| | | | | Moderate | |
|---|---|---|---|---|---|
| | | ACR20 | ACR50 | EULAR | Good EULAR |
| % of | Mtx (n = 566) | 70 | 45 | 84 | 39 |
| patients | Lef (n = 255) | 64 | 34 | 79 | 31 |
| | Slz (n = 38) | 70 | 41 | 79 | 31 |
| | Hcq (n = 29) | 75 | 36 | 75 | 25 |

Overall efficacy was also determined according to prior therapy with biologics. Prior therapy with biologics at study entry is shown below in Table 4:

TABLE 4

| Prior therapy | n = 262 | % |
|---|---|---|
| 1 Biologic | 203 | 77 |
| 2 Biologics | 46 | 18 |
| 3 Biologics | 13 | 5 |
| Infliximab | 166* | 63 |
| Etanercept | 109* | 42 |
| Anakinra | 59* | 23 |

*not exclusively

Table 5 shows ACR and EULAR responses of adalimumab at week 12.

TABLE 5
ACR and EULAR responses of ada at week 12

| | | | | Moderate | |
|---|---|---|---|---|---|
| | | ACR20 | ACR50 | EULAR | Good EULAR |
| % of | No prior | 67 | 39 | 81 | 34 |
| patients | biologic | | | | |
| | Prior | 57 | 30 | 77 | 22 |
| | biologic | | | | |

In summary, the efficacy results show that there was a significant reduction in signs and symptoms already within 12 weeks of treatment with adalimumab. 20% achieved DAS28<2.6 (clinical remission). In addition, there was rapid onset of action. There was also significant improvement in physical function (5% of patients with HAQ <0.5). Efficacy of adalimumab treatment for RA was confirmed, regardless of the number of concomitant DMARDs, the type of concomitant DMARDs, or prior therapy with biologics. This study also showed that adalimumab was safe, as there were few adverse events reported. The study provides an open-label adalimumab trial with “real life” character. Efficacy confirmed in broader clinical practice setting, with various DMARDs and combinations examined, as well as patients who had failed prior biologics. Safety was consistent with pivotal and extension trials, as there were no new alerting signals and the profile was similar to adalimumab used in a routine clinical setting.

### EXAMPLE 2

### Efficacy Evaluation of Adalimumab (HUMIRA) in Patients Switching from Prior Biologic DMARD Therapies

The following study was an open-label, multi-center study (Study A) which was performed to assess the safety and efficacy of the fully human, anti-TNF monoclonal antibody adalimumab when added to insufficient standard antirheumatic therapies in patients with active rheumatoid arthritis. The study design and inclusion criteria are described above in Example 1. Patients available for efficacy analysis included n=1,636 patients with concomitant DMARD information, of which 16% (n=262) had used prior biologics. Prior therapy with biologics at study entry are described above in Table 5. Baseline demographics are described below in Table 6:

TABLE 6

| | Without | With |
|---|---|---|
| | Prior Biologics | Prior Biologics |
| | (n = 1374) | (n = 248*) |
| Gender (% female) | 80 | 81 |
| Age (yrs) | 53 | 52 |
| Disease duration (yrs) | 11 | 12 |

*14 patients with prior biologic experience did not have demographic information available at time of analyses
Mean Values

Baseline disease severity is provided below in Table 7:

TABLE 7

| | Without | With |
|---|---|---|
| | Prior Biologics | Prior Biologics |
| | (n = 1374) | (n = 248*) |
| No. of prior DMARDs | 2.8 | 4.9 |
| DAS28 | 6.0 | 6.3 |
| TJC (0-28 joints) | 13 | 15 |
| SJC (0-28 joints) | 10 | 12 |
| HAQ (0-3) | 1.6 | 1.8 |
| CRP (mg/L) | 26 | 30 |

*14 patients with prior biologic experience did not have demographic information available at time of analyses
Mean Values

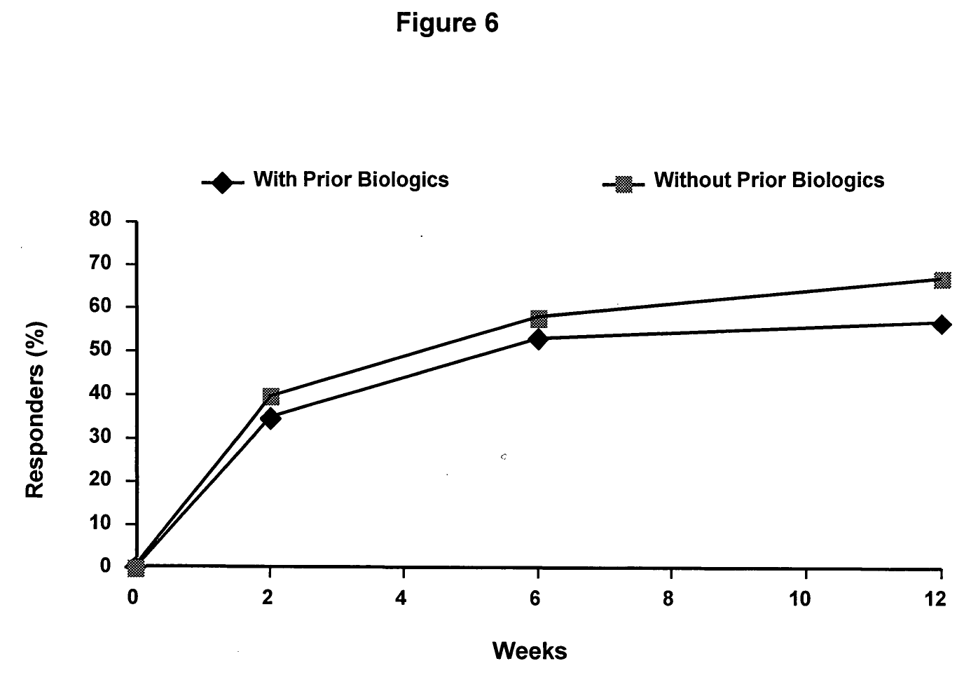

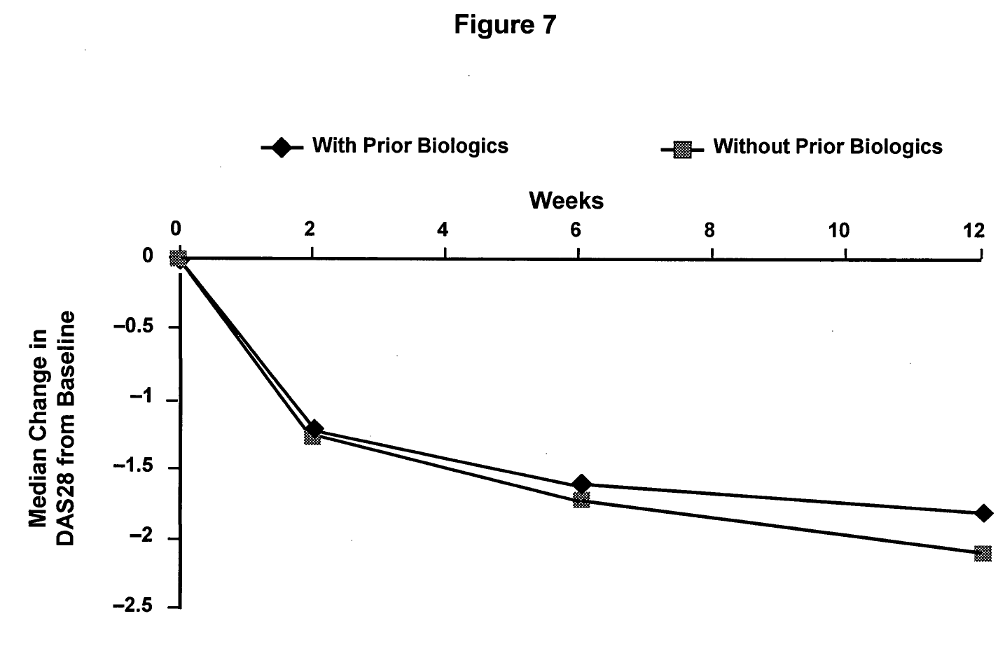

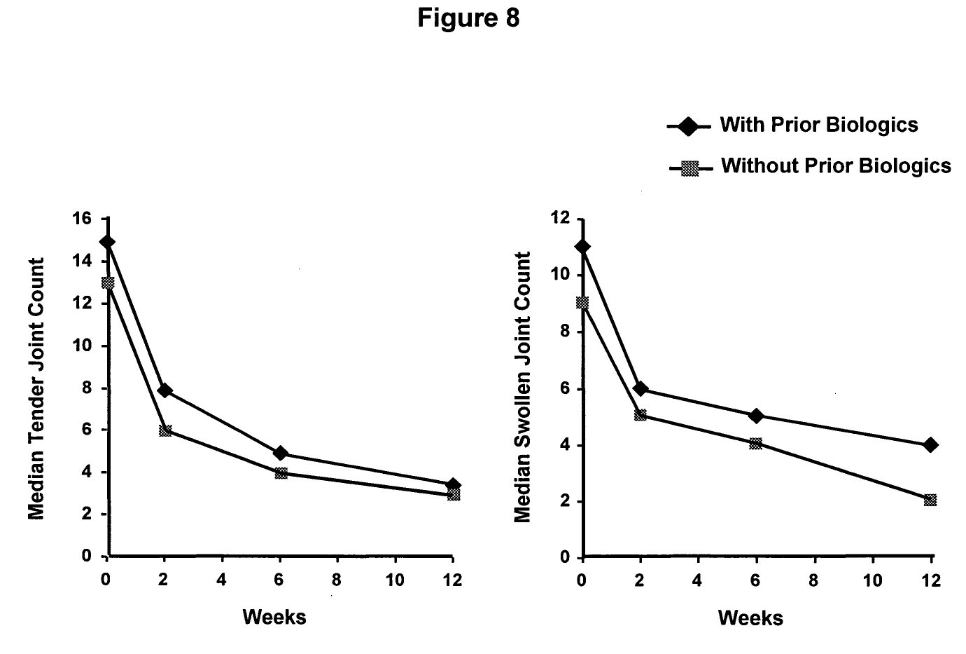

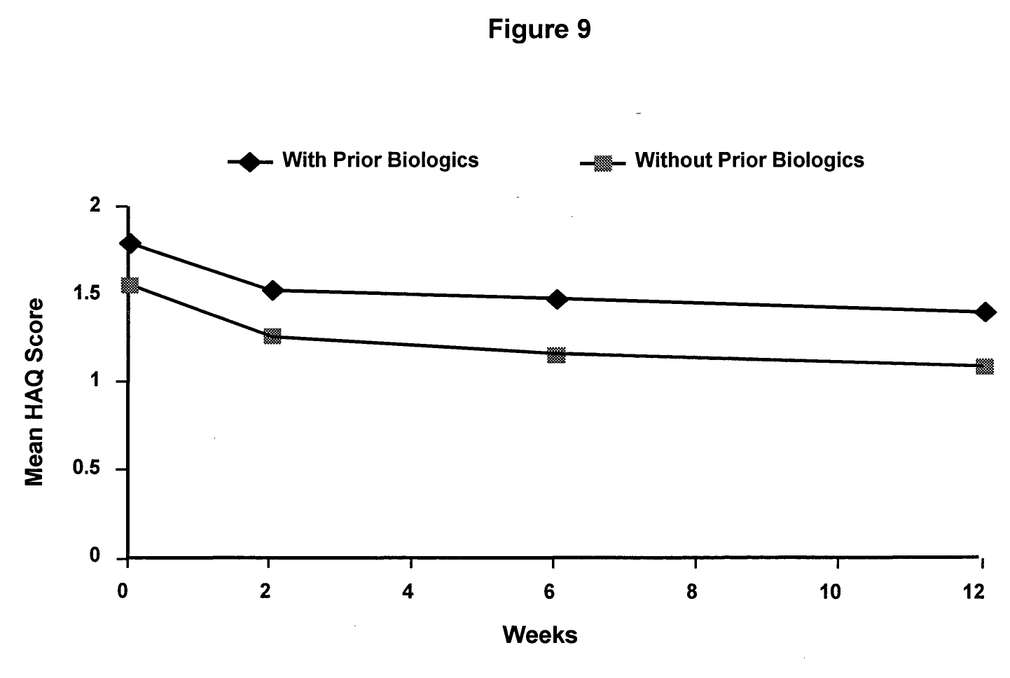

The percentage of responders is shown in FIG. 6, where an improvement in the ACR20 response for both patients w/ and w/o prior biologics was observed. Additional improvements were observed in these patient populations for the DAS28 score (see FIG. 7), the SJC and TJC response (see FIG. 8); and the HAQ response (see FIG. 9). Table 8 describes baseline demographics by number of prior biologics:

TABLE 8

| | None | 1 PB | 2 PB | 3 PB |
|---|---|---|---|---|
| | n = 1374 | n = 203 | n = 46 | n = 13 |
| # Prior DMARDs | 2.8 | 4.5 | 5.7 | 6.7 |
| Pt Assessment DA | 61 | 66 | 65 | 84 |
| SJC | 10 | 12 | 12 | 14 |
| HAQ | 1.6 | 1.8 | 1.7 | 1.9 |
| DAS28 | 6.0 | 6.3 | 6.3 | 7.2 |
| CRP (mg/L) | 26 | 27 | 23 | 53 |

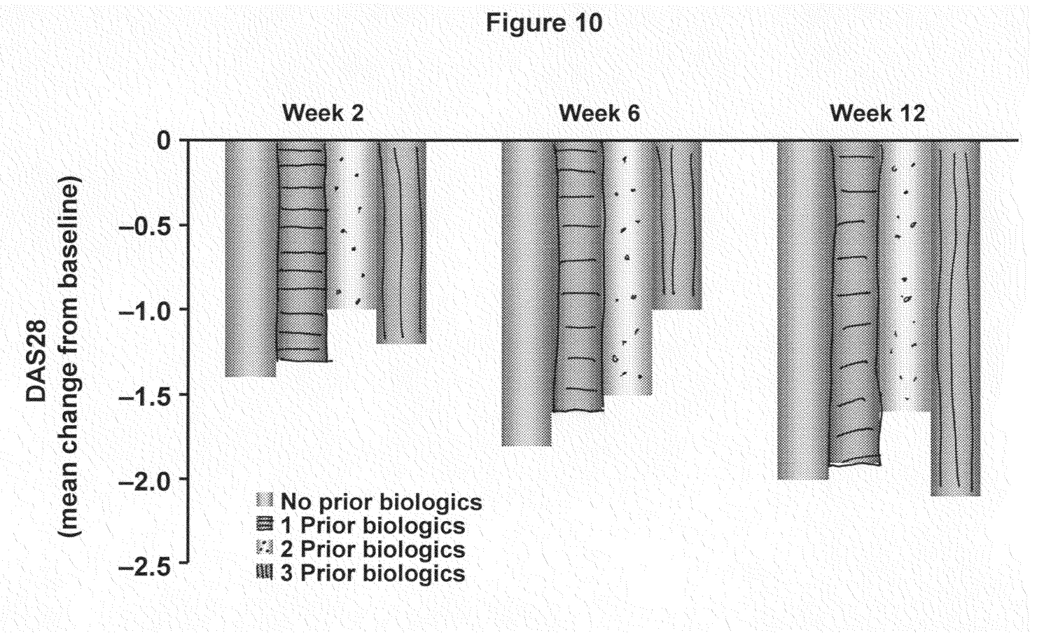

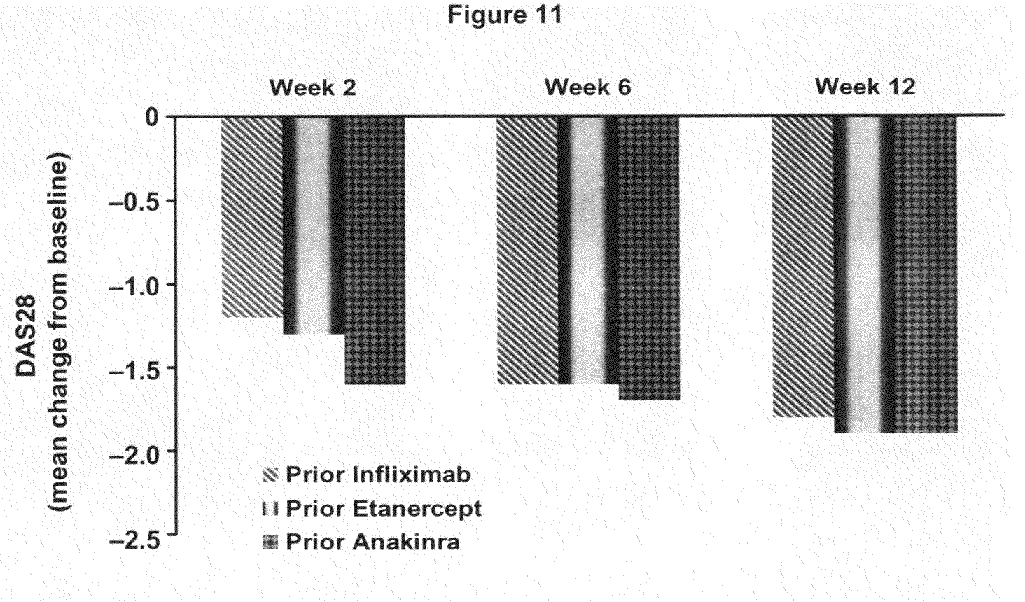

The ACR20 response at week 12 by the number of prior DMARDs was as follows: 67% 0 prior biologic DMARDs (n=1374), 58% 1 prior biologic DMARD (n=203), 54% 2 prior biologic DMARDs (n=46) and 58% 3 prior biologic DMARDs (n=13). The percentage of patients who achieved an ACR50 response was as follows: 39% 0 prior biologic DMARDs (n=1374), 30% 1 prior biologic DMARD (n=203), 26% 2 prior biologic DMARDs (n=46) and 33% 3 prior biologic DMARDs (n=13). FIG. 10 shows the DAS28 improvement by number of biologic. Table 9 describes the response by type of biologic, and FIG. 11 describes the DAS28 improvement by type of biologic.

TABLE 9
Improvement in adalimumab patients according
to type of prior TNF inhibitor

| | | | | Moderate | |
|---|---|---|---|---|---|
| | | ACR20 | ACR50 | EULAR | Good EULAR |
| % of | Infliximab | 61 | 31 | 80 | 22 |
| patients | (n = 114) | | | | |
| | Etanercept | 51 | 30 | 76 | 25 |
| | (n = 56) | | | | |
| | Anakinra | 69 | 25 | 75 | 36 |
| | (n = 33) | | | | |

Additional data relating to clinical response and prior biologic use is described below in Table 10.

TABLE 10
Clinical response by number and type of prior biologic

| Efficacy | 0 | 1 | 2 | 3 | INF only | ETA only |
|---|---|---|---|---|---|---|
| Criteria | (n = 1374) | (n = 203) | (n = 46) | (n = 13) | (n = 114) | (n = 56) |
| ACR20 (%) | 67 | 58 | 54 | 58 | 61 | 51 |
| ACR50 (%) | 39 | 30 | 26 | 33 | 31 | 30 |
| Moderate EULAR | 81 | 78 | 76 | 75 | 80 | 76 |
| response (%) | | | | | | |
| Good EULAR | 34 | 25 | 11 | 8 | 22 | 24 |
| response (%) | | | | | | |
| DAS28* | −2.0 | −1.9 | −1.6 | −2.1 | −1.8 | −1.9 |
| TJC* (0-28) | −8.1 | −8.0 | −8.1 | −11.3 | −7.8 | −8.4 |
| SJC* (0-28) | −6.4 | −6.4 | −6.9 | −6.6 | −6.8 | −6.6 |

*Mean change from baseline, p ≦ 0.01 vs. baseline for all subgroups

In summary, treatment with adalimumab 40 mg every other week showed significant improvements independent of the number and type of prior biologic DMARDs. The addition of adalimumab to insufficient concomitant DMARD therapy provided substantial improvement in the signs and symptoms of RA. Efficacy of adalimumab in real-life clinical practice appears to be identical to efficacy observed in pivotal trials

### EXAMPLE 3

### Three Years of Adalimumab (HUMIRA) Plus Methotrexate Therapy Sustains Radiographic Inhibition of Structural Damage in Patients with Long-Standing Rheumatoid Arthritis

RA is a chronic, autoimmune disease which is characterized by joint inflammation, structural joint damage, extra-articular manifestations, and reduced life expectancy. Adalimumab has been shown to inhibit radiographic progression when used to treat patients with moderate to severe RA. The following study was performed to determine long term radiographic and clinical efficacy, as well as safety, in patients with long-standing RA who had an inadequate response to methotrexate (mtx).

The following study was conducted to assess the sustained response to adalimumab treatment for 3 years. Patients with a confirmed diagnosis of RA who were older than 18 years were eligible for the study. In addition, patients had to have been treated with mtx for at least 3 months prior to enrollment in the study. Stable mtx dose for at least 4 weeks prior screening visit was also required.

In a one year double blind, randomized placebo-controlled trial (RCT), adalimumab plus methotrexate (mtx) was superior to placebo plus mtx in inhibiting structural damage in patients with long-standing RA. The study design included a 12 month, double blind, randomized, placebo-controlled trial. Patients received one of the following regimens: adalimumab 20 mg weekly plus mtx (n=212); adalimumab 30 mg eow plus mtx (n=207); and placebo plus mtx (n=200). Patients who completed the RCT were eligible to enroll in an open label extension (OLE) study, during which they received adalimumab 40 mg eow plus mtx.

Analyses included taking x-rays at baseline (start of RCT), month 12 (end of RCT), and month 36 (post 2 years OLE). Evaluation of all X-rays were performed by 2 readers blinded to sequence of films. Periodic ACR component evaluations were also performed throughout the study. Outcome measures included the following: Total Sharp Score (TSS), including joint erosions (JE) and joint space narrowing (JSN); ACR20, ACR50, and ACR70 response rates; and individual components of ACR responses criteria, including swollen joint count (SJC) and tender joint count (TJC). Efficacy and safety evaluations were conducted at regularly scheduled visits.

619 patients with long-standing RA who had inadequate responses to mtx enrolled in the study, and 467 patients completed the 12-month blinded phase of the study. 457 patients enrolled in the OLE and 363 (79%) completed the 3 year study. Reasons for withdrawal included adverse events (n=31), lack of efficacy (n=11), and other (n=52). Patients entering the blinded and the OLE phase had moderately to severely active RA, and had similar baseline characteristics, as shown in Table 11.

TABLE 11
Baseline patient demographics and disease characteristics

| | Patients | |
|---|---|---|
| | enrolled in 12 month | Patients enrolled in OLE |
| Baseline characteristic | study (n = 619) | (n = 457) |
| Age (yrs) | 57 | 57 |
| % female | 75 | 74 |
| Duration of RA (yrs) | 11 | 11 |
| MTX dose (mg/wk) | 17 | 16 |
| TJC (0-68) | 28 | 28 |
| SJC (0-66) | 19 | 19 |
| HAQ (0-3) | 1.5 | 1.4 |
| CRP (mg/dL) | 1.7 | 1.6 |
| TSS* (0-398) | 68 | 70 |

Mean values at baseline of placebo-controlled trial.
*Based on radiographic readings conducted at the end of the blinded RCT

Of 457 patients enrolled in the OLE (original randomization: 323 adalimumab, 134 placebo), 363 (79%) continued for 2 years. Mean changes for baseline radiographic scores in 129 patients receiving adalimumab 40 mg eow plus mtx in RCT and the OLE demonstrated sustained inhibition of disease progression At one year, 72% of these patients had no radiographic progression (defined as change from baseline in TSS of ≦0.5 units), and, at 3 years, 28% of patients showed radiographic improvement (defined as change from baseline in TSS of ≦−0.5 units) (see Table 12).

TABLE 12
Mean change from baseline in 129 patients treated* for 3 years

| Radiographic assessment | At year 1 | At year 3 |
|---|---|---|
| Total Sharp Score | 0.0 | 0.3 |
| Joint erosion score | 0.0 | 0.1 |
| Joint space narrowings score | 0.0 | 0.2 |

*adalimumab 40 mg eow plus mtx

For three years, adalimumab plus mtx was shown to control radiographic progression. The mean change from baseline in the total sharp score (TSS) over the three years for patients taking ada 40 mg eow and mtx was 0.0 at 12 months (blinded) and 0.3 at 36 months (open-label) vs. 2.8 at 12 months and 3.0 at 36 months for the placebo+mtx group. The mean change from baseline in the joint erosion (JE) score over the three years for patients taking ada 40 mg eow and mtx was 0.0 at 12 months (blinded) and 0.1 at 36 months (open-label) vs. 1.7 at 12 months and 1.7 at 36 months for the placebo+mtx group. In addition, the mean change from baseline in the joint space narrowing (JSN) score over the three years for patients taking ada 40 mg eow and mtx was 0.0 at 12 months (blinded) and 0.2 at 36 months (open-label) vs. 1.2 at 12 months and 1.3 at 36 months for the placebo+mtx group. TSS, JE, and JSN results for patients initially randomized to 20 mg weekly were similar to the results for patients initially randomized to 40 mg eow.

No radiographic progression was defined as ≦0.5 units increase from baseline. The percentage of patients with no radiographic progression following 3 years of adalimumab therapy included the following: 62% total sharp score, 71% joint erosion score, and 73% joint space narrowing score (including only patients who were initially randomized to adalimumab 40 mg eow).

Radiographic improvement was defined as a change from baseline of less than −0.5 units. The percentage of patients with radiographic improvement following three years of adalimumab therapy included the following: 28% total sharp score, 29% joint erosion score, and 20% joint space narrowing score (including only patients who were initially randomized to adalimumab 40 mg eow). Tender and swollen mean joint counts decreased steadily during the 3 year study in the patient population receiving adalimumab, where at the last visit 21% of patients had 0 tender joints and 22% of patients had 0 swollen joints (observed values in all patients who received ada (by duration of treatment with adalimumab+mtx).

In addition ACR responses were as follows: at month 12, 69% of patients had ACR20, 48% of patients had ACR50, and 26% of patients had ACR70. At month 24, 63% of patients had ACR20, 43% of patients had ACR50, and 28% of patients had ACR70. At month 36, 58% of patients had ACR20, 42 of patients had ACR50, and 24% of patients had ACR70.

Significant clinical responses were sustained over time, with ACR20/50/70 response rates of 58/42/23% seen in patients who had received adalimumab for 3 years. Patients randomized to placebo in the RCT had significant disease progression at year 1. After these patients were treated during the OLE with adalimumab, progression was inhibited and clinical responses improved comparably. No unexpected safety events were seen in the OLE. Rates and types of adverse events reported were similar to those seen in the RCT, as shown in Table 13.

TABLE 13
Serious adverse events during treatment with adalimumab

| | | Blinded-controlled |
|---|---|---|
| | | and OLE |
| | Blinded-controlled period | periods |
| | 365.7 patient years | 1317.5 patient years |
| Serious Adverse Events | events/100-PY | events/100-PY |
| Serious infections | 4.4 | 4.1 |
| Pneumonia | 1.4 | 1.1 |
| Urinary tract infections | 0.3 | 0.3 |
| Septic arthritis | 0 | 0.2 |
| Tuberculosis | 0.3 | 0.2 |
| Histoplamosis | 0.3 | 0.2 |
| Demyelinating disease | 0.3 | 0.2 |
| Lymphoma | 0.3 | 0.3 |
| SLE/lupus-like syndrome | 0 | 0 |
| pancytopenia | 0.5 | 0.2 |

*rates were obtained using MedDRA coding
OLE = open label extension
100-PY = 100 patient years

In conclusion, adalimumab plus mtx inhibited structural damage of disease progression over 3 years in patients with long-standing RA who previously had an incomplete response to mtx. 62% of patients treated with adalimumab 40 mg plus mtx for 3 years showed no progression in TSS and 71% showed no progression in joint erosions. Adalimumab provided significant, sustained improvement in the signs and symptoms of RA over 3 years of treatment. Adalimumab was well-tolerated. Finally, improvements from baseline in radiographic scores at 3 years suggest that structural repair may be occurring in some patients.

### EXAMPLE 4

### Major Clinical Response and Sustained Remission Over 4 Years in Patients with Rheumatoid Arthritis Treated with Adalimumab (HUMIRA) Plus Methotrexate

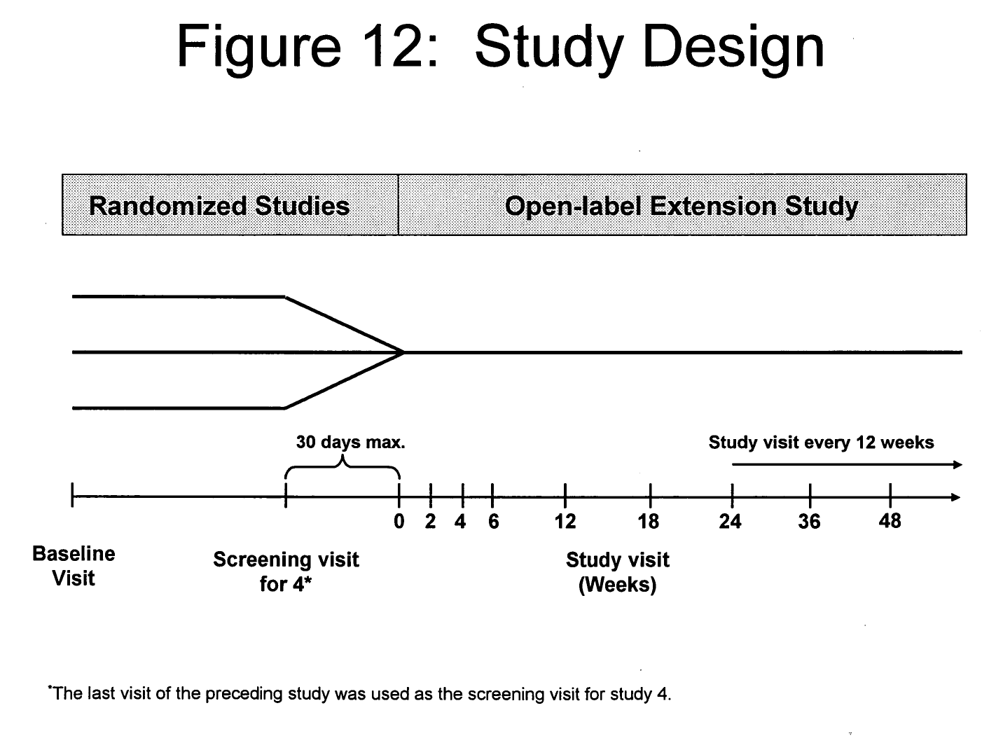

The following study was performed to evaluate sustained remission in patients with RA treated with adalimumab and mtx. The study was an open-label extension study, where the last visit of the preceding study was used as the screening visit for Study 4. The study design for Study 4 is described in FIG. 12. The percentage of patients remaining on treatment over time from the first dose of adalimumab remained relatively constant. 846 patients enrolled over time. At the time of the analysis, 635 patients (75%) remained on therapy and 211 (25%) had withdrawn (6% for lack of efficacy, 9% for adverse reactions, and 10% for other reasons). Baseline demographics and disease characteristics for all patients prior to adalimumab exposure (n=846) are described below in Table 14.

TABLE 14
Baseline demographics

| Characteristic | Baseline |
|---|---|
| Age (yrs) | 55 |
| % female | 78 |
| Disease Duration of RA (months) | 99 |
| TJC (0-28)* | 25 |
| SJC (0-28)* | 18 |
| HAQ (0-3) | 1.4 |
| CRP (mg/dL)* | 10.0 |
| DAS28 | 5.7 |

Median values (except % female)
*TJC/SJC baseline data available for n = 746; CRP baseline data available for n = 739

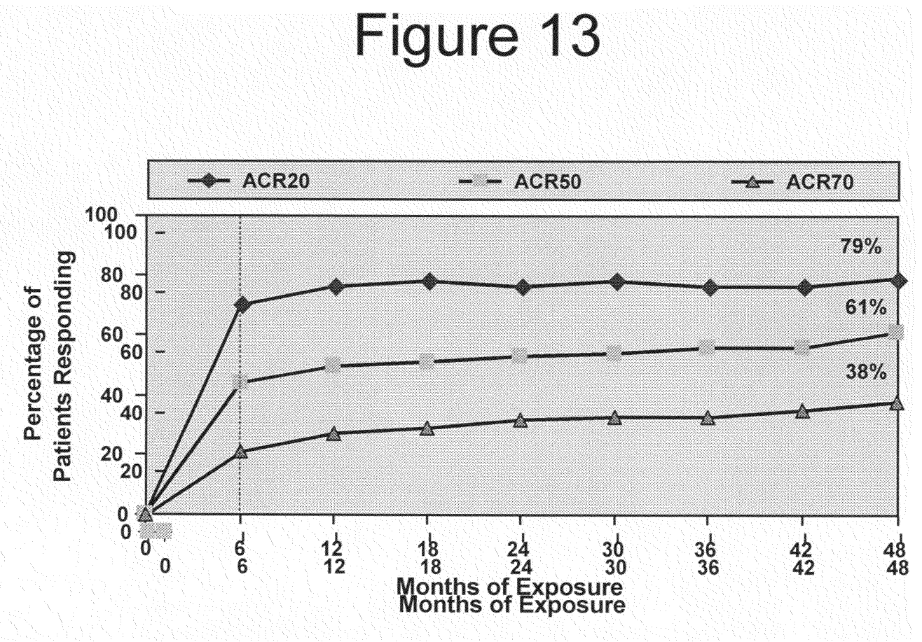

ACR response rates and Major Clinical Response (MCR) remained constant through the fourth year of adalimumab treatment. Major Clinical Response (MCR) was defined as patients maintaining an ACR70 response for at least 6 continuous months over a 2-year period. The percentage of patients who maintained ACR responses and MCR through the fourth year of adalimumab treatment is shown in FIG. 13. At the end of the 4 year time period, 79% of patients had maintained an ACR20, 61% maintained an ACR50 response, and 38% maintained an ACR 70 response. Of the 436 patients who had at least 3 years of exposure to adalimumab, 134 (31%) achieved MCR.

Percentage change of mean clinical improvement from baseline us describe in Table 15 below.

TABLE 15
% change of mean clinical improvement from baseline*

| | Months of exposure | | | | |
|---|---|---|---|---|---|
| Criteria | 6 | 12 | 24 | 36 | 48 |
| N | 742 | 539 | 661 | 398 | 101 |
| DAS28 | 39 | 44 | 45 | 47 | 50 |
| TJC (0-68) | 64 | 69 | 72 | 75 | 78 |
| SJC (0-66) | 58 | 67 | 69 | 68 | 61 |
| CRP | 14 | 33 | 29 | 28 | 37 |
| HAQ | 45 | 43 | 47 | 47 | 50 |

*All values significant at p ≦ 0.001 vs. baseline except CRP at 6 months

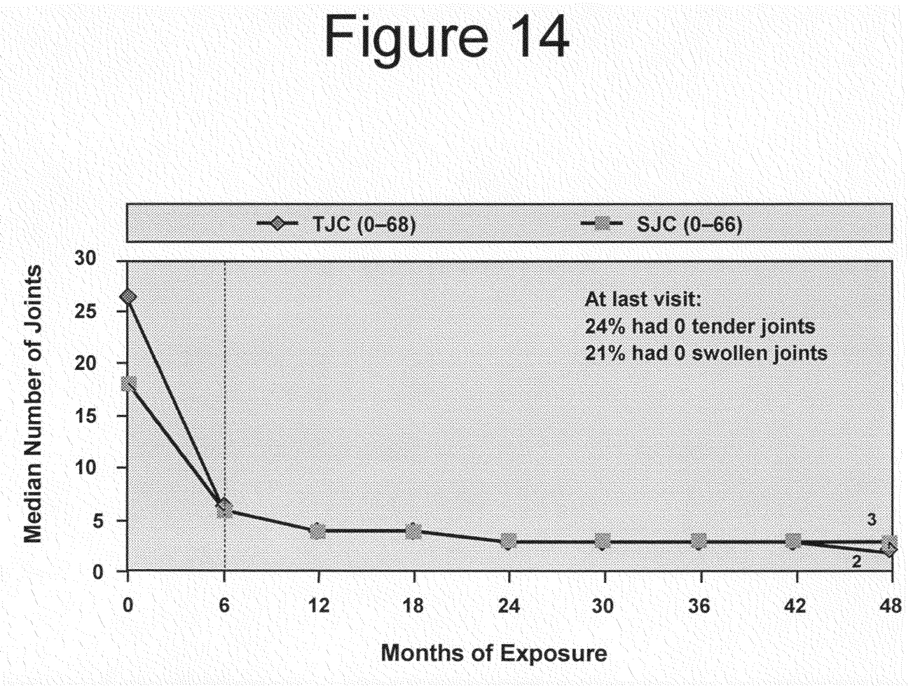

Tender joint count (TJC) (0-68) and swollen joint count (SJC) (0-66) scores through the fourth year of adalimumab treatment are shown in FIG. 14. At the last visit, 24% of participants had 0 tender joints and 21% of participants had 0 swollen joints. The median number of tender joints for patients taking adalimumab at the end of the four years was 2, and the median number of swollen joints was 3.

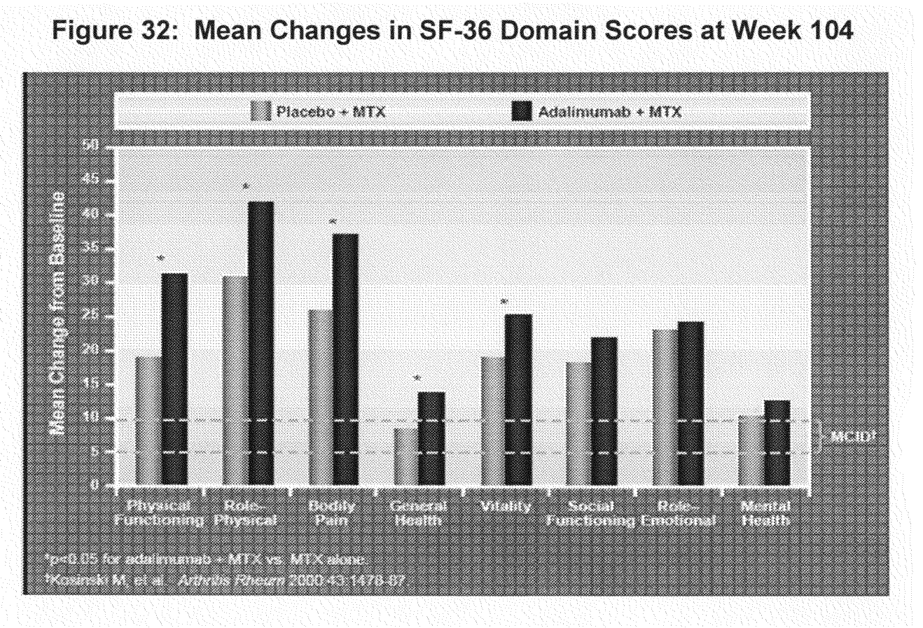

DAS28 scores were also maintained at a low score through the fourth year of adalimumab treatment, as shown in FIG. 32. DAS28 represents disease activity, taking into consideration the TJC 28, SJC 28, CRP, and patient general health (based on VAS of 100 mm) (see Prevoo et al. (1995) Arthritis Rheum 38:44-48 and Van Gestel et al. (1999) J Rheumatol 26:705-711, each of which is incorporated by reference herein). DAS28 scores greater than or equal to 5.1 represent severe activity disease activity, less than 5.1 to 3.2 indicate moderate disease activity, less than 3.2 to 2.6 indicate low disease activity, and scores below 2.6 represent remission. At the end of the fourth year, the median DAS28 score was 2.7 (low disease activity). In addition, clinical remission based on a DAS28<2.6 was observed in 49% of patients at the end of the 48 month period. The largest increase in percentage of patients in clinical remission was seen between months 0-6, where an increase in the percentage of patients in remission went from 0% at month 0 to about 27% at month 6. Over the 48 month period, the percentage of patients in remission (DAS28<2.6) increased steadily to 49%.

The rates of sustained remission and time to remission were consistent across durations of the disease. Remission rates were defined as greater than or equal to 6 months by baseline disease duration. Rates of sustained remission and time to remission are described below in Table 16.

TABLE 16

| | Disease Duration at Study Entry | |
|---|---|---|
| | ≦2 years | >2 years |
| Criteria | (n = 36, 31%*) | (n = 209, 29%*) |
| Baseline | | |
| Age (yrs) | 52 | 54 |
| Disease Dur (months) | 11 | 150 |
| DAS28 | 5.8 | 5.4 |
| TJC (0-28) | 16 | 13 |
| SJC (0-28) | 13 | 12 |
| CRP concentration (mg/L) | 21 | 16 |
| Morning stiffness (min) | 111 | 79 |
| Months until remission | 10 | 10 |
| Months in remission | 25 | 25 |
| During remission | | |
| % of visits with ACR70 | 71 | 64 |
| % of visits with TJC = 0 | 77 | 70 |
| % of visits with SJC = 0 | 64 | 54 |
| % of visits with CRP <10 mg/L | 94 | 95 |

Mean values
*% of all patients with disease duration that falls in this category

Remission rates and time to remission depended on baseline disease severity, as shown in Table 17. Remission rates were defined as greater than or equal to 6 months by baseline disease duration.

TABLE 17

| | DAS28 < 5.1 | DAS28 ≧ 5.1 |
|---|---|---|
| Criteria | (n = 105, 50%*) | (n = 140, 22%**) |
| Baseline | | |
| DAS28 | 5.0 | 6.0 |
| TJC (0-28) | 9 | 17 |
| SJC (0-28) | 9 | 14 |
| CRP concentration (mg/L) | 8 | 23 |
| Months until remission | 8 | 11 |
| Months in remission | 27 | 23 |

Mean values
*% of all patients with DAS28<5.1
**% of all patients with DAS28≧5.1

In addition, the median HAQ disability index score through the fourth year of adalimumab treatment was 0.5.

Treatment with adalimumab in RA patients also served to decrease the dosing of concomitant corticosteroids. The mean corticosteroid dose (mg/day) at baseline was 5.56, where the mean corticosteroid dose (mg/day) at the final dose was 3.57 (p<0.0001 (determined by Wilcoxon's signed rank test)). For patients taking concomitant corticosteroids (n=294 (patients on systemic steroids at any time during the study), and 24% were able to discontinue steroids, 29% had a dose reduction of steroids. For 40% of the patients, the dose did not change, and for only 6% the dose increased.

There was also a change in the dosing of concomitant methotrexate for RA patients in the study being treated with adalimumab. The mean mtx dose (mg/week) for patients at baseline was 15.79, where at the final dose of adalimumab the mean was 13.81 (p<0.0001 (determined by Wilcoxon's signed rank test)). For patients taking concomitant mtx (n=546, patients on mtx from their first dose of adalimumab), 32% saw a dose reduction, and for 60% of the patients the dose of mtx did not change. For only 8% of patients was the dose of mtx increased.

Serious adverse events observed in all patients treated with adalimumab are described in Table 18 below.

TABLE 18
Adverse events: events per 100 patient years (E/100-PY)

| | Pivotal trials | All exposure |
|---|---|---|
| | 793 patients-years | 2485 patient-years |
| Serious Adverse Events | E/100-PY | E/100-PY |
| Serious infections | 4.16 | 2.62 |
| Pneumonia | 1.13 | 0.6 |
| Urinary tract infections | 0.5 | 0.28 |
| Septic arthritis | 0.38 | 0.12 |
| Tuberculosis | 0.13 | 0.04 |
| Histoplasmosis | 0.13 | 0.00 |
| Demyelinating disease | 0.13 | 0.04 |
| Lymphoma | 0.25 | 0.16 |
| SLE/lupus-like syndrome | 0.13 | 0.00 |
| Pancytopenia | 0.25 | 0.00 |

In sum, patients with long-standing RA maintained clinical improvements and experienced low or no disease activity over 4 years. Adalimumab+MTX induced remission (DAS28<2.6) in ⅓ of patients, usually in the first year of treatment. One third (about 30%) of patients in remission were able to reduce their MTX doses and maintain remission, and one third (about 30%) of patients achieved MCR (ACR70≧6 mos). The majority of patients were able to reduce their use of steroids and MTX when treated with adalimumab, while maintaining disease control. In addition, Adalimumab was safe and well-tolerated over 4 years of therapy, as no new safety signals were observed.

### EXAMPLE 5

### Low-Field Magnetic Resonance Imaging for Follow-Up Analysis of Finger Joint Inflammation in Patients with Active Rheumatoid Arthritis Receiving Adalimumab

The objective of this study was to demonstrate the value of low-field magnetic resonance imaging (MRI) in the follow-up evaluation of patients with active rheumatoid arthritis (RA) receiving adalimumab, a fully human monoclonal antibody targeted against tumor necrosis factor.

The method included the following. Selected patients with active RA who received adalimumab 40 mg every other week in Study A were evaluated. Proximal interphalangeal and metacarpophalangeal joints of the dominant hands of these patients were examined by 0.2-Tesla MRI (C-Scan, Esaote, Italy). Clinical examinations and MRI were performed at baseline and during therapy for a mean period of 86 days (range: 42-126 days). The used sequences were coronal T1-weighted spin-echo; coronal short-tau inversion-recovery gradient-echo; and T1-weighted, 3D gradient-echo before and after administration of Gd-DTPA (0.2 mmol/kg body weight). Erythrocyte sedimentation rate (ESR) and Disease Activity Score 28 (DAS28) were measured at all time points. MRI was evaluated according to an Outcome Measures in Arthritis Clinical Trials or OMERACT-based semiquantitative scoring system. Scores were calculated for erosions (0-5 scale, maximum of 40), synovitis (0-3 scale, maximum of 24), and tenosynovitis (0-3 scale, maximum of 12).

The results from the study included the following. Sixteen patients (mean age 50.1 years; mean disease duration 7.2 years) were evaluated. Nine of the 16 patients received concomitant methotrexate. There was good correlation between clinical response and MRI findings. During adalimumab therapy, decreases in the median synovitis score (from 11 to 9, p<0.05) and median tenosynovitis score (from 3 to 1.5, p<0.05) were evident. As expected, because of a short observation period, the median erosion score did not decrease significantly (from 6.5 to 4.5). Mean ESR declined from 33.0 before therapy to 16.2 mm/hr after therapy (p<0.05). In addition, mean DAS28 decreased from 6.1 to 3.8 (p<0.05), and there was positive correlation between synovitis and DAS28 scores (R²=0.67, p<0.05).

In conclusion, standardized, contrast-enhanced, low-field MRI showed significant reductions in synovitis and tenosynovitis of small finger joints in RA patients treated with adalimumab. Therefore, low-field MRI is a suitable, objective outcome measure for clinical trials in rheumatology.

### EXAMPLE 6

### Effects of Adalimumab Monotherapy on Health Utility and Fatigue in Patients with Long-Standing, Severe Rheumatoid Arthritis (RA)

Clinical trials of tumor necrosis factor (TNF) antagonists in the treatment of rheumatoid arthritis (RA) routinely evaluate effects on quality of life. Rarely is health utility and fatigue assessed. Although optimal use of these biologics is with methotrexate (MTX), some patients are unable to take or do not benefit from MTX. This analysis investigated whether monotherapy with adalimumab (Humira®), the fully human, anti-TNF monoclonal antibody, increases quality of life and health utility, and/or reduces fatigue compared with placebo in patients with long-standing, severe rheumatoid arthritis (RA) who had failed MTX therapy.

### Methods

A health economics companion study to the placebo-controlled, pivotal trial was conducted in which patients with severe RA who had failed MTX received adalimumab 40 mg every other week or placebo without concomitant disease-modifying antirheumatic drugs (DMARD) for 26 weeks. In addition to the SF-36 instrument, the Health Utilities Index Mark 3 (HUI3) and the Functional Assessment of Chronic Illness Therapy-Fatigue (FACIT-F) questionnaires were administered at baseline, 12 weeks, and the end of the study.

The HUI3 scale is 0-1, with “1” denoting perfect health and “0” denoting death. FACIT-F scores range from 0-52, with higher scores representing less fatigue. The SF-36 includes one multi-item scale that assesses eight different dimensions of health, including physical and psychosocial components. For each dimension a value of 0-100 is given, with higher numbers representing less impairment. Changes in HUI3 of ≧0.03, in FACIT-F of ≧4 and in SF-36 of ≧4.4 (physical subscales) and ≧3.1 (mental subscales) are considered clinically meaningful.

### Results

Baseline characteristics were indicative of long-standing, severe RA: age: 53 yrs; disease duration: 11 yrs; TJC (0-68): 34; HAQ: 1.9, CRP (mg/L): 56.6; previous DMARDs: 4 (mean values).

Baseline utility scores (HUI3) were comparable for adalimumab (n=99) and placebo (n=96) (0.27 and 0.28), and much worse than that of the age- and sex-adjusted population norm (0.88). Similarly, baseline FACIT-F scores were comparable (26.1 and 26.3, respectively) and, again, considerably worse than that of the general population (43.6). The physical subscales of the SF-36 for both groups were particularly low (ranging from 11 to 41 and 14 to 40, respectively) representing the character of a long-standing RA cohort.

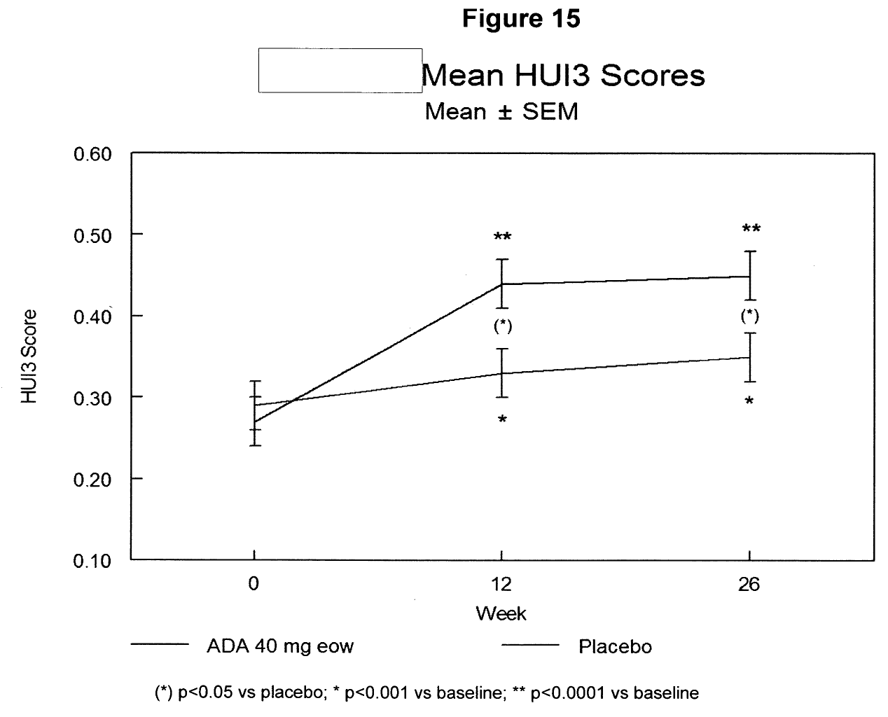

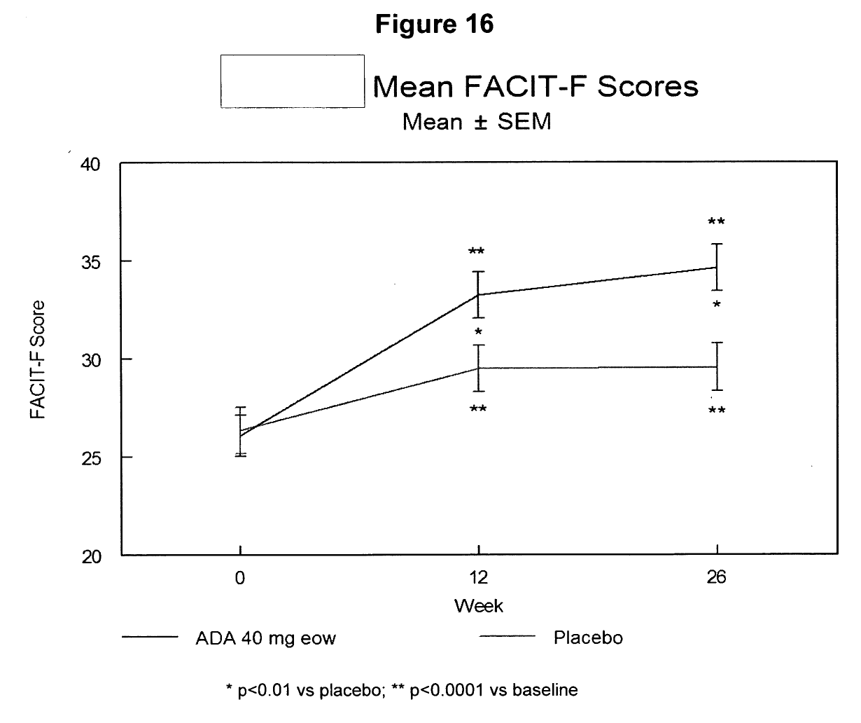

After 26 weeks, mean HUI3 scores increased 0.18 from baseline for adalimumab compared with 0.08 for placebo (p<0.05) (as shown in FIG. 15). Mean FACIT-F scores increased 8.7 for adalimumab compared with 3.3 for placebo (p<0.01) (as shown in FIG. 16). For both scores, HUI and FACIT-F, rapid and statistically significant improvements were seen after 12 weeks and were maintained throughout the trial.

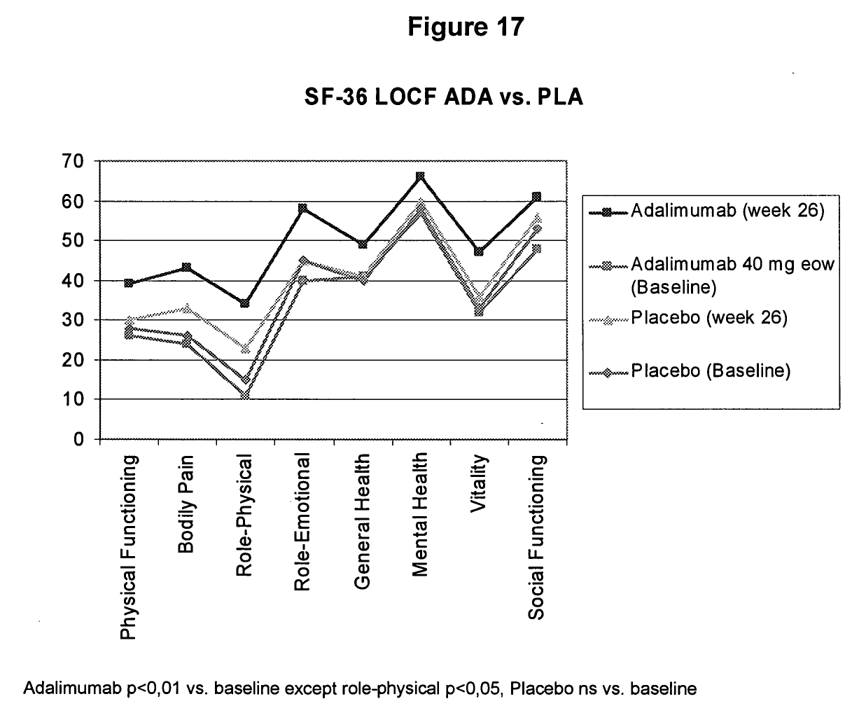

With adalimumab, SF-36 scores of all subscales showed clinically meaningful improvements, whereas placebo remained the same (as shown in FIG. 17). All reported changes were statistically significant.

### Conclusion

Adalimumab therapy without concomitant MTX provided statistically significant and clinically meaningful improvements in health utility and fatigue, as well as quality of life, for patients with severe, long-standing RA who had failed MTX therapy. This effect now has to be evaluated to determine whether these promising effects, with their potential to impact costs (direct and indirect, as well as intangible), are sustained during long-term treatment.

### EXAMPLE 7

### The Efficacy and Safety of Adalimumab (HUMIRA®) Plus Methotrexate vs. Adalimumab Alone or Methotrexate Alone in the Early Treatment of Rheumatoid Arthritis (RA): 1- and 2-Year Results

Early, aggressive intervention in patients with rheumatoid arthritis (RA) appears to provide the most favorable clinical and radiographic outcomes, but the best approach toward achieving this goal is uncertain. This study was designed to directly compare the safety and efficacy of adalimumab plus methotrexate (MTX) vs. either MTX alone or adalimumab alone as first-line therapy in patients with recent-onset RA (Breedveld et al. Ann Rheum Dis 2005; 64(Suppl III):60).

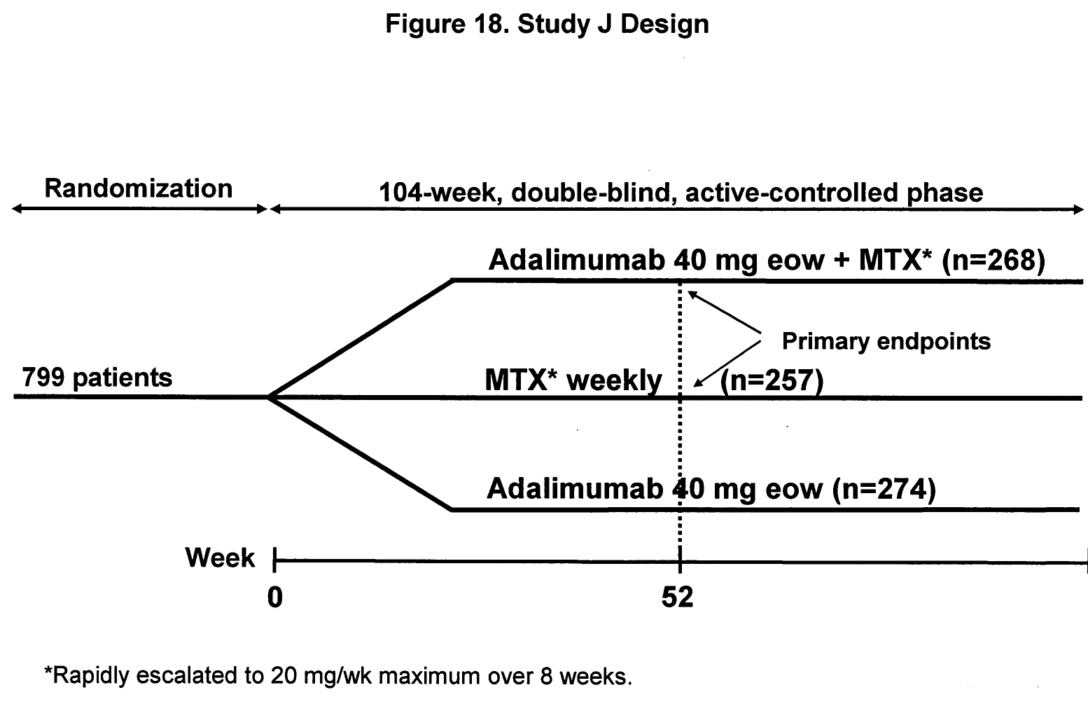

This study was a 2-year, double-blind, active-comparator-controlled Phase III study conducted at 149 sites in North America, Europe, and Australia. FIG. 18 shows the design of this study (Study J). MTX-naïve adult patients with active, early RA (<3 years) were included in the study. Further inclusion criteria included: SJC≧8 and TJC≧10; ESR≧28 mm/hr or CRP≧1.5 mg/dL; and RF-positivity or at least one joint erosion. Patients were randomized to 1 of 3 treatment arms: adalimumab 40 mg every other week (eow)+MTX; adalimumab 40 mg eow; or MTX alone. MTX dosages were rapidly optimized to a maximum of 20 mg weekly.

The co-primary endpoints, comparing MTX alone with the combination at 1 year, were the ACR50 response (using non-responder imputation), and inhibition of radiographic progression, as measured by change in total Sharp score (TSS). In this study, the term “Major Clinical Response” (MCR) meant a continuous ACR70 response for 6 or more consecutive months.

A total of 799 patients enrolled in the study. Baseline characteristics were similar across the 3 arms. The baseline characteristics of the patients included in this study are shown in Tables 19 and 20.

TABLE 19
Baseline characteristics

| | Ada + mtx (n = 268) | Ada alone (n = 274) |
|---|---|---|
| Age (yrs) mean | 52 | 52 |
| % female | 72 | 77 |
| Disease duration (yr) mean | 0.7 | 0.7 |
| % with prior DMARDs | 33 | 33 |
| % corticosteroid use | 36 | 37 |
| % RF-positive | 87 | 83 |

TABLE 20
Baseline characteristics

| | Ada + mtx | Ada alone | mtx alone |
|---|---|---|---|
| | (n = 268) | (n = 274) | (n = 257) |
| SJC (0-68), mean | 23 | 24 | 24 |
| TJC (0-68), mean | 33 | 34 | 34 |
| HAQ, mean | 1.5 | 1.6 | 1.5 |
| DAS28, mean | 6.3 | 6.4 | 6.3 |
| CRP (mg/dL) | 4.7 | 5.0 | 4.6 |
| TSS, mean | 18.1 | 18.8 | 21.9 |
| TSS/duration of RA | 25.6 | 26.7 | 27.4 |
| % with joint erosions | 93% | 94% | 96% |

Mean age was 52 years and mean duration of RA was 0.7 years. DAS28 was 6.3, HAQ was 1.5, TSS was 19.5, and CRP was 4.8 mg/dL (all mean values). In addition, 75% of patients were female, and 83% were RF+. A total of 539 patients (68%) completed 2 years of therapy, including 66% of patients in the MTX arm, 61% in the adalimumab arm, and 76% in the combination arm. Reasons for withdrawal included lack of efficacy (13.9%), adverse events (9.6%), and miscellaneous causes (8.5%). Table 21 shows the disposition of the subjects at 2 years.

TABLE 21
Subject disposition at 2 years

| | Ada + mtx | Ada alone | mtx alone |
|---|---|---|---|
| | (n = 268) | (n = 274) | (n = 257) |
| Completed | 76% | 61% | 66% |
| Withdrawn | 24% | 39% | 34% |
| Reason: | | | |
| AE | 12% | 11% | 8% |
| Lack of efficacy | 5% | 19% | 18% |
| Other | 7% | 9% | 9% |

Outcomes in patients receiving combination therapy were uniformly better than those in patients receiving either monotherapy. Compared with MTX, ACR20/50/70 response rates were statistically significantly greater in the combination arm at 2 weeks, and these differences were sustained throughout the 2 years of the study. The frequencies of adverse events were comparable among all 3 arms.

The primary endpoint for the study was an ACR50 response at Week 52.62% of patients in the ada+mtx group achieved an ACR50 at week 52 vs. 46% of mtx alone group (p<0.001 vs. mtx alone) (non-responder imputation: patients who discontinued were considered non-responders). ACR 20/50/70 at Weeks 52 and 104 are described below in Table 22.

TABLE 22
ACR20/50/70 at weeks 52 and 104 (% of patients)

| | Ada + mtx | Ada alone | mtx alone |
|---|---|---|---|
| ACR20 | 73* | 54 | 63# |
| Week 52 | | | |
| ACR50 | 62* | 42 | 46 |
| ACR70 | 46* | 26 | 28 |
| Week 104 | | | |
| ACR20 | 69* | 50 | 56 |
| ACR50 | 59* | 37 | 43 |
| ACR70 | 47* | 28 | 28 |

Non-responder imputation
*p < 0.001 for adalimumab + mtx vs. ada alone and mtx alone
#p < 0.05 for mtx alone vs. ada alone, others not significant

The mean change in TSS at Week 52 (mean change from baseline) for patients receiving adalimumab and MTX was 1.3* vs. 5.7 for patients receiving MTX alone (*p<0.001 vs. mtx alone). Table 23 shows the change in TSS for all three treatment arms over the course of the study.

TABLE 23
Mean change from baseline TSS for all three treatment arms

| | Ada + mtx | Ada alone | mtx alone |
|---|---|---|---|
| Week 0 | 0 | 0 | 0 |
| Week 26 | 0.8* | 2.1** | 35 |
| Week 52 | 1.3* | 3** | 57 |
| Week 104 | 1.9* | 5.5** | 10.4 |

*p < 0.001 for ada + mtx vs. ada alone and mtx alone
**p < 0.001 for ada alone vs. mtx alone

61% of patients receiving adalimumab and MTX*, 45% of those receiving only adalimumab**, and 34% of those receiving only MTX had no radiographic progression, which was defined as a change in TSS of ≦0.5 (*p<0.01 for ada+mtx vs. ada alone and mtx alone; ** p<0.01 for ada alone vs. mtx alone).

The ACR response and radiographic progression of each treatment arm of the study is shown below in Table 24.

TABLE 24
ACR response and radiographic progression

| | | Ada + mtx | Ada alone | mtx alone |
|---|---|---|---|---|
| Mean | ACR20 | 1.1 | 4.5 | 5.5 |
| change in | responder | | | |
| TSS from | ACR50 | 1.0 | 3.4 | 4.9 |
| baseline | responder | | | |
| | ACR70 | 0.7 | 3.5 | 4.1 |
| | responder | | | |

Clinical remission, defined as DAS28<2.6, is described below in Table 25.

TABLE 25
Clinical remission by DAS28 <2.6

| | | Ada + mtx | Ada alone | Mtx alone |
|---|---|---|---|---|
| % of | Week 52 | 43* | 23 | 21 |
| patients | Week 104 | 49* | 25 | 25 |

*p < 0.001 vs. ada alone or mtx alone

In addition, 49% of those patients in the adalimumab and MTX combination treatment arm* had a major clinical response (defined by the FDA as patients achieving and maintaining ACR70 response for ≧6 continuous months over 2 years) vs. 25% for ada alone and 27% mtx alone (*p<0.001 vs. ada alone or mtx alone).

ACR90 response rates for patients in each of the three treatment arms of the study are shown in Table 26.

TABLE 26
ACR90 response rates

| | | Ada + mtx | Ada alone | Mtx alone |
|---|---|---|---|---|
| % of | Week 52 | 24* | 8 | 13 |
| patients | Week 104 | 27* | 9 | 13 |

*p < 0.001 vs. ada alone or mtx alone

The individual clinical remission criteria as shown by TJC=0, SJC=0 HAQ=0, and AM Stiffness=0 is shown in Table 27.

TABLE 27
Individual clinical remission criteria

| | | Ada + mtx | Ada alone | Mtx alone |
|---|---|---|---|---|
| % of | TJC = 0 | 36** | 15 | 21 |
| patients | SJC = 0 | 39* | 19 | 19 |
| | HAQ = 0 | 33* | 19 | 19 |
| | AM | 53* | 30 | 30 |
| | stiffeness = 0 | | | |

*p < 0.001 vs. ada alone or mtx alone;
**p < 0.05 vs. either monotherapy

Table 28 summarizes the key efficacy results from this study at 1 and 2 years.

TABLE 28
Key Efficacy Results at 1 and 2 Years

| | Ada + MTX | Ada + MTX | Ada alone | Ada alone | MTX alone | MTX alone |
|---|---|---|---|---|---|---|
| | 1 year | 2 years | 1 year | 2 years | 1 year | 2 years |
| ACR50 | 62*† | 59* | 42 | 37 | 46 | 43 |
| (% pts) | | | | | | |
| ACR70 | 46* | 47* | 26 | 28 | 28 | 28 |
| (% pts) | | | | | | |
| ACR90 | 24* | 27* | 8 | 9 | 13 | 13 |
| (% pts) | | | | | | |
| MCR | | 49* | | 25 | | 27 |
| (% pts) | | | | | | |
| DAS28 < 2.6 | 43* | 49* | 23 | 25 | 21 | 25 |
| (% pts) | | | | | | |
| Δ TSS | 1.3*† | 1.9* | 3.0§ | 5.5§ | 5.7 | 10.4 |
| (Mean) | | | | | | |

†Co-prim. endpts.;
*p < 0.001 vs. MTX alone/Ada. alone,
§p < 0.001 vs. MTX alone.

Table 29 summarizes the treatment emergent adverse events that occurred in this study.

TABLE 29

| | Adalimumab + | Adalimumab | MTX |
|---|---|---|---|
| | MTX | Alone | Alone |
| | n = 268 | n = 274 | n = 257 |
| | PYs = 482 | PYs = 435 | PYs = 429 |
| | Events/100-PY | Events/100-PY | Events/100-PY |
| All Infectious AE | 123 | 110 | 119 |
| Serious AE | 18.5 | 21.1 | 15.9 |
| Serious Infectious AE | 2.9* | 0.7 | 1.6 |
| Tuberculosis | 0.2 | 0.0 | 0.0 |
| Malignancies | 0.4 | 0.9 | 0.9 |
| Lymphoma | 0.0 | 0.0 | 0.2 |
| Pancytopenia | 0.0 | 0.0 | 0.2 |
| Demyelination | 0.0 | 0.0 | 0.0 |

*p < 0.05 for adalimumab + MTX vs. adalimumab alone.
PY = patient-years.

Overall, in MTX-naïve patients with recent-onset RA, adalimumab plus MTX was statistically significantly better than either MTX alone or adalimumab alone in alleviating the signs and symptoms of RA and in inhibiting radiographic progression. Remission at 2 years, as measured by DAS28 and major clinical response, was achieved by approximately half of patients receiving combination therapy. Further, at two years, twice as many patients on adalimumab and MTX had no radiographic progression compared with MTX alone. AU treatments were generally safe and well tolerated.

### EXAMPLE 8

### RA Patients in 4 Adalimumab Clinical Trials: 78% have DAS of 5.1 or More, and 90% have a Score of 7 or More on a Continuous Index of 3 Patient Questionnaire Scores for Physical Function, Pain and Global Status

A Disease Activity Score 28 (DAS28) of ≧5.1 is regarded as indicating severe rheumatoid arthritis (RA). In several countries, patients with RA are required to have a DAS28≧5.1, indicative of severe disease, in order to receive tumor necrosis factor (TNF) antagonists. This study analyzed 1,391 patients with RA from 4 clinical trials to determine the percentages of patients who had DAS28≧5.1 or DAS28<5.1, and the percentages of these patients who had a score of ≧5.0 or <5.0 on a routine Apgar-like patient index datasheet (RAPID), an index derived from 3 scales on a patient self-report questionnaire which includes: physical function, pain and global status (Chung C, Sokka T, Pincus T, et al. Ann Rheum Dis 2005; 64(Suppl III): 188 (Poster THU0263).

Data were analyzed from 4 adalimumab trials: Study I: 24 weeks of adalimumab 40 mg every other week (eow)+methotrexate (MTX) or placebo+MTX; Study 2: 52 weeks of adalimumab monotherapy vs. placebo; Study 1: 52 weeks of adalimumab+MTX eow or placebo+MTX; and Study K: 24 weeks of adalimumab+standard disease-modifying antirheumatic drug (DMARD) therapy or placebo+DMARD therapy. All 1,391 patients in these trials were evaluated in this study. The RAPID index is based on patient self-report data on a Health Assessment Questionnaire (HAQ), recalibrated to 0-10, based on the following criteria: physical function (0-3): 0=0-0.25, 1=0.26-0.75, 2=0.76-1.5, and 3=1.51-3.0; pain visual analog scale (VAS) (0-3): 0=0-10, 1=11-30, 2=31-60, and 3=61-100; and patient's global status VAS (0-4): 0=0-10, 1=11-25, 2=26-50, 3=51-75, and 4=76-100. All patients were classified at baseline as having a DAS28<5.1 or ≧5.1 or RAPID <5.0 or ≧5.0, the higher values for each indicating severe disease. DAS28 was calculated using swollen joint count, tender joint count, patient's global assessment of disease activity and C-reactive protein (CRP) concentration. Spearman rank correlations and box plots to compare RAPID scores with DAS28 were also analyzed.

Among 1,391 patients, 74% had severe disease according to DAS28 and RAPID (i.e., DAS28≧5.1 and RAPID≧5.0; 4% had DAS28≧5.1 and RAPID<5.0; 15% had RAPID≧5.0 and DAS28<5.1; and 6% did not have severe disease by either index. Overall, 78% had DAS28 scores indicating severe disease, and 90% had RAPID scores indicating the same. Table 30 shows the number of patients in adalimumab clinical trials with DAS28< or ≧5.1 and RAPID (R326)< or ≧5. RAPID scores are sensitive in clinical trials to recognize differences between results of active vs. placebo treatment.

TABLE 30
Number of patients in ada trial with DAS28 < or > 5.1

| | | | | | | | | | | DAS28 ≧ 5.1 | | DAS28 ≧ 5.1 | | DAS28 < 5.1 | |
|---|---|---|---|---|---|---|---|---|---|---|---|---|---|---|---|
| | Total in | | | | | | | | | AND | | AND | | AND | |
| | Study | DAS28 ≧ 5.1 | | DAS28 < 5.1 | | RAPID ≧ 5.0 | | RAPID < 5.0 | | RAPID > 5.0 | | RAPID < 5.0 | | RAPID < 5.0 | |
| Study I | 128 | 100 | (78%) | 29 | (22%) | 119 | (92%) | 10 | (8%) | 99 | (77%) | 1 | (0.7%) | 9 | (7%) |
| Study 2 | 222 | 216 | (97%) | 6 | (3%) | 216 | (97%) | 6 | (3%) | 212 | (95%) | 4 | (2%) | 2 | (1%) |
| Study 3 | 404 | 305 | | 99 | (25%) | 353 | (87%) | 51 | (13%) | 289 | (72%) | 16 | (4%) | 35 | (9%) |
| Study K | 636 | 468 | (74%) | 168 | (26%) | (88%) | 562 | 74 | (12%) | 436 | (69%) | 32 | (5%) | 42 | (7%) |
| Total | 1391 | 1089 | (78%) | 302 | (22%) | 1250 | (90%) | 141 | (10%) | 1036 | (74%) | 53 | (4%) | 88 | (6%) |

RAPID scores are correlated substantially and statistically significantly with DAS28, confirmed with box plot analysis. Table 31 shows the Spearman rank correlations of changes in RAPID scores with changes in DAS28 scores and changes in ACR-N in 4 adalimumab trials.

TABLE 31

| Trial | Study I | Study 2 | Study 1 | Study K |
|---|---|---|---|---|
| DAS28 | 0.80 | 0.79 | 0.69 | 0.69 |
| ACR-N | 0.77 | 0.86 | 0.70 | 0.68 |

All p < 0.001

Table 32 shows the disease severity of adalimumab patients according to DAS28 and RAPID.

TABLE 32
Disease severity of ada patients according to DAS28 and RAPID

| | DAS28 < 5.1 | DAS28 >/= 5.1 | Total |
|---|---|---|---|
| RAPID < 5.0 | 88 (6%) | 53 (4%) | 141 (10% |
| RAPID ≧ 5.0 | 214 (15%) | 1036 (74%) | 1250 (90%) |
| Total | 302 (22%) | 1089 (78%) | 1391 (100%) |

Overall, the RAPID index of 3 patient questionnaire scores identified 95% of patients with DAS28 of ≧5.1 as having severe disease, and identified 72% of patients who were enrolled in anti-TNF clinical trials as having severe disease despite a DAS28<5.1. The RAPID scores also correlated significantly with DAS28. Thus, along with joint examinations, laboratory tests, and other inclusion criteria, this simplified index could be used to identify patients with RA who might be candidates for inclusion in clinical trials of anti-TNF and other therapies.

### EXAMPLE 9

### C-Reactive Protein Predicts Treatment Response to Adalimumab (HUMIRA®) in Patients with Rheumatoid Arthritis

Elevated C-reactive protein (CRP) is a marker of inflammatory disease activity as well as a predictor of poor outcome (e.g., radiographic damage) in patients with rheumatoid arthritis (RA).

To determine if baseline CRP or changes in CRP during treatment predict therapeutic responses to adalimumab, adult patients with RA were enrolled in a double-blind, randomized, placebo-controlled trial (Cohen, S. B., Kavanaugh, A. F., Emery, P., et al., Ann Rheum Dis 2005: 64(Suppl III):437). Patients who were randomized to receive either adalimumab 40 mg every other week (eow) plus methotrexate (MTX), or MTX alone and completed Week 52 were included in this analysis. 407 patients with active RA enrolled in the 2 study arms. Of these patients, 299 (73.5%) patients completed the study until Week 52 and were evaluated. Table 33 shows the baseline demographics and disease characteristics of the patients enrolled in the study initially.

TABLE 33

| | Adalimumab + MTX | | | MTX | | |
|---|---|---|---|---|---|---|
| | <1 | 1-4 | >4 | <1 | 1-4 | >4 |
| Baseline CRP (mg/dL) | n = 92 | n = 92 | n = 23 | n = 92 | n = 83 | n = 24 |
| Age (years) | 56 | 55 | 57 | 55 | 56 | 56 |
| Gender (% Female) | 70 | 83 | 78 | 67 | 78 | 75 |
| Disease Duration (years) | 10.7 | 11.5 | 9.5 | 9.7 | 12.6 | 8.5 |
| Physician's Global | 57.2 | 65.1 | 69 | 56.8 | 63.9 | 70.0 |
| Assessment (mm) | | | | | | |
| Patient's Global | 46.4 | 53.9 | 72.7 | 45.8 | 60.3 | 68.4 |
| Assessment (mm) | | | | | | |
| TJC (0-28) | 13.8 | 14.2 | 16.5 | 14.7 | 15.6 | 16.2 |
| SJC (0-28) | 11.6 | 14.0 | 14.1 | 12.8 | 13.8 | 14.8 |
| DAS28 | 5.2 | 5.8 | 6.7 | 5.3 | 6.1 | 6.6 |
| HAQ (0-3) | 1.27 | 1.54 | 1.78 | 1.28 | 1.65 | 1.71 |
| Total Sharp Score (TSS) | 66 | 78 | 76 | 57 | 80 | 58 |

Table 34 shows the response criteria in patients who completed Week 52 in 3 levels of CRP at baseline.

TABLE 34

| | Adalimumab 40 mg | | | | | |
|---|---|---|---|---|---|---|
| Baseline CRP | eow + MTX | | | MTX | | |
| (mg/dL) | <1 | 1-4 | >4 | <1 | 1-4 | >4 |
| Patients entering | 92 | 92 | 23 | 92 | 83 | 24 |
| trial* (n) | | | | | | |
| Patients completing | 70 | 69 | 20 | 71 | 54 | 15 |
| trial (n) | | | | | | |
| ACR20 (% | 77 | 75 | 75 | 30 | 36 | 47 |
| responders) | | | | | | |
| ACR50 (% | 48 | 59 | 55 | 13 | 18 | 0 |
| responders) | | | | | | |
| ACR70 (% | 24 | 32 | 45 | 6 | 9 | 0 |
| responders) | | | | | | |
| DAS28 (mean | −2.1 | −2.4 | −3.4 | −1.3 | −1.2 | −1.7 |
| change) | | | | | | |
| HAQ (mean | −0.5 | −0.6 | −1.1 | −0.4 | −0.4 | −0.3 |
| change) | | | | | | |
| TSS (mean change) | −0.6 | 0.5 | 1.5 | 2.1 | 3.0 | 4.5 |
| Erosions (mean | −0.4 | 0.2 | 0.9 | 1.5 | 1.7 | 2.1 |
| change) | | | | | | |

Patients were grouped into three cohorts based on baseline CRP concentrations: low (<1 mg/dL); intermediate (1-4 mg/dL); and high (>4 mg/dL). Analyses were further conducted in two categories of patients according to their change in CRP concentrations at Week 12: those with a ≧20% decrease in CRP from baseline; or those with a <20% decrease in CRP from baseline. The following efficacy and radiographic measures were analyzed at Week 52 in the three baseline CRP categories and in the two Week 12 change in CRP categories: DAS28, ACR 20/50/70, Health assessment questionnaire (HAQ), and Total Sharp score (TSS).

Adalimumab plus MTX therapy demonstrated greater changes in DAS28 scores compared with MTX monotherapy in all baseline CRP concentration categories. Table 35 shows the changes in DAS28 at Week 52 in patients grouped by baseline CRP categories.

TABLE 35
Change in DAS28 at week 52 by baseline CRP categories

| | CRP concentrations at baseline | | | |
|---|---|---|---|---|
| | | <1 mg/dL | 1-4 mg/dL | >4 mg/dL |
| Mean change | Ada + mtx | −2.1* | −2.4* (n = 69) | −34* (n = 20) |
| in DAS28 | | (n = 70) | | |
| score | Mtx | −1.3 | −1.2 | −1.7 (n = 15) |
| | | (n = 71) | (n = 54) | |

*p < 0.01 ada + mtx vs. mtx

Table 36 shows the mean change in TSS at Week 52. As can be seen in this figure, adalimumab plus MTX demonstrated superior inhibition of disease progression compared with MTX monotherapy in all baseline and Week 12 CRP categories.

TABLE 36
Mean change in TSS at week 52 by baseline CRP

| | CRP concentrations at baseline | | | |
|---|---|---|---|---|
| | | <1 mg/dL | 1-4 mg/dL | >4 mg/dL |
| Mean change | Ada + mtx | −0.30 | 0.48 (n = 69) | 1.48 (n = 20) |
| in TSS | | (n = 70) | | |
| | Mtx | 1.8 | 3.25 | 4.23 |
| | | (n = 71) | (n = 54) | (n = 15) |

*p < 0.01 ada + mtx vs. mtx

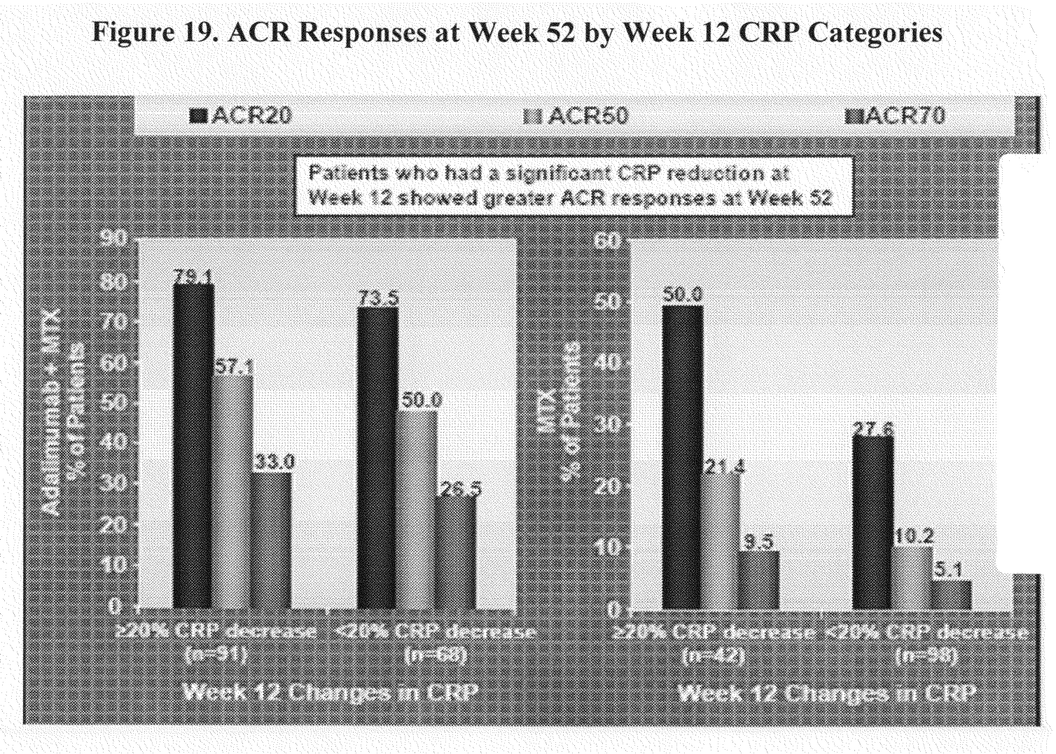

At Week 12, 57% of the patients treated with adalimumab and MTX (n=91) had a ≧20% decrease in CRP compared vs. 33% of MTX monotherapy patients (n=42). In contrast, 43% of the patients treated with adalimumab and MTX (n=68) had a <20% decrease in CRP vs. 67% of MTX monotherapy patients showing a reduction (n=98). As shown in FIG. 19, the ACR responses at Week 52, with patients grouped by Week 12 CRP categories. As can be seen in FIG. 19, adalimumab plus MTX demonstrated superior ACR 20/50/70 responses compared with MTX monotherapy in both Week 12 CRP categories. Patients with a significant reduction in CRP at Week 12 (≧20%) demonstrated a higher ACR response at Week 52. MTX only patients who had a significant reduction in CRP at Week 12 (≧20%) had a 50% greater ACR response at Week 52.

The mean changes in HAQ at Week 52, with patients grouped by Week 12 CRP categories, included the following: the mean change in HAQ in patients in the ada+mtx group who had ≧20% decrease in CRP was −0.72 (n=91) and of those who had <20% decrease in CRP the decrease was −0.55 (n=68) (both p<0.001 vs. mtx). The mean change in HAQ in patients in the mtx alone group who had ≧20% decrease in CRP was −0.48 (n=42) and of those who had <20% decrease in CRP the decrease was −0.29 (n=42). As such, adalimumab plus MTX demonstrated superior functional improvement as measured by HAQ compared with MTX monotherapy in both Week 12 CRP groups of patients. Also, patients who had a significant CRP reduction at Week 12 demonstrated a greater improvement in HAQ.

Overall, this study showed that CRP is clinically useful to demonstrate in patients treated with adalimumab and MTX a greater improvement in disease activity and functional outcomes, as well as a larger inhibition of radiographic progression compared to MTX alone. Further, high CRP concentrations at baseline predict greater improvement in disease activity and functional outcomes while low CRP concentrations predict a larger magnitude of inhibition of radiographic progression at Week 52. A significant improvement in CRP (≧20% decrease) at Week 12 predicts a greater improvement in disease activity and functional outcomes in both treatment arms, and a larger magnitude of inhibition of radiographic progression with adalimumab.

### EXAMPLE 10

### Adalimumab (HUMIRA®) Monotherapy Provides Sustained Long-Term Improvement in Health Utility in Patients with Rheumatoid Arthritis (RA)

Health related quality of life (HRQoL), as measured by utility-based instruments, is a useful outcome measure in clinical research in RA. In adalimumab clinical trials, health utility data were collected directly by using the Health Utilities Index mark 3 (HUI3) questionnaire. The HUI3 is a well established, validated health utility instrument. The widespread use of HUI facilitates the interpretation of results and permits comparisons of disease and treatment outcomes at local, national and international levels.

Health Utilities are preferences or desirability of a health state, with “1” denoting perfect health and “0” denoting death. Utilities can be directly converted into quality-adjusted life years (QALYs). Health utilities and QALYs help policymakers compare the benefits of therapeutic interventions across diseases.

The ability of adalimumab monotherapy compared with placebo to provide sustained, long-term improvement in health utility in patients with severe RA who had failed at least one DMARD was measured. The results were compared with data reported for study 1 (adalimumab+MTX in the treatment of moderate to severe RA) (Dietz et al., Ann Rheum Dis 2005; 64(Suppl III):393).

Data was obtained from a health economics companion study to an adalimumab pivotal study (Study 2), during which patients were followed under double-blind, randomized conditions for the first 26 weeks before rolling into a long-term, open-label extension study (OLE; Study 3). A subset of patients receiving adalimumab 40 mg every other week (eow) were evaluated for up to 170 weeks.

The improvement in utility in the Study 3 RA population was compared with results reported earlier for Study 1 (adalimumab 40 mg eow+MTX). The HUI3 was assessed at baseline, and at several time points during the study. Health Utility Index 3 (scale=0-1) measures preference of a health state, with “1” denoting perfect health and “0” denoting death. HUI3 changes of ≧0.03 are considered clinically important. The HUI3 classification system consists of 8 attributes and 5 or 6 levels on each attribute, ranging from full function to severe impairment. Table 37 shows the HUI3 attributes used in the present study.

TABLE 37

| | |
|---|---|
| Vision | (6 classifications 2 questions) |
| Hearing | (6 classifications 2 questions) |
| Speech | (5 classifications 2 questions) |
| Emotion | (5 classifications 2 questions) |
| Pain | (5 classifications 2 questions) |
| Ambulation | (6 classifications 1 question) |
| Dexterity | (6 classifications 2 questions) |
| Cognition | (6 classifications 2 questions) |

The HUI3 questionnaire used in these adalimumab trials was self-completed by the patients and consisted of questions asking patients to reflect on their health status over the preceding 4 weeks.

The baseline patient demographics and disease severity characteristics are shown in Table 38.

TABLE 38
Basline demographic and disease severity characteristics

| | Adalimumab Study 3 | | Adalimumab Study 1 | |
|---|---|---|---|---|
| | 40 mg eow | Placebo | 40 mg eow + MTX | Placebo + MTX |
| | (N = 99) | (N-96) | (N = 207) | (N = 200) |
| AGE (YEARS) | 53 ± 13.4 | 53 ± 13.6 | 56 ± 13.5 | 56 ± 12.0 |
| Sex (% female) | 76 | 80 | 76 | 73 |
| Disease Duration (Years) | 10 ± 7.0 | 11 ± 9.4 | 11 ± 9.2 | 11 ± 8.8 |
| Tender Joint Count (0-68) | 33.8 ± 16.0 | 34.7 ± 14.5 | 27.3 ± 12.7 | 28.1 ± 13.8 |
| Swollen joint count (0-66) | 21.0 ± 11.0 | 19.7 ± 9.4 | 19.3 ± 9.8 | 19.0 ± 9.5 |
| HAQ (0-3) | 1.85 ± 0.55 | 1.86 ± .065 | 1.45 ± 0.63 | 148 ± 0.59 |
| C-reactive protein, mg/dL | 5.4 ± 3.8 | 5.9 ± 5.0 | 1.8 ± 2.3 | 1.8 ± 2.1 |
| (normal < 0.8) | | | | |
| No. of previous DMARDs | 3.8 | 3.6 | 2.4 | 2.4 |
| Mean ± SD | | | | |

As can be seen in this table, the patient population in Study 3 had more severe disease at baseline than patients in Study 1, which is reflected by the number of previous DMARDs, TJC, SJC, HAQ, and CRP.

Table 39 shows the HUI3 improvement from baseline. As can be seen in this figure, RA patients' baseline utility scores were approximately one-third to half that of the age- and sex-adjusted population norm of 0.88 in both studies.

TABLE 39
HUI3 Improvement from Baseline

| | Baseline | Mean (relative) HUI3 improvement | |
|---|---|---|---|
| | HUI3 | from baseline at | |
| | scores | Week 50^{#/}52* | Week 170 |
| Adalimumab (Study 1) | | | |
| 40 mg eow + MTX | 0.44 | +0.21a, b (+48%) | — |
| Placebo + MTX | 0.39 | +0.07 (+18%) | — |
| Adalimumab (Study 3) | | | +0.19c (+70%) |
| 40 mg eow | 0.27 | +0.17c (+63%) | |
| Placebo | 0.29 | — | — |

aclinically important improvement vs. placebo (≧0.03)
b p < 0.001 vs. placebo
c≧clinically important improvement (0.03)
^{#}Adalimumab (Study 3)
*Adalimumab + MTX (Study 1)
— not assessd

The mean improvement from baseline in HUI3 scores (mean change from baseline) included 0.21 of ada+mtx at one year (study 1), 0.17 ada 1 year, and 0.19 ada at 3 years (minimum clinically important difference (≧0.03)). After one year of therapy, mean changes from baseline were comparable between patients receiving adalimumab monotherapy and those receiving adalimumab and MTX.

Improvements in health utility occurred rapidly and were sustained through Week 170. The percentage of patients who reached the minimum clinically important difference (MCID) of 0.03 change from baseline based on the HUI3 was about 60% from week 12 through week 170. Throughout the observation period of 3 years in Study 3, about 60% of patients taking adalimumab 40 mg eow monotherapy reached or surpassed the MCID of 0.03 change from baseline.

Overall, adalimumab monotherapy provides long-term, clinically important improvements in health-related quality of life, as measured by the HUI3 in patients with severe, active RA who had failed at least one DMARD. Even in those severely compromised RA patients, the improvement in utility compared favorably with the results of adalimumab plus MTX combination therapy. In patients, observed during the open-label extension study, these improvements were sustained for 3 years.

### EXAMPLE 11

### Adalimumab (HUMIRA®) Monotherapy Significantly Improves Fatigue in Patients with Severe Rheumatoid Arthritis (RA)

Fatigue is a common symptom of rheumatoid arthritis (RA), reported at varying degrees of severity in more than 80% of patients. Fatigue has been identified as the most problematic aspect of their disease. Reduction in fatigue correlates with improvement in quality of life and should be a goal of therapy. Although use of tumor necrosis factor (TNF) antagonists is combination therapy with methotrexate (MTX) is optimal, some patients do not tolerate or benefit from MTX.

To determine whether adalimumab monotherapy reduced fatigue in patients with severe, active RA compared with placebo, data were obtained from a health economics companion study to a pivotal trial of adalimumab (Study 2) (Dietz, B. M., Sterz, R., Holtbrugge, W., et al. Ann Rheum Dis 2005; 64(Suppl III):392-3). Subsets of patients receiving adalimumab 40 mg every other week (eow) (n=99) or placebo (n=96) as monotherapy for 26 weeks was evaluated. All patients studied had severe, active RA. Patients' fatigue was measured by the FACIT-F, which is validated for RA.

FACIT-F was administered at baseline, at 1 or 2 time points during the study, and at the end of the study. The questionnaire asked each patient the following “Below is a list of statements that other people with your illness have said are important. By circling one (1) number per line, please indicate how true each statement has been for you during the past 7 days.” The questions in the questionnaire used to determine the FACIT score included the following, where each patient indicated the appropriate number (0-4): I feel fatigued; I feel weak all over; I feel listless (“washed out”); I feel tired; I have trouble starting things because I am tired; I have trouble finishing things because I am tired; I have energy; I am able to do my usual activities; I need to sleep during the day; I am too tired to eat; I need help doing my usual activities; I am frustrated by being too tired to do the things I want to do; I have to limit my social activity because I am tired. FACIT-F scores range from 0-52, with higher scores representing less fatigue. FACIT-F changes of ≧4 are considered clinically meaningful. The results of improvement in fatigue levels in patients receiving adalimumab monotherapy were compared with results reported earlier for the patients who received adalimumab 40 mg eow or placebo+MTX in Study I.

Table 40 shows the baseline demographics and disease severity characteristics for both study populations. At baseline, FACIT-F scores in Study 2 were slightly lower than seen in Study I, an expected result, given the patient population in Study 2 had more severe disease.

TABLE 40
Baseline demographics and disease severity characteristics

| | Study 2 | | Study 1 | |
|---|---|---|---|---|
| | | Adalimumab | | Adalimumab |
| | Placebo | 40 mg eow | Placebo + MTX | 40 mg eow + MTX |
| | (N = 96) | (N = 99) | (N = 62) | (N = 67) |
| Age (years) | 53 ± 14 | 53 ± 13 | 56 ± 77 | 57 ± 11 |
| Sex (% female) | 80 | 76 | 82 | 75 |
| Disease duration | 11 ± 9 | 10 ± 7.0 | 11 ± 8.0 | 12 ± 11 |
| (years) | | | | |
| Tender joint count | 34.7 ± 14.5 | 33.8 ± 16.0 | 28.7 ± 15.2 | 28.0 ± 12.7 |
| (0-68) | | | | |
| Swollen joint | 19.7 ± 9.4 | 21.0 ± 11.0 | 16.9 ± 9.5 | 17.3 ± 8.6 |
| count (0-66) | | | | |
| HAQ (0-3) | 1.86 ± 0.65 | 1.85 ± 0.55 | 1.64 ± 0.63 | 1.55 ± 0.61 |
| C-reactive protein, | 5.9 ± 5.0 | 5.4 ± 3.8 | 3.1 ± 3.9 | 2.1 ± 1.8 |
| mg/dL | | | | |
| (normal < 0.8) | | | | |
| No. of previous | 3.6 | 3.8 | 3.0 | 2.9 |
| DMARDs | | | | |
| Mean ± SD | | | | |

The results from adalimumab monotherapy in Study 2 were compared with previously reported results of adalimumab+MTX in Study I. For Study 2, the mean change from baseline for the FACIT-F score at 6 months was 8.5 for ada 40 mg eow and 3.2 for placebo (p<0.0001). For Study I, the mean change in baseline for the FACIT-F score at 6 months was 8.1 for ada 40 mg eow+mtx and 2.5 for placebo+mtx (p=0.01). The minimum clinical important difference was determined to be ≧4.0. At Week 26, the Study 2 FACIT-F scores for adalimumab demonstrated statistically significant improvements and clinically important compared with the placebo group.

Table 41 shows the FACIT-F improvement from baseline to end of study. Statistically and clinically significant improvements in FACIT-F scores were seen in the Study 2 patient population as early as 12 weeks of adalimumab treatment (p<0.001 vs. placebo, change from baseline of 7.2). Improvement was maintained until the end of the study. The relative changes from baseline further illustrate the favorable results of adalimumab treatment with placebo.

TABLE 41
FACIT-F Improvement from Baseline to End of Study

| | | |
|---|---|---|
| | | Relative |
| Baseline | Mean FACIT-F | FACIT-F |
| FACIT-F | improvement | change from |
| scores | from baseline | baseline |

| | | | |
|---|---|---|---|
| Study I (24 weeks) | | | |
| 40 mg eow + MTX | 28.0 | +8.1a, c | +29% |
| Placebo + MTX | 28.7 | +2.5 | +9% |
| Study 2 (26 weeks) | | | |
| 40 mg eow | 26.1 | +8.5b, c | +33% |
| Placebo | 26.4 | +3.2 | +12% |

Table 42 shows the improvements in RA patients' fatigue in the Study 2 and Study I patient populations.

TABLE 42
Improvements in RA Patients' Fatigue in Study 2 and Study I

| | Adalimumab | |
|---|---|---|
| | 40 mg eow | Placebo |
| Study 2 (adalimumab | | |
| monotherapy) | n = 99 | n = 96 |
| Baseline | 26.1 | 26.4 |
| Mean change from baseline | 8.5 b, c | 3.2 |
| at week 26 | | |
| Study I (ada + mtx) | n = 61 | n = 58 |
| Baseline | 28.0 | 28.7 |
| Mean change from baseline | 8.1 a, c | 2.5 |
| at week 24 | | |

a p < 0.01 vs. placebo
b p < 0.0001 vs. placebo
c ≧clinically meaningful improvement vs. baseline

Overall adalimumab monotherapy treatment provided statistically significant and clinically important improvements in fatigue with severe, active RA, compared with patients receiving placebo. Improvements in fatigue levels achieved by adalimumab monotherapy in Study 2 compared favorably with those observed with adalimumab and MTX combination therapy in Study I. Thus, adalimumab administered either as monotherapy or combination therapy with MTX has been shown to provide statistically significant and clinically important improvements in fatigue, an important clinical symptom in patients with severe RA.

### EXAMPLE 12

### Adalimumab (HUMIRA®) Monotherapy Significantly Improves Health Utility in Patients with Severe Rheumatoid Arthritis (RA)

Health related quality of life (HRQL) can be measured by three types of measurement techniques: generic quality of life instruments (e.g. SF-36); disease-specific quality of life instruments (e.g. HAQ); and health utility instruments (e.g. Health Utilities Index Mark 3 [HUI3]). Adalimumab is the only anti-TNF biologic for RA that collected health utility data during clinical development using the HUI3 questionnaire.

The utilities gained in patients with severe active RA treated with adalimumab monotherapy versus placebo were compared. These results were further compared with data reported from Study I.

Data were obtained from a health economics companion study to a pivotal study of adalimumab (Study 2). A subset of patients receiving adalimumab 40 mg every other week (n=99) was evaluated versus placebo (n=96) for 26 weeks. Results of utility improvement in the Study 2 set of patients with severe RA were compared with results reported earlier from Study I (adalimumab 40 mg eow+MTX). Inclusion criteria for this study included: all patients had severe active disease; and all patients had failed prior DMARD(s).

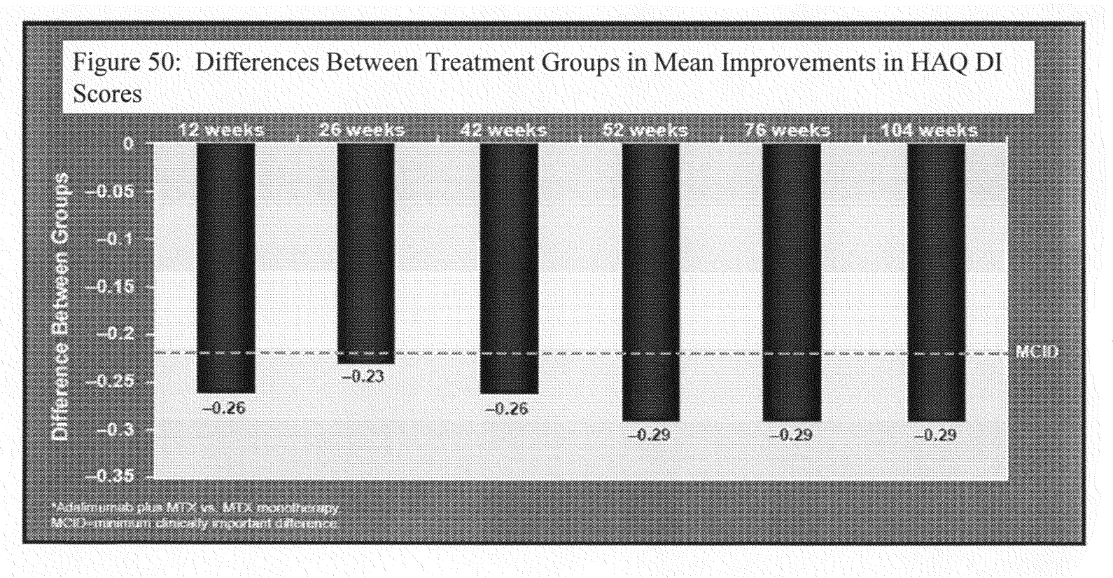

The Health Utilities Index Mark 3 (HUI3) was assessed at three time points. HUI 3 consists of 8 attributes: vision, hearing, speech, ambulation, dexterity, emotion, cognition, and pain. HUI3 score changes of ≧0.03 are considered clinically important. Patient demographics and clinical characteristics showed that in both studies, patients were included with severe RA and disease activity. FIG. 50 shows the baseline demographic and clinical characteristics of patients included in these studies. As can also be seen in this figure, the Study 2 study population started with baseline values reflecting a patient population with a more severe disease activity than in Study I, as documented by TJC, SJC, HAQ, CRP, and number of previous DMARDs.

Table 43 shows the mean improvement from baseline in HUI3 scores at 6 months.

TABLE 43
HUI3 improvement from baseline

| | | Mean HUI3 | Relative |
|---|---|---|---|
| | Baseline HUI3 | improvement | HUI3 change |
| | scores | from baseline | from baseline |
| Study 2 (26 weeks) | | | |
| 40 mg eow | 0.27 | +0.18a, b | +67% |
| placebo | 0.29 | +0.07 | +24% |
| Study I (24 weeks) | | | |
| 40 mg eow + MTX | 0.38 | +0.22a, b | +58% |
| placebo + MTX | 0.40 | +0.04 | +10% |

RA patients' utility scores at baseline in both studies were similar for adalimumab and placebo, and were about ⅓ of the age- and sex-adjusted population norm of 0.88. The mean improvement from baseline in HUI3 scores at 6 months was 0.18 ada 40 mg eow vs. 0.07 placebo for Study 2 (p<0.001) and 0.22 ada 40 mg eow+mtx vs. 0.04 placebo+mtx (p<0.001) for Study I. The minimum clinically important difference was ≧0.03. The Study 2 patient population, however, started with lower baseline utility values, reflecting a patient population with a more severe disease activity than those in Study I. Mean change from baseline revealed similar utility gains in adalimumab- and placebo-treated patients for both studies. All gains relative to placebo were clinically important and statistically significant.

Table 44 shows the health utility improvement from adalimumab monotherapy versus adalimumab and MTX therapy.

TABLE 44
Health Utility Improvement from Adalimumab Monotherapy vs.
Adalimumab + MTX Therapy

| | | Mean HUI3 change from |
|---|---|---|
| | Baseline HUI3 scores | baseline at 6 months |
| Study 2 (26 weeks) | | |
| 40 mg eow | 0.27 | 0.18 a, b |
| Placebo | 0.29 | 0.07 |
| Study I (24 weeks) | | |
| 40 mg eow + mtx | 0.38 | 0.22 a, b |
| Placebo + mtx | 0.40 | 0.04 |

a ≧clinically important improvement vs. placebo
b p < 0.001 vs. placebo

Overall, adalimumab monotherapy provided clinically important and statistically significant improvements in health-related quality of life as measured by the HUI3 in patients with severe, active RA who had failed previous therapies. These results compare favorably with the results seen for the combination therapy of adalimumab plus MTX in moderate to severe RA.

### EXAMPLE 13

### Adalimumab (HUMIRA®) Monotherapy Sustains Long-Term Improvements in Fatigue in Patients with Severe Rheumatoid Arthritis (RA)

The long-term effects of adalimumab monotherapy in reducing fatigue in patients with severe, active RA versus placebo was investigated. These results from this study were compared with data reported from Study 1 (Dietz B M, van de Putte L B A, Holtbrugge W, et al. Ann Rheum Dis 2005; 64(Suppl III):579).

Data were obtained from a health economics companion study to a pivotal trial of adalimumab, which rolled over into a long-term, open-label extension (OLE) study. A subset of patients receiving adalimumab 40 mg every other week (n=99) was evaluated vs. placebo (n=96) for 26 weeks. Improvement in fatigue levels in the more severely compromised RA population in this monotherapy trial was compared to results of Study 1 (adalimumab 40 mg eow+MTX). Patients' fatigue was measured by the FACIT-F questionnaire. FACIT-F was administered at baseline, and at several time points during the study. FACIT-F scores range from 0-52, with higher scores representing less fatigue. Changes in FACIT-F scores of ≧4 were considered clinically meaningful.

Baseline patient characteristics were: female: 80%; age: 53 years; duration of disease: 10 years; TJC (0-68): 34; SJC (0-66) 21; HAQ score: 1.9, C-reactive protein (mg/L): 54; no. of previous DMARDs: 4 (all mean values except % female). Mean changes from baseline at Week 52*/50^{#} (*Study 1/^{#} monotherapy trial/OLE) were similar between the 2 studies. These improvements in fatigue from adalimumab monotherapy were sustained through Week 170. Table 45 shows the results of this study.

TABLE 45
Improvements in Fatigue in the Monotherapy/OLE and Study 1 Trials

| | Study 1 | Monotherapy/OLE |
|---|---|---|
| | 40 mg eow + MTX | 40 mg eow (mono) |
| | Adalimumab (placebo) | Adalimumab (placebo) |
| Baseline | 30.6 (28.9) | 26.1 (26.4) |
| Mean Change from | | |
| Baseline at | | |
| Week 50^{#}/Week 52* | 7.1^{a,b} (3.3) | 7.3^{b} |
| Week 98 | NA | 7.2^{b} |
| Week 146 | NA | 7.1^{b} |
| Week 170 | NA | 7.2^{b} |

^{a}p < 0.001 vs. placebo;
^{b}≧clinically meaningful improvement vs. baseline

Overall, adalimumab monotherapy provided long-term, clinically meaningful improvements in fatigue in RA patients with severe disease who had failed MTX therapy. Twelve-month results in the population with adalimumab monotherapy compared favorably with the results of the combination of adalimumab plus MTX. In the patients observed over the course of the study, these improvements were sustained for 3 years.

### EXAMPLE 14

### Clinical Remission Achieved in the Early Treatment of Recent-Onset Rheumatoid Arthritis (RA)

An increasing body of evidence suggests that early, aggressive treatment of rheumatoid arthritis (RA) leads to the most favorable clinical and radiographic outcomes. Clinical trials of tumor necrosis factor (TNF) antagonists in RA demonstrate that the combination of a TNF antagonist plus methotrexate (MTX) has superior efficacy to MTX alone. Thus, clinical remission and good radiographic outcomes are achievable with aggressive strategies.

Various measurements are used to define clinical remission in RA, including the Disease Activity Score (DAS<1.6 or DAS28<2.6), the Simplified Disease Activity Index (SDAI<5), as well as other indicators of an excellent clinical response (ACR70, no tender joints, Pinals criteria, etc.). This study compared the use of a TNF antagonist plus MTX versus either agent alone in MTX-naïve patients with recent-onset RA. It provides a unique resource for comparing efficacy outcomes according to several different criteria for clinical remission (Emery, P., van Riel, P. L., Cush, J. J., et al., Ann Rheum Dis 2005; 64 (Suppl III):441).

The results of this study were evaluated using multiple criteria for clinical remission. This study (Study J) was a 2-year, double-blind, Phase III study. MTX-naïve adult patients with active, early RA (<3 years) were randomized to 1 of 3 treatment arms: Adalimumab 40 mg every other week (eow)+MTX; Adalimumab 40 mg eow alone+placebo; or MTX alone+placebo. FIG. 18 shows the design of this study.

The primary endpoints for this study were the ACR50 responses and changes in Total Sharp Score (TSS), each comparing the adalimumab and MTX to the MTX alone arms at 1 year. Secondary endpoints included DAS28<2.6 at year 1 and Major Clinical Response (MCR=ACR70 response for ≧6 continuous months during a 2-year period).

A total of 799 patients were enrolled. Baseline demographics and clinical characteristics of the patients enrolled in this study are described in Example 7. Baseline demographics and clinical characteristics were similar among the 3 arms, i.e., ada+mtx, ada alone, or mtx alone. Mean age was 52 years and mean duration of RA was 0.7 years. TJC (0-68) was 24, SJC (0-68) was 34, DAS28 was 6.3, HAQ was 1.5, TSS was 19.5, morning stiffness was 139 minutes, and CRP was 4.8 mg/dL (all mean values). In addition, 75% of patients were female, and 83% were RF+. After 1 and 2 years of therapy, the percentages of patients with ACR90 responses, TJC=0, SJC=0, HAQ=0, morning stiffness=0 minutes, and normal CRP were determined. Data are from the intention-to-treat (ITT) population with imputation used for missing values. Results were compared with the percentages of patients who achieved DAS28<2.6 or a Major Clinical Response (MCR).

By several clinical measures, an excellent clinical response was achieved by significantly higher percentages of patients who had received combination therapy than either monotherapy, i.e., MTX alone, or adalimumab alone. Example 7 describes the ACR 20/50/70 at Weeks 52 and 104. ACR90 rates were as follows: Week 52: ada+mtx 24%, ada alone 8%, and mtx alone 13%; week 104 ada+mtx 27%, ada alone 9%, mtx alone 13% (p<0.001 for ada+mtx vs. ada and mtx). The percentage of patients with DAS28<2.6 at 2 years was 49% ada+mtx, and 25% for both ada alone and mtx alone (p<0.001). The percentage of patients with MCR at 2 years was 49% ada+mtx, 25% for ada alone, and 27% for mtx alone (p<0.001). MCR=major clinical response defined as an ACR70 response for ≧6 continuous months during a 2 year period. Table 46 shows the percent of patients with TJC=0, an SJC=0, an HAQ=0, and an AM stiffness=0 at two years in each of the three arms of the study.

TABLE 46
Percentage of patients from each arm

| | TJC = 0 | SJC = 0 | HAQ = 0 | AM stiffness = 0 |
|---|---|---|---|---|
| Ada + mtx | 36 | 39 | 33 | 53 |
| Ada | 15 | 19 | 19 | 30 |
| mtx | 21 | 19 | 19 | 30 |

†p < 0.001 for adalimumab + MTX vs. either monotherapy.

Normalization of CRP at 1 and 2 years during the study was as follows: at one year, 61% ada+mtx, 34% ada alone, and 35% mtx alone (normal CRP=≦0.8 mg/dL). At 2 years, 55% of ada+mtx, 34% ada alone, and 29% of mtx alone (for both years p<0.001). Table 47 shows the results of selected clinical remission criteria at 1 and 2 years.

TABLE 47
1 and 2-Year Results of Selected Clinical Remission Criteria (% patients)

| | | | Ada | Ada | MTX | MTX |
|---|---|---|---|---|---|---|
| | Ada + MTX | Ada + MTX | alone | alone | alone | alone |
| | 1 year | 2 years | 1 year | 2 years | 1 year | 2 years |
| DAS28 < 2.6 | 43† | 49† | 23 | 25 | 21 | 25 |
| MCR | | 49† | | 25 | | 27 |
| ACR90 | 24† | 27† | 8 | 9 | 13 | 13 |
| TJC = 0 | 26‡ | 36† | 18 | 15 | 14 | 21 |
| SJC = 0 | 32† | 39† | 16 | 19 | 17 | 19 |
| HAQ = 0 | 32† | 33† | 17 | 19 | 19 | 19 |
| Morning | 49† | 53† | 32 | 30 | 31 | 30 |
| Stiffness = 0 | | | | | | |
| Normal CRP | 61† | 55† | 34 | 34 | 35 | 29 |

†p < 0.001 for combination therapy vs. either montherapy,
‡p < 0.05 for combination therapy vs. either monotherapy

Thus, overall in MTX naïve patients with recent onset RA, excellent, remission like clinical responses occurred most frequently in those patients treated with adalimumab and MTX. Results in the two monotherapy arms were comparable. Therefore, combination therapy offers the prospect of clinical remission to patients with recent onset RA.

### EXAMPLE 15

### Inhibition of Radiographic Disease Progression in Patients with Long-Standing Rheumatoid Arthritis Following 3 Years of Treatment with Adalimumab (HUMIRA®) Plus Methotrexate

The 3-year radiographic and clinical efficacy and the 3-year safety profile of adalimumab plus MTX in patients with long-standing RA who had inadequate responses to MTX was evaluated (Keystone E C, Kavanaugh A F, Sharp J T, et al. Ann Rheum Dis 2005; 64(Suppl III):419). Patients with a confirmed diagnosis of RA who were older than 18 years of age were included in the study. Further inclusion criteria required that patients had been taking MTX for at least 3 months prior to enrollment at a stable dose for at least 4 weeks prior to the screening visit.

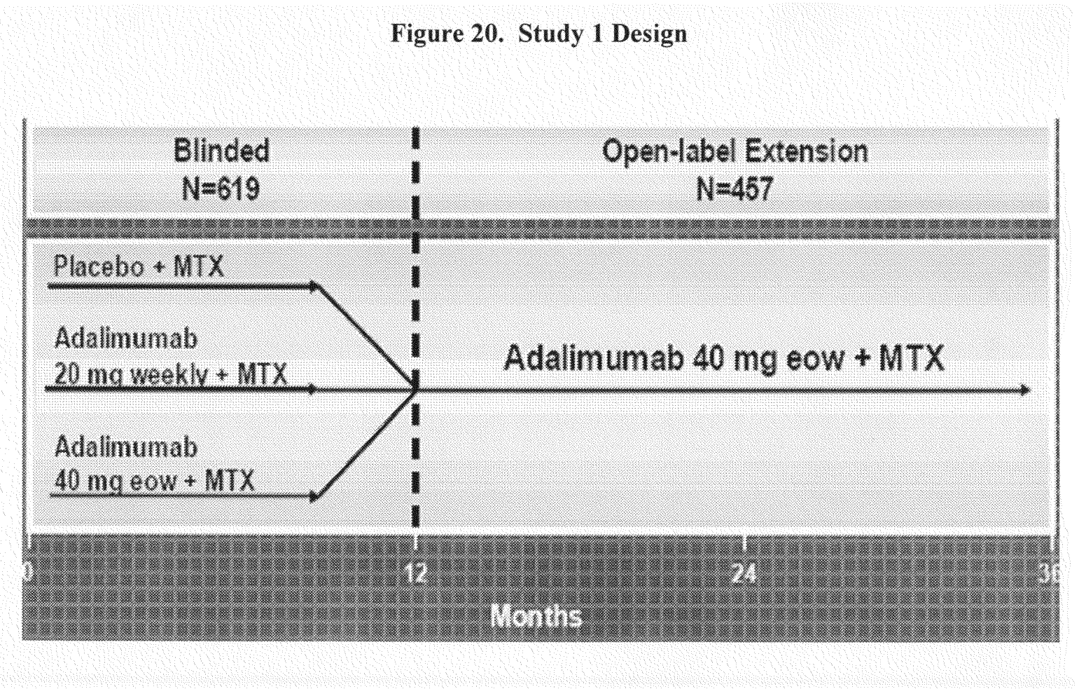

The study was a 12-month, double-blind, randomized, placebo-controlled trial with three treatment arms: Adalimumab 20 mg weekly+MTX (n=212); Adalimumab 40 mg eow+MTX (n=207); and Placebo+MTX (n=200). Patients who completed the 12-month, randomized controlled portion were eligible to receive adalimumab 40 mg eow+MTX in the open-label extension (OLE). Two readers assessed X-rays performed at baseline (start of RCT), 1 year (end of RCT), and 3 years (post 2 years OLE). Changes from baseline in Total Sharp Score (TSS), Joint Erosions (JE), and Joint Space Narrowing (JSN) were calculated. ACR 20, 50, and 70 response rates, as well as TJC and SJC, were also measured. Efficacy and safety evaluations were conducted at regularly scheduled visits. FIG. 20 shows the study design of Study 1, described in this Example. Table 48 shows the baseline patient demographic and disease characteristics.

TABLE 48
Baseline Patient Demographic and Disease Characteristics

| | Patients Enrolled | Patients Enrolled |
|---|---|---|
| Baseline | in Study 1 | in Study 1 OLE |
| Characteristic | (N = 619) | (N = 457) |
| Age (yrs) | 57 | 57 |
| % Female | 75 | 74 |
| Duration of RA (yrs) | 11 | 11 |
| MTX dose (mg/wk) | 17 | 16 |
| TJC (0-68) | 28 | 28 |
| SJC (0-66) | 19 | 19 |
| HAQ (0-3) | 1.5 | 1.4 |
| CRP (mg/dL) | 1.7 | 1.6 |
| TSS* (0-398) | 68 | 70 |

Mean Values at baseline of the placebo-controlled trial
*based on radiograph readings conducted at the end of the blinded RCT

619 patients with long-standing RA who had inadequate responses to MTX enrolled, and 467 patients completed the 12-month blinded phase of the study. 457 patients enrolled in the OLE and 363 (79%) completed Year 3 of the study. The reasons for withdrawal from the OLE included: adverse events 31 (7%); lack of efficacy 11 (2%); and other reasons 52 (11%). Patients entering the blinded and the OLE phases had moderately to severely active RA, and had similar baseline characteristics.

Radiographic analyses were performed without imputation on patients for whom radiographic data were available from the 1-year and 3-year time points (adalimumab+MTX arm: N=130, 129; MTX arm [MTX, Year 1; adalimumab+MTX, Years 2 and 3]: N=106, 101).

Adalimumab treatment provided three year control of radiographic progression of RA by adalimumab and MTX. The mean change in TSS at 12 months for ada 40 mg eow+mtx was 0.0 and 0.3 at 36 months vs. 2.8 at 6 months and 3.0 at 36 months for placebo+mtx. The mean change from baseline in the number of joint erosions in patients treated with adalimumab and MTX eow versus placebo and MTX was 0.0 ada+mtx at 12 months and 0.1 at 36 months vs. 1.7 for placebo at 12 months and 1.7 at 36 months. The mean change from baseline in joint space narrowing in patients treated with adalimumab and MTX eow versus placebo and MTX was ada+mtx 0.0 at 12 months and 0.2 at 26 months vs. placebo+mtx 1.2 at 12 months and 1.3 at 36 months.

The percent of patients with no radiographic progression following three years of adalimumab therapy included 62% TSS, 71% joint erosion score, and 73% joint space narrowing score. The percent of patients with radiographic improvement following three years of adalimumab therapy was 28% TSS, 29% joint erosion score, and 20% joint space narrowing score. In both cases, radiographic improvement was defined as a change from baseline of <−0.5 units.

The ACR 20/50/70 response rates were as follows: month 12 ACR20/50/70 included 69%/48%/26% patients; month 24 ACR20/50/70 included 63%/43%/28% patients; and month 36 ACR20/50/70 included 58%/42%/24% patients. The tender and swollen joint counts were consistently low, i.e. about 5-7 for TJC or SJC at 36 months. In addition, at the last visit, 21% of patients had 0 tender joints, and 22% of patients had 0 swollen joints.

Overall Adalimumab plus MTX inhibited structural damage and disease progression over 3 years in patients with long-standing RA who previously had an incomplete response to MTX. 62% of patients treated with adalimumab 40 mg+MTX for 3 years showed no progression in Total Sharp Score and 71% showed no progression in Joint Erosions. Adalimumab provided sustained improvement in the signs and symptoms of RA over 3 years of treatment. Adalimumab was well-tolerated. The types and rates of adverse events remained stable over 3 years.

### EXAMPLE 16

### Radiographic Improvement in Clinical Responders in the Early Treatment of Recent-Onset Rheumatoid Arthritis (RA)

An increasing body of evidence indicates that early, aggressive treatment of rheumatoid arthritis (RA) leads to the most favorable clinical and radiographic outcomes (Breedveld et al. Ann Rheum Dis 2004; 63:627-33). Recent clinical trials in RA demonstrate that combination therapy with a tumor necrosis factor (TNF) antagonist plus methotrexate (MTX) is superior to therapy with MTX alone (De Vries-Bouwstra et al. Arthritis Rheum 2003: 48:3649; Smolen et al. Ann Rheum Dis 2003: 61(Suppl I):64; and Weinblatt et al. Arthritis Rheum 2003; 48:35-45). Very low disease activity, clinical remission and good radiographic outcomes are achievable with aggressive strategies (De Vries-Bouwstra et al. Arthritis Rheum 2003: 48:3649).

Traditional disease-modifying antirheumatic drugs (DMARDs) such as MTX can have good efficacy against the clinical signs and symptoms of RA, but they are often less efficacious against concomitant progression of radiographic disease. While traditional disease-modifying antirheumatic drugs (DMARDs) such as methotrexate (MTX) can be effective in treating the clinical signs and symptoms of rheumatoid arthritis (RA), they often do not provide equal effectiveness in simultaneously halting radiographic progression. Early, aggressive treatment of RA may provide the most favorable clinical and radiographic outcomes. Study J was the first head-to-head trial of a TNF antagonist with MTX vs. either alone in MTX-naïve patients with recent-onset RA. As such, study J is a unique resource for examining the relationship between clinical and radiographic efficacy in RA.

The objective of this subanalysis of study J was to evaluate the degree of inhibition of radiographic progression in patients with varying degrees of clinical response in Study J (in Landewe et al. (2005) Ann Rheum Dis 64(Suppl III):442).

Study J was a 2-year, double-blind, Phase III study. MTX-naïve adult patients with active, early RA (<3 years) were randomized to 1 of 3 treatment arms: adalimumab 40 mg every other week (eow)+MTX* (*7.5 mg weekly increased to 20 mg over 8 weeks, as tolerated and as needed); adalimumab 40 mg eow and placebo; or MTX alone and placebo. MTX dosages were rapidly optimized to a maximum of 20 mg weekly. Mean changes in total Sharp score (TSS) were measured for patients who achieved American College of Rheumatology (ACR) response (ACR20, ACR50, and ACR70), as well as clinical remission by Disease Activity Score 28 (DAS28<2.6). The primary endpoints were the ACR50 responses and the mean changes in Total Sharp Score (TSS) at 1 year, comparing adalimumab+MTX vs. MTX monotherapy. The study design is shown in FIG. 18.

At 1 and 2 years, the American College of Rheumatology (ACR) responses (ACR20, ACR50, and ACR70), the Disease Activity Score 28 (DAS28), and the change (A) in Total Sharp Score (TSS) were determined for all patients. Results were analyzed for the intention-to-treat (ITT) population using imputation for missing data. Clinical remission was defined as DAS28<2.6. The mean ΔTSS values at 1 year and 2 years (N=791) were determined for patients in each ACR response category (ACR20/50/70) and for patients with DAS28<2.6

A total of 799 patients enrolled in the study. Baseline demographics and clinical characteristics were similar among the 3 arms (see Example 7).

The results showed that there were statistically significantly higher ACR20/50/70 responses (see Example 7) and lower mean changes in TSS (see Example 8) observed for the combination therapy (adalimumab+MTX) arm vs. either monotherapy arm.

At each level of ACR response, significantly less radiographic progression (ΔTSS, ΔJE and ΔJSN) occurred in patients treated with the combination of adalimumab+MTX compared with either agent alone (see Tables 49-53). Little progression occurred in patients on combination therapy (adalimumab+MTX) (see Tables 49-53).

TABLE 49
Changes in TSS at 1 year by level of ACR response

| | | Ada + mtx | Ada alone | Mtx alone |
|---|---|---|---|---|
| Mean change in | ACR20 | 0.8 | 2.3 | 3.9 |
| TSS at 1 year | responder | | | |
| | ACR50 | 0.9 | 1.7 | 3.2 |
| | responder | | | |
| | ACR70 | 0.7 | 1.1 | 2.7 |
| | responder | | | |

TABLE 50
Changes in joint erosion scores at 1 year by level of ACR response

| | | Ada + mtx | Ada alone | Mtx alone |
|---|---|---|---|---|
| Mean change in | ACR20 | 0.4 | 1.0 | 2.0 |
| JE score at 1 | responder | | | |
| year | ACR50 | 0.5 | 0.8 | 2.2 |
| | responder | | | |
| | ACR70 | 0.4 | 0.5 | 1.6 |
| | responder | | | |

Changes in TSS scores at 2 years by level of ACR responder are described in Example 7.

TABLE 51
Changes in joint erosion scores at 2 year by level of ACR response

| | | Ada + mtx | Ada alone | Mtx alone |
|---|---|---|---|---|
| Mean change in | ACR20 | 0.5 | 2.3 | 3.6 |
| JE score at 2 | responder | | | |
| year | ACR50 | 0.3 | 1.5 | 3.0 |
| | responder | | | |
| | ACR70 | 0.1 | 1.6 | 2.6 |
| | responder | | | |

TABLE 52
Changes in joint space narrowing scores at 1 year
by level of ACR response

| | | Ada + mtx | Ada alone | Mtx alone |
|---|---|---|---|---|
| Mean change in | ACR20 | 0.4 | 1.3 | 1.4 |
| JSN at 1 year | responder | | | |
| | ACR50 | 0.4 | 0.9 | 1.0 |
| | responder | | | |
| | ACR70 | 0.3 | 0.6 | 1.1 |
| | responder | | | |

TABLE 53
Changes in joint space narrowing scores at 2 years
by level of ACR response

| | | Ada + mtx | Ada alone | Mtx alone |
|---|---|---|---|---|
| Mean change in | ACR20 | 0.6 | 2.2 | 2.3 |
| JSN at 2 years | responder | | | |
| | ACR50 | 0.7 | 1.9 | 1.8 |
| | responder | | | |
| | ACR70 | 0.5 | 1.9 | 1.4 |
| | responder | | | |

All ada + mtx p < 0.001 for above tables.

Patients who achieved DAS28<2.6 had lower mean ΔTSS with combination therapy (adalimumab+MTX) than with MTX alone. Changes in TSS for patients with DAS28<2.6 (clinical remission) were as follows: ada+mtx 0.9 (year 1) and 1 (year 2); ada alone −0.1 (1 year) and 1.3 (year 2); and mtx alone 1.9 (1 year) and 2.8 (year 2). Patients who had both clinical remission (DAS28<2.6) and no radiographic progression (ΔTSS≦0.5) were 2.73 times more frequent with adalimumab+MTX than with MTX alone. Similar results were observed for the two components of the TSS—joint erosions (JE) and joint space narrowing (JSN).

In sum, in the combination arm, compared with either of the monotherapy arms, ACR20/50/70 responses and the percentages of patients who achieved a DAS28 remission were statistically higher, and changes in TSS, joint erosion scores, and joint space narrowing scores were statistically lower. However, at each level of response, there was less radiographic progression in the combination arm of treatment than in either monotherapy arm. The difference was the greatest between the combination arm and MTX monotherapy arm. The rate of change in TSS in ACR20 responders who received MTX was 7 times the rate seen in ACR20 responders who received combination therapy. Similarly, patients who achieved remission (DAS28<2.6) had lower mean changes in TSS than those who did not achieve remission.

In addition, patients who achieved a DAS28 remission were less likely to progress (ΔTSS≦0.5) in the combination arm (68% and 66% at 1 and 2 years) than patients who received MTX alone (47% and 47%, p<0.01).

Similar results were observed for the two components of the TSS—joint erosions and joint space narrowing.

TABLE 54
Mean and Change in Total Sharp Score by ACR Response and DAS28 < 2.6
(Clinical Remission)
Mean Change in Total Sharp Score by ACR Response and DAS28 < 2.6 (Clinical
Remission)

| | | | Ada | | MTX | |
|---|---|---|---|---|---|---|
| | Ada + MTX | Ada + MTX | alone | Ada alone | alone | MTX alone |
| | 1 year | 2 years | 1 year | 2 years | 1 year | 2 years |
| | CR (ΔTSS) | CR (ΔTSS) | CR (ΔTSS) | CR (ΔTSS) | CR (ΔTSS) | CR (ΔTSS) |
| ACR70 | 46% (0.7) | 47% (0.7) | 26% (1.1) | 28% (3.5) | 28% (2.7) | 28% (4.1) |
| ACR50 | 62% (0.9) | 59% (1.0) | 42% (1.7) | 37% (3.4) | 46% (3.2) | 43% (4.9) |
| ACR20 | 73% (0.8) | 69% (1.1) | 54% (2.3) | 50% (4.5) | 63% (3.9) | 56% (5.8) |
| DAS28 < 2.6 | 43%* (0.9) | 49%* (1.0) | 23% (−0.1) | 25% (1.3) | 21% (2.8) | 25% (1.9) |

CR = Clinical Response;
ΔTSS = Mean Change in Total Sharp Score (TSS)
*p < 0.001 for ada + MTX vs. ada alone and MTX alone; taken at week 52 and week 54 for 1 and 2 years, respectively

In conclusion, in MTX-naïve patients with recent-onset RA, those receiving adalimumab plus MTX achieved significantly better clinical and radiographic outcomes than patients who received MTX alone or adalimumab alone. Patients who received MTX, even when they achieved ACR20/50/70 responses and clinical remission, continued to have significant radiographic progression. At all levels of ACR response (ACR20/50/70) and for patients with DAS28<2.6 (clinical remission), better radiographic outcomes were observed with combination therapy than with MTX alone. Patients who had received MTX alone continued to have significant radiographic progression, including those with ACR70 responses or DAS28 clinical remission scores.

### EXAMPLE 17

### Adalimumab (HUMIRA®) is as Effective when Used with Other Concomitant DMARDS as when Used with Methotrexate in Treating Rheumatoid Arthritis in Widespread Clinical Practice

Methotrexate (MTX) is the most commonly used traditional disease-modifying antirheumatic drug (DMARD) in the treatment of rheumatoid arthritis (RA) and is considered the gold standard. Add-on strategies with 1 or more DMARDs are often employed when the efficacy of a treatment for RA is insufficient or begins to fail Biologic antirheumatic therapies have typically been evaluated with primarily MTX as concomitant medication, but a detailed evaluation of combination therapy with other DMARDs is lacking

The objective of this study was to investigate the effects that concomitant DMARDs, including MTX, leflunomide (LEF), sulfasalazine (SSZ), and chloroquine/hydroxychloroquine (CQ/HCQ), have on key efficacy parameters after 12 weeks of treatment with adalimumab in a large cohort of patients with long-standing RA in real-life clinical practice.

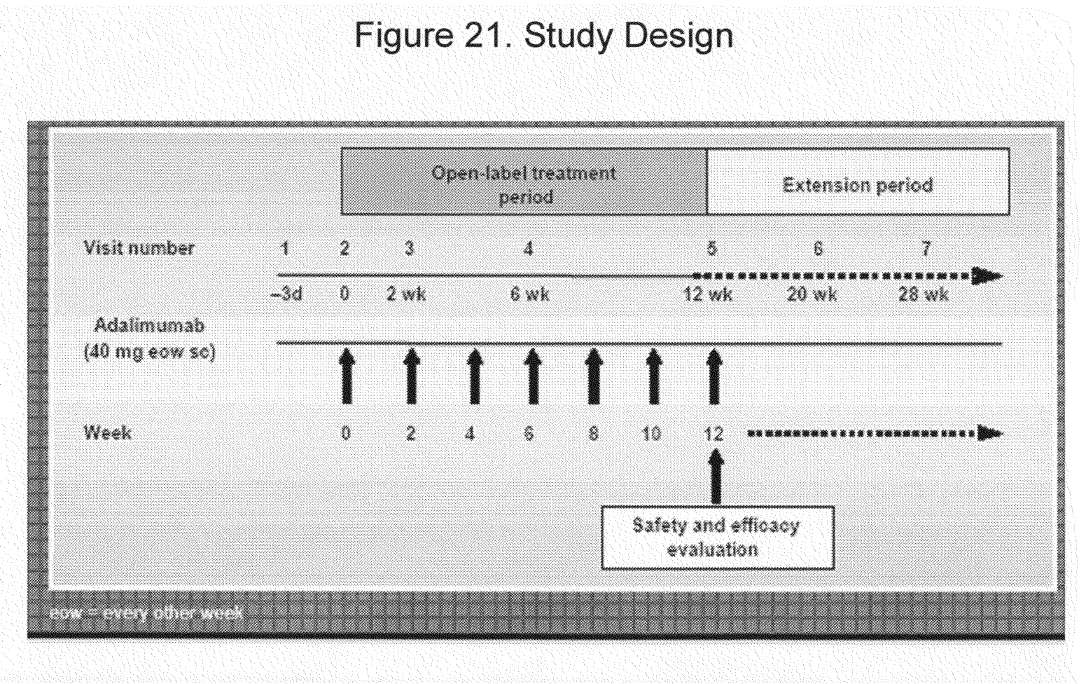

Patients with long-standing, moderate to severe RA received adalimumab in addition to their concomitant but insufficient antirheumatic therapies in Study A (study design shown in FIG. 21). Patients at more than 450 sites in 11 European countries and Australia received 40 mg adalimumab sc every other week in addition to their existing but insufficient therapies (MTX, leflunomide (LEF), sulfasalazine (SSZ), and chloroquine/hydroxychloroquine (CQ/HCQ). American College of Rheumatology Classification Criteria included the following: Age≧18 years; RA (defined by American College of Rheumatology criteria) for ≧3 months; unsatisfactory response or intolerance to at least one prior DMARD; active RA (DAS28≧3.2). Efficacy assessments were performed at Weeks 0 (baseline), 2, 6 and 12; Outcomes measured included DAS28; EULAR response; ACR20, ACR50, ACR70; TJC, SJC; and HAQ.

Routine safety and efficacy evaluations were conducted at 2, 6, and 12 weeks. For this interim analysis, efficacy outcomes were assessed after 12 weeks per type of concomitant DMARD.

Data for 4241 patients were available as of Nov. 2, 2004 for a 12-week interim analysis. The mean baseline age=54; disease duration=11 years; DAS28=6.0; HAQ=1.63; TJC28=13; SJC28=11. Concomitant DMARD usage included 22% no DMARDs (n=956), 57% 1 DMARD (n=2402), 16% 2 DMARDs (n=672) and 5% 3+DMARDs (n=211). Baseline characteristics of patients were comparable across subgroups, as shown in Table 55:

TABLE 55
Baseline Characteristics by Concomitant DMARD (exclusively)

| | | | | | | | MTX + CQ/ |
|---|---|---|---|---|---|---|---|
| Characteristics | MTX | LEF | SSZ | CQ/HCQ | MTX + LEF | MTX + SSZ | HCQ |
| N | 1561 | 557 | 89 | 85 | 123 | 141 | 153 |
| Duration of | 10 | 11 | 11 | 10 | 10 | 9 | 13 |
| RA (yrs) | | | | | | | |
| # Prior | 2.6 | 3.3 | 3.1 | 3.2 | 3.4 | 2.9 | 3.2 |
| DMARDs | | | | | | | |
| % Steroid use | 66 | 70 | 56 | 80 | 80 | 69 | 63 |
| HAQ | 1.6 | 1.5 | 1.6 | 1.7 | 1.6 | 1.6 | 1.6 |
| DAS28 | 5.9 | 6.0 | 6.1 | 6.2 | 6.1 | 5.9 | 5.7 |
| TJC28 | 13 | 13 | 13 | 15 | 14 | 13 | 12 |
| SJC28 | 10 | 10 | 11 | 11 | 11 | 11 | 10 |

*Mean values

Withdrawal rates due to lack of efficacy or to intolerance to adalimumab (all king of side effects) were low and similar in all analyzed combinations.

TABLE 56
Withdrawal Rates Due to Intolerance or Lack of Efficacy by Concomitant
DMARD at Week 12 (%)

| Reasons | | | | | | | MTX + |
|---|---|---|---|---|---|---|---|
| for | MTX | LEF | SSZ | CQ/HCQ | MTX +LEF | MTX + SSZ | CQ/HCQ |
| Withdrawal | (1561) | (557) | (89) | (85) | (123) | (141) | (153) |
| Intolerance | 3.5 | 6.0 | 3.4 | 2.4 | 2.5 | 2.9 | 2.6 |
| Lack of | 1.7 | 1.3 | 1.1 | 1.2 | none | 0.7 | 1.3 |
| Efficacy | | | | | | | |

Of the patients in the study, 22% received adalimumab monotherapy, 57% were taking 1 concomitant DMARD, 16% were taking 2, and 5% were taking 3 or more. Efficacy outcomes following 12 weeks of therapy for patients treated with adalimumab plus MTX, LEF, SSZ or QC/HQC exclusively, and the most frequently used combinations of these DMARDs are shown in the Table 57 below. Efficacy of adalimumab with concomitant LEF, SSZ, QC/HQC was similar to that observed with adalimumab plus MTX.

TABLE 57
Clinical Response to Adalimumab by Type of Concomitant DMARD Therapy

| Efficacy | Concomitant | Concomitant | Concomitant | Concomitant | Concomitant | Concomitant |
|---|---|---|---|---|---|---|
| Criteria | MTX only | LEF only | SSZ only | QC/HQC | MTX + LEF | MTX + SSZ |
| N | 1561 | 557 | 89 | 85 | 123 | 141 |
| ACR20 | 72 | 64 | 59 | 68 | 70 | 75 |
| (%) | | | | | | |
| ACR50 | 45 | 34 | 41 | 36 | 41 | 42 |
| (%) | | | | | | |
| Moderate | 84 | 79 | 75 | 85 | 86 | 89 |
| EULAR | | | | | | |
| response | | | | | | |
| (%) | | | | | | |
| Good | 39 | 31 | 39 | 25 | 29 | 41 |
| EULAR | | | | | | |
| response | | | | | | |
| (%) | | | | | | |
| Change | −2.2 | −2.0 | −1.9 | −2.1 | −2.3 | −2.3 |
| in | | | | | | |
| DAS28* | | | | | | |
| Change | −0.52 | −0.47 | −0.47 | −0.63 | −0.53 | −0.53 |
| in | | | | | | |
| HAQ* | | | | | | |
| Change | −8 | −8 | −7 | −9 | −10 | −8 |
| in | | | | | | |
| TJC (0-28)* | | | | | | |
| Change | −7 | −6 | −6 | −7 | −8 | −7 |
| in | | | | | | |
| SJC (0-28)* | | | | | | |

*Mean change from baseline

As shown above, in patients with only 1 concomitant DMARD, the effect of adding adalimumab to LEF, SSZ, and to CQ/HCQ on ACR response was similar to the effect of concomitant adalimumab and MTX (see Table 58).

TABLE 58

| | | | | | Moderate | Good |
|---|---|---|---|---|---|---|
| | | ACR20 | ACR50 | ACR70 | EULAR | EULAR |
| % of | Mtx | 72 | 45 | 20 | 84 | 39 |
| patients | (n = 1561) | | | | | |
| | Lef | 64 | 34 | 14 | 79 | 31 |
| | (n = 557) | | | | | |
| | Ssz | 59 | 41 | 15 | 75 | 39 |
| | (n = 89) | | | | | |
| | Cq/Hcq | 68 | 36 | 18 | 85 | 25 |
| | (n = 85) | | | | | |

In patients with only 1 concomitant DMARD, the effect of adding adalimumab to LEF, SSZ, and to CQ/HCQ on EULAR response was similar to the effect of concomitant adalimumab and MTX (see Table 58 above). Table 59 shows that in patients with multiple concomitant DMARDs, the effect of adding adalimumab to combinations of MTX+LEF, MTX+SSZ, MTX+CQ/HCQ on ACR response was similar to the effect of concomitant adalimumab and MTX alone.

TABLE 59

| | | | | | Moderate | Good |
|---|---|---|---|---|---|---|
| | | ACR20 | ACR50 | ACR70 | EULAR | EULAR |
| % of | Mtx | 72 | 45 | 20 | 84 | 39 |
| patients | (n = 1561) | | | | | |
| | Lef + mtx | 70 | 41 | 14 | 86 | 29 |
| | (n = 123) | | | | | |
| | Ssz + mtx | 75 | 42 | 20 | 89 | 41 |
| | (n = 130) | | | | | |
| | Cq/Hcq + | 74 | 44 | 18 | 88 | 40 |
| | mtx | | | | | |
| | (n = 153) | | | | | |

Table 59 also shows that in patients with multiple concomitant DMARDs, the effect of adding adalimumab to combinations of MTX+LEF, MTX+SSZ, MTX+CQ/HCQ on EULAR response was similar to the effect of concomitant adalimumab and MTX alone.

The effect of adding adalimumab to LEF, SSZ, CQ/HCQ and to combinations of MTX+LEF, MTX+SSZ, MTX+CQ/HCQ was similar to the effect of concomitant adalimumab and MTX, as measured by the mean change from baseline DAS28. The mean change in DAS28 from baseline at week 12 was −2.2 mtx, −2.0 lef, −1.9 ssz, −2.1 cq/hcq, −2.3 mtx+lef, −2.3 mtx+ssz, and −2.2 mtx+cq/hcq. The effect of adding adalimumab to LEF, SSZ, CQ/HCQ and to combinations of MTX+LEF, MTX+SSZ, MTX+CQ/HCQ was similar to the effect of concomitant adalimumab and MTX as measured by the mean change from baseline HAQ score. The effect of adding adalimumab to LEF, SSZ, CQ/HCQ and to combinations of MTX+LEF, MTX+SSZ, MTX+CQ/HCQ was also similar to the effect of concomitant adalimumab and MTX, as measured by the mean change from baseline TJC28. The effect of adding adalimumab to LEF, SSZ, CQ/HCQ and to combinations of MTX+LEF, MTX+SSZ, MTX+CQ/HCQ was similar to the effect of concomitant adalimumab and MTX, as measured by the mean change from baseline SJC28

In conclusion, adalimumab 40 mg sc eow led to clinically significant improvements at 12 weeks in all major efficacy parameters irrespective of the type of concomitant DMARD. In a large cohort of patients with long-standing RA and an insufficient response to DMARD therapies in real life clinical practice, the addition of ada provided substantial improvement in all key efficacy parameters. In patients with insufficient response to concomitant DMARD therapy, the addition of adalimumab provided substantial improvement in the signs and symptoms of RA.

Adalimumab demonstrated similar efficacy when combined with MTX, LEF, SSZ or QC/HQC, respectively, or combinations of these DMARDs, in short-term treatment. The effect of adding ada to LEF, SSZ, CQ/HCQ, and to combinations of MTX+LEF, MTX+SSZ, MTX+CQ/HCQ was similar to the combination with MTX alone. No differences were observed when ada was added to one compared with two concomitant DMARDs. Adalimumab was efficacious in the real-life clinical practice of treating RA with various combinations of DMARDs. Furthermore, ada was well-tolerated. Withdrawal rates due to lack of efficacy or to intolerance within 12 weeks were low and similar in all analyzed combinations. The above-mentioned study is also described in Mariette et al. Ann Rheum Dis 2005; 64(Suppl III), incorporated by reference herein.

### EXAMPLE 18

### Radiographic Progression During the First 6 Months of Disease in Recent-Onset Rheumatoid Arthritis (RA): Study of Adalimumab (HUMIRA®) Therapy in Early RA

Recent clinical trials of tumor necrosis factor (TNF) antagonists have shown that therapy with a TNF antagonist plus methotrexate (MTX) is superior to MTX alone (De Vries-Bouwstra et al. Arthritis Rheum 2003: 48:3649; Smolen J S, et al. Ann Rheum Dis 2003: 6 (Suppl I):64; and Weinblatt M E, et al. Arthritis Rheum 2003; 48:35-45). Very low disease activity, clinical remission and good radiographic outcomes are achievable with aggressive strategies (De Vries-Bouwstra et al. Arthritis Rheum 2003: 48:3649). Study J was the first trial to directly compare a TNF antagonist plus MTX with the TNF antagonist alone and MTX alone in MTX-naïve patients with recent-onset RA. The ability to predict which RA patients will have destructive disease and need more aggressive therapy would be a practical asset to rheumatology practice. Data on the therapeutic responses of patients with very early RA (<6 months) and early RA (6 months to <3 years) could assist therapeutic decisions in patients with recent onset RA

The ability to predict which RA patients will have destructive disease and deserve more aggressive therapy would be a valuable tool for practicing rheumatologists. In addition, data on the differences between responses in patients with very early RA (<6 months) and patients with early RA (>6 months to <3 years) to aggressive therapy would provide evidence for optimal therapeutic decisions. As has been described in several studies, the combination of a TNF antagonist plus methotrexate (MTX) achieves radiographic and clinical outcomes superior to monotherapy and is considered aggressive therapy.

The object of this subanalysis was to evaluate whether the rate of radiographic progression in Study J differed in early RA patients with respect to variable lengths of disease duration.

Study J was a 2-year, double-blind, active-comparator study. MTX-naïve adult patients with active, early RA (<3 years) were randomized to 1 of 3 treatment arms: adalimumab 40 mg every other week (eow)+MTX* (**7.5 mg weekly increased to 20 mg over 8 weeks, as tolerated and as needed); adalimumab 40 mg eow alone+placebo; or MTX alone+Placebo. MTX dosages were rapidly optimized up to a maximum of 20 mg weekly. The overall study design is shown in FIG. 18.

Radiographic progression was measured by Total Sharp Score (TSS) at baseline, and 1 and 2 years in the intention to treat population (ITT) using imputation for missing data. In this analysis, patients were divided into 1 of 2 categories by disease duration at baseline: <6 months (Group 1) or 6 months to 3 years (Group 2). Mean changes in TSS were reported for both groups by each of the 3 treatment arms from baseline to Year 1 and from Year 1 to Year 2.

A total of 799 patients enrolled in Study J. Baseline demographics and clinical characteristics were similar among the 3 arms (above and below examples describing Study J).

Treatment with the combination of adalimumab+MTX was most effective and MTX alone least effective in inhibiting radiographic progression in the overall population (see above examples, including example 7). Treatment differences were statistically significant by 6 months. Annualized rates of radiographic disease progression indicate that, during Study J, radiographic damage occurred much more rapidly during the first 6 months of disease vs. subsequent 6-month periods (Table 60)

TABLE 60
Annualized Rates (Sharp Score Units per Year) of Radiographic Progression
(ΔTSS)

| | | | | Ada | Ada | Ada | MTX | MTX | |
|---|---|---|---|---|---|---|---|---|---|
| | Ada + MTX | Ada + MTX | Ada + MTX | Alone | Alone | Alone | Alone | Alone | MTX Alone |
| | 0-6 mos | 6 mos-1 yr | 1-2 yrs | 0-6 mos | 6 mos-1 yr | 1-2 yrs | 0-6 mos | 6 mos-1 yr | 1-2 yrs |
| All pts in | 1.6 | 1.0 | 0.6 | 4.2 | 1.8 | 2.5 | 7.0 | 4.4 | 4.7 |
| treatment | | | | | | | | | |
| arm | | | | | | | | | |

Disease severity at baseline (by DAS28) was comparable among patients with RA duration <6 mos (Group 1) and RA for 6 mos to 3 yrs (Group 2).

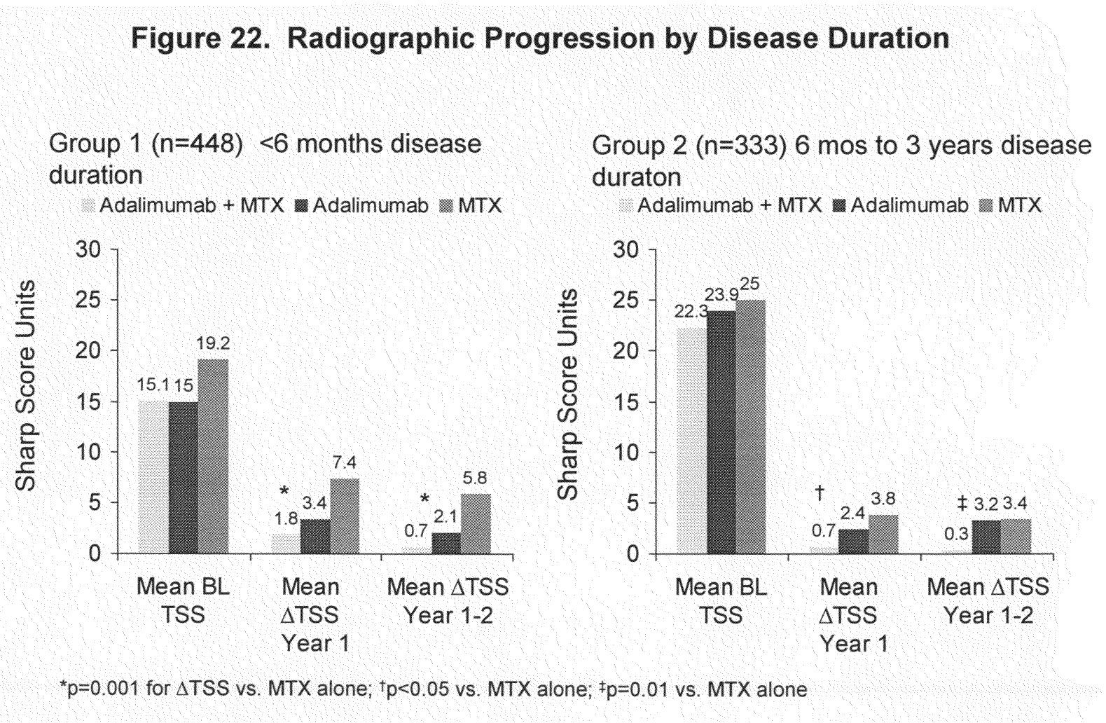

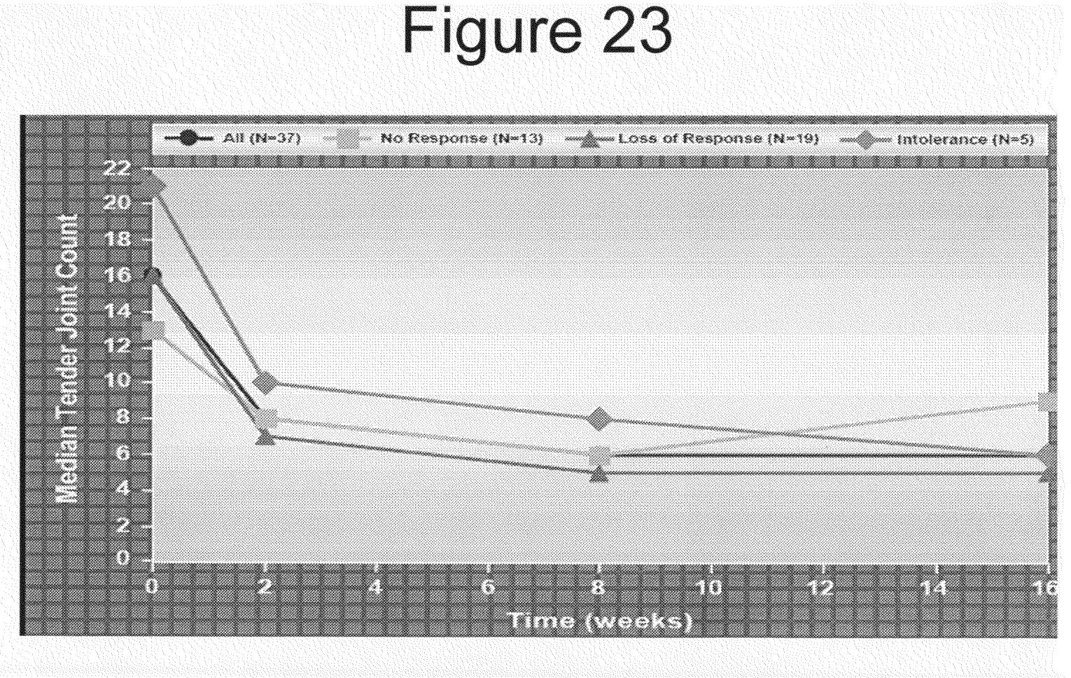

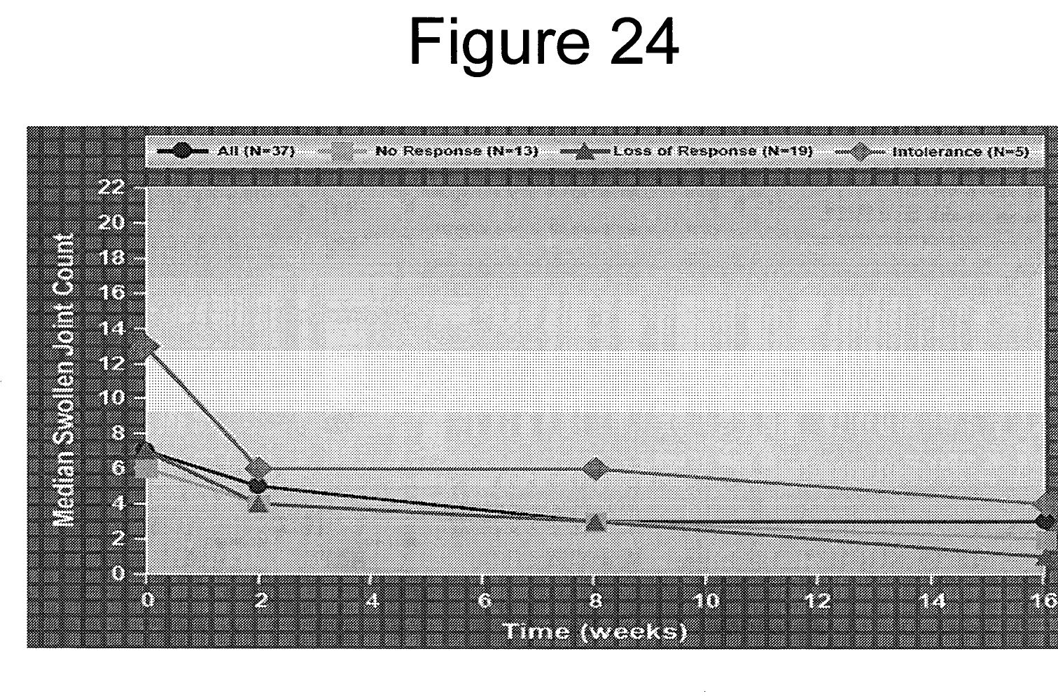

Mean TSS scores at baseline for patients in Group 1 (RA<6 mos) were two-thirds to three-quarters of the values for patients in Group 2 (RA 6 mos to 3 yrs). FIG. 22 shows that mean TSS scores at baseline in Group 1 were approximately two-thirds as high as those in Group 2. The ΔTSS with adalimumab+MTX differed most from the ΔTSS with MTX monotherapy in patients with disease duration <6 months, especially during the first year of therapy (FIG. 22). For patients treated with adalimumab+MTX or MTX alone:
ΔTSS was higher with earlier disease (Group 1 vs. Group 2)
ΔTSS was higher in Year 1 of treatment than in Year 2.

Combination therapy with adalimumab+MTX led to statistically significantly greater inhibition of radiographic progression than did MTX alone during the first and second years of therapy, see Tables 61 and 62.

TABLE 61
Radiographic progression by disease duration <6 months
(n = 448) (given in sharp score units)

| | Ada + mtx | Ada alone | Mtx alone |
|---|---|---|---|
| Mean BL TSS | 15.1 | 15 | 19.2 |
| Mean change TSS | 1.8 | 3.4 | 7.4 |
| during year 1 | | | |
| Mean change TSS | 0.7 | 2.1 | 5.8 |
| during year 2 | | | |

TABLE 62
Radiographic progression by disease duration 6 months-3 years
(n = 333) (given in sharp score units)

| | Ada + mtx | Ada alone | Mtx alone |
|---|---|---|---|
| Mean BL TSS | 22.3 | 23.9 | 25 |
| Mean change TSS | 0.7 | 2.4 | 3.8 |
| during year 1 | | | |
| Mean change TSS | 0.3 | 3.2 | 3.4 |
| during year 2 | | | |

The TSS at baseline and after 2 years by disease duration at baseline were as follows: disease duration less than 6 months: ada+mtx 17.6 TSS (15.1 TSS at baseline and 2.5 change in TSS over 2 years) vs. mtx alone 32.4 (19.2 at baseline and 13.2 change in TSS over 2 years). For disease duration 6 months to 3 years, ada+mtx 23.4 TSS (22.3 TSS at baseline and 1.0 change in TSS over 2 years) vs. mtx alone 32.2 (25.0 at baseline and 7.2 change in TSS over 2 years).

Overall, combination therapy led to the greatest and most predictable inhibition of radiographic progression. Combination therapy led to statistically significantly greater inhibition of radiographic progression vs. MTX during the first and second years of therapy. In addition, the difference between combination and MTX monotherapy was the greatest for patients in Group 1 (<6 months of disease) during the first year of therapy.

TABLE 63
Radiographic Progression By Disease Duration at Baseline (BL)

| | | Mean ΔTSS at 1 | Mean ΔTSS |
|---|---|---|---|
| | Mean BL TSS | year | Yr 1-Yr 2 |
| Group 1 (n = 448) | | | |
| BL Dis Dur <6 mos | | | |
| Combo (n = 260) | 15.1 | 1.8* | 0.7* |
| Ada alone (n = 259) | 15.0 | 3.4 | 2.1 |
| MTX alone (n = 252) | 19.2 | 7.4 | 5.8 |
| Group 2 (n = 333) | | | |
| BL Dis 6 mos to 3 yrs | | | |
| Combo | 22.3 | 0.7† | 0.3‡ |
| Ada alone | 23.9 | 2.4 | 3.2 |
| MTX alone | 25.0 | 3.8 | 3.4 |

*p = 0.001 for ΔTSS vs. MTX alone;
†p < 0.05 vs. MTX alone;
‡p = 0.01 vs. MTX alone.

In conclusion, in MTX-naïve patients with recent-onset RA, the greatest degree of inhibition of radiographic progression was achieved by combination therapy with adalimumab plus MTX, regardless of duration of disease at baseline. This benefit of adalimumab+MTX, compared with MTX alone, was most marked in patients with disease duration of <6 months and was seen at both 1 and 2 years. Further, these results suggest that, in this patient population, the greatest rate of radiographic damage occurs very early in the disease course, and that the best radiographic outcomes are achieved by the initiation of combination therapy as early in the disease course as possible. For patients with very early disease (<6 months), combination therapy provided substantial and statistically significantly greater inhibition of radiographic progression vs. MTX monotherapy at 1 and 2 years.

### EXAMPLE 19

### Adalimumab (HUMIRA®) Plus Methotrexate is Safe and Efficacious in Patients with Rheumatoid Arthritis into the 7^{th} Year of Therapy

In clinical trials, adalimumab, a fully human anti-TNF monoclonal antibody, has been shown to reduce the signs and symptoms and to inhibit radiographic progression of disease in patients with active, moderate to severe rheumatoid arthritis (RA).

The objective of this study was to assess the sustainability of the safety profile and efficacy outcomes of adalimumab 40 mg every other week (eow) plus methotrexate (MTX) in patients with long-standing, moderate to severe RA in two long-term, open-label extension (OLE) studies.

Patients who enrolled in five randomized, controlled trials (RCT) and received adalimumab 40 mg eow plus MTX were evaluated in this analysis. After these trials, patients were eligible to enroll in two separate OLE studies: Study 4 and Study 5. Patients were evaluated for efficacy and safety every 3 months.

A total of 921 patients received adalimumab 40 mg every other week (eow) plus MTX and enrolled in the RCTs leading into the Study 4 OLE. Efficacy outcomes were evaluated as observed data: DAS28; ACR 20/50/70; Tender Joint Count (TJC); Swollen Joint Count (SJC); and HAQ Disability Index. Baseline demographics and disease characteristics were similar in both Study 4 and Study 5 consistent with moderate to severe RA, as shown below in Table 64:

TABLE 64
Baseline demographics and disease characteristics

| | Study 4 | Study 5 |
|---|---|---|
| | N = 921 | N = 43 |
| Age (years) | 55 | 54 |
| % Female | 78 | 79 |
| Duration of RA (Years) | 11 | 11 |
| TJC (0-28) | 13 | 13 |
| SJC (0-28) | 12 | 15 |
| HAQ (0-3) | 1.3 | 1.4 |
| CRP (mg/L) | 1.8 | 2.8 |
| DAS28 | 5.4 | 5.8 |

At the time of this analysis, 617 (66%) patients remain on therapy in the OLE. A total of 304 (33%) patients withdrew: 74 (8%) for lack of efficacy, 106 (11%) for adverse events, and 124 (14%) for other reasons. Eighty-nine patients are into their 5th year of treatment and have demonstrated sustained and consistent clinical improvement over time, as supported by efficacy outcomes and by achievement of clinical remission based on DAS28<2.6. At the last visits of all 921 patients, 23% had 0 tender joints (TJC68), 20% had 0 swollen joints (SJC66), and 42% had a HAQ score ≦0.5—all are parameters of clinical remission.

Forty-three patients randomized in the Phase I RCT leading into the Study 5 OLE study received adalimumab 40 mg eow plus MTX are included in this analysis. At the time of this evaluation, 31 (72%) patients remain in the OLE and 12 (28%) patients withdrew: 1 (2%) for lack of efficacy, 3 (7%) for adverse events, and 8 (19%) for other reasons. At last visits, 9% had 0 tender joints (TJC68), 23% had 0 swollen joints (SJC66), and 26% had a HAQ score ≦0.5. Sustained response is supported by efficacy outcomes in 15 patients who are into their 7th year of adalimumab therapy. In both OLE trials, adalimumab plus MTX was well-tolerated and rates of adverse events, including serious infections, were consistent with those observed in the RCTs.

The Kaplan-Meier curves provide a projection of patients receiving adalimumab (all doses) that will remain on therapy at Year 5 (Study 4) and at Year 7 (Study 5). Improvements in DAS28 achieved at 6 months were sustained over time in Studies 4 and 5, and ACR responses (ACR 20/50/70) were sustained into Year 5 in Study 4 and sustained into Year 7 in Study 5. Over 40% of patients achieved clinical remission (DAS28<2.6) at Year 5 (Study 4) and Year 7 (Study 5), as described below in Table 65.

TABLE 65
Long-term Efficacy in Patients Treated with Adalimumab plus MTX

| | Study 4 - Year 5 | Study 5 - Year 7 |
|---|---|---|
| Efficacy Criteria | n = 89 | n = 15 |
| ACR20 (%) | 72 | 87 |
| ACR50 (%) | 55 | 60 |
| ACR70 (%) | 38 | 33 |
| DAS28 < 2.6 (%) | 41 | 43 |
| DAS28* | 2.9 | 2.7 |
| DAS28 change from | 2.9 | 3.6 |
| baseline* | | |
| HAQ* | 0.5 | 0.9 |
| HAQ change from | 0.8 | 0.6 |
| baseline* | | |

*Median values

Rates and types of serious adverse events demonstrated a consistent safety profile in relation to previously reported adalimumab pivotal trials.

In conclusion, patients with long-standing RA treated with adalimumab 40 mg eow plus MTX achieved sustained and consistent improvements, into their 7th year of continuous therapy, with more than 40% achieving clinical remission (DAS28<2.6). Long-term adalimumab therapy is safe and well-tolerated. Patients with long-standing RA maintained clinical improvements and a significant reduction of disease activity for up to year 7 of continuous treatment with adalimumab 40 mg eow plus MTX.

### EXAMPLE 20

### Safety of Adalimumab (HUMIRA®) in Global Clinical Trials of Patients with Early vs. Long-Standing Rheumatoid Arthritis (RA)

The objective of the study was to assess the safety of adalimumab in the treatment of patients with early vs. long-standing RA who received adalimumab in randomized pivotal trials, open-label trials, or in phase IIIb studies (Schiff et al. Ann Rheum Dis 2005; 64(Suppl III):422).

All patients with RA participating in pivotal, randomized and controlled trials of adalimumab were eligible to enroll in open-label trials in which they received 40 mg adalimumab every other week. Study J was a 2-year randomized controlled trial in patients with early RA (disease duration<3 years). Participants in these clinical trials and in other phase IIIb studies, were routinely evaluated for safety. Reports of serious adverse events (SAE) were tabulated using MedDRA coding by events per 100-patient-years (E/100PY). SAE rates were compared with respect to disease duration between patients with early (<3 yrs) and long-standing RA.

As of Aug. 31, 2004, 10,050 patients (12,066 PY exposure) with long-standing RA had enrolled in adalimumab clinical trials world-wide; 271 of these had been treated with adalimumab for >5 years.

TABLE 66
Adalimumab (Ada) Clinical Exposure

| | All RA Trials | Long-standing RA | Early RA |
|---|---|---|---|
| | 31-Aug-2002* | 31-Aug-2004 | Study J |
| Patients (N) | 2,468 | 10,050 | 542 |
| Exposure (PY) | 4,870 | 12,066 | 917 |

542 patients with early RA were randomized to received adalimumab in Study J for 2 years of double-blind treatment (917 PY exposure). Rates of serious infections (E/100PY) in patients with long-standing RA in adalimumab clinical trials through Aug. 31, 2004 were comparable to rates reported in the RA population (see Schiff et al. Global Safety in Adalimumab (HUMIRA®) Rheumatoid Arthritis Clinical Trials. Poster presented at ACR 2004, San Antonio, Tex.; Doran et al. Arthritis Rheum 2002; 46:2287-93; and Moreland et al. J Rheumatol 2001; 28:1238-44). In patients with early RA (Study J), rates of serious infections were lower. Serious infections in ADA RA clinical trials included 4.8 serious infections (E/100PY) for ada and ada+mtx in long-standing RA (over 12,000 exposure (pt-yrs)) (see Schiff et al. Global Safety in Adalimumab (HUMIRA®) Rheumatoid Arthritis Clinical Trials. Poster presented at ACR 2004, San Antonio, Tex.; Doran et al. Arthritis Rheum 2002; 46:2287-93; and Moreland et al. J Rheumatol 2001; 28:1238-44). 0.7 serious infections (E/100PY) were seen in ada alone for early RA (n=435) and 2.9 were seen in ada+mtx (n=482) in early RA, observed in Study J.

Patients in pivotal trials had baseline characteristics indicative of moderate to severe RA. Rates (E/100PY) of selected SAE among patients with long-standing RA as of Aug. 31, 2004 were comparable to an earlier report of global safety in adalimumab RA clinical trials (Schiff et al. Global Safety in Adalimumab (HUMIRA®) Rheumatoid Arthritis Clinical Trials. Poster presented at ACR 2004, San Antonio, Tex.). The overall rate of serious infections observed in patients with long-standing RA was comparable to rates in published reports of RA patients treated with DMARDs, including TNF antagonists (Doran et al. Arthritis Rheum 2002; 46:2287-93; and Moreland et al. J Rheumatol 2001; 28:1238-44). Rates for serious infections and other SAE of interest were lower in the early RA trial (Table 67). There were no cases of histoplasmosis, demyelinating disease, lymphoma, lupus-like syndrome or pancytopenia in adalimumab-treated patients with early RA.

TABLE 67
Serious Adverse Event Rates in Patients Treated with Adalimumab
(E/100PY)

| Serious Events | Long-standing RA | Early RA |
|---|---|---|
| of Interest | 31-Aug-2004 | Study J |
| N | 10,050 | 542 |
| PY | 12,066 | 917 |
| Serious Infections | 4.79 | 1.85 |
| Pneumonia | 0.87 | 0.55 |
| Urinary Tract | 0.38 | 0.11 |
| Infections | | |
| Septic Arthritis | 0.46 | 0.22 |
| Tuberculosis | 0.24 | 0.11 |
| Histoplasmosis | 0.03 | 0.00 |
| Demyelinating | 0.07 | 0.00 |
| Diseases | | |
| Lymphoma | 0.11 | 0.00 |
| SLE/Lupus-like | 0.06 | 0.00 |
| Syndrome | | |
| Congestive Heart | 0.26 | 0.11 |
| Failure | | |
| Pancytopenia | 0.02 | 0.00 |

Eight cases of demyelinating diseases were identified in patients with long-standing RA enrolled in adalimumab clinical trials through Aug. 31, 2004. No cases of demyelinating diseases were reported in early RA (Study J).

TABLE 68
Cases of Demyelinating Diseases

| | Long-standing RA | Early RA |
|---|---|---|
| | 31-Aug-2004 | Study J |
| N (Exposure) | 10,050 (12,066) | 542 (917) |
| Multiple sclerosis | 4 | 0 |
| Non-specific demyelination | 2 | 0 |
| Guillain-Barre syndrome | 2 | 0 |

Fifteen cases of lymphoma were identified in patients with long-standing RA enrolled in adalimumab clinical trials through August 2004 (Standardized Incidence Ratio=3.2 as of Aug. 31, 2004; 95% CI: 1.8-5.3). The combined SIR for all patients with RA treated in clinical trials with adalimumab was 2.91, 95% CI (1.63-4.80). No cases of lymphoma were reported in patients with early RA.

TABLE 69
Cases of Lymphoma

| | Observed | Expected | SIR | 95% CI |
|---|---|---|---|---|
| Long-standing RA | | | | |
| Total lymphomas | 15 | 4.68 | 3.21 | 1.79-5.29 |
| NHL | 14 | 4.32 | 3.24 | 1.77-5.44 |
| Hodgkin's | 1 | 0.36 | 2.78 | 0.04-15.46 |
| Early RA | | | | |
| Total lymphomas | 0 | 0.37 | 0 | 0-21.5 |
| Combined | | | | |
| Total lymphomas | 15 | 5.16 | 2.91 | 1.63-4.80 |

Rates of congestive heart failure (CHF) were low in North American and European open-label extension trials of adalimumab and were comparable to rates reported in the literature for patients with RA irrespective of treatment with TNF-antagonists or history of CHF.

In conclusion, the safety profile of adalimumab has been stable over time—no new safety signals have been identified. Overall safety outcomes in patients with early RA demonstrated fewer serious adverse events than have been reported for patients with long-standing RA.

### EXAMPLE 21

### Adalimumab (HUMIRA®) is Effective in Treating Patients with Rheumatoid Arthritis who Previously Failed Infliximab Treatment

Limited experience is available on the use of one TNF-antagonist in the treatment of rheumatoid arthritis (RA) following unsuccessful treatment with a previous biologic, often for an unsatisfactory response including either lack or loss of efficacy, or development of intolerance.

The objective of this study was to investigate the safety and efficacy of adalimumab administered to patients with RA who had failed prior treatment with infliximab.

Patients eligible to participate in the study included those with long-standing, moderate to severe RA who had terminated infliximab therapy because of lack or loss of efficacy and/or intolerance/side effects. Patients were treated with adalimumab sc 40 mg every other week as monotherapy or as add-on therapy to a pre-existing DMARD treatment. Follow-up visits for safety and efficacy monitoring were scheduled at 2, 8, and 16 weeks. Adalimumab responders were allowed to continue treatment for 56 weeks. Efficacy parameters included TJC28 and SJC28; DAS28; ACR20, ACR50 Response; Moderate EULAR response; and HAQ. Subgroup analysis by reason for discontinuation included:
in case of discontinuation due to lack or loss of efficacy, patients were counted in the corresponding group regardless of additional cause of intolerance.
The subgroup “Discontinuation due to intolerance” was exclusive.

A total of 41 patients with RA (88% female, mean age 55 years) who had previously failed infliximab participated in this study. 28 patients were treated with concomitant DMARDs, primarily MTX. 13 patients were treated without DMARDs. 37 patients completed Week 16. Mean duration of previous infliximab treatment was 17 months. Mean baseline disease characteristics included: duration of RA, 12 yrs; 5.4 prior DMARDs, including infliximab; TJC28, 15; SJC28, 8; DAS28, 6.1; and HAQ, 1.85. Patients had been treated with infliximab for a mean of 17 months (range 3-67 months) and a median of 13 months.

Reasons for infliximab discontinuation-included lack of efficacy (n=15, 37%), loss of efficacy after initial response (n=221, 51%), and drug intolerance (n=7, 17%) (categories not mutually exclusive). Duration of prior infliximab therapy varies by reason for discontinuation. Mean dosages of prior infliximab therapy were similar in all groups.

TABLE 70
Administration of prior infliximab by reason for discontinuation

| | | No | Loss of | |
|---|---|---|---|---|
| | All | response | Response | Intolerance |
| Administration | (N = 41) | (N = 15) | (N = 21) | (N = 5) |
| Duration of Infliximab | 17 | 9 | 23 | 16 |
| Treatment (months)* | (3-67) | (3-19) | (5-67) | (4-40) |
| | [13] | [8] | [16] | [11] |
| Dose per Infusion | 262 | 264 | 268 | 237 |
| (mg)* | | | | |

Groups were well-matched for baseline characteristics, regardless of reason for discontinuing prior infliximab treatment, as shown below in Table 71:

TABLE 71
Baseline Demographics and Disease Severity Characteristics by
Reason for Discontinuation of Prior Infliximab (Mean values)

| | | | Loss of | |
|---|---|---|---|---|
| | All | No Response | Response | Intolerance |
| N | 41 | 15 | 21 | 5 |
| Gender | 88 | 93 | 86 | 80 |
| (% female) | | | | |
| Age (yrs) | 55 | 55 | 53 | 55 |
| # of Prior DMARDs | 5.4 | 5.5 | 5.5 | 4.2 |
| (including infliximab) | | | | |
| Duration RA (years) | 12 | 12 | 12 | 9 |
| TJC | 15 | 14 | 15 | 19 |
| SJC | 8 | 6 | 9 | 12 |
| DAS28 | 6.1 | 5.8 | 6.2 | 6.5 |
| HAQ | 1.85 | 1.92 | 1.80 | 1.85 |

Of the 41 patients treated enrolled, 37 had completed 16 weeks of adalimumab treatment and their data were available for safety and efficacy analysis (completer analysis). While on adalimumab therapy, 19 of 37 (51%) of the patients received concomitant MTX. Twenty-one of 37 (57%) had a decrease in DAS28 of ≧1.2 (maximum change −4.7) at week 16. Results from the study are shown in FIGS. 21 and 22, where TJC and SJC improvement is maintained in patients who previously failed treatment with infiximab. In addition, change in HAQ at week 16 by reason for discontinuation included the following: −0.25 all (n=37), −0.23 no response (n=13), −0.36 loss of response (n=19), and −0.15 intolerance (n=5). The MCID was determined to be −0.22.

Adalimumab was well-tolerated. Four patients discontinued treatment prematurely. One patient treated previously with infliximab for 5 years developed a non-Hodgkin's lymphoma 3 weeks after enrolling in the study. The other 3 patients withdrew because of RA flare, skin rash, and injection site reactions, respectively.

Patients with previous insufficient response to infliximab improved in all key efficacy parameters (documented by completer analysis). Only one of 41 patients dropped out due to insufficient efficacy (RA flare). Adalimumab therapy resulted in measurable ACR and EULAR responses in patients who discontinued previous infliximab therapy, as shown below in Table 72. Of 37 patients, 57% experienced a substantive improvement of DAS28 Patients with previous unsatisfactory efficacy under infliximab had improvement in physical function when subsequently treated with adalimumab.

TABLE 72
16-Wk Efficacy: Patients Treated with Adalimumab
Following Infliximab Withdrawal

| | | | Loss of | |
|---|---|---|---|---|
| | All | No response to | response to | Infliximab |
| Efficacy | reasons | infliximab | infliximab | intolerance |
| Parameters | (N = 37) | (N = 13) | (N = 19) | (N = 5) |
| ACR20 (%) | 49 | 33 | 61 | 40 |
| ACR50 (%) | 26 | 8 | 39 | 20 |
| Moderate | 65 | 46 | 74 | 80 |
| EULAR | | | | |
| Response (%) | | | | |
| Change in | −1.6 | −1.0 | −2.1 | −1.4 |
| DAS28* | | | | |
| Change in TJC | −7.4 | −5.1 | −8.2 | −10.0 |
| (0-28)* | | | | |
| Change in SJC | −5.2 | −2.8 | −6.1 | −7.8 |
| (0-28)* | | | | |

*Mean change from baseline

Adalimumab was well-tolerated. 4 patients withdrew from the study. 1 patient, who had been treated previously for 5 years with infliximab, developed a non-Hodgkin's lymphoma 3 weeks after enrolling in the study. 3 patients withdrew because of RA flare, skin rash, and injection site reactions, respectively

In conclusion, patients with RA who failed infliximab treatment experienced good outcomes when subsequently treated with adalimumab. Patients with RA who failed infliximab treatment experienced substantive improvements of efficacy parameters when subsequently treated with adalimumab. Patients with a lack of response to infliximab also profited from subsequent adalimumab therapy. Adalimumab therapy was well-tolerated, even by patients who were intolerant to infliximab. Use of adalimumab following infliximab therapy was safe, even in patients who had discontinued infliximab because of intolerance.

### EXAMPLE 22

### Baseline CRP Concentrations Predict Radiographic Progression in MTX-Naïve Patients with Early RA: Subanalysis of the Study J

Rheumatoid arthritis (RA) has a variable course with a wide range of potential outcomes, making it difficult to predict disease progression and magnitude of therapeutic response. It is difficult to predict which patients with rheumatoid arthritis (RA) will have progressive disease or who will respond to therapy. Very low disease activity, clinical remission and good radiographic outcomes are achievable with aggressive treatment strategies (De Vries-Bouwstra et al. Arthritis Rheum 2003: 48:3649). Recent clinical trials of tumor necrosis factor (TNF) antagonists demonstrate that combination therapy of a TNF antagonist plus methotrexate (MTX) is superior to MTX alone (De Vries-Bouwstra et al. Arthritis Rheum 2003: 48:3649; Smolen et al. Ann Rheum Dis 2003: 61(Suppl I):64; and Weinblatt et al. Arthritis Rheum 2003; 48:35-45). C-reactive protein (CRP) concentration may be a predictor of response to therapy (Buch et al. Arthritis Rheum 2005; 52:42-8).

The object of this study was to examine baseline characteristics that might predict radiographic progression in methotrexate-naïve patients with recent-onset RA in Study J.

Study J was a 2-year, double-blind, Phase III study of adult patients with active, early RA (<3 years) who were naïve to methotrexate (MTX). Treatment arms included adalimumab 40 mg every other week (eow)+MTX; adalimumab 40 mg eow alone, or MTX alone. The study design for Study J is shown in FIG. 18. Primary endpoints were ACR50 responses and change in Total Sharp Score (TSS) at 1 year, comparing adalimumab+MTX vs. MTX alone

Radiographic progression was assessed by mean change from baseline in Total Sharp Score (TSS) and stratified into those with (ΔTSS>0.5) and without (Δ≦TSS 0.5). Patients were categorized as either having radiographic progression at 1 year (ΔTSS>0.5) or not (Δ≦TSS 0.5). A logistic regression model was used to identify predictors of radiographic disease progression by testing several baseline characteristics, including age, disease duration, RF status, TJC, SJC, joint erosions, and CRP. For analyses of CRP, patients were categorized as normal (CRP≦0.8 mg/dL) or abnormal (CRP>0.8 mg/dL).

Of the 799 patients who enrolled in the study, baseline demographics and clinical characteristics were similar among the 3 arms (shown in FIG. 17). CRP concentrations and X-rays were available for 585 patients at baseline and 1 year.

No disease progression (ΔTSS≦0.5) was more common with combination therapy (64%) than with adalimumab alone (51%, p<0.01) or MTX alone (38%, p<0.01). Overall, patients with elevated CRP at baseline were 3.5 times more likely to have had disease progression by 1 year than patients who had normal baseline CRP concentrations. The percentage of patients who had no worsening (change in TSS≦0.5) was 81% in patients who had normal baseline CRP levels and 48% in patients with adnormal baseline CRP.

Most patients with a normal CRP at baseline at 1 year had no disease progression at 1 year (84%). Patients with abnormal CRP concentrations at baseline were almost twice as likely to have had disease progression if their CRP concentrations became normal at 1 year (55% vs. 35%).

Approximately 15-20% of patients in each treatment arm had a normal CRP concentration at baseline (Table 73 below).

TABLE 73
Patient Numbers by CRP at Baseline and 1 Year

| | | Adalimumab + | Adalimumab | MTX |
|---|---|---|---|---|
| CRP Categories | | MTX | Alone | Alone |
| Baseline | 1 Year | (n = 211) | (n = 187) | (n = 187) |
| Normal | Normal | 32 | 33 | 21 |
| Normal | Abnormal | 3 | 4 | 7 |
| Abnormal | Normal | 148 | 78 | 84 |
| Abnormal | Abnormal | 28 | 72 | 75 |

Normal CRP = ≦0.8 mg/dL
Abnormal CRP = >0.8 mg/dL

An abnormal baseline CRP concentration was more likely to have become normal at 1 year with adalimumab+MTX in combination (84%) than with adalimumab alone (52%) or MTX alone (53%), as shown below in Table 74:

TABLE 74
Percentages of Patients with Abnormal Baseline CRP
that Became Normal at 1 Year

| | | Adalimumab | |
|---|---|---|---|
| | Adalimumab + MTX | Alone | MTX Alone |
| % of Patients | 84% (n = 176) | 52% (n = 150) | 53% (n = 159) |

In sum, the percentages of patients with no worsening in TSS (ΔTSS≦0.5) at 1 year were significantly higher in patients receiving combination therapy (64%) compared with patients who received either adalimumab alone (51%, p<0.01) or MTX alone (38%, p<0.01). Using logistic regression, baseline predictors of radiographic progression were identified at Year 1. Only baseline CRP (normal/abnormal) had any significant effect on radiographic progression. Patients with elevated CRP at baseline were 3.5 times more likely to have progressed by 1 year than patients who had normal concentrations at baseline. In further analyses, we examined the effect of treatment on CRP at 1 year to assess correlation with CRP normalization and radiographic progression. Patients who had normal CRP at baseline and at 1 year were least likely to progress (84% had no progression overall).

Patients who had abnormal CRP at baseline but normal concentrations at 1 year were less likely to progress than those who had elevated concentrations at both time points (55% vs 35%). Patients with an abnormal CRP at baseline were more likely to normalize their CRP at 1 year if they received combination therapy (84%) than if they received either adalimumab alone (52%) or MTX alone (53%). Further, patients with abnormal CRP at baseline who normalized their CRP at 1 year were less likely to progress if they received combination therapy (68%) than if they received either adalimumab alone (53%) or MTX alone (33%). Patients who had elevated CRP at both baseline and 1 year were still less likely to progress if they received combination therapy (54%) than if they received either adalimumab alone (32%) or MTX alone (31%).

Most patients with normal baseline CRP had no radiographic progression, regardless of which treatment was they had received (see table 75 below). Among patients with abnormal baseline CRP concentrations that became normal at 1 year, an outcome of no radiographic progression was more frequent in those treated with adalimumab+MTX or adalimumab alone than MTX alone. Among patients with abnormal baseline CRP concentrations that remained elevated, an outcome of no radiographic progression was more frequent among those treated with adalimumab+MTX than adalimumab alone or MTX alone

TABLE 75
Patients with No Radiographic Progression at 1 Year

| CRP Concentration | | Abnormal to | |
|---|---|---|---|
| (BL and Year 1) | Always Normal | Normal | Never Normal |
| Adalimumab + MTX | 75% | 68%^{£§} | 54%^{£} |
| (n = 211) | | | |
| Adalimumab alone | 91% | 53%* | 32% |
| (n = 187) | | | |
| MTX alone | 86% | 33% | 31% |
| (n = 187) | | | |
| Overall | 84% | 55% | 35% |
| (n = 585) | | | |

^{£}p < 0.05 ada + MTX vs MTX alone,
^{§}p < 0.05 ada + MTX vs ada alone,
*p < 0.05 ada vs MTX alone.

Furthermore, logistic regression analyses demonstrated that age, disease duration, RF status, TJC, and SJC baseline variables were not associated/correlated with radiographic progression (Table 76)

TABLE 76
Analysis of Radiographic Progression at 1 Year,
Adjusting for Baseline Covariates

| Baseline | Odds Ratio | |
|---|---|---|
| Covariates | Estimate | P-value |
| ADA vs. MTX | 0.547 | 0.8249 |
| Age | 0.997 | 0.6217 |
| Baseline CRP (normal/abnormal) | 1.139 | <0.0001 |
| Baseline Joint Erosion Score | 1.013 | 0.0580 |
| Combination vs. MTX | 0.321 | <0.0001 |
| Disease Duration | 0.751 | 0.0040 |
| RF+ | 0.959 | 0.8455 |
| Swollen Joint Count | 0.998 | 0.7693 |
| Tender Joint Count | 0.993 | 0.2742 |

In Table 76 above, odds ratio estimates >1.0 indicate a positive association (correlation) with the outcome “radiographic progression at 1 year.” Baseline CRP concentration was statistically significantly associated with radiographic progression.

In conclusion, normalization of CRP correlated well with less radiographic progression, and combination therapy with adalimumab plus MTX was the most effective therapy to normalize CRP and inhibit radiographic progression when CRP was elevated at baseline. Approximately two-thirds of patients receiving MTX alone who had elevated baseline CRP developed radiographic progression whether or not their CRP normalized. In recent-onset RA, CRP at baseline and during follow-up may be used to trace those patients who may benefit most from combination therapy using adalimumab and mtx.

### EXAMPLE 23

### Sustained Efficacy after Dose Reduction of Concomitant Methotrexate and/or Corticosteroids in Patients with Rheumatoid Arthritis Treated with Adalimumab (HUMIRA®)

In patients with rheumatoid arthritis (RA) treated with adalimumab, dose adjustments of concomitant methotrexate (MTX) and corticosteroids are a standard tool in routine disease management. Dose reductions are intended to maintain maximum efficacy of TNF inhibition while minimizing side effects of concomitant medications. The initial phase of Study I was a 6-month randomized, controlled trial (Arthritis Rheum 2003; 48:35-45) which demonstrated adalimumab plus MTX significantly reduces signs and symptoms and improve functional outcomes in patients with long-standing RA.

The object of this study was to assess the impact of dose adjustments of concomitant MTX and corticosteroids on efficacy outcomes of patients with RA treated with adalimumab.

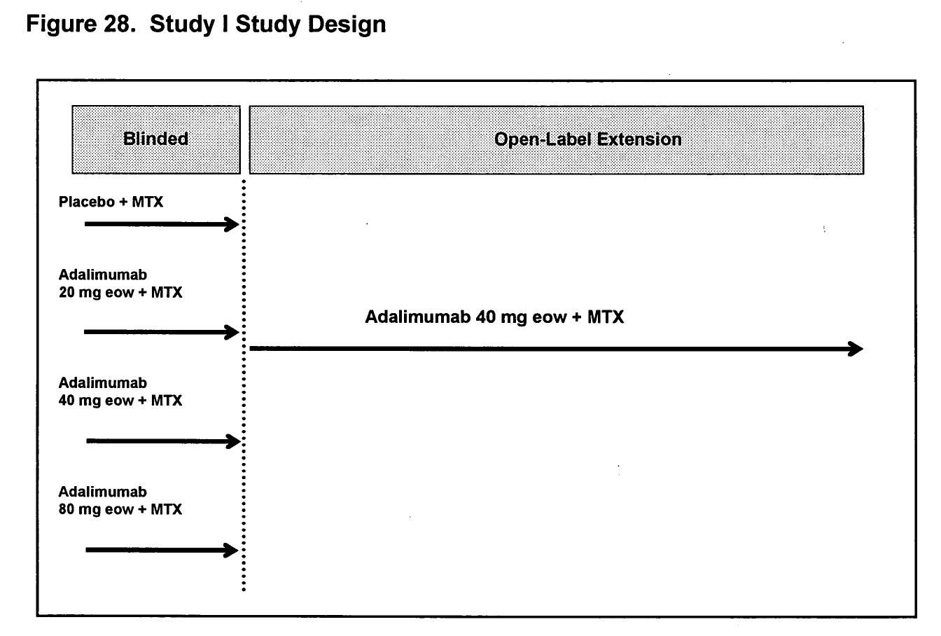

271 patients enrolled in Study I, a double-blind, placebo-controlled trial for 6 months in which adalimumab plus MTX demonstrated a rapid onset of action and significant improvement in the signs, symptoms, and functional outcomes of RA (Arthritis Rheum 2003; 48:25-45). The study design is shown in FIG. 28. Patients who completed the controlled Study I trial were eligible to enroll in the open-label extension study in which all patients received adalimumab 40 mg every other week (eow) plus MTX in addition to their current concomitant corticosteroids. 217 patients who completed the blinded period and participated in the extension trial for a minimum of 6 months were allowed to adjust MTX and/or corticosteroid dosages at the discretion of the principal investigators. Dose tapering was conducted according to standard medical practice with respect to controlling disease activity

This study evaluated 217 patients who enrolled in a long-term, open-label extension study. Patients who discontinued from the trial were included. Patients were grouped according to reduced, maintained, or increased MTX and/or corticosteroid dosages (adjusted to the prednisone equivalent) up to their last visit.

Patients had received adalimumab 40 mg every other week (eow) plus MTX for a minimum of 8 months, and up to 60 months. The changes in the dosing of MTX and corticosteroids (adjusted to the prednisone equivalent) from initial to last visit were evaluated.

Demographic data, age, gender, disease duration, rheumatoid factor and changes in TJC, SJC, CRP, DAS28, HAQ and ACR responses were analysed. Baseline demographics and disease characteristics as well as efficacy measures at last visit (ACR 20/50/70, DAS28, HAQ) were compared between the groups. All patients, including those who discontinued from the trial, were evaluated

The results of this study showed that patients who increased their MTX dose had a longer disease duration compared to those whose dose remained the same or decreased.

TABLE 77
Baseline Demographics and Disease Characteristics by Categories Based on
MTX and Corticosteroid Dose Adjustments

| | MTX | MTX | MTX | Steroid | Steroid no | MTX & steroid |
|---|---|---|---|---|---|---|
| | decrease | no change | increase | decrease | change | decrease |
| | (n = 92) | (n = 110) | (n = 15) | (n = 51) | (n = 29) | (n = 25) |
| Age (years) | 54.5 | 54.0 | 54.6 | 52.0 | 56.6 | 52.9 |
| % Female | 70.7 | 76.4 | 86.7 | 76.5 | 65.5 | 72.0 |
| Duration of | 11.1 | 12.3 | 14.5 | 10.8 | 11.6 | 10.1 |
| RA (years) | | | | | | |
| TJC (0-28) | 14.1 | 15.5 | 16.1 | 14.4 | 15.7 | 13.7 |
| SJC (0-28) | 12.0 | 12.2 | 12.9 | 12.8 | 12.5 | 12.5 |
| HAQ (0-3) | 1.41 | 1.58 | 1.65 | 1.45 | 1.57 | 1.38 |
| CRP | 2.76 | 2.50 | 1.63 | 3.23 | 2.49 | 4.17 |
| (mg/L) | | | | | | |
| DAS28 | 5.7 | 5.9 | 5.5 | 5.8 | 5.9 | 5.7 |

One patient had an dose increase in steroids and is not included in the table.

Of the 217 patients who received adalimumab plus MTX, 92 (42%) patients had a MTX dose reduction, 110 (51%) remained unchanged, and 15 (7%) had a dose increase. The mean initial MTX dose was 16.6 mg/week and the mean dose at last visit was 13.8 mg/week (p<0.0001). Ten patients had the minimum MTX dose of 2.5 mg/week at last visit. 92 (42%) patients had a MTX dose reduction, 110 (51%) remained unchanged, and 15 (7%) had a dose increase (see FIG. 113). 10 patients had the minimum MTX dose of 2.5 mg/week at last visit. The mean time on therapy was 41 months, range 8 to 59 months. 25 (12%) had a dose reduction in both corticosteroids & MTX

Of the 81 patients in this analysis who were on corticosteroid therapy, dose was decreased for 51 (63%; 25 discontinued completely at last visit (32% dose reduced and 31% discontinued)), unchanged for 29 (36%) and increased in one patient (1%). 25 patients (12%) had a decrease in both MTX and corticosteroids. The mean initial dose of corticosteroids was 5.8 mg/day and the mean dose at last visit was 2.7 mg/day (p<0.0001). Of the 51 patients who decreased their corticosteroid dose, 25 (31%) had discontinued corticosteroid use completely at last visit. In addition, for patients who were on methotrexate therapy, 42% saw a dose reduction, 51% maintained the dose, and 7% saw an increase in mtx dose. There was an overall significant change in the dose of mtx between baseline (16.5 mg.week) and the final dose (13.8 mg/week).

ACR responses were maintained over time in patients treated with adalimumab despite decreases in corticosteroid or MTX dosage, as shown below in Table N. Clinical remission (DAS28<2.6) was achieved in patients treated with adalimumab despite decreases in corticosteroid or MTX dosage. Percentages of patients who achieved clinical remission (DAS28<2.6) at last visit by mtx and steroid dose change included: 7.2% mtx decrease; 15.5% mtx no change; 6.7% mtx increase; 21.6% steroid decrease; 17.2% no change in steroid; and 24% mtx and steroid decrease. Improvements in DAS28 were maintained in patients treated with adalimumab despite decreases in corticosteroid or MTX dosage. Changes in DAS28 at last visit by mtx and steroid dose change included the following mean change from baseline: −2.7 mtx decease; −2.5 mtx no change; −1.7 mtx increase; −2.9 steroid decrease; −2.5 steroid no change; −3.0 mtx+steroid decrease (MCID=1.0). Patients who had a decrease in MTX, corticosteroids, or both demonstrated better efficacy outcomes and had the highest percentage of patients achieving remission (DAS28<2.6), compared with those with no dose changes (Table 78).

TABLE 78
Efficacy Parameters At Last Visit

| | | Duration | | | | | | |
|---|---|---|---|---|---|---|---|---|
| | | RA* | | | | DAS28 | | HAQ |
| | N | (yrs) | ACR20 % | ACR50 % | ACR70 % | Change* | DAS28 < 2.6% | Change* |
| MTX | 92 | 11.1 | 75 | 55 | 34 | −2.7 | 27 | −0.73 |
| decrease | | | | | | | | |
| MTX | 110 | 12.3 | 67 | 44 | 24 | −2.5 | 16 | −0.65 |
| no change | | | | | | | | |
| MTX | 15 | 14.5 | 53 | 33 | 20 | −1.7 | 7 | −0.55 |
| increase | | | | | | | | |
| Steroid | 51 | 10.8 | 78 | 55 | 28 | −2.9 | 22 | −0.67 |
| decrease | | | | | | | | |
| Steroid | 29 | 11.6 | 76 | 52 | 24 | −2.5 | 18 | −0.58 |
| no change | | | | | | | | |
| MTX/ster. | 25 | 10.1 | 76 | 48 | 20 | −3.0 | 24 | −0.65 |
| decrease | | | | | | | | |

*Mean values

In conclusion, the majority (>90%) of patients with RA receiving long-term treatment with adalimumab plus methotrexate were able to reduce or maintain their doses of concomitant MTX and/or corticosteroids. Patients with a reduction in MTX and/or corticosteroid doses while receiving adalimumab therapy demonstrated sustained levels of efficacy.

### EXAMPLE 24

### Failure to Inhibit Radiographic Progression within the First 6 Months of Therapy Leads to Worse Radiographic Outcomes at 2 Years

Early, aggressive treatment of rheumatoid arthritis (RA) provides the most favorable clinical and radiographic outcomes (Breedveld F C, et al. Ann Rheum Dis 2004; 63:627-33). Recent clinical trials demonstrate that combination therapy of a tumor necrosis factor (TNF) antagonist plus methotrexate (MTX) is superior to MTX alone for controlling signs and symptoms and inhibiting radiographic progression (De Vries-Bouwstra et al. Arthritis Rheum 2003; 48:3649; Smolen et al. Ann Rheum Dis 2003; 61(Suppl I):64; and Weinblatt et al. Arthritis Rheum 2003; 48:35-45). Studies have shown that delays in initiating therapy with disease-modifying antirheumatic drugs (DMARDs) leads to worse long-term radiographic outcomes in rheumatoid arthritis (RA). This “window of opportunity” to initiate effective therapy may be as short as several months (Breedveld F C, et al. Ann Rheum Dis 2004; 63:627-33).

The objective in this subanalysis of Study J was to evaluate whether inhibition of radiographic progression at Month 6 of treatment would influence radiographic progression at 2 years.

Study J was a 799-patient, 2-year, double-blind study of methotrexate-naïve adult patients with active, early RA (<3 years). Patients were randomized to receive 1 of 3 treatments: combination therapy (adalimumab 40 mg every other week [eow] plus methotrexate [MTX]), adalimumab 40 mg eow alone, or MTX alone. An overview of Study J is shown in FIG. 18. Baseline demographics and clinical characteristics were similar among the 3 arms (see above examples for details).

Mean change from baseline in Total Sharp Score (TSS) was measured at 6 months, 1 year, and 2 years in the intention-to-treat (ITT) population using imputation for missing data. Patients were classified as having radiographic progression (mean change in TSS>0.5) or without radiographic progression (ΔTSS≦0.5). Patients with inadequate data were excluded from this analysis.

Treatment with the combination of adalimumab+MTX was most effective and MTX alone least effective in inhibiting radiographic progression in the overall population. Treatment differences were statistically significant by 6 months.

Overall, combination therapy was the most effective, and MTX was the least effective treatment in inhibiting radiographic progression. At 6 months, more patients who received combination therapy (205 of 268 [76%], p=0.001 vs. either monotherapy) had no progression than patients who received either adalimumab alone (172 pf 271 [63%]) or who received MTX alone (129 of 252 [51%]). Of those who had no progression at 6 months, 74% in the combination arm (151 of 205), but only 61% in the MTX arm (79 of 129), had no further progression by the end of 2 years (p=0.017).

At 6 months, no radiographic progression occurred in 76% of patients treated with the combination of adalimumab+MTX (p=0.001) and 63% with adalimumab alone (p<0.05) vs. 51% with MTX alone (Table 79).

TABLE 79
Radiographic progression by treatment arm and month-6 outcome

| | | Adalimumab | MTX |
|---|---|---|---|
| | Adalimumab + MTX | Alone | Alone |
| Total N* | 268 | 271 | 252 |
| Mo-6 Non-Progressors | 205 (76%) | 172 (63%) | 129 (51%) |
| Yr-2 Non-P | 151 (74%) | 109 (63%) | 79 (61%) |
| Yr-2 Prog | 54 (26%) | 63 (37%) | 50 (39%) |
| Mo-6 Progressors | 63 (24%) | 99 (37%) | 123 (49%) |
| Yr-2 Non-P | 13 (21%) | 13 (13%) | 7 (6%) |
| Yr-2 Prog | 50 (79%) | 86 (87%) | 116 (94%) |

*Excludes 8 of 799 pts because of insufficient radiographic data

For patients who had no radiographic progression at 6 months, mean ΔTSS at 2 years was significantly less with combination therapy or adalimumab alone than with MTX alone (see Table 77 above). For patients who had radiographic progression at 6 months, the mean ΔTSS at 2 years:
Changed relatively little from 6 months in those treated with combination therapy
Was significantly greater in patients treated with MTX alone or adalimumab alone (Table 77 above)

Frequency of no radiographic progression at year 2 according to month-6 radiographic outcomes was as follows:

% of patients with no progression at year 2
No progression at month 6
   74% ada+mtx
   63% ada alone
   61% mtx alone
Progression at month 6
   21% ada+mtx
   13% ada alone
   6% mtx alone

Most patients with no radiographic progression at 6 months remained non-progressors at 2 years. Almost all patients with radiographic progression at 6 months remained progressors and continued to progress further, through 2 years. For both progressors and non-progressors at 6 months, subsequent progression was less frequent and less severe in patients treated with the combination of adalimumab+MTX

TABLE 80
Radiographic Progression Month 6 and Year 2 in Study J

| | No Progression at | No Progression at |
|---|---|---|
| | Month 6 | Month 6 and Year 2 |
| Adalimumab + MTX | 205/268 (76%)* | 151/205 (74%)† |
| Adalimumab alone | 172/271 (63%)‡ | 109/172 (63%) |
| MTX alone | 129/252 (51%) | 79/129 (61%) |

*p = 0.001 v either monotherapy;
†p < 0.05 v either monotherapy;
‡p < 0.05 v MTX alone

In conclusion, patients with no radiographic progression after 6 months of therapy are least likely to have progression at 2 years. In Study J, combination therapy with adalimumab plus MTX provided the best therapeutic option for inhibiting radiographic progression after 6 months and 2 years of therapy. By reducing the frequency and magnitude of radiographic progression by 6 months, adalimumab+MTX prevented radiographic progression that would have otherwise occurred by 2 years. In Study J, combination therapy with adalimumab+MTX was superior to either adalimumab alone or MTX alone in inhibiting radiographic progression. The above study is also described in Pavelka K, Kvien T K, Cohen S B, et al. Ann Rheum Dis 2005; 64(Suppl III):438, which is incorporated by reference herein.

### EXAMPLE 25

### Efficacy Evaluation of Adalimumab (HUMIRA®) by Dose and Administration Route of Concomitant Methotrexate in Widespread Clinical Practice

Methotrexate (MTX), the most common traditional DMARD, is used alone or in combination with other DMARDs and/or biologics in the treatment of patients with rheumatoid arthritis (RA). It is not well understood whether the efficacy of agents used concomitantly with MTX is influenced by the MTX dose or route of administration. Dosage in clinical care varies from less than 7.5 mg to more than 20 mg weekly. Routes of administration include oral, intramuscular (im), subcutaneous (sc), and intravenous (iv) The bioavailability of oral MTX administration has been reported to be 64% of subcutaneous administration and is highly variable between individuals (Hoekstra, M, et al. J Rheumatol 2004; 31:645-8). The bioavailability of oral MTX administration has been reported to be less than MTX sc and is highly variable between individuals. The effect of folic acid, often used to prevent side-effects of MTX, on efficacy is not well-known. The possible effect of dose and/or route of MTX administration on the efficacy of agents used concomitantly with MTX is not well understood.

The objective of this study (Study A) was to examine, in RA patients who have had an incomplete response to MTX monotherapy, whether the efficacy of concomitant adalimumab is affected by the dose or route of administration of MTX. The effect of concomitant folic acid use was also evaluated.

The patient population included patients with moderate to severe RA at more than 450 sites in 11 European countries and Australia received adalimumab sc every other week (eow) in addition to their concomitant but insufficient anti-rheumatic therapies.

The study design for Study A included a 12-week open-label study with optional extension phase. Existing therapy was combined with, or switched to at the physician's discretion, adalimumab 40 mg eow sc

Routine safety and efficacy evaluations were performed at 2, 6, and 12 weeks. For this 12-week interim analysis, dose-response relationships were analysed in patients taking oral MTX exclusively in 3 non-contiguous dose categories (≦7.5, 15, and ≧20 mg). The effects of administration route were compared only for patients taking exclusively 15 mg/week MTX. Dose and route of pre-existing DMARDs was maintained throughout the study. Folic acid, if concomitant with MTX, was maintained throughout the study. Routine safety and efficacy evaluations were conducted at baseline, 2, 6, and 12 weeks, and every 8 weeks in the optional extension phase.

Outcome measures included the following:
Efficacy parameters
ACR20, ACR50, ACR70
EULAR Response
HAQ
Tender and Swollen Joint Count (TJC-SJC)
DAS28
Acute phase proteins

Statistical analysis included a 12-week interim analysis—patients with concomitant MTX (exclusively). Dose-response relationships were also analysed in patients taking oral MTX exclusively in 3 non-contiguous dose categories (≦7.5, 15, ≧20 mg weekly). Effect of route of administration was analysed only in patients taking 15 mg MTX weekly

Of 4241 patients in this interim analysis, 77% were treated with adalimumab and at least one traditional concomitant DMARD. Concomitant MTX was most often administered alone (N=1561) but was also given in combination with other traditional DMARDs (N=710). Routes of administration were oral, n=1689 (74%); im, n=314 (14%); sc, n=200 (9%); and iv, n=68 (3%). Median doses for all routes were 15 mg/wk. The baseline characteristics (age, DAS28, number of prior DMARDs) of all groups were similar.

Efficacy outcomes for concomitant adalimumab and 15 mg MTX were similar across all routes (Table 81 below). ACR and EULAR responses were similar across all 3 MTX dose groups, as shown in Table N. ACR and EULAR response to adalimumab+15 mg/wk MTX were similar regardless of route of administration. Patients taking ≧20 mg of oral MTX had slightly better ACR and EULAR responses.

TABLE 81
Clinical Response to Adalimumab by Dose and Route of
Concomitant MTX*

| Efficacy | MTX | MTX | | | | |
|---|---|---|---|---|---|---|
| Criteria Wk | oral | oral | MTX oral | Sc | Im | Iv |
| 12 | ≦7.5 | 15 mg | ≧20 mg | 15 mg | 15 mg | 15 mg |
| N | 127 | 405 | 216 | 37 | 76 | 23 |
| ACR20 | 72 | 72 | 78 | 78 | 69 | 73 |
| (%) | | | | | | |
| ACR50 | 42 | 45 | 55 | 47 | 46 | 46 |
| (%) | | | | | | |
| ACR70 | 20 | 18 | 29 | 17 | 20 | 18 |
| (%) | | | | | | |
| Moderate | 84 | 86 | 89 | 86 | 80 | 86 |
| EULAR | | | | | | |
| response | | | | | | |
| (%) | | | | | | |
| Good | 36 | 39 | 50 | 25 | 38 | 41 |
| EULAR | | | | | | |
| response | | | | | | |
| (%) | | | | | | |
| Change in | −2.2 | −2.2 | −2.4 | −2.3 | −2.1 | −2.5 |
| DAS28** | | | | | | |
| Change in | −0.56 | −0.50 | −0.52 | −0.53 | −0.55 | −0.54 |
| HAQ** | | | | | | |
| Change in | −8 | −8 | −9 | −10 | −8 | −11 |
| TJC (0-28)** | | | | | | |
| Change in | −7 | −6 | −7 | −7 | −6 | −7 |
| SJC (0-28)** | | | | | | |

*Exclusively MTX, no other DMARD
**Mean change from baseline

60% of the MTX-treated patients received folic acid. The use of folic acid by 60% of the patients had no effect on efficacy results. Furthermore an influence on ACR20 response rates was not found.

In conclusion, in this standard-of-care study of RA patients who responded incompletely to MTX, the addition of adalimumab therapy led to improvements at 12 weeks in all major efficacy parameters regardless of the dose or route of administration of the concomitant MTX. Folic acid had no effect on the efficacy of adalimumab+MTX Formal investigations to determine the optimal dose and route of concomitant MTX are recommended. The above study is also described in Bombardieri et al. Ann Rheum Dis 2005; 64(Suppl III):428, which is incorporated by reference herein.

### EXAMPLE 26

### Adalimumab (HUMIRA®) is Effective in Patients who Have Previously been Treated with TNF-Antagonists (Etanercept and/or Infliximab) in Widespread Clinical Practice: 12-Week Outcomes in Study A

Biologic agents are commonly introduced in the treatment of moderate to severe rheumatoid arthritis (RA) after traditional disease-modifying antirheumatic drugs (DMARDs) fail. Biologic disease-modifying agents are recommended for moderate and severe RA after failure of at least one traditional disease-modifying antirheumatic drug (DMARD). While biologics are generally well-tolerated, therapeutic intolerance or diminished efficacy of one biologic may result in the consideration of another. Limited experience is available on changing from one biologic to another in cases of lack or loss of efficacy, or intolerance.

The objective of this study was to evaluate the impact of prior biologic therapies, in particular TNF-antagonists etanercept (ETA) and infliximab (INF), on key efficacy parameters of adalimumab therapy in patients with long-standing RA in clinical practice. To evaluate the impact of prior biological therapies, patients with moderate to severe RA enrolled at 450 sites in 11 European countries plus Australia (Study A). Patients with moderate to severe RA and an inadequate response to prior DMARD therapies were enrolled in a 12-week open-label study with an optional extension phase. Those with an insufficient response to standard therapies were treated with adalimumab 40 mg sc every other week in addition to their prior traditional DMARD therapies. Patients with previous biologic treatment must have received the last administration at least 8 weeks before enrollment Routine safety and efficacy data were collected at weeks 2, 6, and 12, and every 8 weeks in the extension phase. Efficacy parameters included ACR20, ACR50, ACR70, EULAR Response, HAQ, tender and swollen joint count (TJC28/SJC28), and DAS28. Beyond week 12, patients were allowed to continue on adalimumab therapy, with follow-up visits every 8 weeks. For this interim analysis, efficacy outcomes were evaluated after 12 weeks of therapy per the number and type of prior biologics used: None, any (ETA, INF, anakinra), ETA only, INF only, or ETA and INF but not anakinra.

As of November 2004, 12-week data were available for 4241 patients with long-standing moderate to severe RA who had an insufficient response to prior DMARD therapies. The mean baseline age=54 years; disease duration=11 years; DAS28=6.0; HAQ=1.62. Of all patients analysed, 16% (n=688) had previously been treated with one or more biologic agents and 77% (n=3285) were taking 1 or more concomitant traditional DMARDs, typically methotrexate. Of the 688 patients entering the trial having prior biologic therapy, 545 (79%) were with 1 biologic; 117 (17%) were with 2 biologics; 26 (4%) were with 3 biologics; 486 (71%) were with prior INF therapy (not exclusively); and 223 (32%) were with prior ETA therapy (not exclusively).

Of patients with prior biologic agents, 27% received adalimumab monotherapy, 54% received adalimumab and 1 DMARD, 13% received adalimumab and 2 DMARDs, and 6% were received adalimumab and 3 or more DMARDs. Outcomes after 12 weeks of therapy are given by type of prior biologics (See Table 82).

TABLE 82
12-Week Efficacy By Type of Prior Biologics*.

| | Pts in | Prior Biologic* | | | | |
|---|---|---|---|---|---|---|
| Efficacy Parameter | Study A | None | Any | ETA | INF | ETA + INF |
| at 12 Weeks | (n = 4241) | (n = 3553) | (n = 688) | (n = 114) | (n = 358) | (n = 78) |
| # of | 3.1 | 2.7 | 4.9 | 5.2 | 4.6 | 5.5 |
| Previous | | | | | | |
| DMARDs* | | | | | | |
| ACR20 (%) | 66 | 68 | 57 | 52 | 63 | 42 |
| ACR50 (%) | 38 | 40 | 31 | 30 | 35 | 27 |
| ACR70 (%) | 17 | 18 | 11 | 11 | 12 | 13 |
| Change in | −8 | −8 | −8 | −8 | −8 | −7 |
| TJC** | | | | | | |
| Change in | −6 | −7 | −6 | −7 | −6 | −5 |
| SJC** | | | | | | |
| TJC = 0 (%) | 21 | 22 | 16 | 16 | 17 | 10 |
| SJC = 0 (%) | 24 | 25 | 18 | 21 | 22 | 7 |
| Change in | −0.49 | −0.50 | −0.43 | −0.36 | −0.49 | −0.35 |
| HAQ** | | | | | | |
| HAQ < 0.5 (%) | 24 | 26 | 13 | 9 | 14 | 13 |
| Change in | −2.1 | −2.1 | −1.8 | −1.9 | −2.0 | −1.4 |
| DAS28** | | | | | | |
| DAS28 < 2.6 (%) | 19 | 21 | 12 | 13 | 13 | 5 |

*Mean values,
**Mean change from baseline.

Patients without prior biologic therapy had less severe RA compared to those with prior biologics (See Table 83).

TABLE 83
Baseline Data by Prior Biologic Treatment.

| | Prior Biologic* | | | | |
|---|---|---|---|---|---|
| | None | | | | ETA + |
| Baseline | (n = | Any | ETA | INF | INF |
| Characteristics** | 3553) | (n = 688) | (n = 114) | (n = 358) | (n = 78) |
| Age (yrs) | 54 | 53 | 56 | 53 | 52 |
| # of prior | 2.7 | 4.9 | 5.2 | 4.6 | 5.5 |
| DMARDs | | | | | |
| Duration of | 11 | 12 | 13 | 12 | 12 |
| RA (yrs) | | | | | |
| Steroid use*** | 67% | 76% | 82% | 73% | 77% |
| DMARD use | 78% | 73% | 54% | 79% | 64% |
| HAQ | 1.57 | 1.87 | 1.87 | 1.82 | 1.90 |
| DAS28 | 5.9 | 6.2 | 6.4 | 6.1 | 6.5 |
| TJC28 | 13 | 14 | 15 | 14 | 15 |
| SJC28 | 10 | 11 | 12 | 11 | 12 |

*Interval since last administration≧2 months.
**Mean values.
***Max 10 mg/d prednisolone equivalent allowed.

Compared to patients without prior biologics, withdrawal rates due to intolerance and to lack of efficacy were slightly increased in patients with prior biologic (See Table 84).

TABLE 84
Withdrawal Rates by Reason and by Prior Biologic Week 12.

| | Prior Biologic* | | | | |
|---|---|---|---|---|---|
| | | | | | ETA + |
| | None | Any | ETA | INF | INF |
| | (n = 3553) | (n = 688) | (n = 114) | (n = 358) | (n = 78) |
| Intolerance | 4.2% | 6.3% | 6.2% | 5.9% | 2.6% |
| Lack of | 1.5% | 3.4% | 2.7% | 2.0% | 5.1% |
| Efficacy | | | | | |

Patients who had received prior treatment with a biologic had similar ACR response rates to those patients who had no history of biologic treatment, as shown below in Table 85.

TABLE 85
ACR response rates in pts w/ and w/o prior biologic (% of patients)

| | Prior Biologic | | | | |
|---|---|---|---|---|---|
| | None | Any | ETA | INF | ETA + INF |
| ACR20 | 68 | 57 | 52 | 63 | 42 |
| ACR50 | 40 | 31 | 30 | 35 | 27 |
| ACR70 | 18 | 11 | 11 | 12 | 13 |
| Moderate EULAR | 82 | 75 | 74 | 78 | 61 |
| Good EULAR | 39 | 23 | 21 | 25 | 12 |
| Mean change in | −2.1 | −1.8 | −1.9 | −2.0 | −1.4 |
| baseline in DAS28 | | | | | |
| (week 12) | | | | | |
| Mean change in | −0.5 | −0.43 | −0.36 | −0.49 | −0.35 |
| baseline in HAQ | | | | | |
| (week 12) | | | | | |
| (MCID => −0.22) | | | | | |
| Median TJC | −8 | −8 | −8 | −8 | −7 |
| reduction | | | | | |
| Mediant SJC | −7 | −6 | −7 | −6 | −5 |
| reduction | | | | | |

Patients who had a history of prior biologic treatment had similar EULAR response rates to those patients with no prior biologic treatment (See Table 85). Improvements in DAS28 were similar, regardless the type of prior biologic (See Table 85). Physical function, as measured by HAQ, improved after 12 weeks of adalimumab treatment, regardless the type of prior biologic (See Table 85). Improvements in tender and swollen joint count after 12 weeks of adalimumab treatment were similar, regardless the type of prior biologic (See Table 85).

Overall, data collected from widespread clinical practice in Europe and Australia corroborated efficacy data from controlled pivotal trials. Adalimumab was efficacious in patients with RA who previously failed or had insufficient response to TNF-antagonists etanercept and infliximab. This study shows that change to adalimumab after prior unsatisfactory treatment with biologics leads to improvement of RA, independent of the number and type of prior biologic.

The above study can also be found in Ann Rheum Dis 2005; 64(Suppl III):423-4, which is incorporated by reference herein.

### EXAMPLE 27

### Efficacy and Safety of Adalimumab (HUMIRA®) in Canadian Clinical Practice. A Comparison of the Canadian and European Practice: Study C and the Study A

Patient characteristics, treatment strategies and outcomes observed from randomized controlled trials (RCTs) do not always reflect clinical practice. Large, open-label clinical trials intended to reflect general clinical practice can be used to reaffirm data observed in the RCT setting. Confidence in clinical trial outcomes may be enhanced by consistencies observed in outcome data from open-label trials conducted in different clinical practice settings.

The objective of this study was to evaluate the efficacy and safety of adalimumab in the Canadian practice setting, and to compare it with the results of Study A, a similar trial that was conducted in Europe. The Canadian Adalimumab Clinical Trial (Study C) was an open-label, multi-center, Phase IIIb study conducted in Canada. A total of 879 patients with moderate to severe rheumatoid arthritis (RA) who had an inadequate response to standard therapies, including MTX, were treated with adalimumab 40 mg sc every other week (eow) in addition to their preexisting but inadequate therapies. Efficacy and safety were assessed at baseline, 4, 8, and 12 weeks. Efficacy assessments included tender joint count (TJC 028), swollen joint count (SJC 028), Disease Activity Score 28 (DAS28), Health Assessment Questionnaire (HAQ), and EULAR response. This was a preliminary analysis of 236 patients. The sample size for each outcome variable is based on the data available at the time of the analysis. The data from this preliminary analysis were compared with the Week 12 data from Study A (N=2008).

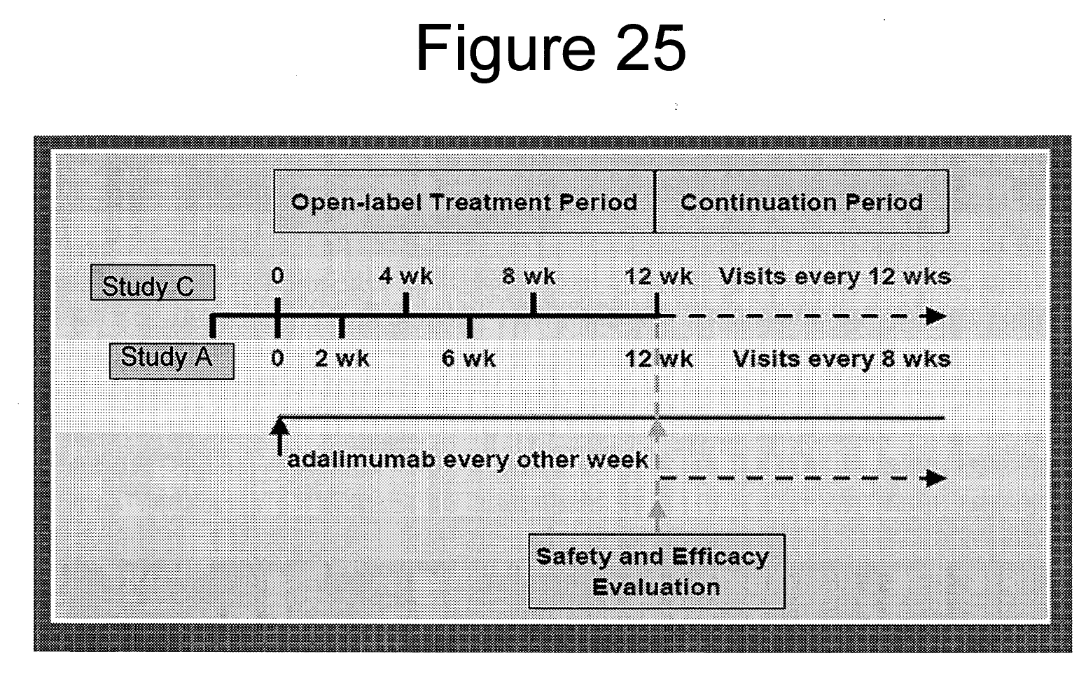

Patients' baseline characteristics and disease severity scores were (mean): age=53.5 years; percentage female=75%; TJC=17; SJC=14; DAS28=6.5; and HAQ=1.4. At Week 12, the mean scores had improved to TJC=8.1, SJC=7.4, HAQ=0.86, DAS28=4.4, all significant (p<0.001) vs. baseline. The comparisons with Study A results are presented in Table 86. A HAQ<0.5 was achieved by 28% of Study C patients and 25% of Study A patients at 12 weeks. Table 87 shows Study C and Study A inclusion criteria. Table 30 below shows the efficacy assessments. FIG. 25 depicts the study design for both Study C and Study A.

TABLE 86
Study C and Study A Trial Results.

| Efficacy Measures at 12 | | |
|---|---|---|
| Weeks | Study C | Study A |
| (Change from Baseline) | (n = Evaluated Patients) | (N = 2008)¹ |
| TJC (0-28)* | −10 (123) | −10 |
| SJC (0-28)* | −7 (123) | −7 |
| DAS28 (mean) | −2.1 (116) | −2.1 |
| HAQ (mean) | −0.55 (122) | −0.49 |
| EULAR response | | |
| % moderate | 78 (121) | 82 |
| % good | 18 (121) | 34 |

*Median values.
¹Burmeister GR, et al. Ann Rheum Dis 2004; 63(Suppl I): 266.

TABLE 87
Study C and Study A Inclusion Criteria.

| Study C | Study A |
|---|---|
| Age ≧ 18 years | Age ≧ 18 years |
| RA defined by ACR criteria for ≧3 | RA defined by ACR |
| months | criteria for ≧3 months |
| Unsatisfactory response or intolerance to at | Unsatisfactory response or |
| least 2 DMARDs | intolerance to at least 1 |
| | DMARD |
| Active RA (≧ swollen joints and one of: | Active RA (DAS 28 ≧ 3.2) |
| positive RF, 1 or more joint erosions, HAQ | |
| score >1) | |

TABLE 88
Efficacy Assessments.

| | Study A |
|---|---|
| Study C | Assessed at baseline, 2, 6, |
| Assessed at baseline, 4, 8, and 12 weeks: | and 12 weeks: |
| DAS28 | DAS28 |
| Eular Response | Eular Response |
| HAQ | HAQ |
| SJC, TJC | SJC, TJC |
| ACR 20, 50, 70 | ACR 20, 50, 70 |

A total of 879 patients were enrolled in Study C and 6229 were enrolled in Study A.

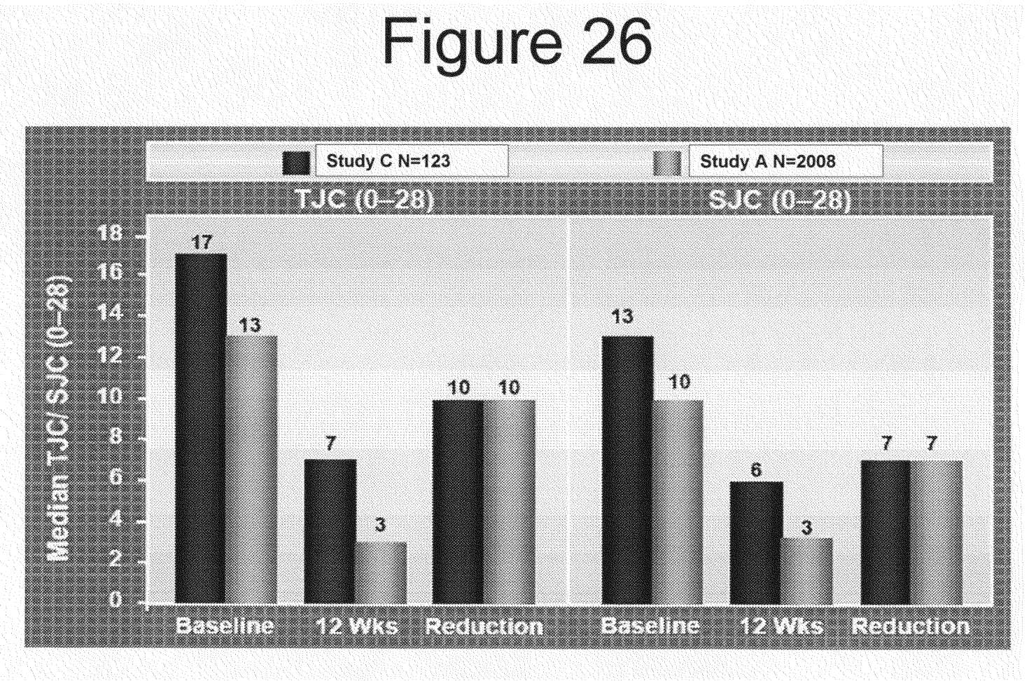

At the time of this analysis, data was available on 236 Study C patients and 2008 Study A patients. Table 89 below shows the baseline demographics and disease severity. FIG. 26 depicts the median TJC/SJC and reduction at 12 weeks.

TABLE 89
Baseline Demographics and Disease Severity.

| | Study C* | Study A* |
|---|---|---|
| | N = 236 | N = 2008 |
| Age (years) | 53.5 | 53 |
| Female (%) | 75% | 80% |
| DAS28 (mean) | 6.5 | 6.0 |
| TJC (median, 0-28 joints) | 17 | 13 |
| SJC (median, 0-28 joints) | 13 | 10 |
| HAQ (mean, 0-3) | 1.4 | 1.6 |
| CRP (mean, mg/dL) | 20.9 | 26 |
| Previous biological experience (%) | | |

*Similar patient populations.

A HAQ score of <0.5 was achieved by 28% of the Study C patients and 25% of the Study A patients at 12 weeks. The improvement in HAQ scores at 12 weeks included −0.55 for Study C and −0.49 for Study A. Clinical remission as measured by a DAS28 score of <2.6 was achieved by 10% of the Study C patients and 24% of the Study A patients by Week 12. The mean decrease in DAS28 score in Study C was −2.1 (32% reduction) and the mean decrease in DAS28 score in Study A was −2.3 (28% reduction). The EULAR responses at 12 weeks included 79% moderate and 18% good in Study C and 82% moderate and 34% good in Study A. Table 90 below shows the medically relevant adverse events.

TABLE 90
Safety-Medically Relevant Adverse Events.

| | | | Study A | All Trials² |
|---|---|---|---|---|
| | | | (3218 | adalimumab |
| | Study C¹ | | PY) | (4870 PY) |
| Type | (n = 879) | Type | N/PY | N/PY |
| Infections | 11 (1.3%) | Any serious | 0.041 | 0.040 |
| | | infection | | |
| Pneumonia (3) | | Pneumonia | 0.008 | 0.008 |
| Post-operative | | TB | 0.002 | 0.003 |
| infections (3) | | Malignancies | 0.006 | 0.006 |
| Infected prosthesis | | | | |
| (1) | | | | |
| Pyelonephritis (1) | | | | |
| Sinusitis (1) | | | | |
| Influenza (1) | | | | |
| Atypical infection | | | | |
| (nodules) (1) | | | | |
| Malignancies | 2 (0.2%) | | | |
| Bilateral papillary | | | | |
| carcinoma | | | | |
| Infiltrating basal cell | | | | |
| carcinoma | | | | |
| Congestive | 1 (0.1%) | | | |
| heart failure | | | | |

¹No reports of death, reactivated TB or lymphoma.
²Data for Registration.
PY = patient-year.

The efficacy of adalimumab was confirmed in a broader clinical setting and was consistent with findings of Study A, a similar trial conducted in Europe. The safety of adalimumab in Study C was consistent with adalimumab's overall safety profile, and there were no new safety concerns. Study C and Study A provide confidence in treatment with adalimumab when used in routine practice. The efficacy and safety profile of adalimumab was not different when adalimumab was used in a routine clinical setting.

The above study can also be found in Ann Rheum Dis 2005; 64(Suppl III):425, which is incorporated by reference herein.

### EXAMPLE 28

### Impact of Screening for Latent TB Prior to Initiating Anti-TNF Therapy in North America and Europe

All tumor necrosis factor (TNF) antagonists have been associated with an increased risk of latent tuberculosis (TB) reactivation. The tuberculin skin test employing Purified Protein Derivative (PPD) is the most widely used method to identify patients with latent TB who may be at risk for reactivation. The following example provides safety information which, in one embodiment, is described on a label in an article of manufacture and is used to indicate to a subject that certain precautions, e.g., pre-screening, may be performed prior to use.

The impact of tumor necrosis factor (TNF) antagonists on outcomes in rheumatoid arthritis (RA) include its significant advance in ability to reduce signs and symptoms, inhibit radiographic progression, and improve physical function. They are also the new “gold standard” of effectiveness, especially in combination with methotrexate (MTX), and are generally safe and well-tolerated.

The role of TNF in TB pathology has been studied in animal models. (Flynn J L, et al. Annu Rev Immunol 2001; 19:93-129). These studies have found that granuloma formation is necessary to contain tuberculosis (TB). Further, TNF is necessary to maintain granuloma homeostasis. In mice infected with TB bacilli, TNF inhibition was associated with shortened survival according to the following studies: TNF deficiency (knockout mice) (Smith S, et al. Infect Immun 2002; 70:2082-9); inhibition by receptor fusion protein (Garcia I, et al. Eur J Immunol 1997; 27:3182-90); and inhibition by anti-TNF antibodies (Flynn J L, et al Immunity 1995; 561-72).

With regard to TB reactivation in patients receiving TNF antagonists, cases of TB have been seen in patients treated with all TNF antagonists (Ellerin T, et al. Arthritis Rheum 2003; 48:3013-22; Keane J, et al. New Eng J Med 345:1098-104; and Manadan A M, et al. Arthritis Rheum 2002; 46(9 Suppl): S166), where most cases felt to represent reactivation and were commonly extrapulmonary. Reports of cases primarily post-marketing and interpretation were limited by the following: spontaneous reporting (vs. facilitated reporting); incomplete clinical information; geographic diversity with different rates of background TB; and numerator/denominator estimated, which could not reliably be used to calculate rates.

The objective of this study was to determine the effect of screening on the incidence of TB cases occurring in US/Canada and Europe/Australia adalimumab clinical trials in rheumatoid arthritis (RA), to assess the effectiveness of isoniazid (INH) prophylaxis in preventing reactivation of latent TB in patients determined to be at higher risk for developing TB, and to assess the impact of screening in reducing the rate of new TB cases. To further this objective, data through December 2004 from all patients enrolled in adalimumab RA trials in North America and Europe were reviewed. The rate of TB was calculated for the period before screening was initiated (primarily Phase I trials) and the period after screening was implemented. The screening program consisted of a clinical interview, a tuberculin skin test, and, in most patients, a chest X-ray (CXR). The definition of PPD-positive depended on local country-specific guidelines. For those people who were PPD-positive, or for whom the investigator felt the risk of TB reactivation was high, INH was given for prophylaxis.

TB screening in adalimumab RA clinical trials was not instituted until after many trials were initiated. The methodology differed among protocols. For instance, in Europe and Australia, it was initially used CXR only, and in later studies, CXR and purified protein derivative skin test (PPD) were both used. By contrast, in US and Canada, CXR and PPD were both used.

Through Dec. 31, 2004, there were 11,440 RA patients (14,544 patient-years [pt-yrs] of exposure) treated with adalimumab in clinical trials in North America and Europe. Of these, 3422 patients, (5918.9 pt-yrs) were from North America, and 8018 patients (8625.2 pt-yrs) were from Europe, including 6610 patients from Study A, the largest trial of anti-TNF therapy in Europe. Before screening was instituted, there were 7 cases of TB in 534 pt-yrs of exposure (rate of 0.013/pt-yrs). None of these subjects received INH prophylaxis. After screening was implemented, there were 14,010 pt-yrs of exposure and 27 cases of TB (rate of 0.0019/pt-yr). Five of these cases were in North America (rate 0.0008), and 22 were in Europe (rate 0.0027). The rate ratio of cases in Europe compared to North America is 1:3.2.

The median time to the development of TB was 167 days (range: 14-1636). There were 19 of 34 (56%) culture-confirmed cases, and extrapulmonary disease occurred in 22 of 34 (65%) cases. The ratio of TB prior to screening to that after screening was initiated represents an 85% reduction in the rate of developing TB in those who were screened. Of the 27 TB cases overall, there were 6 subjects who were PPD-positive as judged by the investigators, and 4 who did not have PPD screening performed. Four of these subjects received INH, but compliance with INH was not measured. A total of 712 subjects from Study A were identified by the investigators as being at high risk for TB (most were PPD-positive) and received INH prior to receiving study drug. In that study, 4 subjects (0.6%) developed TB after INH prophylaxis.

Table 91 below shows the size of the adalimumab RA clinical trial database (through December 2004) in US/Canada and Europe/Australia. Table 92 shows the differences in adalimumab RA clinical trials before and after initiating TB screening.

TABLE 91
Size of Adalimumab RA Clinical Trial Database* in US/Canada and
Europe/Australia.

| | | Exposure |
|---|---|---|
| | Patients | (pt-yrs) |
| US/Canada | 3422 (30%) | 5919 (41%) |
| Europe/Australia | 8018 (70%) | 8625 (59%) |
| Total | 11,440 | 14,544 |

*Through December 2004

TABLE 92
Differences in Adalimumab RA Clinical Trials Before and After
Initiating TB Screening.

| Pre-Screening | Post-Screening |
|---|---|
| Phase I and early Phase II studies | Phase II-III studies |
| No PPD or Chest X-ray (CXR) | TB screening instituted |
| Dose finding studies; many patients | Most received 40 mg |
| received >40 mg eow (some treated with | eow sc dosing |
| IV doses up to 10 mg/kg) | |
| Most in Europe | Geographically diverse |
| | (patients from both North |
| | America and Europe) |

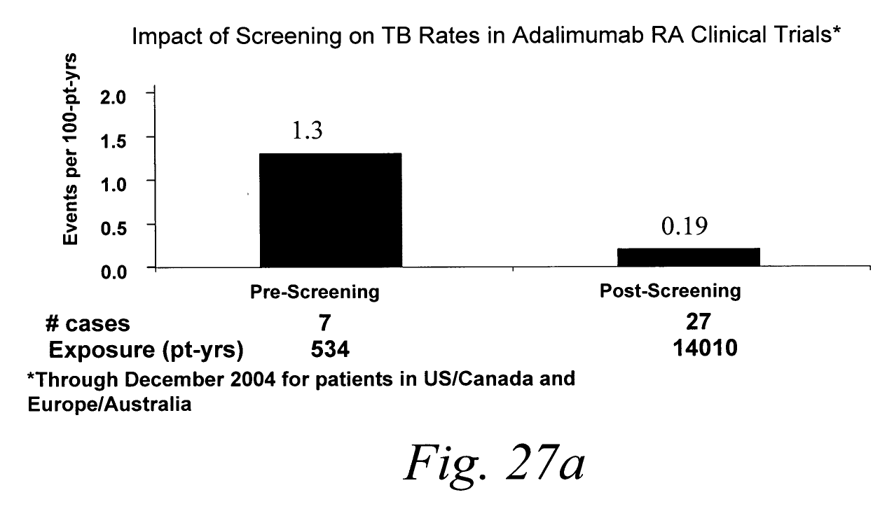

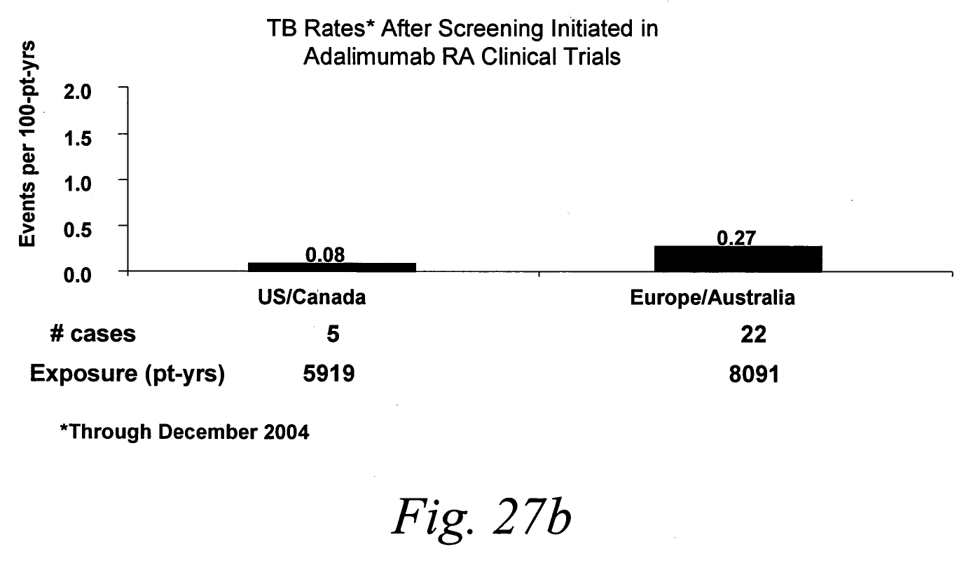

The impact of screening on TB rates in adalimumab RA clinical trials through December 2004 can be seen in FIG. 27a. FIG. 27b depicts TB rates through December 2004 after screening was initiated in adalimumab RA trials. Table 93 below summarizes the cases seen in adalimumab RA clinical trials from US/Canada and Europe through December 2004. Finally, the results of screening for TB, based on data from 6610 patients include 11.6% positive (PPD≧10 mm), 16.4% positive (PPD≧5 mm), and 3.0% abnormal w/chest x-ray.

TABLE 93
Summary of Cases Seen in Adalimumab RA Clinical Trials From
US/Canada and Europe*

| | |
|---|---|
| Total Cases | 34 |
| Culture positive | 62% |
| Mean age | 60.7 years |
| Extrapulmonary | 65% |
| Median months from treatment to diagnosis | 7.2 months (1-54) |
| (range) | |
| Outcome Resolved | 32** |

*Through December 2004.
**One patient died of unrelated causes.

With regard to INH prophylaxis, a dose of Isoniazid of 5 mg/kg/d×9 months (max. 300 mg/d) is recommended when screening tests suggest evidence of latent TB. It is further recommended that INH prophylaxis is initiated when investigators believe their patients are at high risk for TB reactivation (including history of exposure).

In this study, of 835 patients who received INH, 621 (74.4%) had a positive PPD, 121 (14.5%) had a chest X-ray indicative of past TB, 76 (9.1%) had both a positive PPD and a chest X-ray indicative of past TB, and 17 (2.0%) had other reasons. Four patients (0.5%) who received INH prophylaxis in this analysis developed TB. Compliance was not measured. The time between initiation of INH and adalimumab were as follows: 0-14 days (4.5%); 15-28 days (22.3%); 29-42 days (50.2%) and >43 days (23.0%).

In the current analysis, implementation of TB screening resulted in approximately an 85% reduction in the rate of latent TB reactivation. Patients identified as being at high risk for TB and who were given INH prophylaxis prior to treatment with adalimumab had little reactivation of TB. Prior to initiation of any anti-TNF therapy, all patients should be screened for latent TB.

In summary, TNF inhibition can lead to reactivation of latent TB. Appropriate screening significantly reduces the rate of TB reactivation, and it has been shown that INH prophylaxis is effective in preventing reactivation of latent TB in patients receiving anti-TNF therapy. Further, screening for TB is recommended by most health authorities, because it can significantly reduce the rate of TB reactivation, and it should be done prior to initiation of anti-TNF therapy. Appropriate screening includes: PPD (≧5 mm), CXR and relevant exposure history and physical examination. INH prophylaxis, where appropriate, is effective in preventing reactivation of latent tuberculosis in patients who are receiving anti-TNF therapy.

The above study can also be found in Ann Rheum Dis 2005; 64(Suppl III):86, which is incorporated by reference herein.

### EXAMPLE 29

### Routine, APGAR-Like Patient Index Datasheet (RAPID): A Continuous Index of Patient Measures Discriminates Effectively Between Active and Placebo Treatments in 4 Adalimumab Clinical Trials

The ACR improvement criteria for rheumatoid arthritis (RA) in clinical trials are based on differences between baseline and endpoint rather than continuous measures. The Disease Activity Score 28 (DAS 28) provides a continuous scale of 4 Core Data Set measures: swollen joint count (SJC), tender joint count (TJC), laboratory value, and patient's global assessment. While the ACR criteria and DAS28 quantitative scales have greatly advanced RA clinical trials, most patient visits in standard rheumatology care do not include a formal joint count. Generally the only quantitative data other than laboratory tests, which may be normal in up to 40% of RA patients. A patient questionnaire including physical function, pain and patient's global assessment of disease activity, can be administered easily at all visits, and might provide a quantitative continuous scale to assess clinical status.

The objective of this study was to analyze a “routine Apgar-like patient index datasheet” (RAPID), a continuous index scored 0-10, based on the 3 Core Data Set measures on a patient questionnaire, physical function, pain, and patient's global assessment, in clinical trials of the tumor necrosis factor (TNF) antagonist adalimumab (Ann Rheum Dis 2005; 64(Suppl III):423). In order to further this objective, clinical data from the adalimumab arms of the 4 pivotal trials were analyzed, including: adalimumab monotherapy (Study 2) vs. placebo; adalimumab with methotrexate (MTX) vs. placebo with MTX (Study I and Study 1); and adalimumab with other disease-modifying antirheumatic drugs (DMARDs) vs. placebo with other DMARDs (Study K). Several rescalings of the physical function, pain, and patient's global assessment scales were calculated. The physical function scale is scored 0-3, and was rescaled to 0-3. The pain visual analog scale (VAS) is scored 0-10 and was also rescaled to 0-3. The global VAS is scored 0-10 and was rescaled to 0-4. Thus, the total of the 3 scores ranges from 0-10. Scores were calculated for both continuous and categorical indices. The DAS28 was calculated from the SJC, TJC, patient's global score, and C-reactive protein.

RAPID scores discriminated effectively between adalimumab alone, adalimumab plus MTX, or adalimumab plus other DMARDs vs. placebo alone, placebo plus MTX placebo plus other DMARDs. The RAPID t-scores, on average, were 21% lower than t-scores for the DAS28. RAPID scores were completed without computer or calculator in <20 seconds, using rescaled templates on the patient questionnaire. Table 94 shows the analysis of RAPID patient index using adalimumab pivotal trial data. Table 95 shows the Spearman rank correlations of changes in RAPID scores with changes in DAS28 scores and changes in ACR-N in four adalimumab clinical trials.

TABLE 94
Analysis of RAPID Patient Index Using Adalimumab
Pivotal Trial Data.

| | Baseline | | Endpoint | | Difference | | t- | p- |
|---|---|---|---|---|---|---|---|---|
| Index | ADA | PBO | ADA | BPO | ADA | BPO | statistic* | value* |

RAPID (R326)

| | | | | | | | | |
|---|---|---|---|---|---|---|---|---|
| Study I | 7.27 | 7.53 | 3.84 | 6.48 | 3.43 | 1.05 | 5.0 | <0.001 |
| Study 2 | 8.58 | 8.62 | 5.88 | 7.71 | 2.70 | 0.91 | 5.3 | <0.001 |
| Study 1 | 7.09 | 7.22 | 3.86 | 5.85 | 3.22 | 1.37 | 7.9 | <0.001 |
| Study K | 7.00 | 7.05 | 4.22 | 5.87 | 2.79 | 1.17 | 8.0 | <0.001 |

DAS28

| | | | | | | | | |
|---|---|---|---|---|---|---|---|---|
| Study I | 10.3 | 10.2 | 5.0 | 9.3 | 5.3 | 0.9 | 7.8 | 0.018 |
| Study 2 | 12.0 | 11.9 | 8.6 | 10.7 | 3.4 | 1.2 | 5.6 | <0.001 |
| Study 1 | 9.90 | 10.0 | 5.2 | 8.0 | 4.7 | 1.9 | 9.3 | <0.001 |
| Study K | 9.75 | 9.86 | 6.0 | 8.0 | 3.8 | 1.8 | 8.4 | <0.001 |

ADA = adalimumab;
PBO = placebo
*adjusted for baseline

TABLE 95
Spearman Rank Correlations of Changes in RAPID Scores with Changes
in DAS28 Scores and Changes in ACR-N in 4 Adalimumab Clinical
Trials.

| Trial | Study I | Study 2 | Study 1 | Study K |
|---|---|---|---|---|
| DAS28 | 0.80 | 0.79 | 0.69 | 0.69 |
| ACR-N | 0.77 | 0.66 | 0.70 | 0.68 |

All p < 0.001

In conclusion, a quantitative RAPID scale (0-10) based on patient questionnaire data alone appears to provide a useful quantitative measure that discriminated well between active therapy and placebo in 4 adalimumab clinical trials. Further, the DAS28 provides a somewhat more discriminatory measure, but the RAPID index provides significant discriminatory power. RAPID scores can be obtained without a computer or calculator in <20 seconds, using rescaled templates on the patient questionnaire, and could be used as an index of clinical status in all patients seen in standard rheumatology practice.

### EXAMPLE 30

### Effects of Long-Term Adalimumab Therapy on Health Utility and Fatigue in Patients with Long-Standing, Severe Rheumatoid Arthritis (RA)—Results from a 3-Year Follow Up Study

Fatigue, a common symptom of rheumatoid arthritis (RA), is known to cause reduced health-related quality of life (HRQoL) and work productivity. Although, clinical trials of tumor necrosis factor (TNF) antagonists in the treatment of RA routinely evaluate effects on HRQoL, health utility and fatigue are rarely assessed quantitatively.

To investigate the ability of adalimumab, an anti-TNF monoclonal IgG1 antibody, to provide sustained long-term improvement in patients with severe RA, who had failed at least one disease-modifying antirheumatic drug (DMARD), as measured by three important patient-reported outcomes:
HRQoL
Health utility
Fatigue

### Methods

Patients with severe RA, who had failed at least one DMARD, received adalimumab 40 mg every other week (eow) or placebo without concomitant DMARDs for 26 weeks. This study was an open-label extension trial in which patients on adalimumab (40 mg eow) from a previous study were followed up to 170 weeks using the same health economics questionnaires.

In addition to clinical parameters, the SF-36 instrument, the Health Utilities Index Mark 3 (HUI3), and the Functional Assessment of Chronic Illness Therapy-Fatigue (FACIT-F) questionnaires were administered at baseline, and at weeks 12, 26, 50, 74, 98, 122, 146 and 170, respectively. The HUI3 scale ranges from 0-1, with “1” denoting perfect health and “0” denoting death. FACIT-F scores range from 0-52, with higher scores representing less fatigue. SF-36 scores range from 0-100 with higher scores indicating improvement in HRQoL. Changes in HUI3 scores of ≧0.03, 1 FACIT-F scores of ≧4.2 and individual SF-36 domain scores of 5-103 are considered clinically meaningful.

### Results

Baseline characteristics were indicative of long-standing, severe RA: age: 53 years; disease duration: 11 years; TJC (0-68): 34; HAQ: 1.9, CRP (mg/L): 56.6; previous DMARDs: 4 (mean values). Adalimumab-treated RA patients' baseline HUI and FACIT-F scores were comparable to placebo and were approximately one-third of population norms. Rapid and statistically significant improvements vs. placebo were observed by Week 12 and Week 26 (p<0.05)

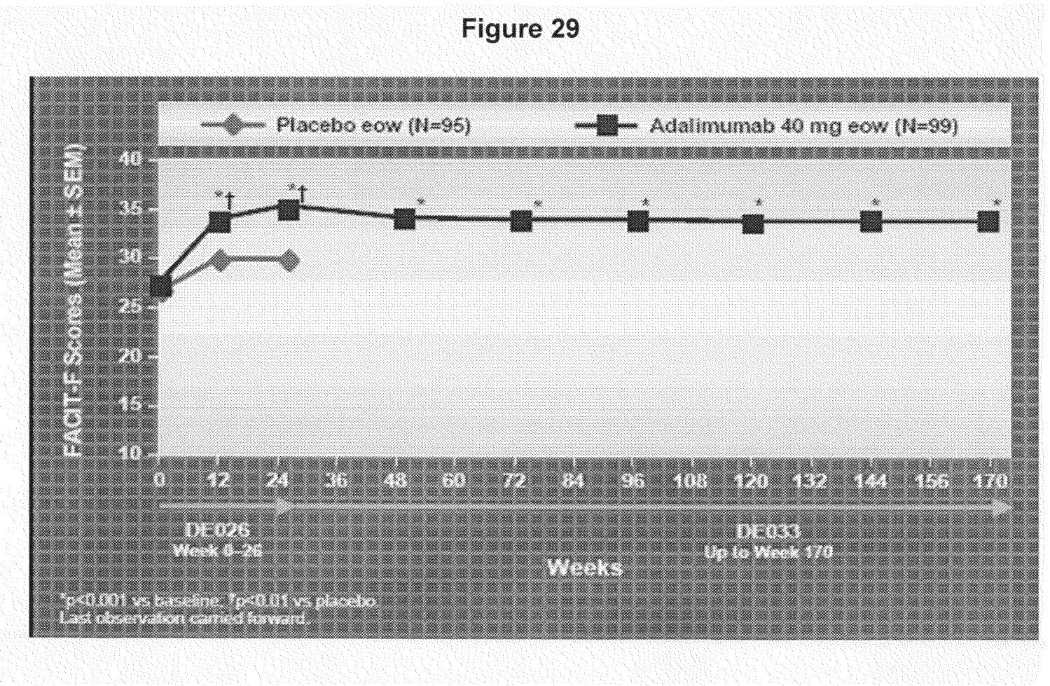

At Week 26, mean change from baseline in adalimumab-treated patients was 0.18 for HUI3 (p<0.001). This improvement was maintained throughout the 170-week follow-up. At Week 26, mean change from baseline in adalimumab-treated patients was 8.54 for FACIT-F (p<0.001) (FIG. 29).

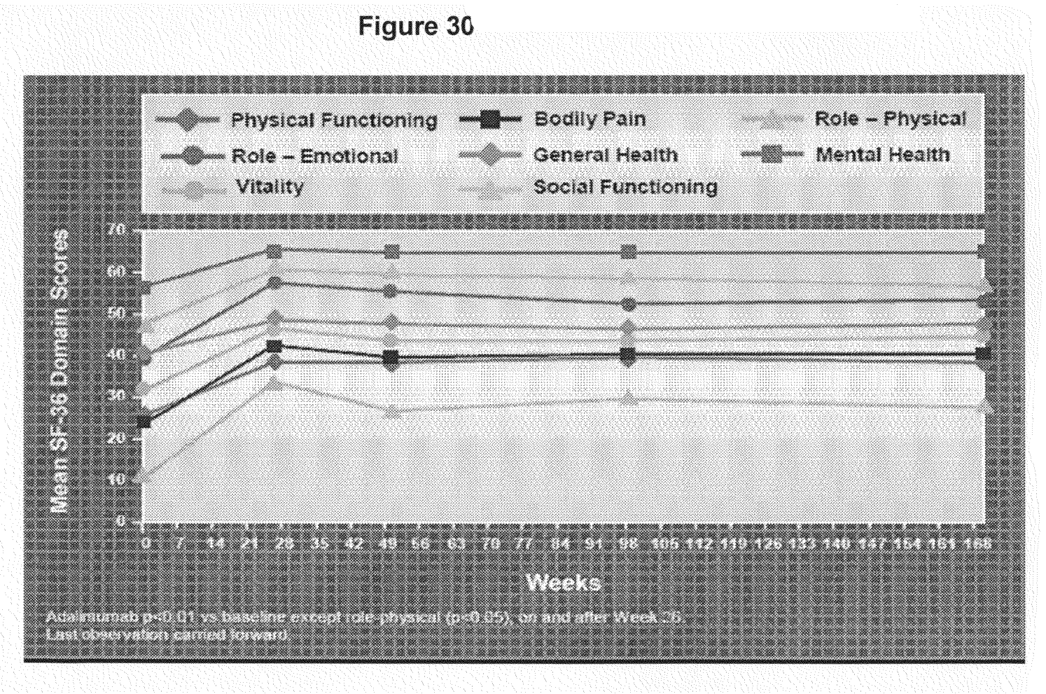

Rapid and statistically significant improvements from baseline were observed by 12 weeks of adalimumab treatment and were maintained through Week 170. With adalimumab, all domains of SF-36 showed clinically meaningful improvements within 26 weeks of treatment and throughout the whole observation period (FIG. 30). All reported changes were statistically significant.

In sum, adalimumab provided clinically important, simultaneous improvements in HRQOL, health utility and fatigue in patients with severe, active RA who had failed at least one DMARD. These improvements were sustained over the 3-year observation period
1. Torrance G et al. Rheumatology 2004; 43:712-8.
2. Cella D et al. J Pain Symptom Manage 2002; 24:547-561.
3. Kosinski M, et al. Arthritis Rheum 2000: 43:1478-87.

### EXAMPLE 31

### Criteria-Based Interpretation of SF-36 Improvements from Adalimumab Plus Methotrexate (MTX) Combination Therapy vs. MTX Alone in Early Rheumatoid Arthritis (RA)

Recent clinical trials of tumor necrosis factor (TNF) antagonists have shown that therapy with a TNF antagonist plus methotrexate (MTX) is superior to MTX monotherapy in the treatment of rheumatoid arthritis (RA)¹⁻³. The Study J was the first trial to directly compare a TNF antagonist plus MTX with the TNF antagonist alone and MTX alone in MTX-naïve patients with recent-onset RA.

The Short Form 36 (SF-36) Health Survey is a generic, patient reported, health-related quality of life (HRQOL) measurement instrument with 8 domains and two summary scores for physical and mental health. Interpretation of the results from health surveys such as the SF-36 can be difficult. A question often asked is, “What do the numbers really mean?” Criteria-based and content-based interpretations are used to gain a better understanding of differences in SF-36 Physical Component Summary (PCS) scores.⁴ Content-based interpretation is based on analyses of the content of individual SF-36 items within the survey for the general US population, such as “Does your health limit you in walking one block?” Criteria-based interpretation is based on external criteria such as predicting job loss due to health problems and is also based on US population norms for PCS scores.

The objective of this study was to assess the impact of adalimumab therapy (used in combination with MTX) on initial and sustained improvement in HRQOL for patients with early RA, and to interpret the findings.

Study J was a 2-year, double-blind, active comparator-controlled, Phase III study conducted at many sites worldwide. MTX-naïve adult patients with early RA (<3 years) were randomized to 1 of 3 treatment arms (FIG. 18):
Adalimumab 40 mg every other week (eow)+MTX*;
Adalimumab 40 mg eow monotherapy+placebo; and
MTX monotherapy+placebo. (*7.5 mg weekly increased to 20 mg over 8 weeks, as tolerated and as needed)

The SF-36 was used to assess the 8 domains of HRQOL—physical functioning, role—physical, bodily pain, general health, vitality, social functioning, role—emotional, and mental health. These 8 domains were aggregated into Physical Component Summary (PCS) and Mental Component Summary (MCS) scores. The SF-36 has a scale of 0-100, with higher scores indicating better health-related quality of life.

Minimum clinically important differences (MCID) are defined as improvements of 5-10 points in the individual domains scores and 2.5-5 points in the PCS and MCS⁵.

All HRQOL domains were measured at baseline, and after 12, 26, 42, 52, 76, and 104 weeks of therapy. In this analysis, mean scores and mean changes in each HRQOL domain are reported, as well as the PCS and MCS, at Weeks 12 and 104. Criteria-based interpretation was used to understand the meaning of differences in PCS scores for work loss and resource use and content-based interpretation for specific SF-36 items.⁴

A total of 799 patients enrolled in the Study J study. Baseline demographics and clinical characteristics were similar between the 3 arms, and indicative of early, erosive RA (Table 96).

TABLE 96
Baseline demographics

| | Adalimumab + MTX | Adalimumab | MTX |
|---|---|---|---|
| | (n = 268) | (n = 274) | (n = 257) |
| Age (yrs), mean (range) | 52 (19-81) | 52 (18-80) | 52 (18-82) |
| % Females | 72% | 77% | 74% |
| % with prior DMARDs | 33% | 33% | 32% |
| % Corticosteroid use | 36% | 37% | 35% |
| % RF-positive | 87% | 83% | 81% |
| SJC (0-66), mean (range) | 23.1 (8-83) | 24.2 (8-58) | 24.2 (8-64) |
| TJC (0-68), mean (range) | 33.1 (11-68) | 34.1 (11-68) | 34.5 (12-66) |
| HAQ DI, mean (range) | 1.5 (0-2.9) | 1.6 (0-3) | 1.5 (0-3) |
| DAS28, mean (range) | 6.3 (3.8-8.6) | 6.4 (3.9-8.4) | 6.3 (4.0-8.4) |
| % Pts with DAS28 >5.1 | 86% | 88% | 91% |
| CRP (mg/dL) | 4.7 | 5.0 | 4.6 |
| Duration since diagnosis | 0.7 | 0.7 | 0.8 |
| of RA (yrs), mean | | | |
| TSS, mean (range) | 18.1 (0.0-137.5) | 18.8 (0.0-110.5) | 21.9 (0.0-149.5) |
| JE, mean (range) | 11.0 (0-98.5) | 11.3 (0.0-67.5) | 13.6 (0.0-75.5) |
| JSN, mean (range) | 7.1 (0.0-68.5) | 7.5 (0.0-74.0) | 8.2 (0.0-84.0) |
| TSS/Duration RA | 25.6 | 26.7 | 27.4 |
| % Pts with JE | 93% | 94% | 96% |

Mean baseline SF-36 domains scores (including the following indices: physical functioning, role—physical, bodily pain, general health, vitality, social functioning, role—emotional, mental health) for patients who received adalimumab plus MTX were comparable to scores for patients who received MTX monotherapy. These scores were well below the norms for the US population,⁴ indicating that patients with early, erosive RA have substantial HRQOL impairment.

HRQOL domain score results were comparable between the 2 monotherapy arms By Week 12, patients on adalimumab plus MTX combination therapy and patients on MTX monotherapy had both achieved substantial improvements in HRQOL domain scores. These improvements were even greater at the end of two years of therapy.

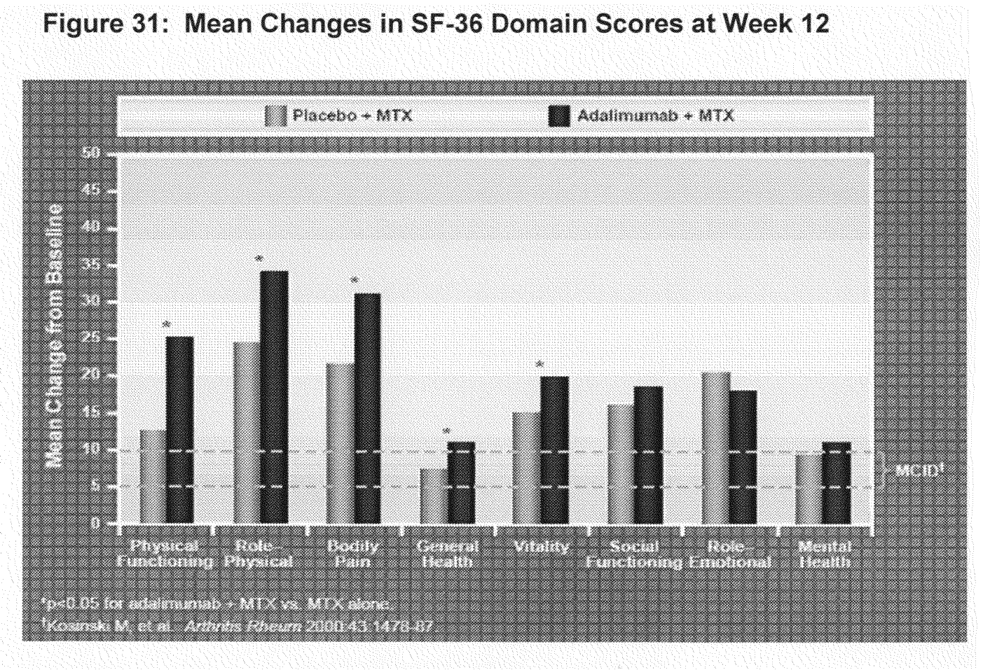

By Week 12, patients on combination therapy had achieved clinically meaningful and statistically significantly greater improvements in 5 of 8 domains vs. patients on MTX monotherapy (FIG. 31). In addition, these improvements had been sustained at the end of 2 years of therapy (FIG. 32).

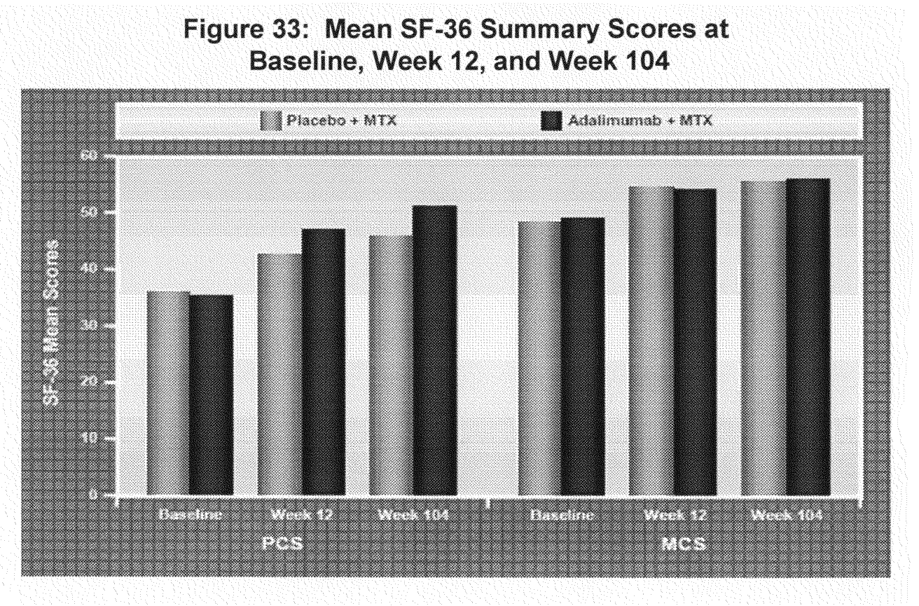

Mean baseline PCS scores for the adalimumab plus MTX and MTX monotherapy groups were 31.7 and 32.2, respectively. The mean PCS score for the combination therapy group at Week 12 had improved to 42.2 vs. 38.3 for the MTX monotherapy group (FIG. 33).

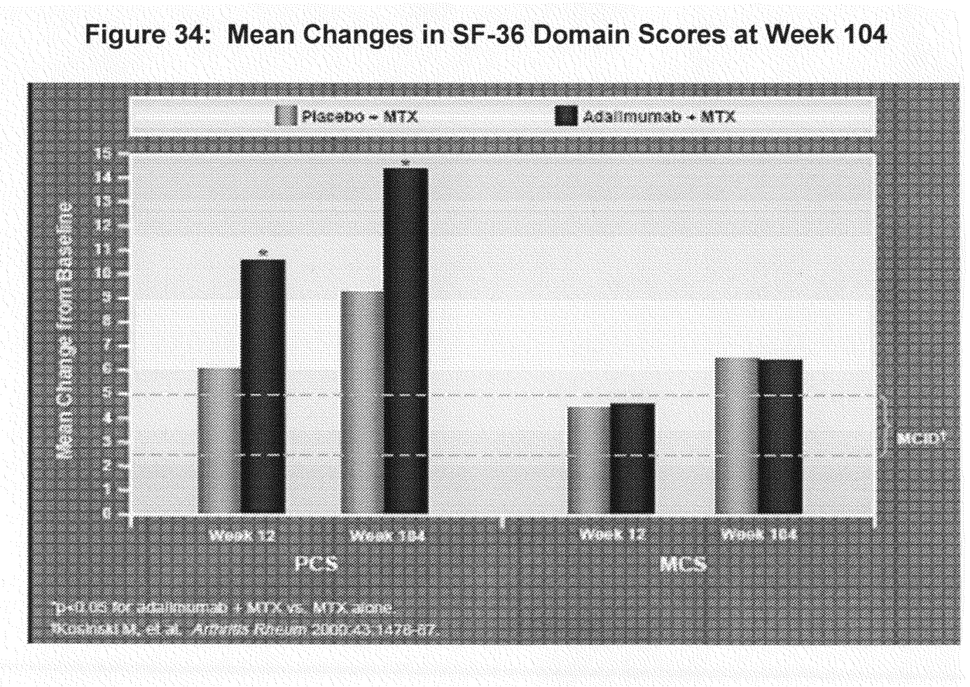

The 4.5 difference between the 2 groups in PCS mean change from baseline at 12 weeks was clinically meaningful and sustained through 2 years (5.1) (p<0.0001) (FIG. 34).

Based on criteria-based interpretation (CrBI) of the PCS for the general US population, the percentage differences between the 2 groups indicate patients on adalimumab plus MTX were less likely to lose their jobs or to be unable to work. In addition, patients receiving adalimumab plus MTX were less likely to be hospitalized or to visit a physician than those on MTX monotherapy. Based on content-based interpretation (CoBI) of the PCS, about half of those in the adalimumab plus MTX group, compared with patients in the MTX monotherapy group, were likely to have their health compromised or their abilities to walk one block or climb one flight of stairs impaired. Also based on CoBI, patients in the adalimumab plus MTX group would be half as likely as the those in the MTX monotherapy group to have had difficulty at work or to have cut down their time at work. In addition, patients in the adalimumab plus MTX group had more energy and were less likely to feel tired.

TABLE 97
Interpretation of SF-36 PCS Scores - Percentages Based on General US
Population Norms

| | MTX alone | | Ada + mtx | |
|---|---|---|---|---|
| | Baseline | Week 104 | Baseline | Week 104 |
| Criteria-based | | | | |
| Hospitalized | 10.7 | 7.7 | 10.9 | 6.6 |
| MD visit | 47.7 | 34.6 | 48.4 | 28.0 |
| Not work | 44.1 | 24.2 | 45.5 | 14.7 |
| Job loss | 30.3 | 20.8 | 30.9 | 16.4 |
| Content-based | | | | |
| Difficulty | 88.1 | 61.2 | 88.8 | 25.0 |
| working | | | | |
| Time off work | 64.6 | 29.8 | 65.9 | 13.7 |
| Walk on block | 44.4 | 21.4 | 46.2 | 10.7 |
| Climb stairs | 66.4 | 30.7 | 68.0 | 17.0 |
| Have energy | 9.7 | 26.6 | 10.1 | 36.3 |

In MTX-naïve patients with rapidly progressive, recent-onset RA, adalimumab plus MTX was superior to MTX monotherapy in providing statistically significant and clinically meaningful improvements in HRQOL in early RA. Patients in the adalimumab plus MTX group were more likely to be active, have more energy, and to be able to walk one block and climb one flight of stairs. In addition, patients in the adalimumab plus MTX group would have had substantially less job loss and have had less difficulty on the job than patients on MTX monotherapy. A significantly lower change in PCS score at 2 years in the MTX monotherapy group may mean patients on MTX monotherapy have greater health care utilization. This is yet another measure that demonstrates that a TNF antagonist plus MTX is superior to MTX monotherapy in the treatment of RA.
1. De Vries-Bouwstra J K, et al. Arthritis Rheum 2003: 48:3649 (LB18).
2. Smolen J S, et al. Ann Rheum Dis 2003: 61(Suppl I):64.
3. Weinblatt M E, et al. Arthritis Rheum 2003; 48:35-45.
4. Ware J E, Kosinski M. SF-36 Physical & Mental Health Summary Scales: A Manual for Users of Version 1. 2nd ed. Lincoln, R I: Quality Metric Incorporated, November 2002.
5. Kosinski M, et al. Arthritis Rheum 2000: 43:1478-87.

### EXAMPLE 32

### Adalimumab (HUMIRA®) is Effective in Treating Patients with Rheumatoid Arthritis who Previously Failed Etanercept and/or Infliximab in Real-Life Clinical Settings

Experience in RA therapy with TNF antagonists after previous failure with other TNF antagonists is limited, and the impact of prior TNF antagonist failure, due to insufficient response or intolerance, on the efficacy and safety of succeeding TNF antagonists has not been evaluated. The objective of the study described herein was to investigate the efficacy and safety of adalimumab after 12 weeks of treatment in patients with RA who failed previous therapy with etanercept (ETA) and/or infliximab (INF) in real-life clinical practice due to lack of response or intolerance (Arthritis Rheum 2005; 52(9)(Suppl): S144).

Patients with long-standing, moderate to severe RA were enrolled in Study A at 450 sites in 12 different countries. The study design for Study A is outlined in FIG. 21. Patients received adalimumab 40 mg every other week (eow) subcutaneously (sc) in addition to their existing but insufficient antirheumatic therapies. Previous treatment with other biologics was allowed up to 8 weeks before enrollment. Patients met the following inclusion criteria: Age≧18 years; RA (defined by American College of Rheumatology criteria) for ≧3 months; active, moderate to severe RA (Disease Activity Score (DAS28)≧3.2); unsatisfactory response or intolerance to at least one prior DMARD, including biologics. Efficacy assessments were performed at weeks 0 (baseline), 2, 6, and 12. The key outcomes measured were: change in DAS28; EULAR responses; ACR20, ACR50, ACR70 responses; changes in Tender Joint Count (TJC) and Swollen Joint Count (SJC); and change in Health Assessment Questionnaire (HAQ).

### Results

Of 6610 enrolled patients, a history of prior DMARD therapy was known for 6532. In all, 819 patients had a history of ETA and/or INF therapy for a median duration of 10 months (up to 58). The median interval between last ETA and/or INF dose and first ADA dose was 4 months (up to 64). Reported reasons for withdrawal from ETA and/or INF included: 171, no response; 260, loss of response; 160, intolerance (categories not mutually exclusive).

Baseline characteristics are shown in Table 98. Mean baseline characteristics of patients without/with anti-TNF history included: prior DMARDs, 2.6/4.8; DAS28, 6.0/6.3; and HAQ, 1.60/1.85. At baseline, patients with prior experience with ETA and/or INF had higher disease activity, longer disease duration, and had been treated with a higher number of previous DMARDs than ETA/INF naïve patients. Patients with prior ETA and/or INF experience, especially those with insufficient efficacy, were more limited in their physical function, as measured by HAQ Disability Index. Patients with previous failure to INF received adalimumab-DMARD combination therapy more frequently than patients who previously failed ETA.

TABLE 98
Baseline Characteristics by Prior ETA and/or INF Experience and by
Exclusive Reasons for Discontinuation

| | No | | | | | | | |
|---|---|---|---|---|---|---|---|---|
| | prior | Prior | | | | | | |
| | ETA | ETA | No | | Loss of | | | |
| | and/or | and/or | response | | efficacy | | Intolerance | |
| | INF | INF | ETA | INF | ETA | INF | ETA | INF |
| Baseline Characteristics* | n = 5711 | n = 899 | n = 57 | n = 100 | n = 45 | n = 249 | n = 36 | n = 128 |
| Age (years)^{†} | 54 | 53 | 57 | 54 | 53 | 54 | 55 | 54 |
| # Prior DMARDs^{†} | 2.7 | 5.0 | 5.1 | 4.6 | 5.0 | 4.6 | 5.1 | 4.4 |
| RA Duration (years)^{†} | 11 | 12 | 14 | 10 | 14 | 13 | 12 | 11 |
| % Concomitant steroid | 70 | 77 | 75 | 72 | 78 | 77 | 75 | 78 |
| use^{‡} | | | | | | | | |
| % Concomitant DMARD | 75 | 69 | 54 | 66 | 40 | 76 | 42 | 76 |
| use | | | | | | | | |
| HAQ^{†} | 1.60 | 1.85 | 2.06 | 2.00 | 1.81 | 1.84 | 1.95 | 1.78 |
| DAS28^{†} | 6.0 | 6.3 | 6.5 | 6.4 | 6.6 | 6.2 | 6.8 | 6.3 |

*Interval since last administration >8 weeks.
^{†}Mean values.
^{‡}Max dose of 10 mg/d prednisolone equivalent allowed.

Key efficacy results at Week 12 were obtained for all parameters. Substantial percentages of patients with previous ETA/INF experience achieved ACR20, ACR50, ACR70, and at least Moderate and Good EULAR response rates with adalimumab therapy, as shown in Table 99.

TABLE 99
ACR and EULAR response rates for patients with and witout
prior ETA and/or INF experience

| | | | | | Moderate | Good |
|---|---|---|---|---|---|---|
| | | ACR20 | ACR50 | ACR70 | EULAR | EULAR |
| % of | No prior | 70 | 41 | 19 | 84 | 35 |
| patients | ETA | | | | | |
| | and/or INF | | | | | |
| | (n = 5711) | | | | | |
| | Prior ETA | 60 | 33 | 13 | 76 | 23 |
| | and/or INF | | | | | |
| | (n = 899) | | | | | |

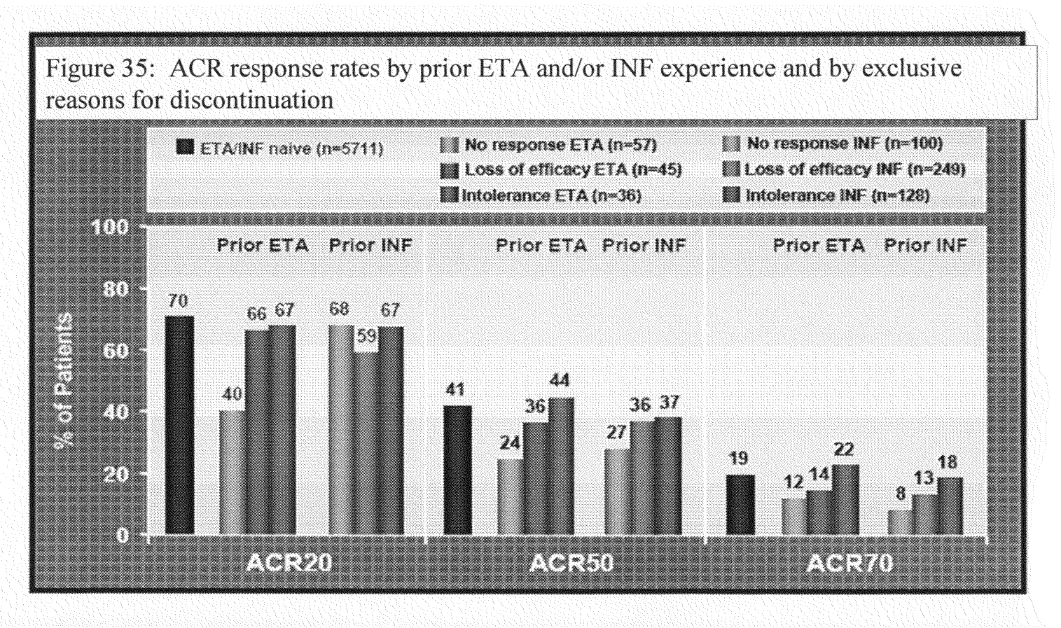

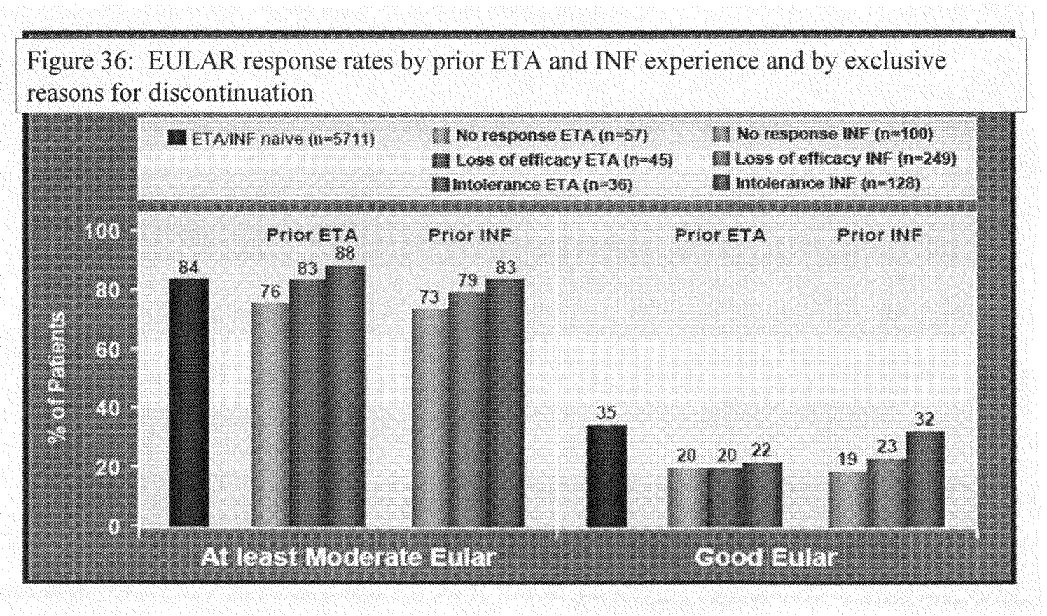

Patients with previous loss of efficacy or intolerance to ETA/INF achieved ACR responses similar to ETA/INF naïve patients. ACR response rates, sorted by prior ETA and/or INF experience and by exclusive reasons for discontinuation are graphically represented in FIG. 35. Patients with previous loss of efficacy or intolerance to ETA/INF achieved EULAR response rates similar to ETA/INF naïve patients. EULAR response rates, sorted by prior ETA and INF experience and by exclusive reasons for discontinuation are graphically represented in FIG. 36.

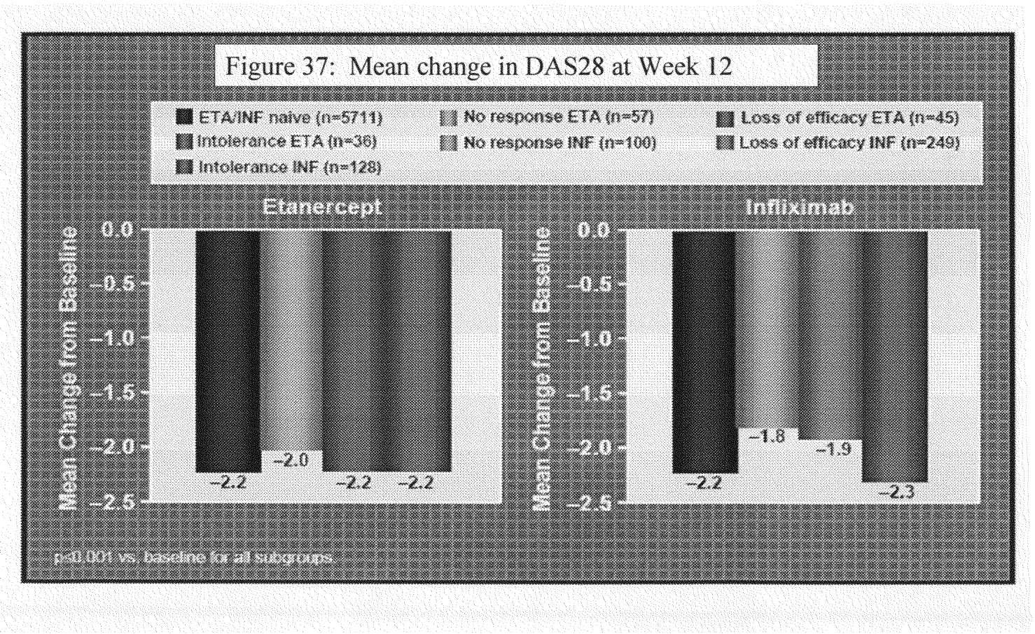

A total of 15 patients in Study A had no prior response to ETA and INF. After 12 weeks of adalimumab therapy, 25% of these patients achieved ACR50, and 50% achieved a moderate EULAR response. The mean relative changes in TJC and SJC in patients with a lack of response to ETA and INF were −44% and −42%, respectively (p<0.01). Patients with previous ETA/INF experience achieved TJC and SJC improvements similar to ETA/INF naïve patients. Mean DAS28 scores improved significantly in patients with previous ETA and/or INF failure, as shown in FIG. 37.

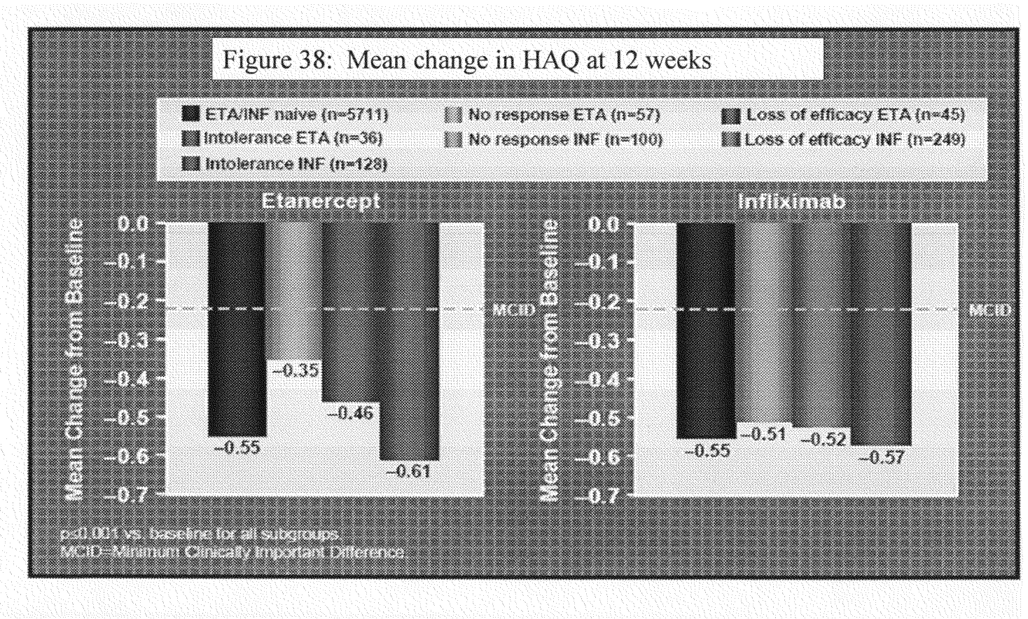

Physical function, as measured by mean change in HAQ, improved significantly in patients with previous ETA and/or INF failure after 12 weeks of adalimumab therapy, as shown in FIG. 38. At Week 12, withdrawal rates of patients who previously failed ETA and/or INF were low. However, overall withdrawal rates were higher compared to TNF-antagonist naïve patients, as shown in Table 100. Up to Week 12, withdrawals (%) among patients without/with anti-TNF history included lack of efficacy 1.2/2.2, and adverse events (AE) 4.0/4.8, respectively. Patients who had previously discontinued ETA or INF because of intolerance discontinued adalimumab more frequently because of adverse events than lack of efficacy, as shown in Table 101. Although patients with ETA and/or INF history were more severely ill, all subgroups did profit from ADA therapy irrespective of reason for discontinuing ETA and/or INF therapy (data is shown in Table 102). Of 14 patients treated unsuccessfully with both ETA and INF, 3 had ACR50 responses to ADA.

TABLE 100
Withdrawal Rates at Week 12 in Patients With of Without History of anti-
TNF Therapy (Multifold Answers Allowed)

| Reason for withdrawal | No prior ETA and/or INF | Prior ETA and/or INF |
|---|---|---|
| n (%) | n = 5711 | n = 899 |
| Total withdrawals | 381 (6.7) | 89 (9.9) |
| Adverse event | 234 (4.1) | 50 (5.6) |
| Lack of efficacy | 68 (1.2) | 26 (2.9) |

TABLE 101
Withdrawal Rates at Week 12 of Adalimumab Therapy by Reason for
Discontinuation of Prior ETA/INF (Multifold Answers Allowed)

| | No | | Loss of | | | |
|---|---|---|---|---|---|---|
| | response | | efficacy | | Intolerance | |
| Reason for withdrawal | ETA | INF | ETA | INF | ETA | INF |
| n (%) | n = 57 | n = 100 | n = 45 | n = 249 | n = 36 | n = 128 |
| Total withdrawals | 6 (10.5) | 6 (6.0) | 3 (6.7) | 20 (8.0) | 5 (13.9) | 11 (8.6) |
| Adverse event | 2 (3.5) | 4 (4.0) | 1 (2.2) | 14 (5.6) | 2 (5.6) | 8 (6.3) |
| Lack of efficacy | 3 (5.3) | 2 (2.0) | 1 (2.2) | 4 (1.6) | none | 2 (1.6) |

TABLE 102
Efficacy of Adalimumab at 12 Weeks per Prior TNF-antagonist Therapy and
by Exclusive Reasons for Discontinuation

| | | ETA | | | | | | |
|---|---|---|---|---|---|---|---|---|
| | No ETA | and/or | No | | Loss of | | | |
| | or INF | INF | response | | response | | Intolerance | |
| | history | history | ETA | INF | ETA | INF | ETA | INF |
| Outcomes* | (N = 5713) | (N = 819) | (N = 47) | (N = 84) | (N = 29) | (N = 186) | (N = 31) | (N = 86) |
| ACR20 (%) | 70 | 61 | 33 | 60 | 72 | 72 | 67 | 67 |
| ACR50 (%) | 41 | 33 | 17 | 29 | 32 | 40 | 44 | 38 |
| ACR70 (%) | 19 | 14 | 10 | 7 | 12 | 14 | 18 | 17 |
| ΔDAS28^{†} | −2.2 | −1.9 | −1.7 | −1.9 | −2.0 | −2.0 | −2.3 | −2.3 |
| DAS28 ≦ 2.6 | 21 | 13 | 18 | 11 | 8 | 13 | 11 | 19 |
| (%) | | | | | | | | |
| ΔHAQ^{†} | −0.55 | −0.49 | −0.29 | −0.49 | −0.47 | −0.56 | −0.65 | −0.60 |

Adverse events were collected throughout the treatment period. Numbers of patients with serious adverse events are shown in Table 103. The median duration of exposure to adalimumab was 211 days. The overall safety profile of patients with previous exposure to other TNF-antagonists was good. No serious demyelinating diseases were reported, and no serious systemic lupus erythematosus was observed. Among patients with anti-TNF history, the most frequent severe AE were musculoskeletal disorders (2%), infections (1%), and skin disorders (0.6%). Of the 160 ETA and/or INF-intolerant patients, 8 discontinued ADA due to AE, 1 of them due to hypersensitivity.

TABLE 103
Numbers of Patients with Serious Adverse Events

| | N (%) |
|---|---|
| SAE | 160 (17.8) |
| Musculoskeletal disorders | 50 (5.6) |
| RA-related SAEs | 33 (3.7) |
| Serious infections | 39 (4.3) |
| Pneumonia | 7 (0.8) |
| Sepsis | 8 (0.9) |
| Tuberculosis | 2 (0.2) |
| Opportunistic infection (CMV pneumonia) | 1 (0.01) |
| Fractures or ligament/tendon ruptures | 14 (1.6) |
| Joint surgeries | 5 (1) |
| Cardiac SAE | 11 (1.2) |
| Congestive heart failure | 2 (0.2) |
| Serious general disorders | 10 (1.1) |
| Pyrexia | 6 (0.7) |

### Conclusions

In patients with RA treated in real-life clinical practice, ADA was effective and well-tolerated irrespective of prior ETA and/or INF therapies or reasons for discontinuing them. RA patients with prior ETA and/or INF failure demonstrated statistically significant improvements in key efficacy parameters when treated with adalimumab in real-life clinical practice. The response of patients naïve to TNF-antagonists was slightly better overall than for patients who failed prior TNF-antagonist therapies, who generally had more severe RA at study entry. The reasons for discontinuing prior TNF-antagonists (lack of efficacy, loss of efficacy over time, and intolerance) did not affect the outcome of treatment with adalimumab.

### EXAMPLE 33

### Adalimumab (HUMIRA®) is Effective and Safe in Treating Rheumatoid Arthritis (RA) in Real-Life Clinical Practice: 1-Year Results

Recent clinical trials demonstrated that the combination of TNF antagonists and methotrexate (MTX) is superior to MTX alone in the treatment of RA (De Vries-Bouwstra J K et al. Arthritis Rheum 2003: 48:3649 (Lb18), Smolen J S, et al. Ann Rheum Dis 2003: 61 (Suppl 1): 64, Weinblatt M E, et al. Arthritis Rhem 2003: 48:35-45). The efficacy and safety of TNF-antagonists in treating patients with active RA have been confirmed in clinical trials; however, treatment of a broad RA patient population with TNF antagonists in combination with different DMARDs, with enrollment requirements close to national recommendations for anti-TNF therapy, has not been evaluated in clinical trials.

This prospective evaluation examined the efficacy and safety of adalimumab (ADA) in a large cohort of patients with active, insufficiently treated RA, various co-morbidities, a broad range of antirheumatic co-medications, and varied social care systems, in real-life settings. The objectives of this evaluation were to investigate the efficacy and safety of adalimumab when combined with a variety of concomitant antirheumatic drugs in the treatment of a large patient population with active RA, and to investigate the maintenance of efficacy and safety of adalimumab in a real-life adapted setting over 52 weeks of treatment.

Patients with active RA received adalimumab 40 mg every other week (eow) for 12 weeks subcutaneously (sc) either in addition to or as replacement of their pre-existing antirheumatic therapy in Study A (Study A is outlined in FIG. 21). Moderately to severely active RA was defined by Disease Activity Score 28 (DAS28)≧3.2 at baseline. Unsatisfactory response or intolerance to at least one prior DMARD was required for enrollment in Study A. A 12-week study period was followed by an optional extension phase with efficacy and routine safety evaluations performed at Weeks 2, 6, 12, 20, and every 8 weeks thereafter. Patients discontinued the study when they stopped receiving adalimumab or received commercial HUMIRA®. Key efficacy parameters include ACR20, 50, and 70 responses; EULAR responses; and changes in DAS28, Tender Joint Count (TJC), Swollen Joint Count (SJC), C-Reactive Protein (CRP), and Health Assessment Questionnaire Disability Index (HAQ). Efficacy outcomes at 52-weeks were determined using the last observation carried forward (LOCF) approach. Adverse events (AE) were collected throughout the entire treatment period, also beyond Week 52.

### Results

Study A was the largest adalimumab clinical trial, with 6,610 patients enrolled in 11 European countries plus Australia at 450 sites. Mean baseline characteristics include: age, 54 yrs; disease duration, 11 yrs; DAS28, 6.0; HAQ, 1.64; and 3 prior DMARDs. Positive rheumatoid factor was present in 73% and a positive PPD Mantoux in 13% of patients.

Baseline demographic data and clinical characteristics are shown in Table 104. At the treating physician's discretion, ADA was prescribed alone (25%) or with prior DMARD(s), leading to 45 ADA-DMARD combinations. A subset of patients (N=4,879, 74%) were treated with adalimumab in combination with one or more DMARDs, including MTX 2794 (42% of 6,610), leflunomide (LEF), 842 (13%), sulfasalazine (SSZ), 133 (0.2%), and antimalarials (AM), 148 (0.2%). 4,708 (71%) patients were taking concomitant corticosteroids (max 10 mg/day prednisolone equivalent). 1,002 patients (15%) patients were <40 years old and 238 (0.04%) patients were ≧75 years. 2,906 (44%) patients had RA for >10 years, and 1,288 (19%) patients had a DAS28>7.0 at baseline. As of Apr. 7, 2005, 6202 (94%) patients had completed Week 12. Patients who opted to continue treatment beyond Week 12 numbered 4084 at Week 28, 2983 at Week 36, and 1243 at Week 52. The number of patients with data available at various timepoints throughout the study is shown in Table 105.

TABLE 104
Baseline Demographics and Clinical Characteristics

| Characteristics | N = 6610 |
|---|---|
| Age (Years)* | 54 (13) |
| % Female | 81 |
| % RF positive | 73 |
| Duration RA (Years)* | 11 (9) |
| # previous DMARDs* | 3.0 (1.8) |
| DAS28* | 6.0 (1.1) |
| HAQ DI* | 1.64 (0.68) |

*Mean values (SD)

TABLE 105
Number of Patients in Study A Over Time

| | Baseline | Week 12 | Week 28 | Week 36 | Week 52 |
|---|---|---|---|---|---|
| N | 6610 | 6234 | 4119 | 3021 | 1251 |

The mean exposure to ADA was 233 days, up to a maximum of 120 weeks. The median exposure to adalimumab through Week 12 was 84 days, and the overall median exposure to adalimumab was 211 days. The rate of premature withdrawals was low. Patients treated with adalimumab demonstrated rapid increases in ACR response rates. 40% of patients achieved ACR20 by Week 2. ACR50 and ACR70 responses increased through Week 28 and remained stable through Week 52 (see Table 106).

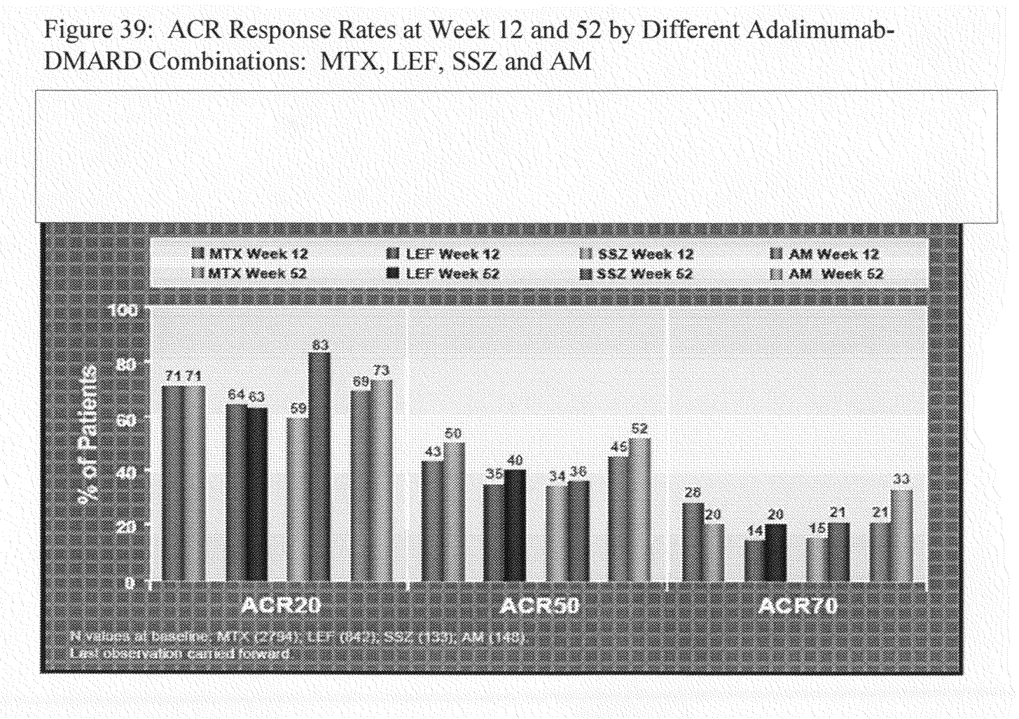

After 12 weeks of adalimumab therapy the functional disability, as measured by mean changes in HAQ, diminished significantly. Mean improvement in HAQ scores was maintained in the open-label extension period. The change in HAQ observed at Weeks 12, 28, and 52 was −0.52 week 12 (n=6235), −0.56 week 28 (n=4119), −0.57 week 52 (n=1251) (MCID about 2.2; statistically significant change in HAQ for each week). MTX was the most commonly used DMARD in combination with adalimumab. Other DMARD combinations achieved similar ACR responses compared to MTX (data is shown in FIG. 39). Key efficacy outcomes are displayed in Table 106.

TABLE 106
Efficacy of Adalimumab up to Week 52 (LOCF)

| Efficacy criteria | Week 12 | Week 28 | Week 36 | Week 52 |
|---|---|---|---|---|
| ACR20 (%) | 66 | 67 | 67 | 67 |
| ACR50 (%) | 38 | 43 | 44 | 45 |
| ACR70 (%) | 17 | 23 | 23 | 24 |
| Moderate EULAR | 81 | 81 | 82 | 82 |
| response (%) | | | | |
| Good EULAR response | 32 | 37 | 37 | 38 |
| (%) | | | | |
| ΔDAS28 (mean) | −2.1 | −2.2 | −2.2 | −2.3 |
| DAS28 ≦ 2.6 (%) | 20 | 27 | 29 | 35 |
| ΔHAQ (mean) | −0.52 | −0.56 | −0.56 | −0.57 |

Adalimumab was well-tolerated. Withdrawal rates reported by the investigator to be due to adverse events or lack of efficacy were low at Week 12 and sustained through Week 99 (data is shown in Table 107). Reasons for withdrawal included lack of efficacy in 446 (6.8%) and adverse events (AE) in 662 (10.1%) of the patients.

TABLE 107
Withdrawal Rates by Treatment Period (Multifold Answers*)

| All patients | | Any time up to Week |
|---|---|---|
| (N = 6610) | Week 12 | 99 |
| Total | 470 (7%) | 1377 (21%) |
| Lack of efficacy | 284 (4%) | 682 (10%) |
| Adverse event | 94 (1%) | 450 (7%) |

*Multifold answers for reason of withdrawal permitted, other reasons are not shown

### Safety

Adverse events (AE) were collected throughout the treatment period and 70 days after the last injection of adalimumab, which is the equivalent of 5 serum half-lives for adalimumab. A total of 14,671 AEs were reported in 4,780 patients (72%) (348.5/100 pt.yrs). There were 1,195 serious adverse events (SAEs) in 882 (13%) patients (28.4/100 pt.yrs); RA related SAEs were the most frequently reported events among them (152, 3.6/100 pt.yrs). Numbers of patients with SAEs are shown in Table 108.

The number of patients with serious adverse events was consistent with the results from previous pivotal adalimumab clinical studies. The standardized incidence ratio of malignancies for patients treated with adalimumab was 0.74 (compared to SEER rates). The standardized mortality ratio for patients treated with adalimumab was 0.85. The most frequent AE leading to withdrawal were 3.4 infections, 3.1 musculoskeletal, 2.7 skin, and 2.2 general disorders per 100 patient years (pt-yrs). There were 5.3 serious infections and 0.6 malignancies per 100 pt-yrs. No new safety signals were observed.

TABLE 108
Numbers of Patients with Serious Adverse Events

| | N (%) |
|---|---|
| Serious infections | 203 (3.1) |
| Malignancies | 44 (0.7) |
| Lymphoma | 2 (0.03) |
| Demyelinating diseases | 4 (0.06) |
| Congestive heart failure | 18 (0.3) |
| SLE/lupus-like syndrome | 2 (0.03) |

### Conclusions

Adalimumab led to clinically significant and stable improvements over 1 year of treatment in all key efficacy parameters. The Study A data confirmed the results observed in earlier pivotal trials of adalimumab with fewer patients. Adalimumab significantly reduced the signs and symptoms of severe and long-standing RA. The study drug was efficacious when administered alone or with a variety of DMARD combinations. The efficacy of adalimumab was confirmed in a broad clinical setting and maintained over time. Long-term administration of adalimumab was well-tolerated. No new safety signals were observed in a large RA population. The benefit-risk ratio in real-life clinical settings was found to be positive.

### EXAMPLE 34

### The Relationship of Radiographic Progression to Clinical Response in Patients with Early RA Treated with Adalimumab (HUMIRA®) Plus MAX or MTX Alone

Study J was a 2 year study of MTX-naïve patients with aggressive, early RA. It is the only study of early RA to compare a treatment with a TNF blocker+MTX to either treatment given alone (Arthritis Rheum 2005; 52(9)(Suppl):S451). Patients developed less radiographic progression over 2 years when treated with adalimumab+MTX than with MTX alone. Radiographic efficacy of treatment in RA does not always correlate with clinical efficacy. The objective of the study described herein was to determine whether the radiographic efficacy of adalimumab+MTX in early RA was greater than that of MTX monotherapy at various levels of clinical response.

The Study J study design is outlined in FIG. 18. The primary endpoints of Study J were the ACR50 and the mean change in Total Sharp Score at 1 year, comparing Combination therapy with MTX monotherapy. In keeping with this focus, the analysis presented herein was limited to patients in these same 2 treatment arms, using outcomes at 6 months to assess early effects, and 2 years to assess longer-term changes. Patients in Study J were all MTX-naïve and only ⅓ had used a previous DMARD. At baseline, mean disease duration was about ¾ of a year. Additional baseline demographics and clinical characteristics are shown in Table 109. Patients had very active arthritis and rapidly progressive joint destruction, with mean total Sharp scores of approximately 18 to 22. Mean baseline Sharp Scores for the ITT cohort, 6 month and 2 year observed cohorts are shown in Table 110.

TABLE 109
Study J Baseline Demographics and Clinical Characteristics

| | Adalimumab + MTX | MTX |
|---|---|---|
| | (N = 268) | (N = 257) |
| Age (yrs), mean (range) | 52 (19-81) | 52 (18-82) |
| % with prior DMARDs | 33% | 32% |
| SJC (0-66), mean (range) | 23.1 (8-63) | 24.2 (8-64) |
| TJC (0-68), mean (range) | 33.1 (11-68) | 34.5 (12-66) |
| DAS28, mean (range) | 6.3 (3.8-8.6) | 6.3 (4.0-8.4) |
| Duration since diagnosis of | 0.7 | 0.8 |
| RA (yrs), mean | | |
| TSS, mean (range) | 18.1 (0.0-137.5) | 21.9 (0.0-149.5) |

TABLE 110
Mean Baseline Sharp Scores for the ITT cohort, 6 month and 2 year
observed cohorts

| | Total (ITT) | | 6 months | | 2 years | |
|---|---|---|---|---|---|---|
| | N | TSS | N | TSS | N | TSS |
| Ada + MTX | 268 | 18.1 | 240 | 18.2 | 199 | 18.1 |
| MTX | 257 | 21.9 | 213 | 21.8 | 166 | 22.7 |

ITT = Intention to treat

Patients in the Ada+MTX and the MTX arms were classified according to level of clinical response (non-responder (<ACR20), ACR20, ACR50, or ACR70) and radiographic progression (Yes or No (ΔTSS≦0.5)). The ΔTSS was analyzed overall, and for ACR subgroups. This included determination of mean ΔTSS and probability plots. Analyses used observed data from patients completing 6 months or 2 years of treatment (no imputation).

Among patients who completed 6 months of treatment with adalimumab+MTX, the ACR scores were higher than in patients who completed 6 months of MTX alone, especially at the ACR50 and ACR 70 levels. The percentage of patients taking ada+mtx who achieved an ACR20/50/70 response was 82.1%/69.2% O/148.8%, respectively (statistically significant) vs. the percentage of patients taking mtx alone who achieved an ACR20/50/70 response which was 79.3%/51.6%/28.6%. After 2 years of treatment, responses improved further, with ACR 70s seen in 62% of patients on combination therapy, versus 43% on MTX. The mean change in total Sharp score indicates that treatment with adalimumab+MTX gave significantly better control of radiographic progression than treatment with MTX alone, from 6 months to 2 years (ada+mtx the mean change in TSS was 0.6 at 26 weeks, 1.0 at 52 weeks, and 1.1 at 104 weeks vs. mtx alone 3.4 at 26 weeks, 5.2 at 52 weeks, and 6.4 at 104 weeks (ada results statistically significant).

To visualize the individual measurements that underlie these mean values, and to examine their relationship to ACR responses, cumulative probability plots were prepared for these results. The first plots of data examined were those corresponding to the 6 month time point, denoted as 26 weeks. Cumulative probability plots graph the ΔTSS results for all patients in a population, allowing easy recognition of both the percentages of patients in whom TSS progresses, and the severity of progression in those patients.

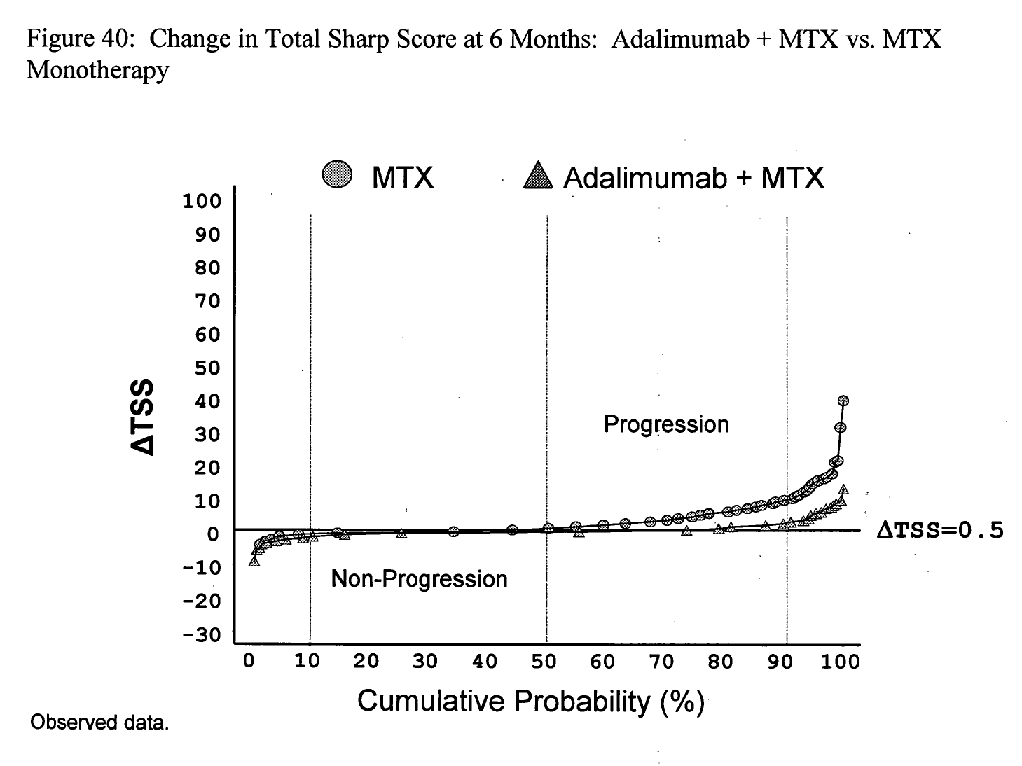

FIG. 40 shows the distribution of the change-in-total Sharp score results for patients who were treated for 6 months. The left end represents the patients whose Sharp scores improved. The flat middle section represents patients whose Sharp scores were largely unchanged. The longer curve indicates that disease progression was controlled in more patients by adalimumab+MTX than by MTX alone. The right end of the curves is most important in assessing joint destruction, as it represents the patients whose Sharp scores increased over 6 months. As defined by a Sharp score increase of at least half a unit, indicated by the horizontal line, these patients are radiographic progressors. The orange MTX curve increases earlier, and rises higher than the green adalimumab+MTX curve, indicating more severe progression.

Patients represented in the graph in FIG. 40 were divided into 4 groups according to their level of ACR response, in order to address the question of whether the frequency or severity of radiographic progression varied with the level of ACR response. The frequency of radiographic progression at 6 months based on the level of ACR response in patients treated with adalimumab and MTX, and MTX alone, is shown in Table 111.

TABLE 111
Frequency of radiographic progression at 6 months based
on level of ACR response

| | | Non- | | | |
|---|---|---|---|---|---|
| | | responder | ACR20 | ACR50 | ACR70 |
| % of patients | Ada + mtx | 37** | 24* | 23* | 23* |
| | Mtx alone | 59 | 55 | 51 | 49 |

*p < 0.001,
**P = 0.054 vs. mtx alone
Radiographic progression = ΔTSS > 0.5; non-responder = <ACR20

Table 112 depicts the mean ΔTSS at 6 months in radiographic progressors. In terms of these two parameters, frequency and severity of progression, ACR70 responders on MTX fared worse than non-responders on combination therapy.

TABLE 112
Mean change in TSS at 6 months in radiographic progression

| | | Non- | | | |
|---|---|---|---|---|---|
| | | responder | ACR20 | ACR50 | ACR70 |
| Mean change | Ada + mtx | 3.2** | 3.3* | 3.2 | 2.8 |
| TSS | Mtx alone | 10.8 | 4.8 | 3.8 | 3.4 |

*p < 0.001,
**p < 0.05 vs. mtx alone
Radiographic progression = ΔTSS > 0.5; non-responder = <ACR20

The probability plots looking at change in TSS by 6 months by level of ACR response (<ACR20, ACR20, ACR50, and ACR70) showed that, for every level of ACR response, radiographic progression was more frequent and more severe with MTX than with combination therapy. This difference is most striking for patients with a less-than ACR20 response, where large increases in TSS were frequent with MTX. At every level of clinical response, adalimumab+MTX gave better radiographic outcomes than MTX monotherapy after 6 months of treatment. Patients treated with adalimumab+MTX displayed both less frequent and less severe progression than patients treated with MTX alone.

It was also of interest whether the early disparity between the radiographic efficacy of combination therapy and MTX monotherapy was maintained over 2 years. The frequency of radiographic progression at 2 years based on level of ACR response is shown in Table 113.

TABLE 113
Frequency of radiographic progression at 2 years based on level of ACR
response

| | | Non- | | | |
|---|---|---|---|---|---|
| | | responder | ACR20 | ACR50 | ACR70 |
| % of patients | Ada + mtx | 38** | 33* | 31* | 28* |
| | Mtx alone | 75 | 62 | 60 | 57 |

*p < 0.001,
**P < 0.05 vs. mtx alone
Radiographic progression = ΔTSS > 0.5; non-responder = <ACR20

After 2 years of treatment, radiographic progression was approximately twice as frequent among patients who received MTX alone, at all levels of clinical response. Furthermore, it was significantly higher among ACR70 responders on MTX than non-responders on combination therapy. The mean ΔTSS at 2 years in radiographic progressors is shown in Table 114.

TABLE 115
Mean change in TSS at 2 years in radiographic progressors

| | | Non- | | | |
|---|---|---|---|---|---|
| | | responder | ACR20 | ACR50 | ACR70 |
| Mean change | Ada + mtx | 5.9 | 4.4 | 4.4 | 3.9 |
| TSS | Mtx alone | 15.5 | 9.4 | 8.5 | 7.5 |

All ada results statistically significant
Radiographic progression = ΔTSS > 0.5; non-responder = <ACR20

In addition, at every level of clinical response, the magnitude of progression among MTX progressors was approximately twice that observed in patients who progressed on combination therapy. After 2 years of treatment, disease progression remained more frequent and more severe at all levels of clinical response for those treated with MTX, relative to those treated with MTX+adalimumab. The 2-year ACR70 results (cumulative probability analysis) indicated that even at this high level of clinical response, progression was twice as frequent and considerably more severe following treatment with MTX than with combination therapy. These patients can differ markedly in their risk of joint damage. Cumulative probability analysis determined that for any given level of ACR response, radiographic progression was less frequent and, on average, less severe with adalimumab+MTX than with MTX monotherapy. Only among patients achieving a high-level remission-like response, such as ACR100 or SJC=0, did MTX control joint destruction as well as adalimumab+MTX.

Adalimumab+MTX led to less frequent and less severe disease progression than MTX at essentially all levels of clinical response. ACR score was a poor predictor of radiographic efficacy, therefore, radiographic monitoring may be warranted in patients regardless of their clinical response. Anti-TNF therapy may be needed to prevent joint damage in patients with early RA, including some with a good clinical response with MTX monotherapy.

### EXAMPLE 35

### Serious Infections in Patients with Rheumatoid Arthritis who Participated in Adalimumab (HUMIRA®) Clinical Trials

Patients with rheumatoid arthritis (RA) are known to have an increased risk of developing infections compared with patients who do not have RA (Doran et al. Arthritis Rheum 2003; 46:2287-93). With the advent of anti-TNFα therapy, there has been concern that RA patients would experience more infections given the role that TNF-α plays in host defense (Ellerin et al. Arthritis Rheum 2003; 48:3013-22; Keystone E C. J Rheumatol 2005; 32 Suppl 74:8-12). Adalimumab, a fully human monoclonal antibody targeting TNF, is approved for treating patients with RA (US, EU, and Latin America) and psoriatic arthritis (US and EU). In earlier reports of overall safety of adalimumab in long-term RA clinical trials, serious infection rates observed were similar to what has been reported in RA patients not on anti-TNF therapy (Schiff M H, et al. Arthritis Rheum 2004; 50(9 Suppl):S562). The purpose of the following study was to retrospectively analyze the incidence of serious infections (SI) in adalimumab (ADA) clinical trials of rheumatoid arthritis (RA) in North America (NA) and Europe (EU), with consideration of patient characteristics at the time of the infection.

The ADA pivotal trials were Study I, Study 1 (both ADA+MTX in MTX partial responders), Study 2 (ADA monotherapy) and Study K (ADA added to standard of care). Patients from the Phase I-III controlled clinical trials of ADA in RA, including the pivotals, were allowed to receive ADA as long-term, open-label therapy in 3 extension studies: 1) Study 18, for ADA monotherapy in the EU, 2) Study 20, for ADA with MTX in NA, and 3) the extension of Study 1, in NA. In the adalimumab open-label trials, adverse events were coded using the Medical Dictionary for Regulatory Affairs (MedDRA). Adverse events are classified as “serious” adverse events based on the following regulatory criteria: fatal, life-threatening, requires inpatient hospitalization, prolongs hospitalization, causes congenital anomaly/birth defect, results in persistent or significant disability/incapacity, an important medical event that jeopardizes the patient and requires medical/surgical intervention to prevent another serious outcome. Serious infections (SI) are defined as “serious adverse events” that are coded under “Infections and Infestations” in the MedDRA coding system. Serious infections reported from first adalimumab exposure to Mar. 1, 2005 were analyzed in the report described herein. Non-serious infections were not included in this analysis. Serious infections reported during these trials were tabulated and rates were calculated as events per patient-year (E/PY). SI events were evaluated for duration of adalimumab therapy prior to first SI, age at the time of event, history of diabetes mellitus (DM), concomitant medications at time of event, disease modifying anti-rheumatic drugs (DMARDs), and systemic steroids. Rates of SI were determined for subsets of patients who, at the time of the SI, had diabetes, or were using concomitant steroids or concomitant MTX.

### Results

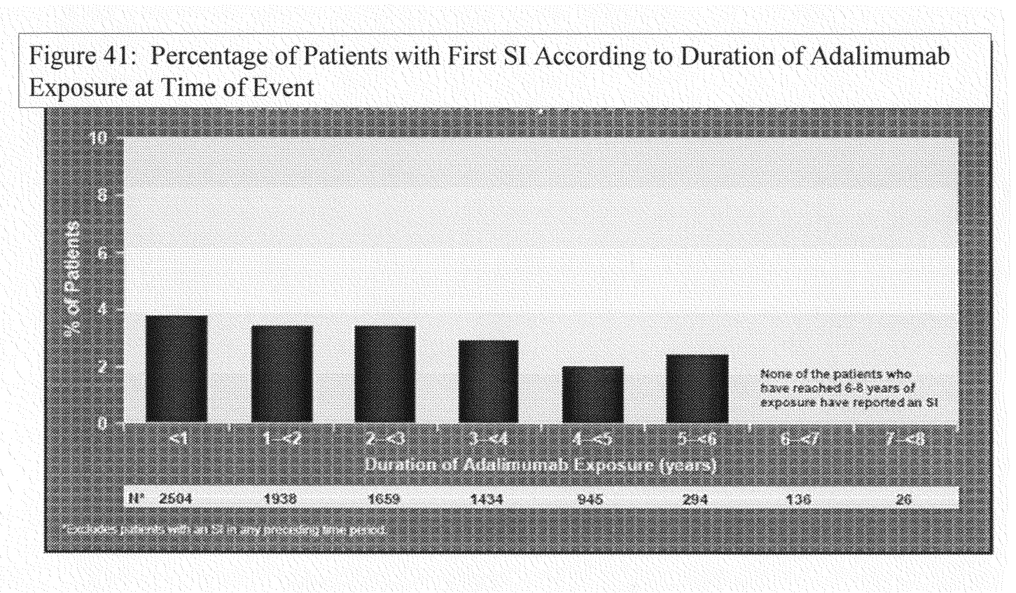

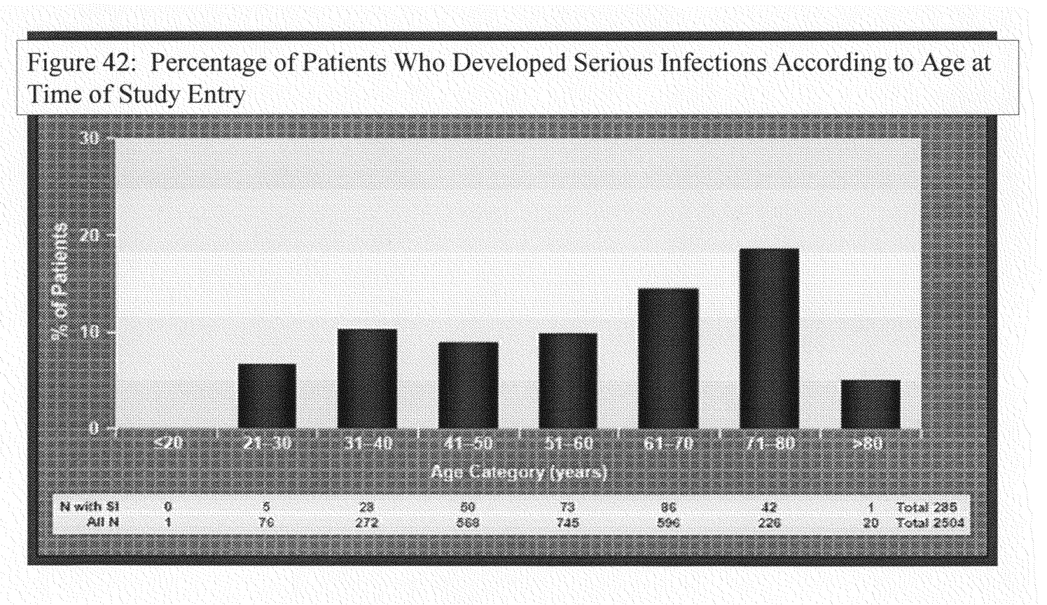

As of Mar. 1, 2005, 2504 patients with RA who were treated with adalimumab for 7951 patient-years (PY) in NA and EU were eligible for this analysis. 337 patients had received adalimumab for 5 years or more. Baseline demographic and disease characteristics were consistent with long-standing moderate to severe RA. In this group, 357 SI were recorded in 285 patients, for a rate of 0.045/PY (data is shown in Table 28). Fifty-five patients (19%) had more than one event. The percentage of patients with a first SI, according to the duration of adalimumab exposure at the time of event, is shown in FIG. 41. The percentage of patients who developed SI, according to age at the time of study entry, is shown in FIG. 42. The 3 most frequently reported SI in the open-label trials were pneumonia (64 events), septic arthritis (37), and cellulitis (29), which are representative of events commonly experienced by RA patients (data is shown in Table 117; see also Doran M F, et al. Arthritis Rheum 2003; 46:2287-93). No predominance of unusual types of infections was observed.

The onset of the first SI was, on average, 694 days after start of ADA therapy. Among patients sustaining a SI, 25% stayed on therapy without interruption, 48% temporarily interrupted therapy, and 23% permanently discontinued therapy, with 4% categorized as “not applicable”. 51% of SI events occurred while patients were taking concomitant DMARDs. Methotrexate (MTX) was the most commonly used concomitant DMARD. Steroid use is associated with an increased risk of infections (Singh G, et al. Arthritis Rheum 1999; 42(Suppl):S242). 74% of serious infections observed occurred in patients who were taking concomitant steroids vs less than 30% for those taking no steroids. At the time of the SI, steroids were being used in 77% and MTX in 46% of cases. At the time of starting ADA, steroids were used by 58% and MTX by 51% of the 2504 patients. The types and rates of infections seen in this population were similar to those reported in the literature for patients with RA treated with traditional DMARDs or other TNF-antagonists, the respective rates being (0.03-0.10/PY) [Doran M F, et al. Arthritis Rheum 2003; 46:2287-93; Singh G, et al. Arthritis Rheum 1999; 42(Suppl):S242] and (0.05/PY) [Moreland L W, et al. J Rheumatol 2001; 28:1238]. The rate of SI for all adalimumab-treated patients is within the range of what is expected in the general RA population not on anti-TNF therapy, and is similar to earlier reports (Schiff M H, et al. Arthritis Rheum 2004; 50(9 Suppl):S562; Schiff M H, et al. Ann Rheum Dis 2003; 62(Suppl I): 184).

The rate of SI is similar in adalimumab-treated RA patients with and without a history of diabetes mellitus (DM), i.e., 4.5 events per 100 PY for all patients vs. 4.5 events per 100 PY without DM vs. 4.9 events per 100 PY with DM. Among the 146 patients (427 PY of ADA) with diabetes, 21 SI occurred (0.049/PY). The blinded controlled phases of the pivotal trials (1380 ADA patients) yielded a similar SI rate, 0.042/PY. In patients with DM, 17 out of the 21 (81%) SI events occurred while patients were taking concomitant steroids. In patients without DM, 247 out of the 336 (74%) SI events occurred while patients were taking concomitant steroids.

TABLE 116
Summary of Serious Infections Observed in Open-Label Trials

| | Adalimumab-treated patients |
|---|---|
| | open-label trials |
| Summary observations | N = 2504, 7951 PY |
| Patients with serious infections n(%) | 285 (11.4%) |
| Total number of SI events | 357 |
| Rate of SI (E/100PY) | 4.5 |
| No. of patients with >1 SI event | 55 |
| Mean duration of adalimumab treatment | 694 |
| prior to first SI event (days) | |

TABLE 117
Most Common Serious Infections Observed in Open-label Trials

| | Number and Rate E(E/100PY) |
|---|---|
| Serious Infections | N = 2504, 7951 PY |
| Pneumonia | 64 (0.8) |
| Septic Arthritis | 37 (0.5) |
| Cellulitis | 29 (0.4) |

### Conclusions

In adalimumab open-label trials, only 11% (285) of 2504 patients experienced a serious infection. There is a downward trend in the proportion of patients who develop their first serious infection with increasing duration of adalimumab exposure. The majority of the serious infections observed while on adalimumab occurred while patients were taking concomitant steroids. The incidence of serious infections in adalimumab clinical trials of RA in North America and Europe has not increased over time and is comparable to that reported in patients with RA treated with traditional DMARDs. The rate of serious infections is similar regardless of history of diabetes mellitus. Over ¾ of the SI events occurred in patients while taking steroids.

### EXAMPLE 36

### Adalimumab (HUMIRA®) Plus MTX Prevents Nearly All Severe Radiographic Progression Observed With Methotrexate Monotherapy in Early, Aggressive Rheumatoid Arthritis

Adalimumab is a fully human, anti-tumor necrosis factor (anti-TNF) monoclonal antibody indicated for the treatment of moderate to severe rheumatoid arthritis (RA)—both as first-line treatment and in the treatment of DMARD failures. In Study J, a study of MTX-naïve patients with early RA, adalimumab plus methotrexate (MTX) prevented radiographic progression more effectively than MTX alone. Severe radiographic progression occurs in a subset of patients with RA. Cumulative probability plots can be used to assess 2 dimensions of treatment efficacy: 1) the percentage of patients who progress, and 2) the severity of progression in these patients. The objective of the study described herein was to determine whether the greater efficacy of adalimumab plus MTX vs. MTX alone in controlling structural damage in early, aggressive RA occurs equally at all levels of radiographic progression.

Study J was a 2-year, double-blind, active comparator-controlled, Phase III study. MTX-naïve adult patients with early RA (<3 years) were randomized to 1 of 3 treatment arms (Study J outlined in FIG. 18): adalimumab 40 mg every other week (eow)+MTX; Adalimumab 40 mg eow; or MTX (dosage of MTX increased over 8 weeks to 20 mg weekly, as tolerated and as needed). Primary endpoints were ACR50 responses and changes in Total Sharp Score (TSS) at 1 year, comparing adalimumab plus MTX vs. MTX alone. For each patient, radiographs of hands and feet were taken at baseline, 6 months, 1 year and 2 years and evaluated for joint erosions (JE) and joint space narrowing (JSN) by 2 blinded radiologists. Changes from baseline in the modified total Sharp score (TSS, 0-398) were calculated for patients in each treatment arm. Cumulative probability plots were generated by estimating the probability of exceeding a given change in TSS. For each treatment, we calculated the percentages of patients whose ΔTSS exceeded specified thresholds, and the probability of having a given ΔTSS (using cumulative probability plots). For each of 4 cumulative probability ranges, 0-10%, 10-50%, 50-90% and 90-100% (higher numbered ranges=greater ΔTSS), mean ΔTSS values were calculated. In previous reports, Study J radiographic analyses employed linear imputation. In the present analysis, only observed data were employed, with no imputation.

### Results

A total of 799 patients with early RA (mean 0.7 years) enrolled in Study J. Baseline demographics and clinical characteristics were similar among the 3 arms. At baseline, patients in the 6-month evaluation groups had mean TSS values of 18.3 (combination therapy) and 21.7 (MTX alone), and mean disease durations of 0.73 and 0.82 yrs. Of 525 patients who enrolled in the adalimumab plus MTX or MTX monotherapy arms, 458, 433, and 374, respectively, had the required data at 6 months, 1 year, and 2 years (data is shown in Table 119).

TABLE 119
Patient Continuation in Study J: Patients with Radiographic Data Available
for Observed Analysis

| | Total (ITT) | 6 months | 1 year | 2 years |
|---|---|---|---|---|
| Ada + | 268 | 240 | 229 | 202 |
| MTX | | | | |
| Ada | 274 | 230 | 205 | 166 |
| MTX | 257 | 218 | 204 | 172 |

ITT = intention-to-treat

In patients treated for 6 months, adalimumab plus MTX controlled joint destruction better than MTX alone, and did so across the entire spectrum of radiographic progression. The most pronounced benefit from adalimumab plus MTX occurred in patients with the most aggressive disease (ie, in the 50-90% and 90-100% ranges of the probability plot) (data is shown in Table 120). Control of radiographic progression with adalimumab alone was superior to MTX alone in the middle probability ranges (10-50% and 50-90%) (see Table 120). Adalimumab plus MTX controlled radiographic progression significantly better than MTX alone in patients treated for 2 years, especially in those with the most rapidly progressive disease (50-90% and 90-100% ranges) (data is shown in Table 121). Table 122 provides a summary of the mean change in TSS by cumulative probability range over 2 years. For the 10-50% range, mean ΔTSS were low for both treatments, and were significantly lower for combination therapy, which yielded negative ΔTSS values that were stable over 2 yrs

TABLE 120
Mean Changes in TSS at 6 Months by Cumulative Probability Range

| | |
|---|---|
| All points | Cumulative Probability Range |

| | (0-100) | 0-10 | 10-50 | 50-90 | 90-100 |
|---|---|---|---|---|---|
| Ada + MTX | 0.6^{†‡} | −3.2* | −0.8^{†} | 0.6^{†} | 5.8^{†} |
| Ada | 2.1* | −3.4 | −0.2^{†} | 2.0^{†} | 13.6 |
| MTX | 3.4 | −2.0 | 0.2 | 4.4 | 16.5 |
| Δ (Ada + MTX | 0.5 | 1.2 | 1.0 | 3.8 | 10.8 |
| vs. MTX) | | | | | |

Observed data
^{†}p ≦ 0.001 vs. MTX alone;
^{‡}p < 0.001 vs. ada alone;
*p < 0.05 vs. MTX alone

TABLE 121
Mean Change in TSS at 2 Years by Cumulative Probability Range

| | All points | Cumulative Probability Range | | | |
|---|---|---|---|---|---|
| | (0-100) | 0-10 | 10-50 | 50-90 | 90-100 |
| Ada + MTX (n = 202) | 1.1†‡ | −3.4 | −0.9† | 0.8† | 9.7† |
| Ada (n = 166) | 4.7 | −2.8 | −0.1† | 4.7† | 28.9 |
| MTX (n = 172) | 6.4 | −3.9 | 0.5 | 7.6 | 31.4 |
| Δ (Ada + MTX vs. MTX) | 5.3 | −0.5 | 1.4 | 6.8 | 21.7 |

Observed data
^{†}p < 0.001 vs. MTX alone;
^{‡}p < 0.001 vs. ada alone

TABLE 122
Mean ΔTSS by Cumulative Probability Range

| Cumulative | 6 Months | | | | | 2 Years | | | |
|---|---|---|---|---|---|---|---|---|---|
| Prob. Range | 0-10 | 10-50 | 50-90 | 90-100 | | 0-10 | 10-50 | 50-90 | 90-100 |
| Ada + MTX (n = 240) | −3.2* | −0.8^{†} | 0.6^{†} | 5.8^{†} | Ada + MTX (n = 202) | −3.4 | −0.9^{†} | 0.8^{†} | 9.7^{†} |
| MTX alone (n = 218) | −2.0 | 0.2 | 4.4 | 16.5 | MTX alone (n = 172) | −3.9 | 0.5 | 7.6 | 31.4 |

*p < 0.05 vs. MTX alone;
^{†}p < 0.001 vs. MTX alone

Following 2 years of treatment, severe radiographic progression (ΔTSS>10) occurred in >6 times as many patients who received MTX alone vs. patients who received adalimumab plus MTX (see Table 123).

TABLE 123
Percentages of Patients with Large Increases in TSS at 2 Years

| | | >4 | | |
|---|---|---|---|---|
| | | change TSS | >10 change TSS | >20 change TSS |
| % of | Ada + mtx | 12.4 | 3.5 | 0.5 |
| patients | Ada alone | 30.7 | 17.5 | 5.4 |
| | Mtx alone | 41.3 | 23.3 | 7 |

This trend was significant at 6 months (data is shown in Table 124).

TABLE 124
Percentages of Patients with Large Increases in TSS at 6 Months

| | | >4 | | |
|---|---|---|---|---|
| | | change TSS | >10 change TSS | >20 change TSS |
| % of | Ada + mtx | 6.7 | 19.1 | 29.8 |
| patients | Ada alone | 0.5 | 6.5 | 10.6 |
| | Mtx alone | 0 | 1.7 | 2.3 |

### Conclusions

For MTX-naïve patients with rapidly progressive, early RA, adalimumab plus MTX showed greater radiographic efficacy than MTX monotherapy across the entire spectrum of radiographic progression. Adalimumab plus MTX prevented nearly all severe radiographic progression observed with MTX alone, with this effect being clearly established within 6 months. Efficacy was most pronounced among patients with worse disease progression, for whom combination therapy largely prevented the marked increases in TSS that frequently occurred for patients on MTX. Early identification by radiographic assessment of patients who rapidly progress during MTX monotherapy is important because: 1) They are at risk for marked additional joint destruction, and 2) they have the most to gain from combination therapy with adalimumab plus MTX.

### EXAMPLE 37

### The Clinical and Radiographic Efficacy of Every-Other-Week vs. Weekly Dosing Frequency of Adalimumab (HUMIRA®) in the Treatment of Early Rheumatoid Arthritis (RA)

Adalimumab is a fully human, anti-tumor necrosis factor (anti-TNF) monoclonal antibody indicated for the treatment of moderate to severe rheumatoid arthritis (RA) and of psoriatic arthritis (PsA). The recommended dosage of adalimumab is 40 mg subcutaneously every other week (eow), with or without concomitant methotrexate (MTX). As monotherapy, adalimumab 40 mg weekly is allowed. In RA clinical trials, the clinical efficacy of adalimumab 40 mg eow equaled that of 80 mg eow when each was used with MTX, and was slightly lower than that of 40 mg weekly when given as monotherapy. Study J was a 2-year study of MTX-naïve patients with severe, early RA that compared adalimumab plus MTX vs. adalimumab alone and vs. MTX alone. The protocol mandated that the dosage of injectable medicine be changed from eow to weekly in ACR20 non-responders on or after Week 16. The analysis described herein evaluated the clinical and radiographic benefits achieved in patients who were required by protocol, after optimizing their MTX dosage, to increase their adalimumab dosing frequency from eow to weekly during Study J.

Study J was a 2-year, double-blind, active comparator-controlled, Phase III study. MTX-naïve adult patients with early RA (<3 years) were randomized to receive either: adalimumab 40 mg every other week (eow)+MTX, adalimumab 40 mg eow, or MTX. In patients receiving ADA+MTX or MTX monotherapy, the 7.5 mg MTX weekly dosage increased to 20 mg over 8 weeks, as tolerated and as needed. Monotherapy patients received either placebo pills (adalimumab arm) or placebo injections (MTX arm). Efficacy outcomes measured included ACR response, DAS28 remission, major clinical response, and radiographic change from baseline. Primary endpoints were ACR50 responses and changes in Total Sharp Score (TSS) at 1 year, comparing adalimumab plus MTX vs. MTX alone. Per protocol, all patients who failed to respond, or lost response, on or after 16 weeks of treatment were mandated to increase their injectable therapies (adalimumab or placebo) to weekly dosing.

Protocol guidelines for dosage escalation of injectable medicine (adalimumab or placebo) were established in Study J. Escalation was mandated for all patients who, during 2 consecutive visits ending on or after Week 16 and at least 2 weeks apart, had <ACR20 response. MTX optimization was required before starting weekly injections (to 20 mg weekly, maximum). Once initiated, weekly dosing of injectable medicine was to be maintained until end of study. Criteria for identification of dosage escalators were a sequence of 4 consecutive injections for which the interval for every two consecutive injections was ≦10 days, and/or that the date of escalation, defined as the second of the 4 injections, had to be on or after Week 16 in Year 1. Three dosage escalator categories were defined, based on best ACR response rate achieved any time prior to escalation: Group A=ACR non-responders (never achieved ACR20 prior to dosage escalation), Group B=Partial responders (achieved ACR20 or ACR50 at least once prior to escalation, but never ACR70), and Group C=ACR70 responders (achieved ACR70 at least once prior to escalation). Data are from the intention-to-treat (ITT) population, with missing data analyzed by non-responder imputation (NRI) for clinical measures and by linear imputation for total Sharp scores (TSS).

### Results

A total of 799 patients enrolled in Study J: 268 in the combination arm, 274 in the adalimumab alone arm, and 257 in the MTX alone arm. Baseline demographics and clinical characteristics were similar among the 3 arms. The percentages of patients who increased injectable medication during year 1 (either adalimumab or placebo) were 11% in the combination arm, 25% in the adalimumab arm, and 20% in the MTX arm (the dose increase in the MTX arm was an increase in placebo) (data is shown in Table 125). Most dosage escalation occurred between Weeks 16-30. Nearly all dosage escalation occurred when the patients were an ACR non-responder (ie. <ACR20). The percentages of patients who never had an ACR20 response prior to weekly dosing and had a clinical response post-escalation were low, and were similar between patients whose adalimumab was escalated and patients whose placebo was escalated (MTX arm) (data is shown in Table 126).

TABLE 125
Patients in Whom the Dosage of Injectable Medicine was Escalated

| | Treatment Arm | | |
|---|---|---|---|
| | Ada + MTX | Ada Alone | MTX Alone* |
| | N = 268 | N = 274 | N = 257 |
| | n (%) | n (%) | n (%) |
| Dosage Escalators Weeks | 27 (9) | 59 (22) | 44 (17) |
| 16-30 | | | |
| Dosage Escalators Year 1 | 29 (11) | 69 (25) | 52 (20) |
| (total) | | | |

At time of dosage escalation, 93% (ada + MTX), 94% (ada alone) and 90% (MTX alone) of dosage escalators were ACR non-responders (ie, <ACR20).
*MTX monotherapy patients escalated placebo injections

TABLE 126
Clinical Responses Following Dosage Escalation by Patients
With No Prior ACR20 Response

| | Ada + MTX | | Ada Alone | | MTX Alone | |
|---|---|---|---|---|---|---|
| | (n = 268) | | (n = 274) | | (n = 257) | |
| | N (%)* | N (%)* | N (%)* | N (%)* | N (%)* | N (%)* |
| | 1 year | 2 years | 1 year | 2 years | 1 year | 2 years |
| | n = 12 | n = 12 | n = 20 | n = 20 | n = 25 | n = 25 |
| ACR20 | 2 (0.7) | 4 (1.5) | 5 (1.8) | 7 (2.6) | 9 (3.5) | 9 (3.6) |
| ACR60 | 2 (0.7) | 3 (1.1) | 4 (1.5) | 1 (0.4) | 3 (1.2) | 5 (1.9) |
| ACR70 | 1 (0.4) | 2 (0.7) | 0 (0) | 1 (0.4) | 2 (0.8) | 3 (1.2) |
| DAS28 < 2.6 | 3 (1.1) | 2 (0.7) | 1 (0.4) | 1 (0.4) | 0 (0.0) | 2 (0.8) |

*Percentages express the portion of responses, by NRI, that occurred in patients whose injectable dosages were escalated during Year 1 relative to all 268, 274 or 257 patients in their respective treatment arms.

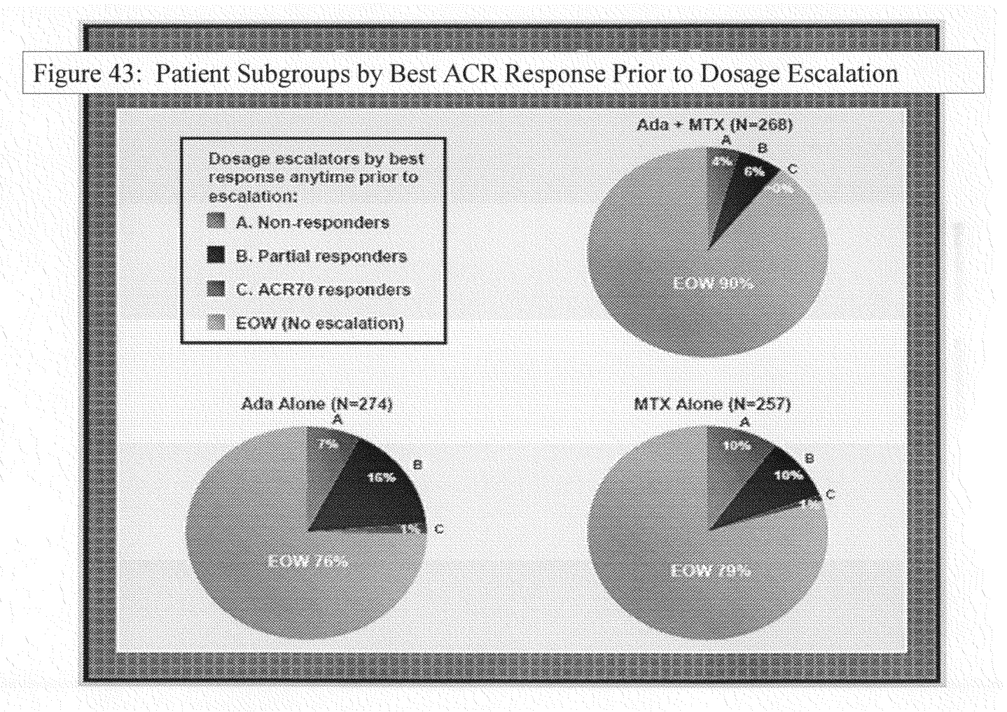

Division of patient subgroups by best ACR response prior to dosage escalation is shown in FIG. 43. The effect of dosage escalation on overall efficacy was similar between patients who escalated dosage of placebo vs. adalimumab, regardless of prior ACR response history.

For patients whose dosages were escalated to weekly adalimumab, ACR 50 and ACR 70 response rates were low and similar to those of placebo escalators (MTX). ACR 50 and ACR 70 response rates in patients whose dosages of adalimumab or placebo were escalated are shown in Table 127. Overall, an increase of adalimumab dosing to weekly had an effect on efficacy similar to or less than that of placebo (data is summarized in Table 128). Radiographic progression was significantly less frequent in patients who received adalimumab plus MTX compared with adalimumab alone or MTX alone, whether or not weekly dosing was used.

TABLE 127
ACR 50/70 Response Rates in Patients Whose Dosages of Adalimumab
or Placebo Injections Were Escalated

| | | Ada + mtx | Ada alone | Mtx alone |
|---|---|---|---|---|
| % of responders | ACR50 | | | |
| | Year 1 | 21 | 17 | 17 |
| | Year 2 | 24 | 16 | 21 |
| | ACR70 | | | |
| | Year 1 | 10 | 6 | 8 |
| | Year 2 | 14 | 10 | 12 |

TABLE 128
Percentages of Patients Who Became Responders* After a Protocol-
Mandated Increase to Weekly Dosing

| | | | | | MTX | |
|---|---|---|---|---|---|---|
| | Adalimumab* | | Adalimumab | | Monotherapy | |
| | MTX | | Monotherapy | | (with placebo) | |
| | (n = 268) | | (n = 274) | | (n = 257) | |
| | 1 year | 2 years | 1 year | 2 years | 1 year | 2 years |
| ACR20 | 1% | 1% | 2% | 3% | 4% | 4% |
| ACR50 | 1% | 1% | 1% | 0% | 1% | 2% |
| DAS26 < 2.6 | 1% | 1% | 0% | 0% | 0% | 1% |
| (remission) | | | | | | |
| Major Clinical | 0% | | 1% | | 1% | |
| Response* | | | | | | |

*ACR70 for ≧6 consecutive months over a 2-year period

TABLE 129
No radiographic progression at 2 years: eox vs. weekly dosing

| | | Ada + mtx | Ada alone | Mtx alone |
|---|---|---|---|---|
| % of | eow | 61.5 | 48.8 | 36.6 |
| patients | weekly | 58.6 | 31.9 | 21.2 |

### Conclusions

In MTX-naïve patients with early RA, optimal efficacy was achieved through adalimumab 40 mg eow plus MTX in the vast majority of patients. In patients with suboptimal responses, a mandated adalimumab dosage increase provided additional benefit to a small percentage of patients, but the increased benefit was similar to what was observed for patients who increased their injectable placebo. These data indicate that escalating adalimumab dosage to one injection per week is not efficacious for early RA patients who have poorly controlled disease after 16 weeks of eow treatment. Adalimumab 40 mg eow, either alone or in combination with MTX, is the appropriate dosage for the vast majority of RA patients.

### EXAMPLE 38

### Long-Term Efficacy, Remission, and Safety of Adalimumab (HUMIRA®) Plus Methotrexate (MTX) in Patients with Rheumatoid Arthritis (RA) in Study I

Study I was a 6-month randomized, controlled trial (Arthritis Rheum 2003; 48:35-45) and demonstrated that adalimumab plus MTX significantly reduces signs and symptoms and improves functional outcomes in patients with long-standing RA. Long-term extension studies are essential to confirm that the efficacy and safety of TNF antagonists observed in short-term studies are sustained over the long run. The objectives of the study described herein were to assess the sustained efficacy of adalimumab in combination with MTX, to determine if clinical efficacy was maintained in patients who reduced MTX and/or corticosteroid dosages, and to confirm the long-term safety and tolerability of this regimen.

Patients were eligible to enroll in Study I if they met ACR criteria for diagnosis of RA. Once patients completed the 24-week, blinded portion of the trial, all patients (including those originally on placebo) were permitted to enter an open-label extension study and receive the standard adalimumab dose of 40 mg eow in combination with MTX (Study I is outlined in FIG. 28). Efficacy outcomes were assessed at pre-specified intervals as observed data. Treatment time was calculated beginning with the first subcutaneous injection of adalimumab at any dose, excluding time on placebo. Improvements in signs and symptoms of RA were evaluated for ACR20, ACR50, and ACR70 criteria, 28-joint count Disease Activity Score (DAS28) using the CRP-based formula, and Health Assessment Questionnaire (HAQ). Patients with changes in dosage of MTX or corticosteroids were evaluated for at least 6 months; these patients were eligible for the extension study for at least 6 months and were eligible for discretionary dose tapering. Patients were monitored for adverse events (AEs) during the entire length of the study, from signed informed consent through last visit.

### Results

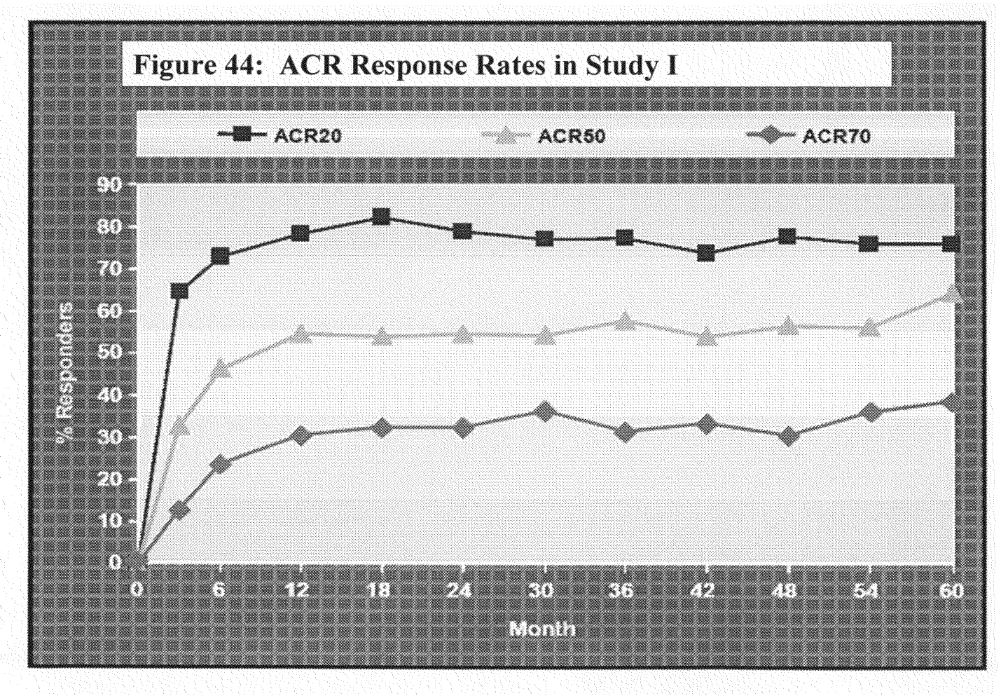

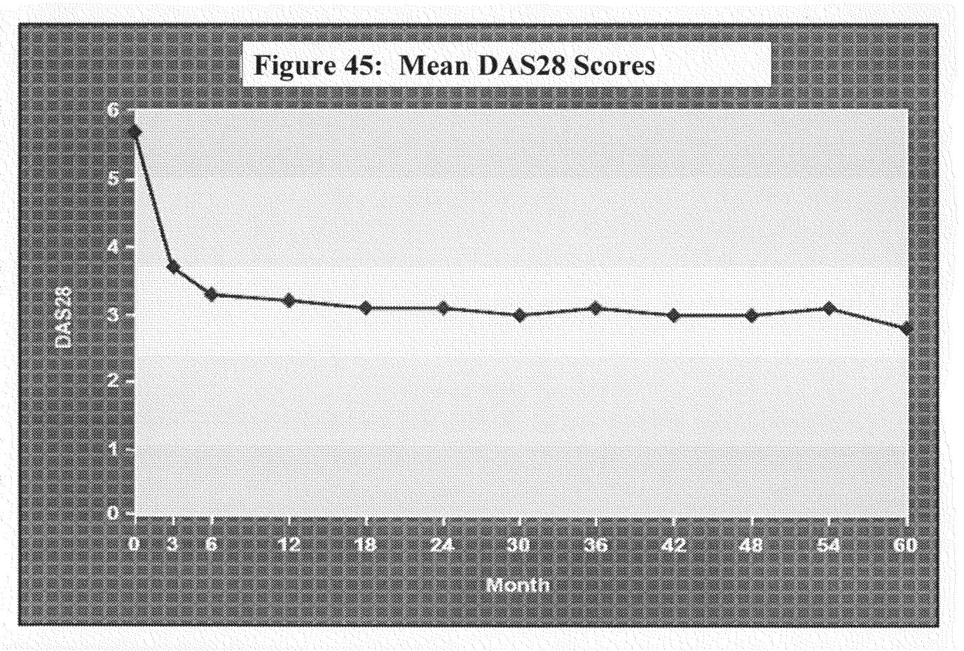

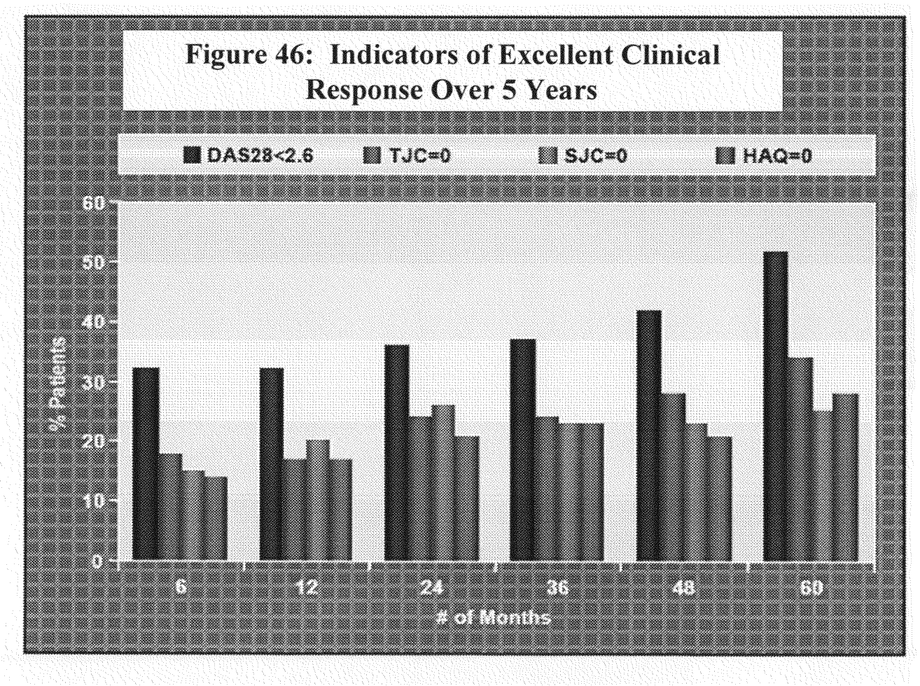

Of 271 patients in the original Study I trial, 262 patients received at least one dose of adalimumab and were evaluated. Demographic and baseline disease characteristics of RA patients were consistent with moderate to severe RA (n=262) (data is shown in Table 130). At the time of analysis, 160 (61%) patients had remained in the study. Withdrawals were for lack of efficacy (8%), adverse events (15%), and other reasons (16%). The Kaplan-Meier curve provides a projection of patients receiving adalimumab that will remain on therapy at Year 5. For 67 patients who had completed 5 years of therapy, efficacy improvements achieved at 6 months were sustained over time. In Study I, both ACR responses and improvements in DAS28 were sustained into Year 5 (ACR response rates and mean DAS28 scores are shown in FIGS. 44 and 45, respectively). At 5 years, 76%, 64%, and 39% achieved ACR20/50/70; 52% achieved excellent clinical response (DAS28<2.6); and 28% had no physical limitations (HAQ=0). The percentage of patients achieving excellent clinical response over 5 years as measured by DAS28<2.6, TJC=0, SJC=0, and HAQ=0 is shown in FIG. 46. Table 68 shows the progressively greater percentages of available patients meeting remission outcome parameters.

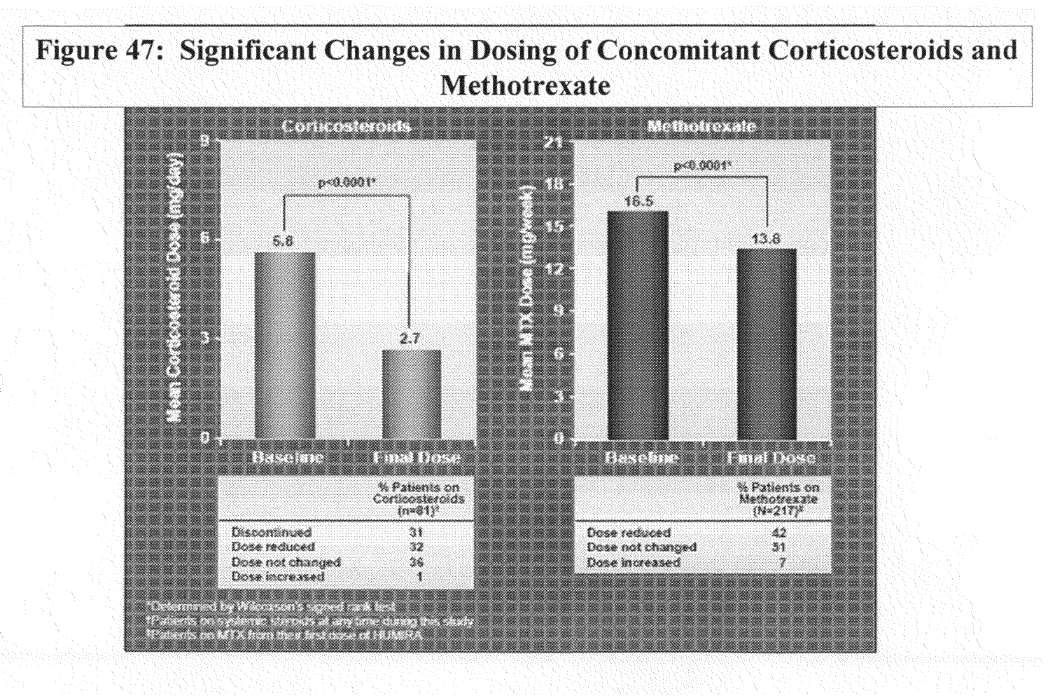

Of the 217 patients who had received adalimumab plus MTX and were eligible for MTX or steroid reduction, substantial percentages of patients were able to decrease dosages of corticosteroids (63%), MTX (42%), or both (12%) while sustaining efficacy (data is shown in FIG. 47). Serious adverse events during open-label therapy were comparable to those seen in the controlled phase (data is shown in Table 132). The rate of serious infections was 2.2 events per 100-patient years vs. 2.30 during the blinded period. There was one reported case of multiple sclerosis and congestive heart failure and no cases of tuberculosis, systemic lupus erythematosus, pancytopenia, lymphomas, and other opportunistic infections.

TABLE 130
Baseline Demographics and Disease Characteristics

| Characteristic | Value |
|---|---|
| Age, years (mean + SD) | 55.2 ± 11.8 |
| Gender, % female | 76 |
| Disease duration, years (mean + SD) | 12.1 ± 9.5 |
| Tender joint count, (0-68 joints) (mean + SD) | 14.2 ± 7.1 |
| Swollen joint count, (0-68 joints) (mean + SD) | 11.4 ± 5.8 |
| HAQ disability index, (0-3 scale) (mean + SD) | 1.5 ± 0.7 |
| Disease Activity Score 28 (DAS28) (mean + SD) | 5.7 ± 1.1 |
| C-reactive protein, mg/dl (normal = <0.8) (mean + SD) | 2.9 ± 3.0 |
| Rheumatoid factor, % positive | 81.3 |
| Number of previous DMARDs (mean) | 3.0 ± 1.3 |

TABLE 131
Percentages of Patients Achieving Efficacy Measures by Duration of
Treatment

| | Month | | | | | |
|---|---|---|---|---|---|---|
| | 6 | 12 | 24 | 36 | 48 | 60 |
| N | 221 | 178 | 198 | 180 | 151 | 67 |
| DAS28 < 2.6 | 32 | 32 | 36 | 37 | 42 | 52 |
| ACR70 | 24 | 31 | 33 | 32 | 31 | 39 |
| TJC = 0 | 18 | 17 | 24 | 24 | 28 | 34 |
| SJC = 0 | 15 | 20 | 26 | 23 | 23 | 25 |
| CRP < 0.8 mg/dL | 66 | 79 | 73 | 70 | 74 | 79 |
| HAQ = 0 | 14 | 17 | 21 | 23 | 21 | 23 |

TABLE 132
Serious Adverse Events

| | ARMADA | ARMADA |
|---|---|---|
| | Blinded Period* | Total Exposure^{†} |
| Serious Adverse Event | Events/100-Pt-Years | Events/100-Pt-Years |
| Serious infections | 2.30 | 2.2 |
| Pneumonia | 2.30 | 0.5 |
| Urinary tract infections | 0.00 | 0.1 |
| Septic arthritis | 0.00 | 0.1 |
| Tuberculosis | 0.00 | 0.0 |
| Histoplasmosis | 0.00 | 0.0 |
| Demyelinating diseases | 0.00 | 0.1 |
| Lymphoma | 0.00 | 0.0 |
| SLE/Lupus-like syndrome | 0.00 | 0.0 |
| Congestive heart failure | 0.00 | 0.1 |
| Pancytopenia | 0.00 | 0.0 |

*87 pt-years of total exposure
^{†}936 pt-years of total exposure (as of Apr. 15, 2005) MEDRA coding

### Conclusions

Patients with long-standing RA maintained clinical improvements and a significant reduction of disease activity for up to 5 years of continuous treatment with adalimumab 40 mg eow plus MTX. Adalimumab plus MTX induced clinical remission (based on DAS28<2.6) in over 50% of patients into the 5th year of therapy. The safety profile observed during the first 6 months was similar to the profile after 5 years of follow-up. Patients were able to substantially reduce corticosteroid and/or MTX dosages without adversely affecting long-term efficacy.

### EXAMPLE 39

### Adalimumab (HUMIRA®) Plus Methotrexate is Superior to MTX Alone in Improving Physical Function, as Measured by the SF-36, in Patients with Early Rheumatoid Arthritis

Recent clinical trials of tumor necrosis factor (TNF) antagonists have shown that therapy with a TNF antagonist plus methotrexate (MTX) is superior to MTX monotherapy in the treatment of rheumatoid arthritis (RA) (De Vries-Bouwstra J K, et al. Arthritis Rheum 2003: 48:3649 (LB18); Smolen J S, et al. Ann Rheum Dis 2003: 61(Suppl I):64; Weinblatt M E, et al. Arthritis Rheum 2003; 48:35-45). The Study J study was the first trial to directly compare a TNF antagonist plus MTX with the TNF antagonist alone and MTX alone in MTX-naïve patients with recent-onset RA. Adalimumab is a monoclonal IgG1 antibody that contains only human peptide sequences. It binds with high specificity and affinity to soluble and membrane-bound TNF, thereby neutralizing the biological activities of this cytokine.

The Short Form 36 (SF-36) Health Survey is a generic, patient-reported, health-related quality of life (HRQOL) measurement instrument with 8 domains and two summary scores for physical and mental health. Interpretation of the results from health surveys such as the SF-36 can be difficult. Criteria-based and content-based interpretations are used to gain a better understanding of differences in SF-36 Physical Component Summary (PCS) scores (Ware J E, Kosinski M. SF-36 Physical & Mental Health Summary Scales: A Manual for Users of Version 1. 2nd ed. Lincoln, R I: QualityMetric Incorporated, November 2002). Content-based interpretation is based on analyses of the content of individual SF-36 items within the survey for the general US population, such as “Does your health limit you in walking one block?” Criteria-based interpretation is based on external criteria such as predicting job loss due to health problems and is also based on US population norms for PCS scores. The objective of the study described herein was to assess the impact of adalimumab therapy (used in combination with MTX) on initial and sustained improvement in HRQOL for patients with early RA, and to interpret the findings.

Study J was a 2-year, double-blind, active comparator-controlled, Phase III study conducted at 149 sites in North America, Europe, and Australia. MTX-naïve adult patients with early RA (<3 years) were randomized to 1 of 3 treatment arms: adalimumab 40 mg every other week (eow)+MTX, adalimumab 40 mg eow monotherapy+placebo, or MTX monotherapy+placebo. The SF-36 was used to assess the 8 domains of HRQOL—physical functioning, role-physical, bodily pain, general health, vitality, social functioning, role-emotional, and mental health. These 8 domains were aggregated into Physical Component Summary (PCS) and Mental Component Summary (MCS) scores. The SF-36 has a scale of 0-100, with higher scores indicating better health-related quality of life. Minimum clinically important differences (MCID) are defined as improvements of 5-10 points in the individual domains scores and 2.5-5 points in the PCS and MCS (Kosinski M, et al. Arthritis Rheum 2000: 43:1478-87). All HRQOL domains were measured at baseline, and after 12, 26, 42, 52, 76, and 104 weeks of therapy. In this analysis, we report mean scores and mean changes in each HRQOL domain, as well as the PCS and MCS, at Weeks 12 and 104. We used criteria-based interpretation to understand the meaning of differences in PCS scores for work loss and resource use and content-based interpretation for specific SF-36 items (Weinblatt M E, et al. Arthritis Rheum 2003; 48:35-45).

### Results

A total of 799 patients enrolled in the Study J. Baseline demographics and clinical characteristics were similar between the 3 arms, and indicative of early, erosive RA. Mean baseline SF-36 domains scores for patients who received adalimumab plus MTX were comparable to scores for patients who received MTX monotherapy. These scores were well below the norms for the US population (Ware J E, Kosinski M. SF-36 Physical & Mental Health Summary Scales: A Manual for Users of Version 1. 2nd ed. Lincoln, R I: QualityMetric Incorporated, November 2002), indicating that patients with early, erosive RA have substantial HRQOL impairment. HRQOL domain score results were comparable between the 2 monotherapy arms. By Week 12, patients on adalimumab plus MTX combination therapy and patients on MTX monotherapy had both achieved substantial improvements in HRQOL domain scores. Mean SF-36 domain scores at Week 12, and mean changes in SF-36 domain scores at Week 12. These improvements were greater at the end of two years of therapy. By Week 12, patients on combination therapy had achieved clinically meaningful and statistically significantly greater improvements in 5 of 8 domains vs. patients on MTX monotherapy (data is shown in Table 133). These improvements were sustained at the end of 2 years of therapy (data is shown in Table 134).

Mean baseline PCS scores for the adalimumab plus MTX and MTX monotherapy groups were 31.7 and 32.2, respectively. The mean PCS score for the combination therapy group at Week 12 had improved to 42.2 vs. 38.3 for the MTX monotherapy group. The 4.5 difference between the 2 groups in PCS mean change from baseline at 12 weeks was clinically meaningful and sustained through 2 years (5.1) (p<0.0001).

Based on criteria-based interpretation (CrBI) of the PCS for the general US population, the percentage differences between the 2 groups indicate patients on adalimumab plus MTX were less likely to lose their jobs or to be unable to work. In addition, patients receiving adalimumab plus MTX were less likely to be hospitalized or to visit a physician than those on MTX monotherapy. Based on content-based interpretation (CoBI) of the PCS, about half of those in the adalimumab plus MTX group, compared with patients in the MTX monotherapy group, were likely to have their health compromised, or their abilities to walk one block or climb one flight of stairs impaired. Also based on CoBI, patients in the adalimumab plus MTX group would be half as likely as the those in the MTX monotherapy group to have had difficulty at work, or to have cut down their time at work. In addition, patients in the adalimumab plus MTX group had more energy and were less likely to feel tired. Criteria-based and content-based interpretation of SF-36 PCS scores are reported in Table 134.

TABLE 133
SF-36 Scores Following Treatment with MTX Monotherapy vs.
Adalimumab + MTX

| | MTX | | | ADA + MTX | | |
|---|---|---|---|---|---|---|
| Endpoint | Baseline | Week 12 | Week 104 | Baseline | Week 12 | Week 104 |
| Physical Functioning | 38.9 | 51.1 | 58.9 | 35.8 | 60.3* | 68.8* |
| Role-Physical | 16.4 | 40.4 | 48.6 | 18.3 | 51.6* | 62.1* |
| Bodily Pain | 29.9 | 51.0 | 57.0 | 29.5 | 59.9* | 68.4* |
| General Health | 49.7 | 58.9 | 68.3 | 50.7 | 61.1† | 64.7* |
| Vitality | 37.1 | 51.6 | 56.9 | 35.9 | 55.3* | 62.5* |
| Social Functioning | 56.5 | 72.0 | 75.5 | 56.6 | 74.4† | 78.8 |
| Role-Emotional | 41.1 | 60.5 | 64.5 | 46.5 | 63.7 | 71.6 |
| Mental Health | 62.1 | 71.2 | 72.9 | 61.2 | 71.5 | 74.2 |
| PCS | 32.2 | 38.3 | 41.2 | 31.7 | 42.2* | 45.9* |
| MCS | 43.5 | 49.0 | 49.9 | 44.2 | 48.5 | 50.3 |

LOCF;
*p < 0.01 and
†p < 0.05 for change from baseline vs. MTX

TABLE 134
Interpretation of SF-36 PCS Scores - Percentages Based on General US
Population Norms

| | MTX Alone | | Adalimumab + MTX | |
|---|---|---|---|---|
| | Baseline | Week 104 | Baseline | Week 104 |
| Criteria-Based | | | | |
| Hospitalized | 10.7 | 7.7 | 10.9 | 6.6 |
| MD visit | 47.7 | 34.6 | 43.4 | 28.0 |
| Not work | 44.1 | 24.2 | 45.5 | 14.7 |
| Job loss | 30.3 | 20.8 | 30.9 | 15.4 |
| Content-Based | | | | |
| Difficulty working | 88.1 | 51.2 | 88.8 | 25.0 |
| Time off work | 64.8 | 29.8 | 65.9 | 13.7 |
| Walk on block | 44.4 | 21.4 | 46.2 | 10.7 |
| Climb stairs | 66.4 | 30.7 | 68.0 | 17.0 |
| Have energy | 9.7 | 26.6 | 10.1 | 36.3 |

Ware JE, Kosinski M. SF-36 Physical & Mental Health Summary Scales: A Manual for Users of Version 1. 2nd ed. Lincoln, RI: Quality Metric Incorporated, November 2002

### Conclusions

In MTX-naïve patients with rapidly progressive, recent-onset RA, adalimumab plus MTX was superior to MTX monotherapy in providing statistically significant and clinically meaningful improvements in HRQOL in early RA. Patients in the adalimumab plus MTX group were more likely to be active, have more energy, and to be able to walk one block and climb one flight of stairs. In addition, patients in the adalimumab plus MTX group would have had substantially less job loss and have had less difficulty on the job than patients on MTX monotherapy. A significantly lower change in PCS score at 2 years in the MTX monotherapy group may mean patients on MTX monotherapy have greater health care utilization. These results may mean patients who receive combination therapy are able to lead more active lives and enjoy better health. This measure demonstrates that a TNF antagonist plus MTX is superior to MTX monotherapy in the treatment of RA.

### EXAMPLE 40

### Improvements in Quality of Life Measures from Adalimumab (HUMIRA®) Plus Methotrexate (MTX) Translate into Improved Physical Function and Less Fatigue in Patients with Early Rheumatoid Arthritis (RA)

Recent clinical trials of tumor necrosis factor (TNF) antagonists have shown that therapy with a TNF antagonist plus methotrexate (MTX) is superior to MTX monotherapy in the treatment of rheumatoid arthritis (RA) (De Vries-Bouwstra J K, et al. Arthritis Rheum 2003: 48:3649 (LB18); Smolen J S, et al. Ann Rheum Dis 2003: 61(Suppl I):64; Weinblatt M E, et al. Arthritis Rheum 2003; 48:35-45). Study J was the first trial to directly compare a TNF antagonist plus MTX with the TNF antagonist as monotherapy and MTX monotherapy in MTX-naïve patients with recent- onset RA. Adalimumab is a monoclonal IgG1 antibody that contains only human peptide sequences. It binds with high specificity and affinity to soluble and membrane-bound TNF, thereby neutralizing the biological activities of this cytokine.

The Health Assessment Questionnaire Disability Index (HAQ DI) is a disease-specific tool that measures patient-reported physical function in 8 categories corresponding to normal activities in daily living. Fatigue is an important yet still often overlooked systemic symptom of RA. The Functional Assessment of Chronic Illness Therapy-Fatigue (FACIT-F) tool, measured by a 13-item, validated scale, assesses the impact of a disease and its therapy on fatigue. The objective of the study described herein was to assess the ability of adalimumab therapy (used in combination with MTX) to improve physical function and reduce fatigue in patients with early rheumatoid arthritis (RA).

Study J is described in detail in the above examples. The Health Assessment Questionnaire Disability Index (HAQ DI) and Functional Assessment of Chronic Illness Therapy-Fatigue (FACIT-F) were assessed at baseline, and following 12, 26, 42, 52, 76, and 104 weeks of therapy. Changes in scores were computed by subtracting the baseline from post-treatment score for each patient; mean changes were reported for each treatment group. The HAQ DI is assessed on a scale of 0-3, with higher numbers indicating greater disability. The minimum clinically important difference (MCID) is defined as changes ≧0.22 (Goldsmith C H, et al. J Rheumatol 1993; 20-561-5). The FACIT-F is assessed on a scale of 0-52, with higher scores indicating less fatigue. Changes of ≧4 are considered clinically meaningful (Cella D, et al. J Pain Symptom Manage 2002: 24:547-61).

### Results

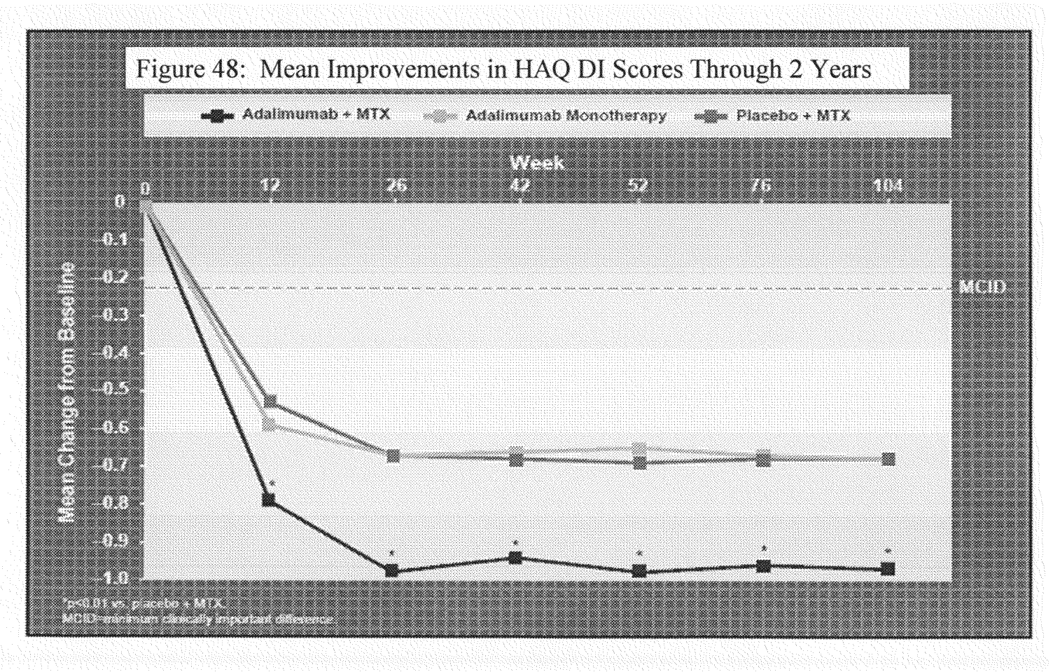

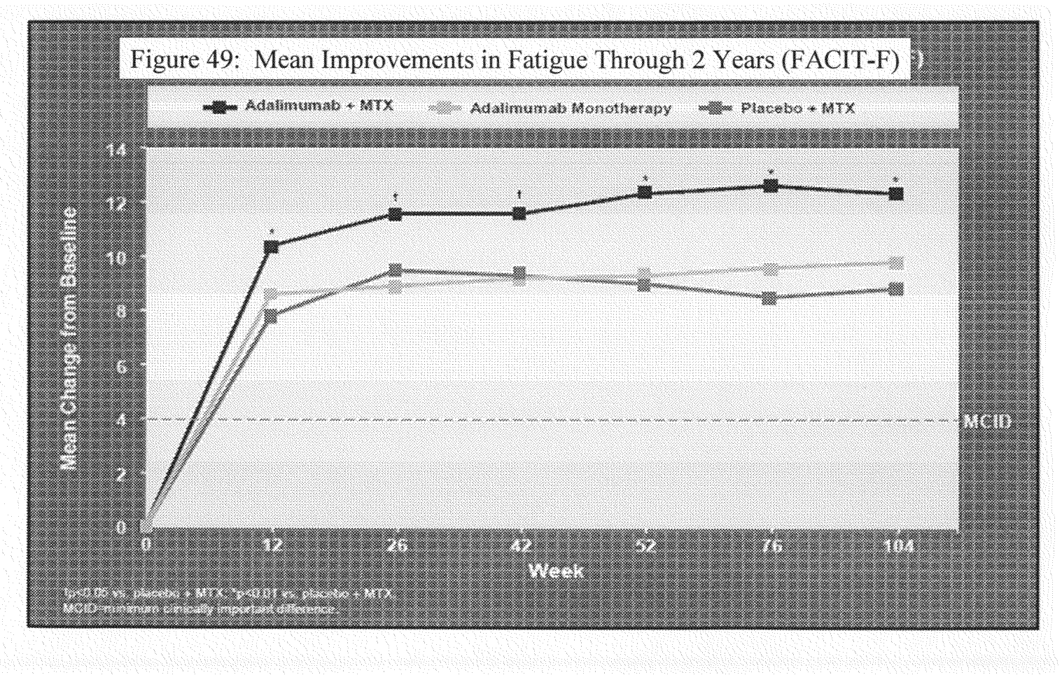

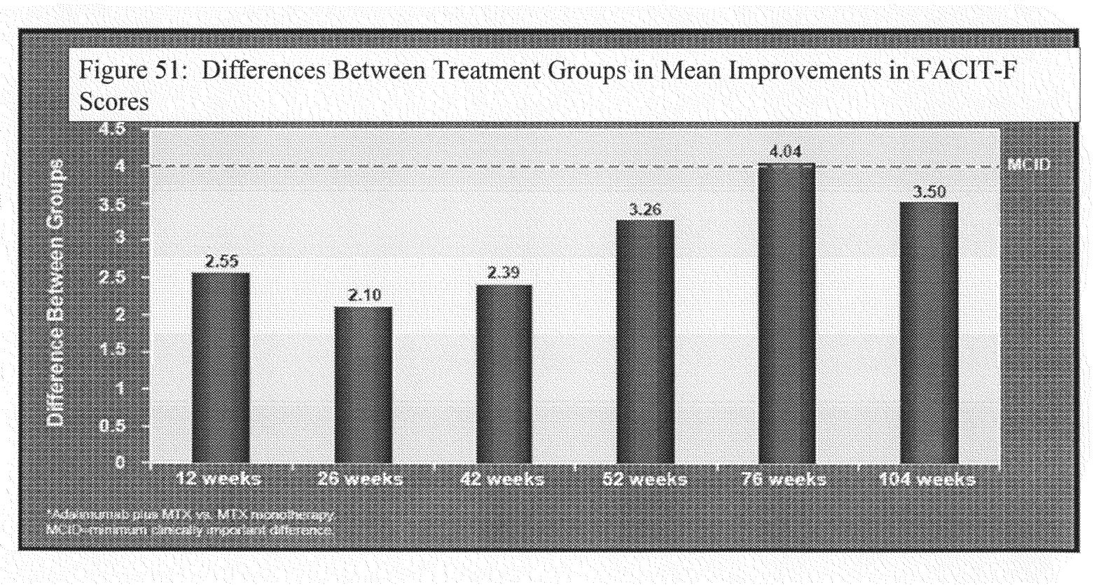

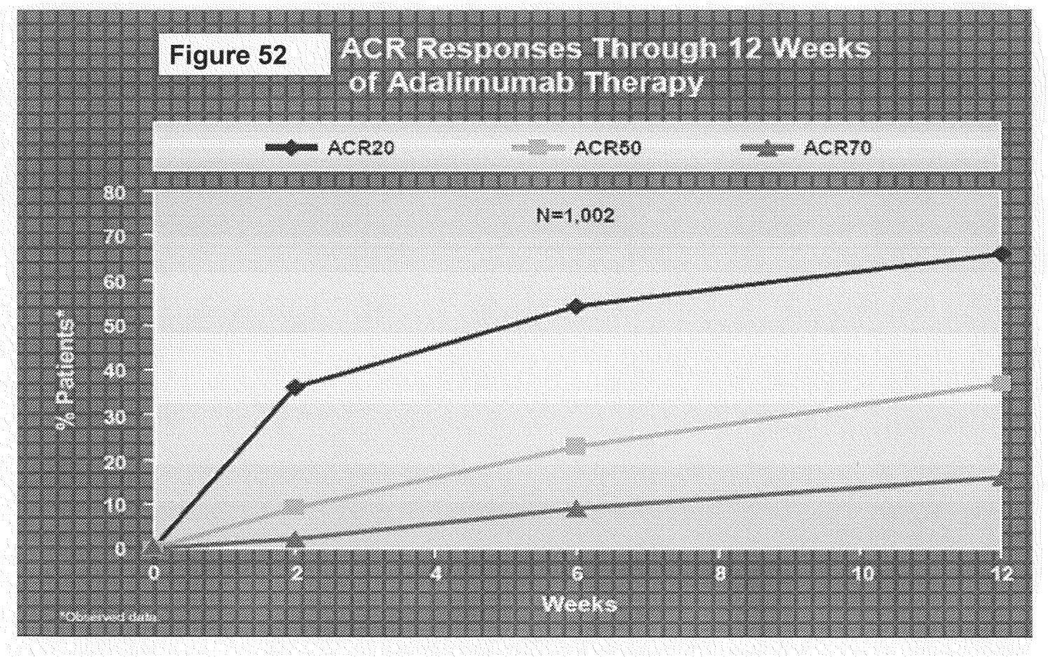

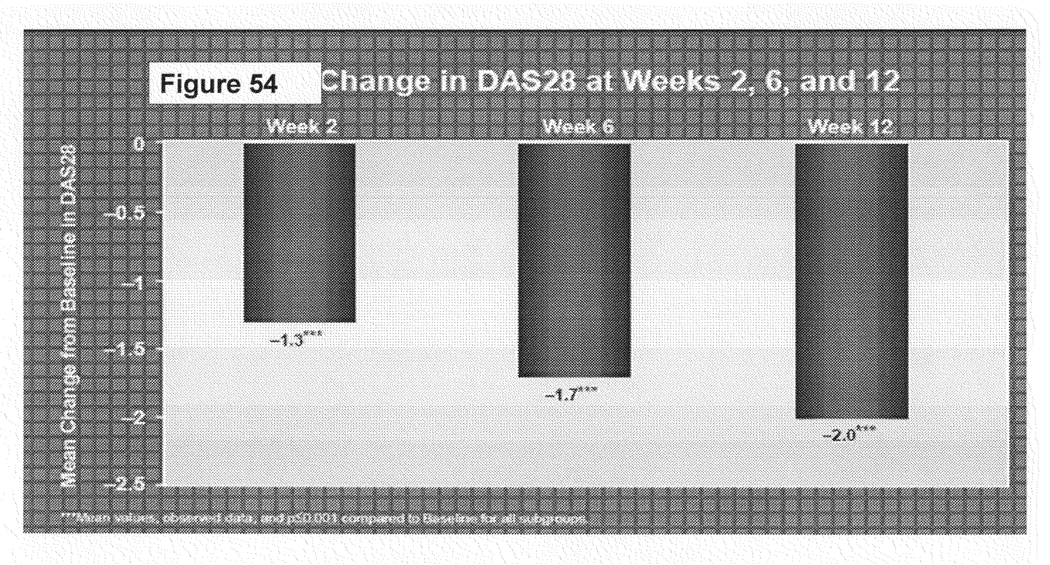

A total of 799 patients enrolled in Study J. Mean baseline HAQ and FACIT-F scores for patients who received adalimumab in combination with MTX were comparable to scores for patients who received either monotherapy. During the course of treatment, HAQ DI and FACIT-F scores were comparable for the two monotherapy groups. Improvements from baseline in the adalimumab plus MTX group for HAQ DI and FACIT-F were significantly greater than changes from baseline in the MTX monotherapy group at all time points. Mean improvements in HAQ DI scores and mean improvements in FACIT-F through 2 years are shown in FIG. 51 and FIG. 52, respectively. Improvements from baseline in both groups were clinically meaningful. The differences between the mean changes in the adalimumab plus MTX group and the MTX monotherapy group in HAQ DI were clinically meaningful throughout the study, while the mean changes in FACIT-F reached MCID by Week 76. Differences between treatment groups in mean improvements in HAQ DI scores are shown in FIG. 48. Differences between treatment groups in mean improvements in FACIT-F scores are shown in FIG. 54. Mean changes in physical function and fatigue scores for adalimumab plus MTX vs. MTX monotherapy are summarized in Table 135.

TABLE 135
Mean Changes in Physical Function and Fatigue Scores for Adalimumab
Plus MTX vs. MTX Monotherapy

| Endpoint | Baseline | ΔWeek 12 | ΔWeek 26 | ΔWeek 42 | ΔWeek 52 | ΔWeek 76 | ΔWeek 104 |
|---|---|---|---|---|---|---|---|
| ADA + MTX | | | | | | | |
| HAQ DI | 1.47 | −0.79* | −0.90* | −0.94* | −0.98* | −0.96* | −0.97* |
| FACIT-F | 28.4 | 10.4* | 11.6^{†} | 11.6^{†} | 12.3* | 12.6* | 12.3* |
| MTX Alone | | | | | | | |
| HAQ DI | 1.48 | −0.53 | −0.67 | −0.68 | −0.69 | −0.68 | −0.68 |
| FACIT-F | 29.0 | 7.8 | 9.5 | 9.3 | 9.0 | 8.5 | 8.8 |

LOCF;
*p < 0.01 and
†p < 0.05 for change from baseline vs. MTX

### Conclusions

Adalimumab plus MTX was superior to MTX alone in providing significant and clinically meaningful differences in physical function and fatigue, as measured by the HAQ DI and FACIT-F. These improvements were sustained over the 2-year observation period.

### EXAMPLE 41

### Adalimumab (Humira®) in Patients with Rheumatoid Arthritis Improves Quality of Life: Results from Study A in France

Health-related quality-of-life is a key goal in the treatment of rheumatoid arthritis (RA). Adalimumab, a fully human IgG1 monoclonal antibody, has been shown to significantly reduce signs and symptoms and inhibit disease progression in patients with RA when given alone or in combination with other DMARDs. Study A was a Phase IIIb study where enrollment requirements were similar to national BDMARD treatment guidelines. The objective of this study was to investigate the ability of adalimumab to reduce the impact of RA on daily life activities for patients with RA enrolled Study A in France

### Study Design

Patients with active RA received adalimumab 40 mg every other week (eow) subcutaneously (sc) either in addition to or as replacement for their pre-existing antirheumatic therapy in Study A. Moderately to severely active RA was defined by Disease Activity Score 28 (DAS28)≧3.2 at baseline. Unsatisfactory response or intolerance to at least one prior DMARD was required for enrollment in Study A. A 12-week study period was followed by an optional extension phase, with an efficacy assessment performed at Weeks 2, 6, 12, 20, and every eight weeks thereafter. Patients discontinued the study when they stopped receiving adalimumab or when they received HUMIRA®.

### Key Efficacy Parameters:

ACR20/50/70 responses
Moderate/Good EULAR responses
Change in DAS28, Tender Joint Count, Swollen Joint Count, C-Reactive

Protein levels, and the Health Assessment Questionnaire-Disability Index (HAQ)

### HAQ and AIMS2-SF

The HAQ and the Arthritis Impact Measurement Scale Short Form (AIMS2-SF) are both extensively validated health-related quality-of-life outcome measures in RA (Guillemin et al. Arth Rheum 1997; 40(7): 1267-1274). Both measures have been proven useful in cohort studies and clinical trials (Guillemin et al). The HAQ and the AIMS2-SF are effective in detecting both long-term and short-term health status changes (Guillemin et al). In France, Study A patients completed the French AIMS2-SF, a tool that measures disease impact on physical function and overall quality-of-life in five components. Study A began in France began in December 2002. Completion of the AIMS2-SF at Study Entry Visit 1, Week 12, and Week 28 (if patient participated in the continuation period) was a component added in March 2003 as a France-specific amendment to the study protocol. Because AIMS2-SF was introduced later into the study, the number of French patients answering the AIMS2-SF is smaller than the overall French Study A population

### Results

86 French sites enrolled 1,002 patients. 266 French patients completed the AIMS2-SF. Baseline demographics and clinical characteristics of those who completed the AIMS2-SF were consistent with those of the larger Study A.

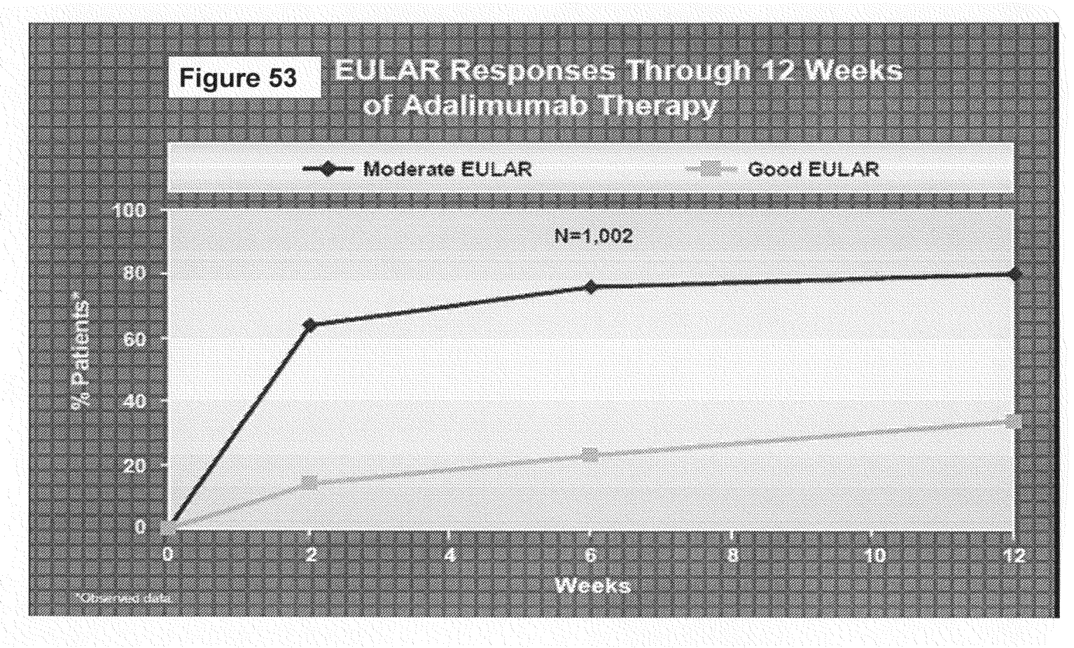

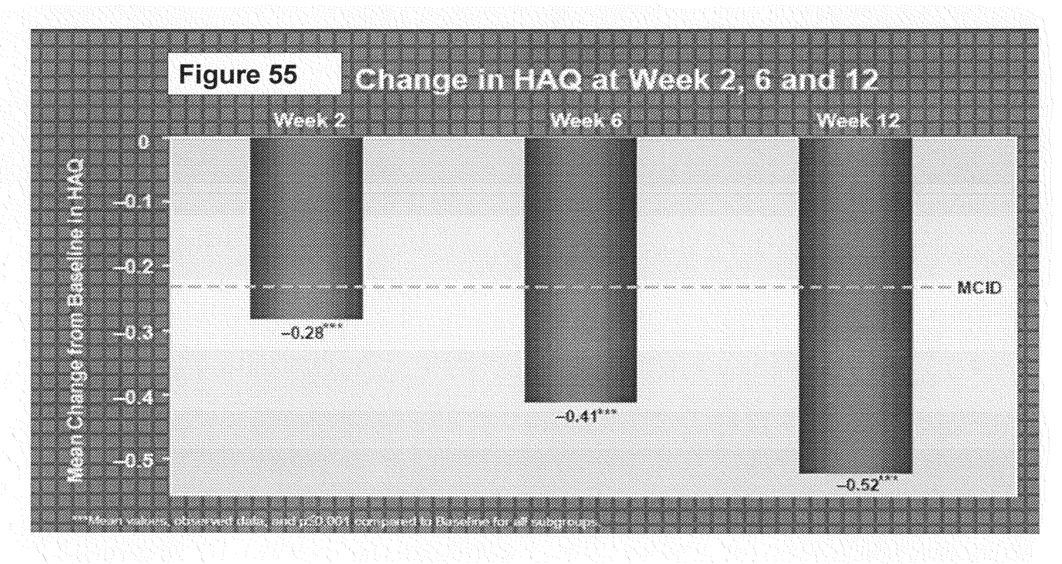

Thirty-six percent of all French patients achieved ACR20 by Week 2, and 66% achieved ACR20 by Week 12. ACR50 and ACR70 responses increased steadily to Week 12 (see FIG. 52). Week 2, 64% of the patients had achieved at least a Moderate EULAR response. By Week 12, 80% had achieved at least a Moderate EULAR response. Good EULAR response increased steadily from Week 2 to Week 12 (see FIG. 53). As shown in FIG. 54, mean DAS28 Score improvement was highly statistically significant. After 12 weeks of adalimumab therapy, functional disability, as measured by mean changes in HAQ scores, were decreased by a highly statistically significant margin, as shown in FIG. 55.

A total of 266 patients out of 1,002 Study A patients in France completed the AIMS2-SF. AIMS2-SF responders recorded a significant improvement in quality-of-life. Highly statistically significant improvements were noted in four of the five AIMS2-SF domains (Physical, Symptom, Affect, Work; all p≦0.05).

TABLE 136
AIMS2-SF Results at Baseline and Week 12

| | Patients | | | | |
|---|---|---|---|---|---|
| | Completing | | Week | Mean | % |
| Component | Component | Baseline | 12 | Change* | Change* |
| Physical | 225 | 3.9 | 2.9 | −1.0^{§} | −25.0^{§} |
| Symptom | 252 | 6.7 | 3.5 | −3.2^{§} | −43.4^{§} |
| Social | 250 | 5.3 | 5.3 | −0.1 | 1.6 |
| Affect | 226 | 4.7 | 3.6 | −1.1^{§} | −11.8^{§} |
| Work | 133† | 4.2 | 3.3 | −1.0^{§} | −16.3^{‡} |

*Mean change of individual patient differences.
†Those patients who were not working did not respond to the Work component of the questionnaire.
‡p ≦ 0.05
§p ≦ 0.001

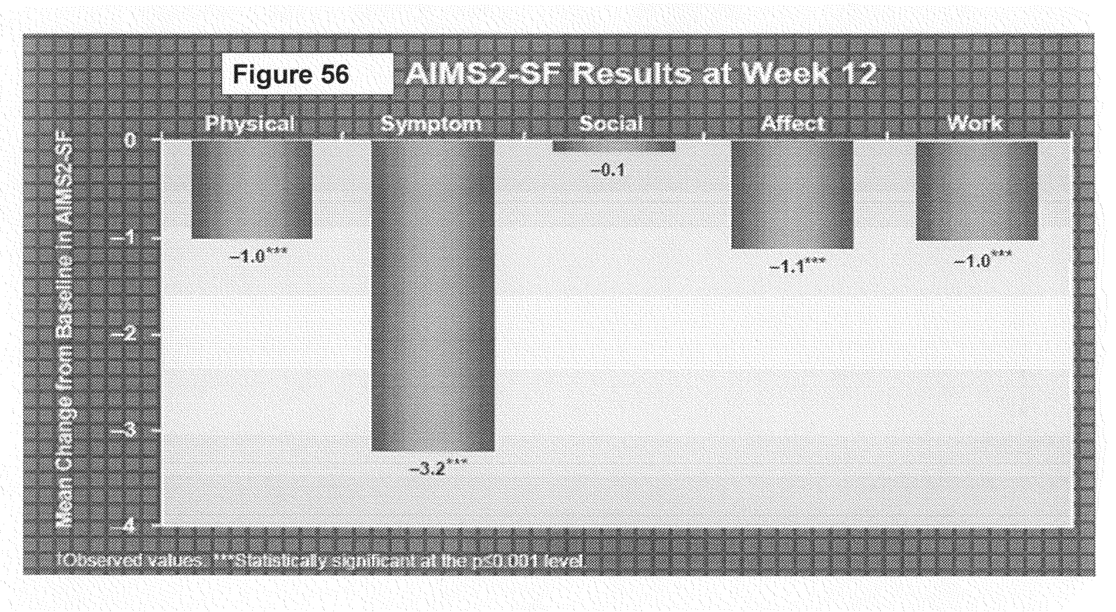

Responders recorded the most dramatic changes in the Symptom category of the AIMS2-SF, as shown in FIG. 56.

In conclusion, in a cohort of patients from France, adalimumab significantly reduced the signs and symptoms of severe and long-standing RA. The results of the AIMS2-SF for a subset of Study A patients demonstrated significant improvement in quality-of-life after 12 weeks of treatment with adalimumab.

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

1. A method of determining the efficacy of a human TNFα antibody, or antigen-binding portion thereof, for treating rheumatoid arthritis (RA) in a subject comprising
determining an ACR response of a patient population having RA and who was administered the human TNFα antibody, or antigen-binding portion thereof,
wherein an ACR response selected from the group consisting of an ACR 20 response in at least about 33% of the patient population, an ACR50 response in at least about 30% of the patient population, an ACR70 response in at least about 19% of the patient population, and an ACR90 response in at least about 8% of the patient population, indicates that the human TNFα antibody, or antigen-binding portion thereof, is an effective human TNFα antibody, or antigen-binding portion thereof, for the treatment of RA in a subject.

2-5. (canceled)

6. The method of claim 1, further comprising administering the effective human TNFα antibody, or antigen-binding portion thereof, to a subject for the treatment of RA.

7. A method of treating RA in a subject comprising administering an effective human TNFα antibody, or antigen-binding portion thereof, wherein the effective human TNFα antibody, or antigen-binding portion thereof, was identified as providing an ACR response selected from the group consisting of an ACR20 response in at least about 33% of a patient population, an ACR50 response in at least about 30% of the patient population, an ACR70 response in at least about 19% of the population, and an ACR90 response in at least about 8% of the patient population who received the effective human TNFα antibody for the treatment of RA.

8-10. (canceled)

11. A method for determining the efficacy of a human TNFα antibody, or antigen-binding portion thereof, for treating RA in a subject comprising determining a EULAR response of a patient population having RA and who was administered the human TNFα antibody, or antigen-binding portion thereof,
wherein a moderate EULAR response in at least about 65% of the patient population or a good EULAR response in at least about 11% of the patient population indicates that the human TNFα antibody, or antigen-binding portion thereof, is an effective human TNFα antibody, or antigen-binding portion thereof, for the treatment of RA.

12. (canceled)

13. The method of claim 11, wherein the effective human TNFα antibody, or antigen-binding portion thereof, is administered to a subject for the treatment of RA.

14. The method of claim 1, wherein the patient population had previously failed a different TNFα inhibitor.

15-19. (canceled)

20. A method for treating a human subject having long-standing rheumatoid arthritis (RA) who has failed Disease-Modifying Anti-Rheumatic Drug (DMARD) therapy comprising administering to the subject a human TNFα antibody, or antigen-binding portion thereof, such that RA is treated.

21. The method of claim 20, wherein the human subject has severe RA.

22. The method of claim 21, wherein the human subject has had RA for at least about 11 years.

23. (canceled)

24. A method of treating a subject having RA who has failed a prior biologic comprising administering a human TNFα antibody, or antigen-binding portion thereof, to the subject such that RA is treated.

25. The method of claim 24, wherein the prior biologic is selected from the group consisting of etanercept, infliximab, and anakinra.

26. A method of treating a subject having recent-onset RA comprising administering a human TNFα antibody, or antigen-binding portion thereof, to the subject such that recent-onset RA is treated.

27. The method of claim 26, further comprising inhibiting radiographic progression in the subject.

28. A method of achieving a major clinical response in a subject having RA comprising administering a human TNFα antibody, or antigen-binding portion thereof, to the subject such that the major clinical response is achieved.

29. A method for inhibiting radiographic progression of rheumatoid arthritis (RA) in a subject having early or recent-onset RA comprising administering a human TNFα antibody, or antigen-binding portion thereof, to a subject having early or recent-onset RA, such that radiographic progression is inhibited.

30. A method for testing the efficacy of a combination of a TNFα antibody, or antigen-binding portion thereof, and a disease-modifying anti-rheumatic drug (DMARD) for inhibiting radiographic progression of rheumatoid arthritis (RA) in a subject having early or recent-onset RA comprising determining a radiographic progression score of a population who was administered the combination of the TNFα antibody, or antigen-binding portion thereof, and the DMARD,
wherein no radiographic progression in at least about 61% of the patient population indicates that the combination of the TNFα antibody, or antigen-binding portion thereof, and the DMARD is an effective combination for the treatment of early or recent-onset RA in combination.

31. The method of claim 30, wherein no radiographic progression is defined as ΔTSS≦0.5.

32. The method of claim 30, wherein the subject having early or recent-onset RA has had a disease duration of less than 3 years.

33. The method of claim 30, wherein the DMARD is methotrexate.

34. A method for identifying a patient having RA who is a candidate for treatment with a TNFα inhibitor, comprising determining whether the patient has a DAS28 score of at least about 5.1 and a RAPID score of at least about 5.0, wherein said DAS28 score and said RAPID score indicate the patient having RA is a candidate for treatment with a TNFα inhibitor.

35. A method for predicting the efficacy of a TNFα inhibitor for treating a subject having RA comprising comparing the C-reactive protein (CRP) level of the subject prior to treatment with the TNFα inhibitor to the CRP level of the patient following treatment with the TNFα inhibitor, wherein a decrease in the CRP level of at least about 20% indicates the TNFα inhibitor will be efficacious at treating RA.

36. A method for testing the efficacy of a TNFα inhibitor and a disease-modifying anti-rheumatic drug (DMARD) for inhibiting radiographic progression of rheumatoid arthritis (RA) in a subject having long-standing RA comprising determining a using a radiographic progression score of a patient population having early or recent-onset RA following administration of the TNFα inhibitor and the DMARD,
wherein no radiographic progression in at least about 62% of the patient population indicates that the TNFα inhibitor is an effective TNFα inhibitor for the treatment of early or long-standing RA in combination with a DMARD.

37. The method of claim 36, wherein the radiographic progression is determined using either a mean Total Sharp Score or a mean joint erosion score.

38. A method of inhibiting reactivation of latent tuberculosis in a patient receiving a human TNFα antibody, or antigen-binding portion thereof, comprising delivering isoniazid (INH) prophylaxis to the subject, such that reactivation of latent tuberculosis is inhibited.

39. A method for predicting whether a subject having recent-onset RA will be responsive to treatment with a TNFα inhibitor for inhibition of radiographic progression associated with RA, using the mean baseline CRP level of the subject wherein an abnormal CRP level at baseline indicates that the subject will not be responsive to treatment with the TNFα inhibitor.

40. A method for predicting whether a subject having recent-onset RA will be responsive to treatment with a TNFα inhibitor for inhibition of radiographic progression associated with RA, using the mean baseline CRP level of the subject wherein a normal CRP level at baseline indicates that the subject will be responsive to treatment with the TNFα inhibitor.

41. The method of any one of claims 34, 36, 39 and 40, wherein the TNFα inhibitor is selected from the group consisting of a TNFα antibody, or an antigen-binding portion thereof, a TNFα fusion protein, or a recombinant TNFα binding protein.

42. The method of claim 41, wherein the TNFα fusion protein is etanercept.

43. The method of claim 41, wherein the TNFα antibody, or antigen-binding portion thereof, is selected from the group consisting of a chimeric antibody, a humanized antibody, a human antibody, and a multivalent antibody.

44. The method of claim 41, wherein the TNFα antibody, or antigen-binding portion thereof, is selected from the group consisting of adalimumab, infliximab or golimumab.

45. The method of any one of claims 1, 7, 20, 24, 26, 28, 29, 30 and 38 and 43, wherein the human antibody, or antigen-binding portion thereof, dissociates from human TNFα with a Kd of 1×10−8 M or less and a Koff rate constant of 1×10−3 s−1 or less, both determined by surface plasmon resonance, and neutralizes human TNFα cytotoxicity in a standard in vitro L929 assay with an IC50 of 1×10−7 M or less.

46. The method of any one of claims 1, 7, 20, 24, 26, 28, 29, 30 and 38 and 43, wherein the human antibody, or antigen-binding portion thereof, has the following characteristics:
a) dissociates from human TNFα with a Koff rate constant of 1×10−3 s−1 or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

47. The method of claim 43, wherein the human antibody, or antigen-binding portion thereof, has a light chain variable region (LCVR) comprising the amino acid sequence of SEQ ID NO: 1 and a heavy chain variable region (HCVR) comprising the amino acid sequence of SEQ ID NO: 2.

48. (canceled)

49. An article of manufacture comprising
a) a packaging material;
b) a human TNFα antibody, or antigen-binding portion thereof; and
c) a label or package insert contained within the packaging material, wherein the label or package insert provides information selected from the group consisting of
a) that the TNFα antibody is safe for the treatment of both early and long-standing rheumatoid arthritis (RA);
b) that in studies of the TNFα antibody for the treatment of rheumatoid arthritis (RA) serious adverse events (SAEs) included a disorder selected from the group consisting of tuberculosis, lymphomas, congestive heart failure, demyelinating disease, systemic lupus erythematosus, opportunistic infections, and blood dyscasias;
c) that the TNFα antibody, or antigen-binding portion thereof, can be used for the treatment of rheumatoid arthritis in patients who have failed methotrexate therapy;
d) that the TNFα antibody may be administered in combination with methotrexate, wherein the methotrexate is administered via a route selected from the group consisting of oral, intramuscular (im), subcutaneous (sc), and intravenous (iv);
e) an indication that patients with rheumatoid arthritis (RA) who previously failed therapy with etanercept or infliximab may benefit from treatment of RA with the human TNFα antibody; and
f) an indication that patients with rheumatoid arthritis (RA) taking the human TNFα antibody and concomitant corticosteroids have a higher risk of developing a serious infection.

50. (canceled)

51-55. (canceled)

56. The article of claim 49, wherein the human TNFα antibody, or antigen-binding portion thereof, dissociates from human TNFα with a Kd of 1×10−8 M or less and a Koff rate constant of 1×10−3 s−1 or less, both determined by surface plasmon resonance, and neutralizes human TNFα cytotoxicity in a standard in vitro L929 assay with an IC50 of 1×10−7 M or less.

57. The article of claim 49, wherein the human TNFα antibody, or antigen-binding portion thereof, has the following characteristics:
a) dissociates from human TNFα with a Koff rate constant of 1×10−3 s−1 or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

58. The article of claim 49, wherein the human TNFα antibody, or antigen-binding portion thereof, comprises a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprises a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11.

59. The article of claim 49, wherein the human TNFα antibody, or antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2.

60. The article of claim 49, wherein the human TNFα antibody, or antigen-binding portion thereof, is adalimumab.
